SEQUENCE LISTING (88 sequences)

SEQ ID NO: 1 - protein, 140 aa, Mus musculus
MVLLRSLFILQVLVRMGLTYNFSNCNFTSITKIYCNIIFHDLTGDLKGAKFEQIEDCESKPACLLKIEYYTLNPIPGCPSLPDKTFARRTREALNDHCPGYPETERNDGTQEMAQEVQNICLNQTSQILRLWYSFMQSPE

SEQ ID NO: 2 - protein, 159 aa, Homo sapiens
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 3 - DNA, 4723 bp, synthetic construct
  source 1..4723: mol_type = other DNA
ttacttccagaacacaaagagaaccattcttccaggaaaggtctactgttaaacataaaactctaccaaactaaggttgttgtctgtcgtcaacatttcttactacaaagaagggactctccacccatttcttcaataaaaatctgtctgctatcactcccattttgaagtttgtgatttctataatttaatgtggcttaacaataaagcagagtttccatagaaatttggctgtctcaaacttaaactgagttttgtttgaagaacggactgactgctttaagtacactggacaagctaagtatggtgatttgtcccaggaagctacccaattgaggataacctggggaagaaccacttgaaaaagaaaagaaaaaaagaggtttgaaaagagggaagagtgccgggcagtggtggcgcacacctttaatcccagcacctgggaggcagaggcaggcggatttctgagttcaaggccagcctagtctacagagtgagttccaggacagccagggctacacagagaaaccctgtcttgaaaaaaaaaaaaaccaacccccccccccaaaaaaaagcaagaaaagagagaagagaaagatgagagaaaagaaaaatgaagttcaggaccaattaaagaagcaaggtaaagaagtaaatttaggaagagggagaaaatcagacatgtagttcattccggtcatcccagtactttcaaggatgtgacaggagaatccccacaaccaggaggccagcctgggataaagagtgagacactatgtcaaatatatctttctagaaggagaaagaggagggagaagtaaacaaagcacaaagaatgagaactatcattaatataagaaataaaaattaagaaagcaagtgaatgtttttctagtgaaagtgggaaaaaggatggttacagcatgggtcatcttctggtctccctgggtaagaaaattaccaaactccctgagtagtcacacagctccaatgacatcacttctatttcctaccaaagagaaggtgtcccagtcttaatccaacctaggatttcccaaactgcacatgtagatactgttcattccttcagcattaagtatttggattaagataaaaccaggaagctcttcagcccacaggaatttccaaaaatataccttggcccagtggttgctccaggtaagcctaagtagattccaagaaggtggcagcagtgagctaccaaaagaaatctccgtagcaagcttgtttcagtgggagacatccctgccgtggctttccggatatcagtagatctgaggaaactcagtttccccttctcgtgtgaataagctgcagaccttgctgtcgtctgcactgcctttcagtggtttgaaacctgaattactccgttgtctcagttgtctttttccccagttctaataatgatttctctatgtcctccctgtacctgctcacacttccttgtcccttgattccgttcttatcttcagtaggttttgtttgcctgattgcttccttgttttgtattttttttgggggggggcacttcgacgttattatattcacataaatgcttcagagcagagttaatgattactggacaaatcagttattacagaacatgccgggggggggattgaagaggggggggggggagagaaggagggatggatggagagagagagagagagagagagagagagagaaatatgatgtaattaaacatcattaatgaaaaccccactgtacaaataggacgagcactgcgcaactcaaatcaacacctaagaaagtgagagtgtggaaggagtcaaaggaaaatatgaatagctacacaggctgatcccttgagggtatgtgacatctctcctgcagttccccaaccctggaatatgcatgacactccactgcagctctcttagagactctcccttctcctcccttcacatttagaatcccaccctggatttagtgtaaccagtgacttaagaaggtaccgcatatgggagacaaagatacaaaaatcctgaaagggttctggattattgggctcaggactcaattcatccgtgttatcacaattaaaagtagtctttccttaaaaaaagccttggtttctgcatctctgtgatcaaaatcccataacaaggtttggaagaggcaagtttgggaaaatttcagagtgtattaacttagaatactgttggagggaagcctgggtaaataaaggagataaggttagaaagaagacttgaagtcaacatgggagtgatgagtgagaatcttaaagtaactgagtctaccaaaagtcaatataattgaaatgacttaagatgtcacatcattaccagtaaggtagctggatgctatggtgtaggtgatgtgcttagcaaagagatgccttctaaaaatccctgaagggggccccatgcctgcctcagatttacctacacatacataaactatagacacactttaaaggagaaaccaaaaatggcaggtaggctgggtgcaccccaatgggtgccaagccaaaacttatgggggtcaggggacaggttgtctgttgctgtctgacattcttgcccccatcagcaattattcctgggcactgcaacacatgaatctacccaaaagattcgggcggagaggcaatatacatgaagtgactttaaagaccacgtgtttaccaataaagaagtgggttccctacaggggaaggcaagtgaatgaagatggcaaaatcagctgccatttcctttcttttgtctcttggaactatcccaattcagtgaccacatctggatctctacattgcttctgcctatgcaatatctagctgctgatcagaatcatatctgatgtcacgccagatgaatcaggctttggcatcttcccttatcactgtaagaagtagagatgggaagacgccatgatccagacatggtatcataacctaaatttaaatcttgcaggactccagaaaagtccgtctctaaagtcatcagcaaagcagaaactttctgagcctcctgccaccgctacaatcttttattcctcatcctaatgccagagaactgggtccagctgtgctgctccagctgttgaaggccttctgggaaaacttcacctctgactccagtctgtgctttcccccgaatagaatcatttaccaatccctgtgctcgctccttccctggctcagcgtggtctgtgacattttcagggactcacgtggagcacccaacatcatcgttctgagcagtgactcctaggaacttcccgaagacgagactgatgcaggctctgacacgcaaaagtggggagagtgaactgggtctcaggagggcctggggcagctggctgagctccaggagagtaggggttgggttcgtgtcaacagctgggcctttctttcctgctcccagtactgtactggcgctgctccaatcagaaggctgcgagacatcctctcaggctatccctgactcacttggctacttttatcttgtacttcctttcaaaccccaaccaggggagcgcaaatcttaacccaacccaccatccagcttctttctccatccctgacaatcgtgctgctgggacgcatgcctggggccatccaacgatttactggctgagagtctgagctgacacagctcaacaggtcagaagctgttcctcccctaggaggagagcatggtggacaggtctctctctagtggcttagacctgcaacagcaccatagcaccatacaccttaggagcccccactactcctggtaaggcatctttactccactgagacctaaataatgagtttcgagggcggctggatgcttgacttcatcattttaaaaatcttagtcactctgtagaccaggctggccttgaactcagaaatccatctgcctctgagtcccaagagctgggattaaaggcgtgcgccaccaccgcccagctaccagttttctttaatcaagcttaggcactcaccctgattctgagtttttgaagatgagactaactggtccttttctcatatatttcaatttctcattgttcctgtttccagtattctgacaacaactgcccggttccagtgaaatgccttcaacaaaagttacgttatcccaaggctgcattcattctccaaaatctgtcatacaggaacactgcgtttctcggtagccacgaagaggaacactgccagttcaaactggacaaaggagatagatggtcagggtgtgcatggtggaacagcatcagtagcaaacccctaaagtgactgcgggtgttagaaggtgtttttccaagcagaaaaaaaatcagtcatagaaactgcccagtaggaaaaagatgtcaaaatgatgacatggtatcatctctaaaagcatatcgaagcatgtagcaagtgtttagggcagagctaaaaaataaataaataaataaaaataaaataaaataaaaggaaaggaaaaaggtgagggaaattcctgatgattttgctaaagttaaaattccatagatttggctggctttatttcttttttttttttttttttttttttacatcatcaatttagaattctataaagaaagaatgacatcaaggaaaatcattggcctaggggaagagagcccgtaggcgtttaggtgttataaatatggaggcagagaacactggaggatcaggaagactccacgttcaggcgacagc

SEQ ID NO: 4 - DNA, 4041 bp, synthetic construct
  source 1..4041: mol_type = other DNA
cctccctgcaggagaaaaggtcttcataagtagttgcctcttggacaaatgactggagtgcatttttttcaaatatttgcaccagtcactccctcccactgtgaatctttcttcacctcagaatagataacacaggtgaaaatgaacagtgggtgttaaattcattcctgcacacctctggtaaaacaccctacctcttgccctcagaatcttctgagcattgctagcaaaggcaaccttggctgcagagctcaggccaagtaagagtagatgtaaacagctaacctgctcctccaccctacacacactctaagaagagatgttcacttgaatactgttttgaaggttagaactaacccattaatgaaaagaaaagctgagtgtccccaaacctgtcttacttgttgggagcgaccctgtttgaatgttaactgccttgtcagccataagtgcttacttacaaagtcttgaccttagtggaaaagtactagcttagttgagatttctgtgggaaaagttgaagcctttgtaggaaagtactacccccagttaagaacaaatagttgtgctcactttggcagcacatatactaaaattggaacgatacagagaagattagcatggcccctgcgcaaggatgacacgcaaattcgtgaagtgttccatattttttgaagctgggacgaaaggacggaccatctagtgattgccatatccagggatccatcccataatcagcttccaaacgcttgacacactagcaagattttgctgaaaggacccagatatagctgtctcctgtgagactatgccggggcctagcaaacacataagtggatgctcacagtcagctattggatggatcacagggcccccaatggaggagctagagaaagtacccaaggaactaaagggaactgcaaccctataggtggaacaacaatatgaactaaccagtacccgggagctcttgtctctagctgcatatgtatcaaaagttggcctagtcggccatcactggaaagagaggcccattggacttgcaaactttatatgccccagtacaggggaacaccagagccaaaaagggggagtgggtgggtaggggagtcgggggatgggtatgggggacttttgggatagtattggaaatgtaaacgaggaaaatacctaataaaattttttttttaaaaagtaaaaaaaaaaaaaaaaaaaagaacaaatagctatagatcttgtggacaggtacctagcaacccattctgttctgttcctcctgctgaactttttacctagccagtatcctgcttttggaacaggtgcattcccccagaaacaaagcgattctgcatcgtccccctccacatatcctgcttctgtgggtataaaacctgcctgggaaaaataaaatttgttagcttgatcagaatctttgatttgctgttcgttttttgtgtttcttgccccgccccccttctctctgcaggtggttcctcagaccctgttcaactgtcccgcatcagggcattacttgtcaacaaagagctacttatgagcaccaagtaaatagttacaaagtgcccactgtgggccaactttcctgaggtgaagtctgtgttaaacccatagttacaaaagtaagtaagacagagctcatatccagagaagctcagagtggaactggataatcagttgtctgtagtccttaacaaattggccagtgagtgttcctttgatttgagtaaaatcaagacaggcacactttcaaaaatcttcctctaaattccttacccagagcttttaagcaccaccctaagaaaactccactgggtctagaaaaggcagcaatcatcaattctttgaatagaagtgtggaggcctgatattttaaatgtattaactctgccttactacaaattcattctcccttttactaaatcatgataaaaggtattatagcatttttcttaatccctttagacccaattgccctaaaagtgacttctacccatttggtagagttcataggacagagtaccaaaggaaaggagtgctctgaggaggagaccattagaagataactcctgttattgaggacagcaataccaagcacatgccttaagaaaactgcactggagagatggggaaacatctggacaacaagagggactagtgtccattgctcactgcaagccagggaaatgagctgtgctcaccaggcagaatggaaaattctgtaacccatcaggctataagtagggtactgtgctgactagtttaatggcaacttgacacaaactagaaacatcagagaggaggaaacctcagctgagaaaatgcctttataatattcaggcatatggcattttcttaattagtgatcaatatgggaagggtcaacccattatgggtggggtcatccctgggctggttctaaaaaagcaggctgagaaagccatgggaagcaagcagcttcccatcatggcctcatattagctcctgccttcaggttcctgccctgcttgagtttctgtccttactttctttgatgatgaagagtgatgtgaaaatgtaggccaaataaaccctttcatcctcaacttgcttttttgtcatggtatttcatcccaaatatagaaaccccaagacatgtgcttaaaaacatcttacctgtgcatggaagtatcgttagaccaaggctaatggctgcaacgatctaacttaatgaatttaaaaaaaaataatacttaaaagaatcagttcctaagtaacttagctgtatttcacaacaaaccacaagggtgtttatgaagtaaagaatgtctcacacacatgcgaatgtatccactcaaatatatatatatataattaaaataaatctttaaagaatgaaagaaaaaagaaaacaaaaggggagagagggggaaggagtaagagaggatcttgaggacagaagagctgtaagaactattgtgtcctgttagggaaggtggcacaccttttatctagagtcagaaagcaggcaaatctttatgaagacgaacttcatctatacagtttcaggccagccaagctatacagtgagagcatgtctcaaaaataaggaggaaaatatggtgctgtagtataaaaagtaccactaactcaaaactaacatagaaggtagaattaataagtgaaacattaaattaattattataatgttgagaacaagcaaaagaaacttatcctaagttaaccattcccttttcagactccttttaattgtagtgagaaactaaaatcaaaatcccaggccctaggggagcttggaaattcctaacagctgaacagtttctattttaaggaaacagttgtccaagtccagatagctcagggacaacttctccatcttgctagtaagatcaaactaagttcaggtttccaggcccagaaacctacttctattcttattgatagaaactcccttgttcaacttcttatgtcaacatatgattggacaatgttatagtctaccctgctccccctcacttcacagttttgattccattctttaaataggctgtacagtgtcccttcagagttgcagctcagcacccaagtctgttctttggccctaactagtagacacttaattacaagaaaattttgccatctgcatggtgtttgaattatgttgtatttaagcagaccccacaacaataactcaagatatttaggaaacatagaagatacaagcacagattctagatatgaaaattatgtgtaaaataaatacacagtgaatagttttaattgggggttgggcattaaaatatttgaactagaccaatacccacccaaatgctacagcctggatgctccccaggagatccaaattgaccaaggacacaagtgacaatttcacccaaatacccatgccagctggaacacccacacagcccagctgacatgggacctacacccccaactctccaccctctatcccacccctctgagatccaccttccttctgatccaattcctcatccagaccaggtc

SEQ ID NO: 5 - DNA, 4184 bp, synthetic construct
  source 1..4184: mol_type = other DNA
atgttcccttttgccttactatatgttctgtcagtttctttcaggaaaatcttcatcttacaacttgtagggctggtgttaacttacgacttcactaactgtgactttgagaagattaaagcagcctatctcagtactatttctaaagacctgattacatatatgagtggggtaagtgaagaagcttttttaaaacaaatgtattttcatcagaggagtcggcatacacacactctacaatttaactttgtaggaaagaaaaataatttagaaaaaatcatggccccacattttgtcaaggattcttacaagtgatattcaaatatctaatctaaaatgattatctagaaattggcacattctaagtgtgcagatgctgatgaggagcaggtattgatagacagcgcgttatgcgtcaaaggatgtctatcctttgctaaagtgttactctgactatgctgtaaaaagcaggaggtaagagcttaagaaagaggagtaaaagagataattctcatgagataaactctaaggattgatgctgtgctccaggtctctccagtgttttagatgtttcaggatgctatttattacagaatatggtgtacttggaattttttttaacatacagtagtaatcattttcctgattaacctaatttctagacagagtttgcattcatgaatggccacagtacagatgcggacatccaaaggatggcattattactcacaagcatagtgctatgtgcagttatggcttgagggaagggaggggggaggtcgccctctgagacctgaaccttttggtgtggtttcaagcactaaccagcactatctaatggctatttcactgccttgtcaatgacataggaaaaaggtacctgagtggaaactgttttcagggcacctttaaagcctgggagcaaagggtggagggatgattttccttgtggacttaaaagtctttaccctctttgtcctatttttctttcttccagaccaaaagtaccgagttcaacaacaccgtctcttgtagcaatcgggtgagtagagagttcagtgctgctggctttctccagggagacgccaggcattttggagagggagtatcctgctacgtgcagaactccgagaggtgcctgggctccgggacgccgccgccgggggaaaggggacatctgggctgtcagagcggggctgcgcctagcttgggacaacacttctgttccaatttagggagaggaagtctctatccggaggaaaggcaaattgggaactgggacgagggaacgttgttaggggcaccacctgctggggtccggcgcctccgcgctcgggctcggaattttggcagcctccgccccctggagacttgggaggagcgagcgtgggtgacagtcttttcgcgacgagtgccctccgccaccctcgccacgcccctgctcccccgcggttggttcttccttgctctactcaaccctgacctcttctctctgactctcgacttgtgttccccgctcctccctgaccttcctcccctcccctttcactcaattctcaccaactctttctctctctggtgttttctccttttctcgtaaactttgccgcctatgagcagccacattgccttactgaaatccagagcctaaccttcaatcccaccgccggctgcgcgtcgctcgccaaagaaatgttcgccatgaaaactaaggctgccttagctatctggtgcccaggctattcggaaactcaggtaagcccgaagcctcagacgtttgctgtaccttggggctaacctcaaattaaactggggctttggtgcagaagtcgttctcttatttttatttaggttttatctttcgaagagcaaacgagccgggtaaaagtggtaggatgtcagttagacccacgttgatacccggaatcaaactcacctatttctacggttctgatactgttttggctgaattatggttctaaaccttagggcaatgtttcaagctatgatgagtgagacttctatatcagaatgttttgattgctggagcataagagtatggctgctaaaaatgccaattcccaggtactcaacccagaccttcaacattaaaatctcagattatggggctccttaagagattcttgtccagtccaaagtttgagcaacacctcttgttcttatcacttaattattgtgtgcttatttgctaaatgtataattacattatacataaaatctctatcctatgtttgcttaattgcttgtgtgggcgctattgctgtctctttacacatttttgcacatgtagttatctgcatttgaatgctcgtgtagcattaaatatggagatagtgtagtggaaagttaggcacaggaactctggagacaacctgcctgactttgaatcctggccctataacttctgtgaagacttagttaaattacttagcctccgtgtactgtagcttcatgggtaaagtaagtatcatatcagttagtcttatacaggttgtttctgaggattaaattagtcaacacatgtaaatgcagttggaacagtgcctggtacacaacaggcactcaatatttatttcagtcagcaagtagaggatttatcttcatggtgacaagtttaaggaacagagagagacaagtgcagatatgtttgattgctccttattagcctagtggactttatatgtctacagtctaggtagatggacacgactgtcactttttttttttttttttttttgagacagaatctcgctctgttacccaggctggaatgcagtggcacgatctcagctcactgcaacctccatctcctgggttcaagcctcagcctcccgagtagctggaactacaggtgcccgccaccacgcctggctaacttttgtatttttagtagagacggggtttcaccatattggccaggctggtctcgaattcctgaccttgtgatctgcctgcctcggcctcccaaagtgctggaattacaagcatgagccaccatgcccagccaaaactgtcactttctagaggttgaggattgaagccatagcgctgatctgggttgagcttgaattagaaactcaataccagacagccatatgggaaacctatttggcttcatgccttcttatgaaggagaccctggcaaatctgcagatggctacaataaaattcatttaaataagagcacaaacaaaaagctagatcaagttcttggacagcatgtgagaaagggagagtttggagaaatttatttcagtccctcccaagcccaaatggagagtctaagactaataataatgattttgcaggtttttttaagatttgtgcttaataaccctgtgactttattaatttgcataccatgtgtctaggaggcccagtgtactactcaaaggtaattcagataaaggtatatactgcaatcctctttaaaataagccctcagatgtctgtgacacatctagacaatggggcaggggagggggaaggatggggagcaggagcatgcattttgggtccaaaaaatagactaggtttattgaatgatgtctataaacaggtataagatagctcttgcccatgaggaacttgtgatcttgtcagggaggtcttgaaatcagcaatttattcatttacttaatcactcaacaaatattcagtgtttcctatgattaagacactgtattcagtgctatggggaatacctatgatgcaatataaagaaaagcatgttaagtgagagccaagttaaatgacacacactcttaagtactggaagagtttccaaaagcaaggtctgagcaattagtggaggctttttgaaggaggtggtgcttggccttgaagcaaaagtaggtgggtacagaaacaggaaggcattcccctggaaaaggcacatgctagcacatagtaagcaggtgctttggagacacactgaaagatggatttgcatagagaaggcaattaaacctgctctcaacagttactaaagatagtgaaaagtaattttgactattgattcttatattctgcagataaatgctactcaggcaatgaagaagaggagaaaaaggaaagtcacaaccaataaatgtctggaacaagtgtcacaattacaaggattgtggcgtcgcttcaatcgacctttactgaaacaacagtaa

SEQ ID NO: 6 - RNA, 1200 nt, synthetic construct
  source 1..1200: mol_type = other RNA
cacgttcaggcgacagcatgttcccttttgccttactatatgttctgtcagtttctttcaggaaaatcttcatcttacaacttgtagggctggtgttaacttacgacttcactaactgtgactttgagaagattaaagcagcctatctcagtactatttctaaagacctgattacatatatgagtgggaccaaaagtaccgagttcaacaacaccgtctcttgtagcaatcggccacattgccttactgaaatccagagcctaaccttcaatcccaccgccggctgcgcgtcgctcgccaaagaaatgttcgccatgaaaactaaggctgccttagctatctggtgcccaggctattcggaaactcagataaatgctactcaggcaatgaagaagaggagaaaaaggaaagtcacaaccaataaatgtctggaacaagtgtcacaattacaaggattgtggcgtcgcttcaatcgacctttactgaaacaacagtaaaattagctttcagcttctgctatgaaaatctctatcttggttttagtggacagaatactaagggtgtgacacttagaggaccactggtgtttattctttaattacagaagggattcttaacttattttttggcatatcgcttttttcagtataggtgctttaaatgggaaatgagcaatagaccgttaatggaaatatctgtactgttaatgaccagcttctgagaagtctttctcacctcccctgcacacaccttactctagggcaaacctaactgtagtaggaagagaattgaaagtagaaaaaaaaaattaaaaccaatgacagcatctaaaccctgtttaaaaggcaaggatttttctacctgtaatgattcttctaacattcctatgctaagattttaccaaagaagaaaatgacagttcgggcagtcactgccatgatgaggtggtctgaaagaagattgtggaatctgggagaaactgctgagatcatattgcaaatccagctgtcaaagggttcagacccaggacagtacaattcgtgagcagatctcaagagccttgcacatctacgagatatatatttaaagttgtagataatgaatttctaatttattttgtgagcacttttggaaatatacatgctactttgtaatgaatacatttctgaataaagtaattctcaagtttgaaaaaaaaaa

SEQ ID NO: 7 - protein, 359 aa, Mus musculus
MAWALAVILLPRLLAAAAAAAAVTSRGDVTVVCHDLETVEVTWGSGPDHHGANLSLEFRYGTGALQPCPRYFLSGAGVTSGCILPAARAGLLELALRDGGGAMVFKARQRASAWLKPRPPWNVTLLWTPDGDVTVSWPAHSYLGLDYEVQHRESNDDEDAWQTTSGPCCDLTVGGLDPVRCYDFRVRASPRAAHYGLEAQPSEWTAVTRLSGAASAASCTASPAPSPALAPPLLPLGCGLAALLTLSLLLAALRLRRVKDALLPCVPDPSGSFPGLFEKHHGNFQAWIADAQATAPPARTEEEDDLIHTKAKRVEPEDGTSLCTVPRPPSFEPRGPGGGAMVSVGGATFMVGDSGYMTL

SEQ ID NO: 8 - protein, 371 aa, Homo sapiens
MGRLVLLWGAAVFLLGGWMALGQGGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKFILISSLAILLMVSLLLLSLWKLWRVKKFLIPSVPDPKSIFPGLFEIHQGNFQEWITDTQNVAHLHKMAGAEQESGPEEPLVVQLAKTEAESPRMLDPQTEEKEASGGSLQLPHQPLQGGDVVTIGGFTFVMNDRSYVAL

SEQ ID NO: 9 - DNA, 3887 bp, synthetic construct
  source 1..3887: mol_type = other DNA
gagatcctggtgtgaccaagctcctgttatcctgggatcctggaatcctaagatcctggtcgcgttacagtgcctggaagtggtgtctcctctggggaccatagggctgtctggtgtgtttgaaaccaaggtataccagcacctatcagaaggaacccgaccgctggtcagtcagggctcctgtgtccttgctcctgctgtcacaggcccatcaagattgttttggaacatgctacatccactccagtgaagtctgcatgacaggcctaaaagcctggaagcagtcatgaggcaaagagttttacggaaacttagcctcctggaaccttggcattcccatagcacatacactcaaaacctgtccccgagttagtctggtgtatggatccctgtgctttctttctgtattgttttaagtgcctttaaggattgattttgacatatagataagcctctaccagtgttccaatgtcctagaaagtccttgaagcctaccctgagcttgcaattcagtaagaatgtttcctattcagtaacattcaaatttacctctagtagagacagtgaaaataatcaggcattactatttactagaacagagctagaaaagctcaaggctagtctgcatagtaattacttaaaacaatagccaagtcacatccataaacaggaatacacaatggcctaggaaataggtgggttgcggtattattcatgaaagggttagtaaaatggaattcccctggtgcaagttgttcactaggcccagaggaataggataatcaggtatttactaaaggagccctggtggtgggtttaacaatagactagcattgcatcagagcattcacctggctcctgtgtttagctgatcttaattagggttgttcctattctcagttgtaaacactgcctggctcccgccatctttttatgtatgcattttctttgttttgtgttaagaaacagtgtaccttgatgaatgtatccatcagctttcttgtttttcttctgtattataagactggtgctgagcgtggtggcacacgcctctaatcccagcactggggaggcagaggcaggtggatttctgagtttgaggccagcttggtctacaaagtgagttccaggacagccagggctatacaaagaaaccctgtctcgaaaaaaaatcaaaaaacaaaaaccaaccaaacaaaaagactggtgcttgctttgagaaaataatccagatacacactcccttgtgtccatatttatttgttatccatgccaactccttgcttgcctgtaagtactggaacctcgttccctgcagatcgatgacccaattgaggtttgtctgcagcaggaacagatgttttgttccactcaccagtgatcctaagattgtgtggagagtcctctggggaccatgggaccgtctgcagagtttgtgcacaaggtgacccggagctggtgccgactggaagggctgcagtgccctttcttttttctttctttctttcttttctttctttttttttttttttttttggttttttgagacagggtttctctgtgtagccctggctgtcctggaactcactttgtagaccaggctggcctcaaaatcacaagaacaacgcacctagaggtgtcactgttcacagaaagtagggtaaccttatattaatcattaattaagtacatggcttactcacaaaaccacaggtcaatatgatggtagccatttctcaattgagatccccggttctcaacatcttcaggtttgtgtccttatgaatgaaaccattttccaccaaactttagtaattggtgtaacaatgcacagtgctgccctgtttgtaagttcaaatttgtatattacttaaaatctttgtatgattaagtacgacttttgttaagacaaagaaaaagatatgttcactattatctaatatttacataccatttaagtttcaacaaagcgatttacttttgtgctgggccacctagagactgggaaagggagaagcagaagtgacctggaacagaaagtcacacgatggccagccacctccatcttggtgaggtcaatgtgcaaccaaatgacagaacaaagaagtagagaggaacagacatatgggagcaacacctcccttctgtttttggataaaagacacacaatttttattatttacaagtaagccttaaaaagcaccacaagagccgggcagattagatgggcgggggcggggccttggaggtttgggggcggggacaggccgcgaggggcgtggtcggggcgcacgcggagtccacccccccccccccgccccccccccgcttccccctatccgggtgattgatgggaagaggaagccgggccacgcccatgtcggtgagtcgccgcatccgtttattgccgtggccccgccccgtcgccccaacccccgcccccggcttcccgttttcggctctaagcggcctgggcgccctcgactcggaccggctcggaccgaaccagctgtcaatcactgcagcgtccgcggccccgccggcgacatggcatgggcactcgcggtcatcctcctgcctcggctccttgcggcggcagcggcggcggcggcggtgacgtcacggggtgaggagtgagcgggggcggggctgcctgtcaatcgccgcggtgggcggggcccgagcaagagctaccaagttgcttttcgtcccatcattgcttttcgtcccatcatgaatatgcaaataaggcctctggccctcctaagggcgatcggatagcgcttcgtttgcatattcattttgatctttgcgtatgcatgagccccgccctccccccctacgctcggcgctttctcctcagtaatatgcaaatgagacctaaaccccgccttgacctcattagcatagtgctgccgccacaatctcgctcctcctcctgaatatgcaaataaggcctctgggccgctccttctttgcatattcatatacagctttcccgcttatatgcaaataacgcttcgcccctaccgagttctcactcatcgcttctcatttgcatatccatcgggagatacacatattcatgagcgatgtatttctgtcttccatcccctcatgaatatagaattgatgccctgtccatatggatcactatgcatttgcatattttccccacgatttacatatgcacaagcctcaacgtcttgctccaacgtccctgatcatgaatatgcagatgagacctcagatctccgaaattgaatctgcccgctcctcatcatatactgctattctcatatgcaccagccacaaagtcttccatccttttcccctcatgaatatgcaaagaatgcttccccagtccatctccactctggttcactgcctgctcatttgtgtatccattggtcgctttgcatagtggtgagccccgcttccctaccgctttctttgcgatcatgtatattcaaatgaggctccgacttgctagctgctcatttgcatattcgagaagcgctttccatattcatgagcccccaatccttttccttttctctgtttgtgaatatgcaaatgaggtgtcgcccaccgctacagttcttgggcctctcgtttacatattcattccctatatttgcgtacgagccagaatgtcttcctctgtcttcccctgcctagtgcatatgcaaatgaggcccggcctgctcccagcgccctagccaagggtctgagtcagggaaatggctgcgatggctcttctggaacgcgctgacgtcacgggctgctcccctgatccccgccctgcccccgcaggtgatgtcacagtcgtctgccatgacctggagacggtggaggtcacgtggggctcgggccccgaccaccac

SEQ ID NO: 10 - DNA, 3941 bp, synthetic construct
  source 1..3941: mol_type = other DNA
ggcgccaacttgagcctggagttccggtgagtgaaggggggcggggcagaacttctggtctgtgacttcctgtcggaatcattgaatgatatatgggagggggcgggatttcctctcattcctcttcactcacttgctgttcattcttttaaacgcctgaaagagtgggcgggacttcctgtctaattaatgattgacaaatgattagggacggtacttcctttcagactctgatgaatgacaggaaggagcggtacttcctttctgcccatcactttctgtttgaggtgttgaaattcagatgctcgtgggcgggacttctggtccatcacttcccatccagctgatccttcccccttaatccccgactctgcagttatggcactggcgccctgcaaccctgcccgcgatatttcctgtccggcgctggtgtcacttccgggtgcatcctccccgcggcgagggcggggctgctggagctggcactgcgcgacggaggcggggccatggtgtttaaggctaggcagcgcgcgtccgcctggcgtgagtggtgtgggcgtgacatggagggagggggcggggccttgggcggaagtacgcaggaagttgcctgatgatgtctcttcaactcccactgagtggggcaaacaggaactgggcggcgcttagacgggaagttggtctttgataccggaattgggctccaggggttaggcagtgctctgatgatgtcacagctgcgcctttggactccggtgggcggagcatagacagaaagtggttcttaggaatgggaagtaggcgtgtcctgctctctgaagtgcgccccttccctcatcccgatgggcatggccttcatgaaacaggcgggggtgggacagaaagtgatgtcaccgccacaacacccagactctcggtgggtgtggattaagccaaatgaggtcacagcagctcctcccacccccaccttccccaccgcagtgaagccccgcccaccttggaatgtgacgctgctctggacaccagacggggacgtgactgtctcctggcctgcccactcctacctgggcctggactacgaggtgcagcaccgggagagcaatgacgatgaggacgcctggcaggcgagtgaattcatggggggcagggccggcccctccgtgacctctgaccccacctgcgacctcctgcccccgcacactgcgttttccaaccctaacaatagatgcatggcctttgaccccacccatgacatttgaatccttcccgcctcagcccgacctctctcgcctctggccccaaactgtgacctctgaaccctccagtgacctccgaccccctacaacccccccctctccgcagacgacctcagggccctgctgtgacttgacagtgggcgggctcgaccccgtacgctgctatgacttccgggttcgggcgtcgccccgggccgcgcactatggcctggaggcgcagcctagcgagtggacagcggtgacaaggctttccggggcagcatccgcgggtgagggcttggcctggggttgtcaaggcttcactgcgggtgggtggttggcagggcctgggtgactgaaagacgggtgggcgtgacctgcgatgacgtcattgataccagggatgattgataagcgaatgggcggggcttggaagtgatcgagggttacatcatcagtgacattatcctgtgcacttgggtgactgacaggcgggtggtggttggggtctgggtaagatgaactcatcggctcctggatgcttgacaggtgggtgggcgtggtggcgatgacatcattggtgacccggatgactgccgggcagtgggcggtgcccctggtgatgtcaccacgccactcgactcacaggtgacccctgcgccgcccaccttccccccctagcctcctgtaccgcaagccccgccccatccccggccctggccccgcccctcctgcccctgggctgcggcctagcagcgctgctgacactgtccctgctcctggccgccctgaggcttcgcaggtgaggggtctccgaggagtcatggggtcatggtggggtaattgcgatgtctacggcgatgacgtcaccactgtgatgacgtcatgttcgggtcgggggcatggcatgggggtgtcaggtgaaccctgacccctgaccctgaaccccagggtgaaagatgcgctgctgccctgcgtccctgaccccagcggctccttccctggactctttgagaagcatcacgggaacttccaggtgcgcggggggggggggggggtcaggatcgctgtggggggtcacttcctgtccccggagagtgagggcatgaggcagggggatgatgggagtgacaggcgtcacgggccactgcctgactccaggcaccgctcccctccccctccccccacccccttccctccaccccccccctccacacacacaacccctttcgctgcaggcctggattgcggacgcccaggccacagccccgccagccaggaccgaggaggaagatgacctcatccacaccaaggctaagagggtggagcccgaggacggcacctccctctgcaccgtgccaaggccacccagcttcgagccaagggggccgggaggcggggccatggtgtcagtgggcggggccacgttcatggtgggcgacagcggctacatgaccctgtgaccttgaagtcactgccagtctatacttcaggctgaggtcacttcctgtctttaaataattcaaactcacaaatcctgtgcctgtctgtatgcaaatgtggtcacaaatattcaaataaaatgcaaatgctatgctaatgcggccagccatcattaattcctgaccagaaagggcggggcttgaggagcatcccagcgtctagtgggggtaacatcagggtgacatcatctccccatccatctgggcggggcaatgagcaaatgtacctcaagaattcgcaggtatgtaaacgagtttcatgaatatgctgtcctggtggccctgagggagagaaagggtaagaaccaagggaggacagaactgcgggtggtgagtaccacctcgcccaggccccgccctgggatctaggccccacccacacatgctggcaactcccctgtgactgtaagtgatgtcacttcctgtcttttcctctgtgttctctgcttcctgtgctctctgcaggccccgcccaactcacccaggccctgccctgatgcccgggccacaccccatagtctggtggtgtccttgtctgacagtgacggacctttccctgacatcacttcctgtccctgcccctgtgtccgcccaccctgcccgtgaccccaccccctgaacactgaccccctccccccgaccacggacccctcccaacccagcaggacatggtgaactcgcaccggggcctggccttcctgaaagcagcaaaggagttccactagcgttacatcaccactgtgagtggacacgccccgatacgcccccaaccacgggacatgcccagaccccgagatcccccccccccccccccccccgctcacagccctgagatttccgcccataatccaaggccacaaacacctggcgttcagccacacccacacagtcccaggccaaacctcaggtgctctgtgaatctcttgactgccctgaccatgcccctgactctactcatagctgttaggttcactatgaatccctctgaccctgcccagactccgcccatagcccttggccccgcccataaagactttacctgattcctgccttggccccgccctagccctgcccaattccccacagccccgcccctagtgccaggccacactcatagccatggctccacctccgaccctgtgaccccggctcctcccccatgtgactcagaggatcttctacacagtgaaccagtcatggtctg

SEQ ID NO: 11 - DNA, 1385 bp, synthetic construct
  source 1..1385: mol_type = other DNA
gaaccagctgtcaatcactgcagcgtccgcggccccgccggcgacatggcatgggcactcgcggtcatcctcctgcctcggctccttgcggcggcagcggcggcggcggcggtgacgtcacggggtgaggagtgagcgggggcggggctgcctgtcaatcgccgcggtgggcggggcccgagcaagagctaccaagttgcttttcgtcccatcattgcttttcgtcccatcatgaatatgcaaataaggcctctggccctcctaagggcgatcggatagcgcttcgtttgcatattcattttgatctttgcgtatgcatgagccccgccctccccccctacgctcggcgctttctcctcagtaatatgcaaatgagacctaaaccccgccttgacctcattagcatagtgctgccgccacaatctcgctcctcctcctgaatatgcaaataaggcctctgggccgctccttctttgcatattcatatacagctttcccgcttatatgcaaataacgcttcgcccctaccgagttctcactcatcgcttctcatttgcatatccatcgggagatacacatattcatgagcgatgtatttctgtcttccatcccctcatgaatatagaattgatgccctgtccatatggatcactatgcatttgcatattttccccacgatttacatatgcacaagcctcaacgtcttgctccaacgtccctgatcatgaatatgcagatgagacctcagatctccgaaattgaatctgcccgctcctcatcatatactgctattctcatatgcaccagccacaaagtcttccatccttttcccctcatgaatatgcaaagaatgcttccccagtccatctccactctggttcactgcctgctcatttgtgtatccattggtcgctttgcatagtggtgagccccgcttccctaccgctttctttgcgatcatgtatattcaaatgaggctccgacttgctagctgctcatttgcatattcgagaagcgctttccatattcatgagcccccaatccttttccttttctctgtttgtgaatatgcaaatgaggtgtcgcccaccgctacagttcttgggcctctcgtttacatattcattccctatatttgcgtacgagccagaatgtcttcctctgtcttcccctgcctagtgcatatgcaaatgaggcccggcctgctcccagcgccctagccaagggtctgagtcagggaaatggctgcgatggctcttctggaacgcgctgacgtcacgggctgctcccctgatccccgccctgcccccgcaggtgatgtcacagtcgtctgccatgacctggagacggtggaggtcacgtggggctcgggccccgaccaccac

SEQ ID NO: 12 - DNA, 1315 bp, synthetic construct
  source 1..1315: mol_type = other DNA
ggcgccaacttgagcctggagttccggtgagtgaaggggggcggggcagaacttctggtctgtgacttcctgtcggaatcattgaatgatatatgggagggggcgggatttcctctcattcctcttcactcacttgctgttcattcttttaaacgcctgaaagagtgggcgggacttcctgtctaattaatgattgacaaatgattagggacggtacttcctttcagactctgatgaatgacaggaaggagcggtacttcctttctgcccatcactttctgtttgaggtgttgaaattcagatgctcgtgggcgggacttctggtccatcacttcccatccagctgatccttcccccttaatccccgactctgcagttatggcactggcgccctgcaaccctgcccgcgatatttcctgtccggcgctggtgtcacttccgggtgcatcctccccgcggcgagggcggggctgctggagctggcactgcgcgacggaggcggggccatggtgtttaaggctaggcagcgcgcgtccgcctggcgtgagtggtgtgggcgtgacatggagggagggggcggggccttgggcggaagtacgcaggaagttgcctgatgatgtctcttcaactcccactgagtggggcaaacaggaactgggcggcgcttagacgggaagttggtctttgataccggaattgggctccaggggttaggcagtgctctgatgatgtcacagctgcgcctttggactccggtgggcggagcatagacagaaagtggttcttaggaatgggaagtaggcgtgtcctgctctctgaagtgcgccccttccctcatcccgatgggcatggccttcatgaaacaggcgggggtgggacagaaagtgatgtcaccgccacaacacccagactctcggtgggtgtggattaagccaaatgaggtcacagcagctcctcccacccccaccttccccaccgcagtgaagccccgcccaccttggaatgtgacgctgctctggacaccagacggggacgtgactgtctcctggcctgcccactcctacctgggcctggactacgaggtgcagcaccgggagagcaatgacgatgaggacgcctggcaggcgagtgaattcatggggggcagggccggcccctccgtgacctctgaccccacctgcgacctcctgcccccgcacactgcgttttccaaccctaacaatagatgcatggcctttgaccccacccatgacatttgaatccttcccgcctcagcccgacctctctcgcctctggccccaaactgtgacctctg

SEQ ID NO: 13 - DNA, 2941 bp, synthetic construct
  source 1..2941: mol_type = other DNA
ggaagcggagctactaacttcagcctgctgaagcaggctggagacgtggaggagaaccctggacctatggggcggctggttctgctgtggggagctgccgtctttctgctgggaggctggatggctttggggcaaggaggagcagcagaaggagtacagattcagatcatctacttcaatttagaaaccgtgcaggtgacatggaatgccagcaaatactccaggaccaacctgactttccactacagattcaacggtgatgaggcctatgaccagtgcaccaactaccttctccaggaaggtcacacttcggggtgcctcctagacgcagagcagcgagacgacattctctatttctccatcaggaatgggacgcaccccgttttcaccgcaagtcgctggatggtttattacctgaaacccagttccccgaagcacgtgagattttcgtggcatcaggatgcagtgacggtgacgtgttctgacctgtcctacggggatctcctctatgaggttcagtaccggagccccttcgacaccgagtggcagtccaaacaggaaaatacctgcaacgtcaccatagaaggcttggatgccgagaagtgttactctttctgggtcagggtgaaggctatggaggatgtatatgggccagacacatacccaagcgactggtcagaggtgacatgctggcagagaggcgagattcgggatgcctgtgcagagacaccaacgcctcccaaaccaaagctgtccaaatttattttaatttccagcctggccatccttctgatggtgtctctcctccttctgtctttatggaaacttcgcagggtgaaagatgcgctgctgccctgcgtccctgaccccagcggctccttccctggactctttgagaagcatcacgggaacttccaggcctggattgcggacgcccaggccacagccccgccagccaggaccgaggaggaagatgacctcatccacaccaaggctaagagggtggagcccgaggacggcacctccctctgcaccgtgccaaggccacccagcttcgagccaagggggccgggaggcggggccatggtgtcagtgggcggggccacgttcatggtgggcgacagcggctacatgaccctgtgaccttgaagtcactgccagtctatacttcaggctgaggtcacttcctgtctttaaataattcaaactcacaaatcctgtgcctgtctgtatgcaaatgtggtcacaaatattcaaataaaatgcaaatgctatgctaatgcggccagccatcattaattcctgaccagaaagggcggggcttgaggagcatcccagcgtctagtgggggtaacatcagggtgacatcatctccccatccatctgggcggggcaatgagcaaatgtacctcaagaattcgcaggtatgtaaacgagtttcatgaatatgctgtcctggtggccctgagggagagaaagggtaagaaccaagggaggacagaactgcgggtggtgagtaccacctcgcccaggccccgccctgggatctaggccccacccacacatgctggcaactccccattaagggttccggatcctcggggacaccaaatatggcgatctcggccttttcgtttcttggagctgggacatgtttgccatcgatccatctaccaccagaacggccgttagatctgctgccaccgttgtttccaccgaagaaaccaccgttgccgtaaccaccacgacggttgttgctaaagaagctgccaccgccacggccaccgttgtagccgccgttgttgttattgtagttgctcatgttatttctggcacttcttggttttcctcttaagtgaggaggaacataaccattctcgttgttgtcgttgatgcttaaattttgcacttgttcgctcagttcagccataatatgaaatgcttttcttgttgttcttacggaataccacttgccacctatcaccacaactaactttttcccgttcctccatctcttttatattttttttctcgagggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgacatggtaagtaagcttgggctgcaggtcgagggacctaagtactgtcgac

SEQ ID NO: 14 - DNA, 4818 bp, synthetic construct
  source 1..4818: mol_type = other DNA
ggaagcggagctactaacttcagcctgctgaagcaggctggagacgtggaggagaaccctggacctatggggcggctggttctgctgtggggagctgccgtctttctgctgggaggctggatggctttggggcaaggaggagcagcagaaggagtacagattcagatcatctacttcaatttagaaaccgtgcaggtgacatggaatgccagcaaatactccaggaccaacctgactttccactacagattcaacggtgatgaggcctatgaccagtgcaccaactaccttctccaggaaggtcacacttcggggtgcctcctagacgcagagcagcgagacgacattctctatttctccatcaggaatgggacgcaccccgttttcaccgcaagtcgctggatggtttattacctgaaacccagttccccgaagcacgtgagattttcgtggcatcaggatgcagtgacggtgacgtgttctgacctgtcctacggggatctcctctatgaggttcagtaccggagccccttcgacaccgagtggcagtccaaacaggaaaatacctgcaacgtcaccatagaaggcttggatgccgagaagtgttactctttctgggtcagggtgaaggctatggaggatgtatatgggccagacacatacccaagcgactggtcagaggtgacatgctggcagagaggcgagattcgggatgcctgtgcagagacaccaacgcctcccaaaccaaagctgtccaaatttattttaatttccagcctggccatccttctgatggtgtctctcctccttctgtctttatggaaacttcgcagggtgaaagatgcgctgctgccctgcgtccctgaccccagcggctccttccctggactctttgagaagcatcacgggaacttccaggcctggattgcggacgcccaggccacagccccgccagccaggaccgaggaggaagatgacctcatccacaccaaggctaagagggtggagcccgaggacggcacctccctctgcaccgtgccaaggccacccagcttcgagccaagggggccgggaggcggggccatggtgtcagtgggcggggccacgttcatggtgggcgacagcggctacatgaccctgtgaccttgaagtcactgccagtctatacttcaggctgaggtcacttcctgtctttaaataattcaaactcacaaatcctgtgcctgtctgtatgcaaatgtggtcacaaatattcaaataaaatgcaaatgctatgctaatgcggccagccatcattaattcctgaccagaaagggcggggcttgaggagcatcccagcgtctagtgggggtaacatcagggtgacatcatctccccatccatctgggcggggcaatgagcaaatgtacctcaagaattcgcaggtatgtaaacgagtttcatgaatatgctgtcctggtggccctgagggagagaaagggtaagaaccaagggaggacagaactgcgggtggtgagtaccacctcgcccaggccccgccctgggatctaggccccacccacacatgctggcaactccccattaagggttccggatcctcggggacaccaaatatggcgatctcggccttttcgtttcttggagctgggacatgtttgccatcgatccatctaccaccagaacggccgttagatctgctgccaccgttgtttccaccgaagaaaccaccgttgccgtaaccaccacgacggttgttgctaaagaagctgccaccgccacggccaccgttgtagccgccgttgttgttattgtagttgctcatgttatttctggcacttcttggttttcctcttaagtgaggaggaacataaccattctcgttgttgtcgttgatgcttaaattttgcacttgttcgctcagttcagccataatatgaaatgcttttcttgttgttcttacggaataccacttgccacctatcaccacaactaactttttcccgttcctccatctcttttatattttttttctcgagggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgacatggtaagtaagcttgggctgcaggtcgagggacctagtcgacggtatcgataagcttgatatcgaattccgaagttcctattctctagaaagtataggaacttcaggtctgaagaggagtttacgtccagccaagctagcttggctgcaggtcgtcgaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcctgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttcgaagttcctattctctagaaagtataggaacttcatcagtcaggtacataatggtggatcc

SEQ ID NO: 15 - DNA, 759 bp, synthetic construct
  source 1..759: mol_type = other DNA
atggggcggctggttctgctgtggggagctgccgtctttctgctgggaggctggatggctttggggcaaggaggagcagcagaaggagtacagattcagatcatctacttcaatttagaaaccgtgcaggtgacatggaatgccagcaaatactccaggaccaacctgactttccactacagattcaacggtgatgaggcctatgaccagtgcaccaactaccttctccaggaaggtcacacttcggggtgcctcctagacgcagagcagcgagacgacattctctatttctccatcaggaatgggacgcaccccgttttcaccgcaagtcgctggatggtttattacctgaaacccagttccccgaagcacgtgagattttcgtggcatcaggatgcagtgacggtgacgtgttctgacctgtcctacggggatctcctctatgaggttcagtaccggagccccttcgacaccgagtggcagtccaaacaggaaaatacctgcaacgtcaccatagaaggcttggatgccgagaagtgttactctttctgggtcagggtgaaggctatggaggatgtatatgggccagacacatacccaagcgactggtcagaggtgacatgctggcagagaggcgagattcgggatgcctgtgcagagacaccaacgcctcccaaaccaaagctgtccaaatttattttaatttccagcctggccatccttctgatggtgtctctcctccttctgtctttatggaaa

SEQ ID NO: 16 - DNA, 318 bp, synthetic construct
  source 1..318: mol_type = other DNA
cttcgcagggtgaaagatgcgctgctgccctgcgtccctgaccccagcggctccttccctggactctttgagaagcatcacgggaacttccaggcctggattgcggacgcccaggccacagccccgccagccaggaccgaggaggaagatgacctcatccacaccaaggctaagagggtggagcccgaggacggcacctccctctgcaccgtgccaaggccacccagcttcgagccaagggggccgggaggcggggccatggtgtcagtgggcggggccacgttcatggtgggcgacagcggctacatgaccctgtga

SEQ ID NO: 17 - DNA, 57 bp, synthetic construct
  source 1..57: mol_type = other DNA
gctactaacttcagcctgctgaagcaggctggagacgtggaggagaaccctggacct

SEQ ID NO: 18 - RNA, 1540 nt, synthetic construct
  source 1..1540: mol_type = other RNA
gcccccggcttcccgttttcggctctaagcggcctgggcgccctcgactcggaccggctcggaccgaaccagctgtcaatcactgcagcgtccgcggccccgccggcgacatggcatgggcactcgcggtcatcctcctgcctcggctccttgcggcggcagcggcggcggcggcggtgacgtcacggggtgatgtcacagtcgtctgccatgacctggagacggtggaggtcacgtggggctcgggccccgaccaccacggaagcggagctactaacttcagcctgctgaagcaggctggagacgtggaggagaaccctggacctatggggcggctggttctgctgtggggagctgccgtctttctgctgggaggctggatggctttggggcaaggaggagcagcagaaggagtacagattcagatcatctacttcaatttagaaaccgtgcaggtgacatggaatgccagcaaatactccaggaccaacctgactttccactacagattcaacggtgatgaggcctatgaccagtgcaccaactaccttctccaggaaggtcacacttcggggtgcctcctagacgcagagcagcgagacgacattctctatttctccatcaggaatgggacgcaccccgttttcaccgcaagtcgctggatggtttattacctgaaacccagttccccgaagcacgtgagattttcgtggcatcaggatgcagtgacggtgacgtgttctgacctgtcctacggggatctcctctatgaggttcagtaccggagccccttcgacaccgagtggcagtccaaacaggaaaatacctgcaacgtcaccatagaaggcttggatgccgagaagtgttactctttctgggtcagggtgaaggctatggaggatgtatatgggccagacacatacccaagcgactggtcagaggtgacatgctggcagagaggcgagattcgggatgcctgtgcagagacaccaacgcctcccaaaccaaagctgtccaaatttattttaatttccagcctggccatccttctgatggtgtctctcctccttctgtctttatggaaacttcgcagggtgaaagatgcgctgctgccctgcgtccctgaccccagcggctccttccctggactctttgagaagcatcacgggaacttccaggcctggattgcggacgcccaggccacagccccgccagccaggaccgaggaggaagatgacctcatccacaccaaggctaagagggtggagcccgaggacggcacctccctctgcaccgtgccaaggccacccagcttcgagccaagggggccgggaggcggggccatggtgtcagtgggcggggccacgttcatggtgggcgacagcggctacatgaccctgtgaccttgaagtcactgccagtctatacttcaggctgaggtcacttcctgtctttaaataattcaaactcacaaatcctgtgcctgtctgtatgcaaatgtggtcacaaatattcaaataaaatgcaaatgctatgctaa

SEQ ID NO: 19 - protein, 358 aa, synthetic construct
MGRLVLLWGAAVFLLGGWMALGQGGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKFILISSLAILLMVSLLLLSLWKLRRVKDALLPCVPDPSGSFPGLFEKHHGNFQAWIADAQATAPPARTEEEDDLIHTKAKRVEPEDGTSLCTVPRPPSFEPRGPGGGAMVSVGGATFMVGDSGYMTL

SEQ ID NO: 20 - DNA, 945 bp, synthetic construct
  source 1..945: mol_type = other DNA
cctcaaaatcacaagaacaacgcacctagaggtgtcactgttcacagaaagtagggtaaccttatattaatcattaattaagtacatggcttactcacaaaaccacaggtcaatatgatggtagccatttctcaattgagatccccggttctcaacatcttcaggtttgtgtccttatgaatgaaaccattttccaccaaactttagtaattggtgtaacaatgcacagtgctgccctgtttgtaagttcaaatttgtatattacttaaaatctttgtatgattaagtacgacttttgttaagacaaagaaaaagatatgttcactattatctaatatttacataccatttaagtttcaacaaagcgatttacttttgtgctgggccacctagagactgggaaagggagaagcagaagtgacctggaacagaaagtcacacgatggccagccacctccatcttggtgaggtcaatgtgcaaccaaatgacagaacaaagaagtagagaggaacagacatatgggagcaacacctcccttctgtttttggataaaagacacacaatttttattatttacaagtaagccttaaaaagcaccacaagagccgggcagattagatgggcgggggcggggccttggaggtttgggggcggggacaggccgcgaggggcgtggtcggggcgcacgcggagtccacccccccccccccgccccccccccgcttccccctatccgggtgattgatgggaagaggaagccgggccacgcccatgtcggtgagtcgccgcatccgtttattgccgtggccccgccccgtcgccccaacccccgcccccggcttcccgttttcggctctaagcggcctgggcgccctcgactcggaccggctcggaccgaaccagctgtcaatcactgcagcgtccgcggccccgccggcgacatg

SEQ ID NO: 21 - DNA, 1756 bp, synthetic construct
  source 1..1756: mol_type = other DNA
gcatgggcactcgcggtcatcctcctgcctcggctccttgcggccccagcggcggcggcggcggtgacgtcacggggtgaggagtgagcgggggcggggctgcctgtcaatcgccgcggtgggcggggcccgagcaagagctaccaagttgcttttcgtcccatcattgcttttcgtcccatcatgaatatgcaaataaggcctctggccctcctaagggcgatcggatagcgcttcgtttgcatattcattttgatctttgcgtatgcatgagccccgccctccccccctacgctcggcgctttctcctcagtaatatgcaaatgagacctaaaccccgccttgacctcattagcatagtgctgccgccacaatctcgctcctcctcctgaatatgcaaataaggcctctgggccgctccttctttgcatattcatatacagctttcccgcttatatgcaaataacgcttcgcccctaccgagttctcactcatcgcttctcatttgcatatccatcgggagatacacatattcatgagcgatgtatttctgtcttccatcccctcatgaatatagaattgatgccctgtccatatggatcactatgcatttgcatattttccccacgatttacatatgcacaagcctcaacgtcttgctccaacgtccctgatcatgaatatgcagatgagacctcagatctccgaaattgaatctgcccgctcctcatcatatactgctattctcatatgcaccagccacaaagtcttccatccttttcccctcatgaatatgcaaagaatgcttccccagtccatctccactctggttcactgcctgctcatttgtgtatccattggtcgctttgcatagtggtgagccccgcttccctaccgctttctttgcgatcatgtatattcaaatgaggctccgacttgctagctgctcatttgcatattcgagaagcgctttccatattcatgagcccccaatccttttccttttctctgtttgtgaatatgcaaatgaggtgtcgcccaccgctacagttcttgggcctctcgtttacatattcattccctatatttgcgtacgagccagaatgtcttcctctgtcttcccctgcctagtgcatatgcaaatgaggcccggcctgctcccagcgccctagccaagggtctgagtcagggaaatggctgcgatggctcttctggaacgcgctgacgtcacgggctgctcccctgatccccgccctgcccccgcaggtgatgtcacagtcgtctgccatgacctggagacggtggaggtcacgtggggctcgggccccgaccaccacggcgccaacttgagcctggagttccggtgagtgaaggggggcggggcagaacttctggtctgtgacttcctgtcggaatcattgaatgatatatgggagggggcgggatttcctctcattcctcttcactcacttgctgttcattcttttaaacgcctgaaagagtgggcgggacttcctgtctaattaatgattgacaaatgattagggacggtacttcctttcagactctgatgaatgacaggaaggagcggtacttcctttctgcccatcactttctgtttgaggtgttgaaattcagatgctcgtgggcgggacttctggtccatcacttcccatccagctgatccttcccccttaatccccgactctgcagttatggcactggcgccctgcaaccctgcccgcgatatttcctg

SEQ ID NO: 22 - DNA, 2860 bp, synthetic construct
  source 1..2860: mol_type = other DNA
gggcggctggttctgctgtggggagctgccgtctttctgctgggaggctggatggctttggggcaaggaggagcagcagaaggagtacagattcagatcatctacttcaatttagaaaccgtgcaggtgacatggaatgccagcaaatactccaggaccaacctgactttccactacagattcaacggtgatgaggcctatgaccagtgcaccaactaccttctccaggaaggtcacacttcggggtgcctcctagacgcagagcagcgagacgacattctctatttctccatcaggaatgggacgcaccccgttttcaccgcaagtcgctggatggtttattacctgaaacccagttccccgaagcacgtgagattttcgtggcatcaggatgcagtgacggtgacgtgttctgacctgtcctacggggatctcctctatgaggttcagtaccggagccccttcgacaccgagtggcagtccaaacaggaaaatacctgcaacgtcaccatagaaggcttggatgccgagaagtgttactctttctgggtcagggtgaaggctatggaggatgtatatgggccagacacatacccaagcgactggtcagaggtgacatgctggcagagaggcgagattcgggatgcctgtgcagagacaccaacgcctcccaaaccaaagctgtccaaatttattttaatttccagcctggccatccttctgatggtgtctctcctccttctgtctttatggaaacttcgcagggtgaaagatgcgctgctgccctgcgtccctgaccccagcggctccttccctggactctttgagaagcatcacgggaacttccaggcctggattgcggacgcccaggccacagccccgccagccaggaccgaggaggaagatgacctcatccacaccaaggctaagagggtggagcccgaggacggcacctccctctgcaccgtgccaaggccacccagcttcgagccaagggggccgggaggcggggccatggtgtcagtgggcggggccacgttcatggtgggcgacagcggctacatgaccctgtgaccttgaagtcactgccagtctatacttcaggctgaggtcacttcctgtctttaaataattcaaactcacaaatcctgtgcctgtctgtatgcaaatgtggtcacaaatattcaaataaaatgcaaatgctatgctaatgcggccagccatcattaattcctgaccagaaagggcggggcttgaggagcatcccagcgtctagtgggggtaacatcagggtgacatcatctccccatccatctgggcggggcaatgagcaaatgtacctcaagaattcgcaggtatgtaaacgagtttcatgaatatgctgtcctggtggccctgagggagagaaagggtaagaaccaagggaggacagaactgcgggtggtgagtaccacctcgcccaggccccgccctgggatctaggccccacccacacatgctggcaactccccattaagggttccggatcctcggggacaccaaatatggcgatctcggccttttcgtttcttggagctgggacatgtttgccatcgatccatctaccaccagaacggccgttagatctgctgccaccgttgtttccaccgaagaaaccaccgttgccgtaaccaccacgacggttgttgctaaagaagctgccaccgccacggccaccgttgtagccgccgttgttgttattgtagttgctcatgttatttctggcacttcttggttttcctcttaagtgaggaggaacataaccattctcgttgttgtcgttgatgcttaaattttgcacttgttcgctcagttcagccataatatgaaatgcttttcttgttgttcttacggaataccacttgccacctatcaccacaactaactttttcccgttcctccatctcttttatattttttttctcgagggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgacatggtaagtaagcttgggctgcaggtcgagggaccta

SEQ ID NO: 23 - DNA, 48 bp, synthetic construct
  source 1..48: mol_type = other DNA
tcaggaagactccacgttcaggcgacagcatgttcccttttgccttac

SEQ ID NO: 24 - DNA, 81 bp, synthetic construct
  source 1..81: mol_type = other DNA
gaaagtataggaacttcatcagtcaggtacataatggtggatcccactgggtgcctccctgcaggagaaaaggtcttcata

SEQ ID NO: 25 - DNA, 58 bp, synthetic construct
  source 1..58: mol_type = other DNA
cacacttcctttgatctggtatcaacaagcttgatatcgaattccgaagttcctattc

SEQ ID NO: 26 - DNA, 68 bp, synthetic construct
  source 1..68: mol_type = other DNA
tataggaacttcatcagtcaggtacataatggtggatcccactgggtgcctccctgcaggagaaaagg

SEQ ID NO: 27 - DNA, 24 bp, synthetic construct
  source 1..24: mol_type = other DNA
tacaagtccagcatgacatagcca

SEQ ID NO: 28 - DNA, 22 bp, synthetic construct
  source 1..22: mol_type = other DNA
tgccacctaattgcagaggcga

SEQ ID NO: 29 - DNA, 24 bp, synthetic construct
  source 1..24: mol_type = other DNA
aagacctgacagtgttgttcaagg

SEQ ID NO: 30 - DNA, 22 bp, synthetic construct
  source 1..22: mol_type = other DNA
ttccggtggcctgtaggacatt

SEQ ID NO: 31 - DNA, 21 bp, synthetic construct
  source 1..21: mol_type = other DNA
ggatcggccattgaacaagat

SEQ ID NO: 32 - DNA, 22 bp, synthetic construct
  source 1..22: mol_type = other DNA
cagaagaactcgtcaagaaggc

SEQ ID NO: 33 - DNA, 25 bp, synthetic construct
  source 1..25: mol_type = other DNA
gccactcagccagccgttaaagata

SEQ ID NO: 34 - DNA, 51 bp, synthetic construct
  source 1..51: mol_type = other DNA
ctcgggccccgaccaccacggaagcggagctactaacttcagcctgctgaa

SEQ ID NO: 35 - DNA, 69 bp, synthetic construct
  source 1..69: mol_type = other DNA
gaaagtataggaacttcatcagtcaggtacataatggtggatccggcgccaacttgagcctggagttcc

SEQ ID NO: 36 - DNA, 78 bp, synthetic construct
  source 1..78: mol_type = other DNA
gcttgggctgcaggtcgagggacctagtcgacggtatcgataagcttgatatcgaattccgaagttcctattctctag

SEQ ID NO: 37 - DNA, 76 bp, synthetic construct
  source 1..76: mol_type = other DNA
aaagtataggaacttcatcagtcaggtacataatggtggatccggcgccaacttgagcctggagttccggtgagtg

SEQ ID NO: 38 - DNA, 22 bp, synthetic construct
  source 1..22: mol_type = other DNA
gtcgccgcatccgtttattgcc

SEQ ID NO: 39 - DNA, 27 bp, synthetic construct
  source 1..27: mol_type = other DNA
gtcacaccacagaagtaaggttccttc

SEQ ID NO: 40 - DNA, 73 bp, synthetic construct
  source 1..73: mol_type = other DNA
gggctgcaggtcgagggacctaagtactgtcgacggcgccaacttgagcctggagttccggtgagtgaagggg

SEQ ID NO: 41 - DNA, 23 bp, synthetic construct
  source 1..23: mol_type = other DNA
ggctcaagttggcgccgtggtgg

SEQ ID NO: 42 - DNA, 27 bp, synthetic construct
  source 1..27: mol_type = other DNA
taggggctcaagttggcgccgtggtgg

SEQ ID NO: 43 - DNA, 23 bp, synthetic construct
  source 1..23: mol_type = other DNA
ccaccacggcgccaacttgagcc

SEQ ID NO: 44 - DNA, 27 bp, synthetic construct
  source 1..27: mol_type = other DNA
aaacccaccacggcgccaacttgagcc

SEQ ID NO: 45 - DNA, 132 bp, synthetic construct
  source 1..132: mol_type = other DNA
gaattctaatacgactcactatagggggtcttcgagaagacctgttttagagctagaaatagcaagttaaaataaggctagtccgttatcaacttgaaaaagtggcaccgagtcggtgcttttaaaggatcc

SEQ ID NO: 46 - DNA, 23 bp, synthetic construct
  source 1..23: mol_type = other DNA
cgccctagccaagggtctgagtc

SEQ ID NO: 47 - DNA, 27 bp, synthetic construct
  source 1..27: mol_type = other DNA
gtctgaaaggaagtaccgtccctaatc

SEQ ID NO: 48 - DNA, 24 bp, synthetic construct
  source 1..24: mol_type = other DNA
gagaatgtcgtctcgctgctctgc

SEQ ID NO: 49 - DNA, 25 bp, synthetic construct
  source 1..25: mol_type = other DNA
aactgatgaatgggagcagtggtgg

SEQ ID NO: 50 - DNA, 25 bp, synthetic construct
  source 1..25: mol_type = other DNA
gcagacactctatgcctgtgtggag

SEQ ID NO: 51 - DNA, 22 bp, synthetic construct
  source 1..22: mol_type = other DNA
cgggtgggcgtgacctgcgatg

SEQ ID NO: 52 - DNA, 22 bp, synthetic construct
  source 1..22: mol_type = other DNA
cggcgcaggggtcacctgtgag

SEQ ID NO: 53 - DNA, 23 bp, synthetic construct
  source 1..23: mol_type = other DNA
cctgcctcggctccttgcggcgg

SEQ ID NO: 54 - DNA, 27 bp, synthetic construct
  source 1..27: mol_type = other DNA
taggcctgcctcggctccttgcggcgg

SEQ ID NO: 55 - DNA, 23 bp, synthetic construct
  source 1..23: mol_type = other DNA
ccgccgcaaggagccgaggcagg

SEQ ID NO: 56 - DNA, 27 bp, synthetic construct
  source 1..27: mol_type = other DNA
aaacccgccgcaaggagccgaggcagg

SEQ ID NO: 57 - DNA, 23 bp, synthetic construct
  source 1..23: mol_type = other DNA
tgggaccgtctgcagagtttgtg

SEQ ID NO: 58 - DNA, 26 bp, synthetic construct
  source 1..26: mol_type = other DNA
tcccattcatcagttccataggttgg

SEQ ID NO: 59 - DNA, 20 bp, synthetic construct
  source 1..20: mol_type = other DNA
caaccctgcccgcgatattt

SEQ ID NO: 60 - DNA, 19 bp, synthetic construct
  source 1..19: mol_type = other DNA
ttgtcaccgctgtccactc

SEQ ID NO: 61 - DNA, 19 bp, synthetic construct
  source 1..19: mol_type = other DNA
cccgttttcaccgcaagtc

SEQ ID NO: 62 - DNA, 20 bp, synthetic construct
  source 1..20: mol_type = other DNA
caccctgacccagaaagagt

SEQ ID NO: 63 - DNA, 22 bp, synthetic construct
  source 1..22: mol_type = other DNA
tcaccatcttccaggagcgaga

SEQ ID NO: 64 - DNA, 21 bp, synthetic construct
  source 1..21: mol_type = other DNA
gaaggccatgccagtgagctt

SEQ ID NO: 65 - protein, 266 aa, Mus musculus
MRPRMKYSNSKISPAKFSSTAGEALVPPCKIRRSQQKTKEFCHVYCMRLRSGLTIRKETSYFRKEPTKRYSLKSGTKHEENFSAYPRDSRKRSLLGSIQAFAASVDTLSIQGTSLLTQSPASLSTYNDQSVSFVLENGCYVINVDDSGKDQEQDQVLLRYYESPCPASQSGDGVDGKKLMVNMSPIKDTDIWLHANDKDYSVELQRGDVSPPEQAFFVLHKKSSDFVSFECKNLPGTYIGVKDNQLALVEEKDESCNNIMFKLSKI

SEQ ID NO: 66 - protein, 270 aa, Homo sapiens
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 67 - DNA, 4218 bp, synthetic construct
  source 1..4218: mol_type = other DNA
aagggcacgcatatatcctttttacaatcagaaaaatatttacctgaaagaaagaaacaaaagaacaaaacaaaacaacaaaacaaacaaacaaaaaaccaactttctccagcaaaggcatcactgagcatctgttttaaaagttacatttaaaagccagtaattaagtatatctctgaaaggacgggtctgtggggaatctgtctcctgggaacagctacacaccctcacattccattcatatcgctccatttgcataactttactcatcagcttctgttaccaaagttgtttaatctgagctaccatataaatagaactgaagcgcgactgtgttagctctccaccggggctcactgcaggaaagtacagcattcaagaccagctatttcctgtctgtattgaggtgagtgccaggtcaatctctctgtttctgaggggaaccaatagtagcatctatcagctctcaaatgattctgatataaagagagaaaagacttgttactggagaaagtggcttgtttatcagaatgggatttgaaaccagttctgggttatacttttagttctgcaattgtgataaagatgctgacagtcttggtcgatgtatctgtccttaatgctcactccttagtttcttatctgttcaaccacgcaggctagtagtcctttagagcaaagttcttctacatgagtttgctcaagctcgtccaagggaggacgaaagggaagggaaggttaggagatcaaataattaactgaagatagagaaaggcaaaggcaggcagggcgtctcagtgaggggctgctggtattgttattatgttaacatgcatgaactctaagtctccaagaagagaggctatcgactgcaacacacaggtttatttaaaagacaattttaatgactagaaaaacattgtttctaggacacatgcaccctctggtgaaagagaaaacagaaatcagcactgtttcaggaagcagctcagaagctcagcactagcagtgagcaagggacctcaattcctcctggtacaggcaggctattatttaaactgtttcctgcccaactgaaaatctgacctttatgtggttagctaatcctattaccccacacagaaggcttaaaaattaactattctgtaaaagctataactctgctttaaaggaaattatgtattctgagccagaaaggatgactgaccattgtggcttagtagtatggattcaagttaccctaaatgatacattcctaagtgaaagccattacattaagggtttcaggcaaagaataaaggccaccatacaccaatgtattcaataactgcattagtcataacacgttcgacacacagaagacacaagaaggaggaggtctctcttgtaggttttgaatgcttgcagcttcttagctttgtgctatgcataaacagcccattctgagagtatcaaatcagatgccattattaaaagaaatggctgtttacacaaaaagcctaagacagccagagataaagtttggctcttgatcattgaccaatgatcaagacaatgacaatattttactcagtgaacgagaagagtacgtgggattatttaacccagccgcagcttcttcagggaactctgccttatgttagttaacatcttccgaaatttattattactatttcaagaatattaatatttcaagaagctggccatttgctttgagtataagaagggctgagtgtttgaaataaaataccaagttctaatccacccgactcccaccccccaagccgcaccccgcacctcctcgaagttgtcaactgatgcagttgttggatgtattggctggcatatggttgcctttgttctttgcctgaaagccttcatgcgactgctttccgcttaggagtgcattgtttagggaaacttgaatcaatgctactaagccatgatcagctatcatcgctggctcagaataaatggttcccttcaaagcagagttgtaacttttatagagtagttaatttttaatccttttgaacaaaagagagagagagagaggatgataatggagggagagagaagagataacagttttagtttcattgggggggtggtacaggataagagataagttctactttcattcttctacacatggatatccagctctcccagtttaaaaaaatttttaaggctatatttttctgatgtgtttttgatgccactagcaagaattagacggttctattggcctgagtatgacataattatattttgatttttaaaaaatttaagaaaaaatagacaatgtgttaaggttgaaacagtgtggggaagataacaagatttaaaactcaaatgtaaaagaagtagataaaattagacagaagagggataaaaatgtttttaaaaatcaaaataataagtaatagttttaaatgagaggaaaataaaaagagagacggaagcacctttccgggtctgtgagcagagggcagagggctagagcttgctcccactaagctggctagaatagcgaggccagttgagagcacccatcctcagcctcagggctgtgtttactctagactgtaaaagcgtagatgctttcttcctttgcacttatggggccatggcatcccccgacgatgtctacattccttaggcttgggcaggttatcattcactgaggctctctggagtccgcgactgtggcaaagtccacctagttcacctcagaatgaagggagcaaacggctctcctagttatgtccagatggaagagcaatcagcctgtcctggtttataagtaatcttcagctacctgcttggaggcctcagtcatccccaatcccaggagctgagtagtcctcgccaccagagggcgggagcactgatcactgtgtcttccttcctcaactgagcaggctccccgtaattgaaacttctcattggttcgttcatccgaaccaatcccttccaggattgcctcatagccggtcactatggcagcgttcccaggcatcctgatttggcacctgtctgtattttctgatcttcagagttgctgctggtcacaacacttttaaactaccggagagtgacggtgtggcgtctttactctaggcagcagtggacaggaactcgcccttagacctaatttctgccctggcttgtgagaactgtactgttaaccatgggtttctcccgctgcgctgcgagcagcgcaattctattcttctggtagcctaaacccagatgaacttgtgattgttgctccctccctcctaatccttcccagtccctctccccatcccccaccaatgcctggaaccgggactggagctctggactgcttcctacttcgtgttatctttttgtttctccatgagttttaattgccctgttgtttttgttgttgttttggttttccccctttatgcccagtgatttctccactagatctccttgggctttggtttgtcctttgttaccctggtctttgaacttgtgagaaaatcggcaaccttactccatttattcttgaggaaccatgcttaatcttgatcactgcctgccattactctttttaaacattcagctggtgccaaatctgacccgaggaaaccctccacggtggctcccacatgcttttgacacaaactcatttagttttttaatagctggacgttttctggcataacgtgttttatggtcattattactttcctgaccacgaatctggagtcaaagttccttccagtggagaaaatatttagaaaataaaatgtgtacacacactgcttgtttaatactgactcaaatctaacatcgcagggcatttgccttggcttctgagttacgtatttatatttcttacccccctacagtgagaaatcatacaagatcagtatcgctattttttttttttttttgctatatactacaacaaaacagtactgagatttcaacacaaacatggcattaacactaagactactcagcctcagatttctctgtgctttcattactttattctttgattctatggctagattctagcctaaatgtagagtcagaatactatgctgaattttattctcccccctcccccctgtatggctatcagtttcatggtgagatagccaaggttgcttctgatgacttcaggtccatatagttggattaatgttatatttcaatcccacagaaacctgaaaa

SEQ ID NO: 68 - DNA, 4081 bp, synthetic construct
  source 1..4081: mol_type = other DNA
tgcagtaaaacgcctgtgcgttctgggttgaatgacttaatgcttccaactgaagaaagggtaacagagagaaagaaagccattcttggcttacgatgttgtggaatgttattatgtagaaaacttcttttatttccttttcttcagctacatgttcaatagactcacagatatatgacttacggcgttggtaaagaaactgaaggagattcagccttgctctttccttttctctgccttgagtcctgtatgaaatcacactcacggacttcagaagagcaggcaccacagtgcatggtttgctttagagagggtccatttcaaaaaccttcataaaaacaatgcaaaacaagaaaacaaccgaacaaaaaaaccacctatttcctggttctaaacaaatgattgtaatactagagcagttagtgggaggaccagctagggggaggatcacctaggggaggaccagctagggggaggaccagctgctgcaaagactgactgtttctcacttataataaaatgccaaatgcctccgcagatgccccaggcaaccctcagatcagccctttctgtgaagagtggcgttacctgtgcttgtttccttcttaaacttccaatttttctcttttaacacatttaacatttaactttaagcaagccagcttacattaggaagtgaaagacattttagttcccacccgtgattgaaatcattgactatatctaacaagcttaaagtctcctgtaagaactgatcaggatatacactaggacatgccaatagaatgggatctcatggtgcagtctgaagccctccaactggagagacgctaacatcatccttctcgctgatttccaaggagctatgactttggatgcatgcatctgcttggatgagatgtctcggctgcttgctttccttatgcacacgttctgttcagcttcacagcagcaatgctcacggtggaatagcttagcttagcttctgccccttctttggttcttttgaccaccatatccgtaacggctctcctactccctcagctttctttctctttgctctgacgtctatatgccaacacacttattccactgtctttaccctgcactgcagaattttacatctacctactggttaccaggttgtcccctgaacaaccttcctttgtgttttactgttattaaagtagtaatatttgtattcaaccatgtagtaatattttaagccactaaaggaatagttttacttatttagacaacagcaatttctactacatttttataagcttaaaacttacatgttttaaaacttaaaacgataaagacaataacaacattgatggagtatgatatgacagttcagaaagggttagctcttatcttccagtcgaggaaacctattgtatacaatagctggaggaatgtatgatcaaagaggccgggaaccgccgtgtaggatcgtacggctgtaacaggtataattgtttcattaatttgtcacagtcttactgtagaggaatgtaaaggcggaatctgcgtcattcctctggaaaccacagtgttgactctgtgaatctgtacgatatctttaaagtagtaactacgtagtcaaatgtgttcttgacgttgttcataactttgaataaaccatttttcaaaaccacgtgtgaccacaaagcgctccaacgttccgagttcttaaatgaaaatgagtataccccagtgcatcattgttgacaagtgaggccggtggctgtttgacaacttagggtgttaagttagactttccggaacacggatgtgtctgtttcaccttcatccccccagctcctccactctctcaaataggagactttcgcaccaaaccaaacatacgcccccatggtcctacatcaaatattaacctgaaattcccgatcccttatttcttttctgttttgctctcctttagaacatattttcagagctgaggcgacattttgagcaagatgaggaccagggttcagaccccaagctcccatatagaagccaagagtggctgtaagccccagggctaaggaggctgatcctgggatgctgctggccctgccagcctaaccaagctggtgagctctgggttctgagactctgtctcagatagaggaagagttctgacattgatctctggtttccacacacgtgcatatgtgcacaccacacacacacacacacacacacacacacacacacacacatacactttccacattactacaatcagtgttattgattaaaatattcaaacatattggcattttgtcatacttaacatgtgcttcatggctaataataaatattactgggaataatctgtccattcaattatatcagaaatgataattttatctgatatctgaaaatttagtcatgtaatgtaatcttgtttatgttctttaggtctttcttgatgtatttggaggatttttgaaaatttttatttaaaaaaatttttttaaagatttatttatttattatatgtaagtacactgtagctgtcctcagatactccaggagagagcatcagatttcattatggatcgttgtgagccaccatgtggttgctgagatttgaactcggaaccttcggaagagcagttggcactctgagccatctcaccagcccaatttaaaatttttttatacaaagttttttgatagtattctcttccctcccctaattcctttcagattttctccacatccactttctgggtttgttttgttttgttattttggggttgtttttttgggggggtgggggggtgagtgtggaggaagtggctctctctttctctaaagaaaaaaaaacataaaatcaaagcagacaaactaaaaaagagaaggtagataagacatcaagacgaaacaaaacacacccacaaaaccatggtgtccgtcttgtgttgctcagctacccctggcacgacgctctgcagtcttgtgcgtgctctcacagtctctgtgggatcctgtgtttatcagcacttcctgtgtctggaatacagtttccttgaggtcctggggacactgcttcctcttcttgatggatccctgattctcaatgggagaggttttgtgaagatatctcacttagggccgagtgctccaaactctccccctctctgtaccttgtccattgtggatctctttgttaattcacatctaccacaagaagaagcttcccaaagaggggttaagtgacactctgctctgtgtgtacagtcatatgtcattaagactcattctgctagccaggtggtggcggtgcacgcctttaatcccagcacttgggagggggatttccgagttcgaggccagcctggtctacaaagtgagttccaggacagccagggctacacagagaaaccctgtcaaaaaaccaaaaacaaacaaaaaaaacaaaaaaaacaccaagaatcattctgctcctttaacagaaaaatactagattttcccgtaggcctctgacctctctagccctaggttcttggccaattcagcagggacaggtatgggctccatctcatgaacccagtttttaaaaatggttggttattctgtaacattgttccacaggtctatcacgcaggcaggtagatcgctgggtaagacttgataattgcttttctcccccagtagactgcaaagtaccttgagaactctgagcgcttgtcagtagggtgaagcttttggttgggcacaggctcaatttcttatgttcagtgacatagagaagctgctgtcttcagtagtagggtcttaccatcgggttacagagagtaagcaactgccttaagcaacagtttgtgctgcggggaggttagcatatggaatcactttggccaacaactcaccaagatttaactcacagaagatgccttgttggggcattgtctcctacattatatggtgactccatttaaattccttttctatatagtacatcttttagaaagcttccgtggtattagttgtgcatctggctttttaaaagttctttattgtgagttatctgtccccgtattctctcctttcccttccc

SEQ ID NO: 69 - DNA, 756 bp, synthetic construct
  source 1..756: mol_type = other DNA
gggcggctggttctgctgtggggagctgccgtctttctgctgggaggctggatggctttggggcaaggaggagcagcagaaggagtacagattcagatcatctacttcaatttagaaaccgtgcaggtgacatggaatgccagcaaatactccaggaccaacctgactttccactacagattcaacggtgatgaggcctatgaccagtgcaccaactaccttctccaggaaggtcacacttcggggtgcctcctagacgcagagcagcgagacgacattctctatttctccatcaggaatgggacgcaccccgttttcaccgcaagtcgctggatggtttattacctgaaacccagttccccgaagcacgtgagattttcgtggcatcaggatgcagtgacggtgacgtgttctgacctgtcctacggggatctcctctatgaggttcagtaccggagccccttcgacaccgagtggcagtccaaacaggaaaatacctgcaacgtcaccatagaaggcttggatgccgagaagtgttactctttctgggtcagggtgaaggctatggaggatgtatatgggccagacacatacccaagcgactggtcagaggtgacatgctggcagagaggcgagattcgggatgcctgtgcagagacaccaacgcctcccaaaccaaagctgtccaaatttattttaatttccagcctggccatccttctgatggtgtctctcctccttctgtctttatggaaa

SEQ ID NO: 70 - protein, 448 aa, synthetic construct
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 71 - protein, 214 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 72 - DNA, 25 bp, synthetic construct
  source 1..25: mol_type = other DNA
agggacatcacggttgtctacatct

SEQ ID NO: 73 - DNA, 25 bp, synthetic construct
  source 1..25: mol_type = other DNA
agggtgttttaccagaggtgtgcag

SEQ ID NO: 74 - DNA, 25 bp, synthetic construct
  source 1..25: mol_type = other DNA
gacaagcgttagtaggcacatatac

SEQ ID NO: 75 - protein, 459 aa, Mus musculus
MMALGRAFAIVFCLIQAVSGESGNAQDGDLEDADADDHSFWCHSQLEVDGSQHLLTCAFNDSDINTANLEFQICGALLRVKCLTLNKLQDIYFIKTSEFLLIGSSNICVKLGQKNLTCKNMAINTIVKAEAPSDLKVVYRKEANDFLVTFNAPHLKKKYLKKVKHDVAYRPARGESNWTHVSLFHTRTTIPQRKLRPKAMYEIKVRSIPHNDYFKGFWSEWSPSSTFETPEPKNQGGWDPVLPSVTILSLFSVFLLVILAHVLWKKRIKPVVWPSLPDHKKTLEQLCKKPKTSLNVSFNPESFLDCQIHEVKGVEARDEVESFLPNDLPAQPEELETQGHRAAVHSANRSPETSVSPPETVRRESPLRCLARNLSTCNAPPLLSSRSPDYRDGDRNRPPVYQDLLPNSGNTNVPVPVPQPLPFQSGILIPVSQRQPISTSSVLNQEEAYVTMSSFYQNK

SEQ ID NO: 76 - protein, 459 aa, Homo sapiens
MTILGTTFGMVFSLLQVVSGESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 77 - DNA, 4438 bp, synthetic construct
  source 1..4438: mol_type = other DNA
taaaagctcagttctcaaggaccacagtgaaaggtttatgtatgaggacagtttacaacacaaaaataagaaccttttctatatgtcttgtctgtttcatagacaccctaaatgattacaaaagcagagacaattccagacacttgcacaaggggtactggaaaatctatgaaatcctccaattctgctcccatacaagatatgtgatcctttctagggattcacatttgaagaacacatgagaaaaggactatagaagaagaataagaaatggaagagatgactgaagagaggagtttgacaatctgagcaaattgctaaggatcaaaaccaaggaataaacccaaggaccttcccttgggtcctggcttctcaggtgcccaaagactcttttttcatcttcctttcaaacaactctgacaaagcagtgccatccatgtctgtcatgtggaggtcagggaggacagataagaaaagggaagttctttgcctgctttcggcttcaaaggatcctacgttaagaaacagagttaccattgctcacccacaatctctagaagtattagtgatgtttctcatttttctttaagcttcccgcactctatttagatttctctctgatgtcctttcagcatctcctgccccacccaaaaggggtaagcacaccagtggaaatcccctgagcaaactagcacatgctgtaccaaaatattatgtcttaactttgttcttttacatcttcacaactaaaggaaagagatacaatcaaaatgatggtccacttaaatgataaacaagagaatttaaagagacatgatacaattgttcaccatggagtgatagcccatgaaaggctgatgaggttggggttagtagatataatacatatgaatttctccttaaacattttcacatagctttgagaatttatcattgtagatttctagaaagcacattcaacttagtgtgcaaaacaggctgattttgcggtatgtttttctgatgaagctttcaaagcaagattacaagccagagtgtaaataattacctgattccccaaatcatttcttttgttgcttgggagaaaatttgaaattctccaaaagaggcccaaaatgcatggcagagatagaatatgctacctccaactgaattctttctctaacatttctttatggattgtagtcaagaacttgatgttataacagatgaaaatcacataacacctctaagatcccctcaacctgaggaggcattttcagagaacagcacaaataatacatctcctttcttttcttattagatcatctcattaagacatgttgagggaagttaagtcaagttgtagtcaaactctaaaactctcaggtactaatgctattcaacctctgggtggcattgatcatctactctaacttagtcattttatagactcaagtaactgattcttccagaaatctagctataacagaagttcataaaatcctatccactcagatgttttgaactccttctattataatcattcttactgcttctttacaaacagtgtgattatgaatcaaaatttaaaaaaaaactgtactctatggtctggatccagatttcttgattccttcaaagaactgaaaaggggccactcatctatctagttatgacaggaccctgataatgaacttaaagaaaataatccaactttatcaaaatctcagccattcttaacaaaaagcaccaattcatcttgaggccatgttaaaaacaaagtggattggcatacaagtttagccacagtctgaataatgactgcctccaacacctatcctacatgccttggagccaataaagtttatgtgaagcctttgttaaagaaaaattgataaccagtatgatgaaactctcttttaaatattctgagattgtacttatgtagctgtttagagatcagaagttttctaaagtcagatggtgtaagttttactatttatatatcatctctttgcccactcatctctactatatgtatgagaacagctgcagatagtacacaacaaatgggtaacattggactccaacaacgaattacttatgaaagaaggtaaaggatcaaattccattcctgagtcataatttgccaacacttgcatttaataatttctccgtgcctgtacttaatagttaagtacaggtgtatactagagatatatagaactcaaaggagaaaaatggctacatttgtacactgaaagattgagtgtaaaaaatatttttaaatgatcttttaatagcttatgaaagatgaaatctttattttgtacatttctttgtttttagaaactgtatatatgtattgaggatattgaaaccataaccaccctcctgtcttccctacctaccgcattccaacacctactctccttctcagtttcatgtcctctagttctttaattatccagactccatatccccattgctaggagtcgtagctagggtcaccctcatagactcctgggagttttcattgcccttgtttctagctagtcccagagatgcccatctcctgtaaccagaaaagacttccaatggaggaactgggacaccaacccactcacaaaatctttagctcatagtttgtcctacctacaagatgtgcgggcataaaagatgaaacagaattagagagaagggcaaaccaatgactggcccagcttgacacctatgccatgagagggagcccactccgacactcttaataatattctcctatacatgcagacaggagcctagcatagctatcatctgagaggtttcacctagcattgatggaaacaaatgcagtgacccacagccaaacgttaagtggaacttggggaatcctatggaagagagtgaggatggattgaagatgcccagaggggtcatggtcacattgagaaatactacggaatcaactaacctgggcacataggttttcatatagactaaactttcaaccagaaatcatgcacaagacaagcctagaccctctgcacatatgtaacagttgtgcagctgggtcttcatgtgggactcctaaaaccgaagccagggctgtctctgactacattgcctgcctttggaagcctttctgctaactgggctgccttttctagtctcgagagaagaagatgtgcctagtcttactgcagcttgatatgtcaaggctggttgatatccaagggaggcctccccttttctgtggagaaatggaagaatgttagagaaggttggagggaagggagaaagagggattgggagcaaaggatgggggggggggcgctgcagtcagaatttaggataaataaataacttaatcaattcttaaaatgatttaagaaaaggatccacagaacctacttagttcagctcttgtgctcatgagtgtataactgcccactagagcttgagcaacctaccatgggacacacttttaaagaaaactaactcttaatcgcctggtatttgtcaacctgccaggagggcctcagctagggatgtggcctcttgaacacctgctctgtccatattggaatgtggattggtgtgatctcatgccagtcttgtgtcacaaccatagcttctgtgaaattatgaacacaaagatcctattatttttataatagataatttcactgtatttctctttggctcctgcctcttctgatctttccttctctcctccatggtgttccctgagttcttgaggggagtgtttcatggctggtactataaatctgaccacgaacccgtgccagggaggtcttagagcataggggaaaacacaggactattgtttgtttaaaagacagagtatcaaactgtcttctagacatttgtccttatacccatagaatagtgcagccttgcctctcattagagaagctcttttctcacttctctctcagagagcagagaactgggcgtgaccattgagggttcagccctaagtgggacaactctatcctagcatcattattcctaaaacaagagcatccccaaccaactgaggagcatcgtaagtgtggattttggccttgtgatcctgttacattggaccctgagatcatctgaagcatcacttgaagtctaccaccttgctcaaaagtaagcagttaagttcaggagcttcagggaataccaagatgcaggaaaaaaaaaagtcacactctacctctccgttttctctacctctgcttacagcagcaatccttttgctcagacttcctgtttctggcacttgccttcttctgtgatgttcatagttccctgtctgtggtttagcaggcacagagctggtttgggtctccctctctctcattcacttgcacatacaagcgtgcttcttctctattctttctctctctctctctctctctctctctctctctctctctctctcaga

SEQ ID NO: 78 - DNA, 3961 bp, synthetic construct
  source 1..3961: mol_type = other DNA
gcctccaccactccacatatcatagagttacagatacatgttatcatacattgctatccaaactcagttcttcaaaattgtatgacaagaggtttatttattggattatttccctaggccatgacgcccccccccaaaaaaagaattctttaagaagtgttttccaaatattttttccacagcctcctaaaggacttagaggtgattacccatgctaacatggagtctatttgatctcatgactttctgcacacaaattcacatgatttttgtattttgctctgtggtagaaccatgccctgtgaattactcagtgttctaggaagttgccctcggcaattttgtattcctaggaccaaaagtgctctaatttgaaaaacgctaccataaaataattttcttgaatagcttagaacgtattcccaatttccactggaattaaagtaaaacctttacttccagtaaagacagtggataagatgacaataccaacagtgagcataaaagaccaggtctcattgtagctccatataaacaccaatactgcctccctgcaaacctcactcttcgctttagggtactttgcaacataattacattgtctcattcacaatagatcgttagcaaggagctctgttctatccactccttaaaccaagagcatgatactgtcagagaaaataaggtgtttgtccagtaacagaaatgttttcacaatctacctcaaataaggtgaaagagttgtttgtgtgccttctccttctgccgtttgtttcaagtatgaatgttctctggttttaggattaaacctgtcgtatggcctagtctccccgatcataagaaaactctggaacaactatgtaagaagccaaaaacggtaattgcttgaggtggggaaagaaacaccataatgttgaaatcttagtctaagaatgattaagactgacactcaacttacggtcttttatatatcacataaatgaaagtccttttaagactctgaagaataaagccaagatatgccacagggcagggggttggggaaaaatcaatatttacttcaaagttggagtatcacagctcagtcagaagtgaagccaactgtcattttttcacatcgtgtgtcaattttacaagaaagtttcgtaaacgttttagtttcctgaatcaaatgtatagcagcgcctctttgccacgcctctaacgcttctgcctttctctgcagagtctgaatgtgagtttcaatcccgaaagtttcctggactgccagattcatgaggtgaaaggcgttgaagccagggacgaggtggaaagttttctgcccaatgatcttcctgcacagccagaggagttggagacacagggacacagagccgctgtacacagtgcaaaccgctcgcctgagacttcagtcagcccaccagaaacagttagaagagagtcacccttaagatgcctggctagaaatctgagtacctgcaatgcccctccactcctttcctctaggtcccctgactacagagatggtgacagaaataggcctcctgtgtatcaagacttgctgccaaactctggaaacacaaatgtccctgtccctgtccctcaaccattgcctttccagtcgggaatcctgataccagtttctcagagacagcccatctccacttcctcagtactgaatcaagaagaagcgtatgtcaccatgtctagtttttaccaaaacaaatgaattataagaaaacccttccatcgacaaccaaatgatcactgagatggaaagtctggaatgcttgctctcccccgtagctcacagaagagaaagtcaacgtgaccttgctacacatcttcagcattctaagaaatcattttgctcttctagctcagaagcatttgcacaaagcaggaagaatctgttttccctgttgttggattagtcataagagtccatatgacccaattaaaattgcaaaactcagttaagtgaagaaagaaagatagacaaaagaagatagaaggatgtggtgaatgcaggaagaagaaaatgaaagatgtgagtggtgggtctatcattcaaattgactatttatccagcactataccactcttctcatttcttcctcacaataatattacaatgtgggcttatccattataacttttattttctttgtcatagatgctgaagttgaaagtagagattttaagtgatatccaaatttttctttcagctacagatgaggcacacattccaacttcaaccctctcttgccatgaacctgtcctattgttgagtgtcaaacatcaccactaagtggatggttatgtagtccattatccaaactgagtcgttttggaaagaaaaagttagacataattaacagtaagcataaactgtatatgtctaagagagatgtggatggatggtcattttacttaaagtggctatagggatgaacatgaaggacaaagtacatttatgggtgtggcataccatgaccatgtgtcaaaggaagtgggaaaaagaaaaaaaaagcaccaagatcatttgattttgttttgttgttttgtttgaaaacaaactcaagaagcaatgagttagaagccgagaagttccagagtcagttatcaagaccatgattttcctgctgctattatccattggcttctctgtgacattgtaggaggaactatggccaatctacaggagttcaacatttaacagtgaatggagtcctcctatgtgagtcctcctatgtgtggagacaccattaagaactaccccaagttctacatctctggatattgcctgaactacagaaaaagggggctgcgcacaccacaatgagtgccctacctgaaactatgctcacagaaacacaaagaagatgggtaagttattcaaattcaaatgttgatttatgactgcaagtcacaattttgaatccctgctgtgtataaccaatctcctgaagaaaacaacaaataactgaaagatactgtggttgggtgccttagcattaaaattctgtttaagtgttgacattgtttatttggattggagtgtctgtccggtcatgtattgtatccatgcattatattcagataaccacaacagctgctaatgcttgattatattctcagggactgcatgcaatgtaacattactggttggttctgccaattttcctcttggtatttataaaggaaaaccaaaactcttggtcagagacaatatgcaaaacagagatgtcaagtactatgtccaaatactgtgaaatataatgagaaataggtaacaaatttatcaatcaactatgtttggatccagggaatctcaagttattcaattcattctctgtaagcctttgtctctctcttcatccagacttttgccttcaaatacaagcatgcgctattttctggaattgatacagcactgttcattttgtgtgtgtgtgtgtgtatgtgtgtgtgtgtgtgtgtgtgtgtgtgtgtgtgtgtgtatgtgagagagagagagagagagagagagagagagagagagagagagagagctcagcagtttttctctggtgttctttaatacatccaccattcactacagtgaagatctctgggttgactgtacactttttaaaatgaattagtagtattctctaatgatgttacacagagaaatgtatggtgtgtgttaccatttcatgttcaactaaatgctaaggtactctgtggctaaaatactctctgtaactgtggcaactacatgtaaataaatgggctctacagttcagcacagacttcttgagctactgaaaatattcttagaccatttaaaaggttaagattgtcagaaagatagttttaaattaattcaataatgaactaatagccatttattgagaagccataatgccttacttaaacactatgctggctactaaacaatggaacgagatactcc

SEQ ID NO: 79 - DNA, 60 bp, synthetic construct
  source 1..60: mol_type = other DNA
cacaatctattcttgctttccaggggagatggatcctgtcttgccaagtgtcaccattct

SEQ ID NO: 80 - RNA, 3227 nt, synthetic construct
  source 1..3227: mol_type = other RNA
gtctgtggtttagcaggcacagagctggtttgggtctccctctctctcattcacttgcacatacaagcgtgcttcttctctattctttctctctctctctctctctctctctctctctctctctctctctcagaatgacaattctaggtacaacttttggcatggttttttctttacttcaagtcgtttctggagaaagtggctatgctcaaaatggagacttggaagatgcagaactggatgactactcattctcatgctatagccagttggaagtgaatggatcgcagcactcactgacctgtgcttttgaggacccagatgtcaacatcaccaatctggaatttgaaatatgtggggccctcgtggaggtaaagtgcctgaatttcaggaaactacaagagatatatttcatcgagacaaagaaattcttactgattggaaagagcaatatatgtgtgaaggttggagaaaagagtctaacctgcaaaaaaatagacctaaccactatagttaaacctgaggctccttttgacctgagtgtcgtctatcgggaaggagccaatgactttgtggtgacatttaatacatcacacttgcaaaagaagtatgtaaaagttttaatgcacgatgtagcttaccgccaggaaaaggatgaaaacaaatggacgcatgtgaatttatccagcacaaagctgacactcctgcagagaaagctccaaccggcagcaatgtatgagattaaagttcgatccatccctgatcactattttaaaggcttctggagtgaatggagtccaagttattacttcagaactccagagatcaataatagctcaggggagatggatcctgtcttgccaagtgtcaccattctgagtttgttctctgtgtttttgttggtcatcttagcccatgtgctatggaaaaaaaggattaaacctgtcgtatggcctagtctccccgatcataagaaaactctggaacaactatgtaagaagccaaaaacgagtctgaatgtgagtttcaatcccgaaagtttcctggactgccagattcatgaggtgaaaggcgttgaagccagggacgaggtggaaagttttctgcccaatgatcttcctgcacagccagaggagttggagacacagggacacagagccgctgtacacagtgcaaaccgctcgcctgagacttcagtcagcccaccagaaacagttagaagagagtcacccttaagatgcctggctagaaatctgagtacctgcaatgcccctccactcctttcctctaggtcccctgactacagagatggtgacagaaataggcctcctgtgtatcaagacttgctgccaaactctggaaacacaaatgtccctgtccctgtccctcaaccattgcctttccagtcgggaatcctgataccagtttctcagagacagcccatctccacttcctcagtactgaatcaagaagaagcgtatgtcaccatgtctagtttttaccaaaacaaatgaattataagaaaacccttccatcgacaaccaaatgatcactgagatggaaagtctggaatgcttgctctcccccgtagctcacagaagagaaagtcaacgtgaccttgctacacatcttcagcattctaagaaatcattttgctcttctagctcagaagcatttgcacaaagcaggaagaatctgttttccctgttgttggattagtcataagagtccatatgacccaattaaaattgcaaaactcagttaagtgaagaaagaaagatagacaaaagaagatagaaggatgtggtgaatgcaggaagaagaaaatgaaagatgtgagtggtgggtctatcattcaaattgactatttatccagcactataccactcttctcatttcttcctcacaataatattacaatgtgggcttatccattataacttttattttctttgtcatagatgctgaagttgaaagtagagattttaagtgatatccaaatttttctttcagctacagatgaggcacacattccaacttcaaccctctcttgccatgaacctgtcctattgttgagtgtcaaacatcaccactaagtggatggttatgtagtccattatccaaactgagtcgttttggaaagaaaaagttagacataattaacagtaagcataaactgtatatgtctaagagagatgtggatggatggtcattttacttaaagtggctatagggatgaacatgaaggacaaagtacatttatgggtgtggcataccatgaccatgtgtcaaaggaagtgggaaaaagaaaaaaaaagcaccaagatcatttgattttgttttgttgttttgtttgaaaacaaactcaagaagcaatgagttagaagccgagaagttccagagtcagttatcaagaccatgattttcctgctgctattatccattggcttctctgtgacattgtaggaggaactatggccaatctacaggagttcaacatttaacagtgaatggagtcctcctatgtgagtcctcctatgtgtggagacaccattaagaactaccccaagttctacatctctggatattgcctgaactacagaaaaagggggctgcgcacaccacaatgagtgccctacctgaaactatgctcacagaaacacaaagaagatgggtaagttattcaaattcaaatgttgatttatgactgcaagtcacaattttgaatccctgctgtgtataaccaatctcctgaagaaaacaacaaataactgaaagatactgtggttgggtgccttagcattaaaattctgtttaagtgttgacattgtttatttggattggagtgtctgtccggtcatgtattgtatccatgcattatattcagataaccacaacagctgctaatgcttgattatattctcagggactgcatgcaatgtaacattactggttggttctgccaattttcctcttggtatttataaaggaaaaccaaaactcttggtcagagacaatatgcaaaacagagatgtcaagtactatgtccaaatactgtgaaatataatgagaaataggtaacaaatttatcaatcaactatgtttggatccagggaatctcaagttattcaattcattctctgtaagcctttgtctctctcttcatccagacttttgccttcaaatacaagcatgcgctattttctggaaaaaaaaaaaaaaaaaaa

SEQ ID NO: 81 - protein, 459 aa, synthetic construct
MTILGTTFGMVFSLLQVVSGESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPVLPSVTILSLFSVFLLVILAHVLWKKRIKPVVWPSLPDHKKTLEQLCKKPKTSLNVSFNPESFLDCQIHEVKGVEARDEVESFLPNDLPAQPEELETQGHRAAVHSANRSPETSVSPPETVRRESPLRCLARNLSTCNAPPLLSSRSPDYRDGDRNRPPVYQDLLPNSGNTNVPVPVPQPLPFQSGILIPVSQRQPISTSSVLNQEEAYVTMSSFYQNK

SEQ ID NO: 82 - protein, 223 aa, Rattus norvegicus
MGAKPNLIGKNRLLSDILVPISNYSCALQQTSLPKRSNVIAVTLKTTCLPVKSIVLGSRGSLCLEGWFQEKQSRTRVSGNIFSLCVSPVLFRYLFILQVVRLALTYNFSNCNFEMILRIYHATIFRDLLKDLNGILFDQIEDCDSRTACLLKIDHHTFNPVPGCPSLPEKAFALKTKAALINYCPGYSETERNGTLEMTREIRNICLNQTSQILGLWLSCIQS

SEQ ID NO: 83 - protein, 360 aa, Rattus norvegicus
MRAVTWAIVAMLLPRVLGAIPTRTPRTGGVGDTLSVAIVCHDLESVEVTWGPGSAHHGLSANLSLEFRYGNQVPQPCPHYFLLDSVRAGCVLPMGKGLLEVVLREGGGAKLFSRKKKASAWLRPRPPWNVTLSWVGDTVAVSCPSHSYPGLEYEVQHRDDFDPEWQSTSAPFCNLTVGGLDPGRCYDFRVRATPQDFYYGPEARPSKWTGVASLQGVGPTGSCTGPTLPRTPGTPTPPLALACGLAVALLTLVLLLALLRMRRVKEALLPGVPDPRGSFPGLFEKHHGNFQAWIADSQAAVPTVPEQDKDDDVIRPQTKGVETQEEDDVIAPGSPCLGGGALMSVGGASFLMGDSGYTTL

SEQ ID NO: 84 - protein, 457 aa, Rattus norvegicus
MMALGRAFAIVFCLLQAASGESGNAQDGDLEDAEPDDHSFWCHSQLEVDGNQHSLTCAFNDPDIKTTNLEFQICGALLGIDCLTLNKLREMYFIKTSKFLLIGNSSVCVKLGKMDVICKILDISTIVKPEAPSNLKVVYRKEANDFLVTFNTSHSTKKYVTALKHDVAYRPERGESNWTHVYLFHTRTTILQRKLQPKAVYEIKVRSIPNHEYFKGFWSEWSPSSTFETPDSKYQGGWDPVLPSIILLSLFSMVLLVILAHVLWKKRIKPVVWPSLPDHKKTLEQLCKKPKKNLNVSFNPESFLDCQIHEVNGIQARDEVESFLQNDLPPRPGELEKQGHRATVHGANWPSEISGSTPETFRRESPLRCLARNLSTCNTPAFLSSRSPDYREGDGNRSHVYQDLLPSSRNTNGTVPQPFPLQSGILIPVSQGQPISTSSVLNQEEAYVTMSSFYQNK

SEQ ID NO: 85 - RNA, 2550 nt, synthetic construct
  source 1..2550: mol_type = other RNA
aagaggaactgcagctgcagaagggagaaatcacggcagaatcatcgagaaacctgaaaaatgaagcctaaaatgaagtattcaaccaacaaaatttccacagcaaagtggaagaacacagcaagcaaagccttgtgtttcaagctgggaaaatcccaacagaaggccaaagaagtttgccccatgtactttatgaagctccgctctggccttatgataaaaaaggaggcctgttactttaggagagaaaccaccaaaaggccttcactgaaaacaggtagaaagcacaaaagacatctggtactcgctgcctgtcaacagcagtctactgtggagtgctttgcctttggtatatcaggggtccagaaatatactagagcacttcatgattcaagtatcacaggaatttcacctattacagagtatcttgcttctctaagcacatacaatgatcaatccattacttttgctttggaggatgaaagttatgagatatatgttgaagacttgaaaaaagatgaaaagaaagataaggtgttactgagttactatgagtctcaacacccctcaaatgaatcaggtgacggtgttgatggtaagatgttaatggtaaccctgagtcctacaaaagacttctggttgcatgccaacaacaaggaacactctgtggagctccataagtgtgaaaaaccactgccagaccaggccttctttgtccttcataatatgcactccaactgtgtttcatttgaatgcaagactgatcctggagtgtttataggtgtaaaggataatcatcttgctctgattaaagtagactcttctgagaatttgtgtactgaaaatatcttgtttaagctctctgaaacttagtgcagtaaaacgcctgtgcgttctgggttgaatgacttaatgcttccaactgaagaaagggtaacagagagaaagaaagccattcttggcttacgatgttgtggaatgttattatgtagaaaacttcttttatttccttttcttcagctacatgttcaatagactcacagatatatgacttacggcgttggtaaagaaactgaaggagattcagccttgctctttccttttctctgccttgagtcctgtatgaaatcacactcacggacttcagaagagcaggcaccacagtgcatggtttgctttagagagggtccatttcaaaaaccttcataaaaacaatgcaaaacaagaaaacaaccgaacaaaaaaaccacctatttcctggttctaaacaaatgattgtaatactagagcagttagtgggaggaccagctagggggaggatcacctaggggaggaccagctagggggaggaccagctgctgcaaagactgactgtttctcacttataataaaatgccaaatgcctccgcagatgccccaggcaaccctcagatcagccctttctgtgaagagtggcgttacctgtgcttgtttccttcttaaacttccaatttttctcttttaacacatttaacatttaactttaagcaagccagcttacattaggaagtgaaagacattttagttcccacccgtgattgaaatcattgactatatctaacaagcttaaagtctcctgtaagaactgatcaggatatacactaggacatgccaatagaatgggatctcatggtgcagtctgaagccctccaactggagagacgctaacatcatccttctcgctgatttccaaggagctatgactttggatgcatgcatctgcttggatgagatgtctcggctgcttgctttccttatgcacacgttctgttcagcttcacagcagcaatgctcacggtggaatagcttagcttagcttctgccccttctttggttcttttgaccaccatatccgtaacggctctcctactccctcagctttctttctctttgctctgacgtctatatgccaacacacttattccactgtctttaccctgcactgcagaattttacatctacctactggttaccaggttgtcccctgaacaaccttcctttgtgttttactgttattaaagtagtaatatttgtattcaaccatgtagtaatattttaagccactaaaggaatagttttacttatttagacaacagcaatttctactacatttttataagcttaaaacttacatgttttaaaacttaaaacgataaagacaataacaacattgatggagtatgatatgacagttcagaaagggttagctcttatcttccagtcgaggaaacctattgtatacaatagctggaggaatgtatgatcaaagaggccgggaaccgccgtgtaggatcgtacggctgtaacaggtataattgtttcattaatttgtcacagtcttactgtagaggaatgtaaaggcggaatctgcgtcattcctctggaaaccacagtgttgactctgtgaatctgtacgatatctttaaagtagtaactacgtagtcaaatgtgttcttgacgttgttcataactttgaataaaccatttttcaaaaccacgtgtgaccacaaa

SEQ ID NO: 86 - DNA, 28 bp, synthetic construct
  source 1..28: mol_type = other DNA
ccattaacggtctattgctcatttccca

SEQ ID NO: 87 - DNA, 28 bp, synthetic construct
  source 1..28: mol_type = other DNA
tgctactcaggcaatgaagaagaggaga

SEQ ID NO: 88 - DNA, 24 bp, synthetic construct
  source 1..24: mol_type = other DNA
gctccaatttcccacaacattagt

(19) United States
(12) Patent Application Publication
(10) Pub. No.: US 2025/0134079 A1
(43) Pub. Date: May 1, 2025

(54) GENETICALLY MODIFIED NON-HUMAN ANIMAL WITH HUMAN OR CHIMERIC GENES

(71) Applicant: Biocytogen Pharmaceuticals (Beijing) Co., Ltd., Beijing (CN)

(72) Inventors: Shujin Zhang, Beijing (CN); Yanhui Nie, Beijing (CN); Jiahui Chang, Beijing (CN)

(21) Appl. No.: 18/693,612

(22) PCT Filed: Sep. 27, 2022
(86) PCT No.: PCT/CN2022/121767

§ 371 Date: Mar. 20, 2024

(30) Foreign Application Priority Data
Sep. 27, 2021 (CN) 202111138918.9
Dec. 6, 2021 (CN) 202111481782.1
Jun. 7, 2022 (CN) 202210638998.2

(51) Int. Cl. A01K 67/0278; C07K 14/54; C07K 14/715

(52) U.S. Cl. CPC A01K 67/0278; C07K 14/54; C07K 14/7155; A01K 2207/15; A01K 2217/052; A01K 2227/105; A01K 2267/0368; A01K 2267/0387

(57) ABSTRACT

Provided herein are genetically modified non-human animals that express a human or chimeric (e.g., humanized) TSLP and/or TSLPR, and methods of use thereof. In some embodiments, the animals also express a human or chimeric (e.g., humanized) IL33 and/or IL7R.

### CLAIM OF PRIORITY

This application claims the benefit of Chinese Patent Application App. No. 202111138918.9, filed on Sep. 27, 2021; Chinese Patent Application App. No. 202111481782.1, filed on Dec. 6, 2021; and Chinese Patent Application App. No. 202210638998.2, filed on Jun. 7, 2022. The entire contents of the foregoing applications are incorporated herein by reference.

### TECHNICAL FIELD

This disclosure relates to genetically modified animals expressing human or chimeric (e.g., humanized) TSLP and/or TSLP receptor proteins, and methods of use thereof. In some embodiments, the animals also express a human or chimeric (e.g., humanized) IL33 and/or IL7R.

### BACKGROUND

The traditional drug research and development typically use in vitro screening approaches. However, these screening approaches cannot provide the body environment (such as tumor microenvironment, stromal cells, extracellular matrix components and immune cell interaction, etc.), resulting in a higher rate of failure in drug development. In addition, in view of the differences between humans and animals, the test results obtained from the use of conventional experimental animals for in vivo pharmacological test may not reflect the real disease state and the interaction at the targeting sites, resulting in that the results in many clinical trials are significantly different from the animal experimental results.

Therefore, the development of humanized animal models that are suitable for human antibody screening and evaluation will significantly improve the efficiency of new drug development and reduce the cost for drug research and development.

### SUMMARY

This disclosure is related to an animal model with human or chimeric TSLP and/or TSLP receptor proteins. The animal model can express human or chimeric TSLP (e.g., humanized TSLP) protein and/or human or chimeric TSLPR (e.g., humanized TSLPR) protein in its body. It can be used in the studies on the function of TSLP and TSLPR genes, and can be used in the screening and evaluation of TSLP/TSLPR signaling pathway modulators (e.g., anti-human TSLP antibodies or anti-human TSLPR antibodies). In addition, the animal models prepared by the methods described herein can be used in drug screening, pharmacodynamics studies, treatments for immune-related diseases, and cancer therapy for human TSLP/TSLPR target sites; they can also be used to facilitate the development and design of new drugs, and save time and cost. In summary, this disclosure provides a powerful tool for studying the function of TSLP/TSLPR protein and a platform for screening cancer drugs.

In one aspect, the disclosure is related to a genetically-modified, non-human animal whose genome comprises at least one chromosome comprising a sequence encoding a human or chimeric thymic stromal lymphopoietin (TSLP). In some embodiments, the sequence encoding the human or chimeric TSLP is operably linked to an endogenous regulatory element (e.g., endogenous 5′ UTR and/or 3′ UTR) at the endogenous TSLP gene locus in the at least one chromosome. In some embodiments, the sequence encoding a human or chimeric TSLP comprises a sequence encoding an amino acid sequence that is at least 70%, 75%, 80%, 85%, 90%, 95%, 99%, or 100% identical to human TSLP (NP_149024.1; SEQ ID NO: 2). In some embodiments, the animal is a mammal, e.g., a monkey, a rodent, a mouse, or a rat. In some embodiments, the animal is a mouse. In some embodiments, the animal does not express endogenous TSLP or expresses a decreased level of endogenous TSLP as compared to TSLP expression level in a wild-type animal. In some embodiments, the animal has one or more cells expressing human or chimeric TSLP. In some embodiments, the animal has one or more cells expressing human or chimeric TSLP, and endogenous TSLP receptor (TSLPR) can bind to the expressed human or chimeric TSLP, inducing downstream signaling pathways (e.g., inducing release of thymus activation-regulated chemokine (TARC)). In some embodiments, the animal has one or more cells expressing human or chimeric TSLP, and human TSLP receptor (TSLPR) can bind to the expressed human or chimeric TSLP, inducing downstream signaling pathways (e.g., inducing release of thymus activation-regulated chemokine (TARC)).

In one aspect, the disclosure is related to a genetically-modified, non-human animal, in some embodiments, the genome of the animal comprises a replacement of a sequence encoding a region of endogenous TSLP with a sequence encoding a corresponding region of human TSLP at an endogenous TSLP gene locus. In some embodiments, the sequence encoding the corresponding region of human TSLP is operably linked to an endogenous regulatory element at the endogenous TSLP locus, and one or more cells of the animal express a human or chimeric TSLP. In some embodiments, the animal does not express endogenous TSLP or expresses a decreased level of endogenous TSLP as compared to TSLP expression level in a wild-type animal. In some embodiments, the replaced sequence encodes the full-length protein of TSLP. In some embodiments, the animal is a mouse, and the replaced endogenous TSLP region comprises a portion of exon 1, exon 2, exon 3, exon 4, and/or a portion of exon 5 of the endogenous mouse TSLP gene. In some embodiments, the animal is heterozygous or homozygous with respect to the replacement at the endogenous TSLP gene locus.

In one aspect, the disclosure is related to a non-human animal comprising at least one cell comprising a nucleotide sequence encoding a humanized TSLP polypeptide, in some embodiments, the humanized TSLP polypeptide comprises at least 50, 100, 110, 120, 130, 140, 150, 155, 156, 157, 158, or 159 contiguous amino acid residues that are identical to the corresponding contiguous amino acid sequence of a human TSLP, in some embodiments, the animal expresses the humanized TSLP polypeptide. In some embodiments, the nucleotide sequence is operably linked to an endogenous TSLP regulatory element of the animal. In some embodiments, the nucleotide sequence is integrated to an endogenous TSLP gene locus of the animal. In some embodiments, the humanized TSLP polypeptide has at least one mouse TSLP activity and/or at least one human TSLP activity.

In one aspect, the disclosure is related to a method for making a genetically-modified, non-human animal, comprising: replacing in at least one cell of the animal, at an endogenous TSLP gene locus, a sequence encoding a region of an endogenous TSLP with a sequence encoding a corresponding region of human TSLP. In some embodiments, the sequence encoding the corresponding region of human TSLP comprises a portion of exon 1, exon 2, exon 3, and/or a portion of exon 4 of a human TSLP gene. In some embodiments, the sequence encoding the corresponding region of human TSLP comprises at least 50, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, or 480 nucleotides of exon 1, exon 2, exon 3, and/or exon 4 of a human TSLP gene. In some embodiments, the sequence encoding the corresponding region of human TSLP encodes a sequence that is at least 80%, 85%, 90%, 95%, 96%, 97%, 98%, 99%, or 100% identical to SEQ ID NO: 2. In some embodiments, the animal is a mouse, and the locus is a portion of exon 1, exon 2, exon 3, exons 4, and/or a portion of exon 5 of the mouse TSLP gene.

In one aspect, the disclosure is related to a method of making a genetically-modified non-human animal cell that expresses a human or chimeric TSLP, the method comprising: replacing, at an endogenous mouse TSLP gene locus, a nucleotide sequence encoding a region of endogenous TSLP with a nucleotide sequence encoding a corresponding region of human TSLP, thereby generating a genetically-modified non-human animal cell that includes a nucleotide sequence that encodes the human or chimeric TSLP, in some embodiments, the animal cell expresses the human or chimeric TSLP. In some embodiments, the animal is a mouse. In some embodiments, the nucleotide sequence encoding the human or chimeric TSLP is operably linked to an endogenous TSLP regulatory region, e.g., promoter.

In some embodiments, the animal further comprises a sequence encoding an additional human or chimeric protein, e.g., TSLP receptor (TSLPR), IL33, IL7R, IL6, IL12, IL23, and/or Tumor necrosis factor alpha (TNF-α). In some embodiments, the additional human or chimeric protein is TSLPR, IL33, and/or IL7R.

In one aspect, the disclosure is related to a genetically-modified, non-human animal whose genome comprises at least one chromosome comprising a sequence encoding a human or chimeric thymic stromal lymphopoietin receptor (TSLPR). In some embodiments, the sequence encoding the human or chimeric TSLPR is operably linked to an endogenous regulatory element at the endogenous TSLPR gene locus in the at least one chromosome. In some embodiments, the sequence encoding a human or chimeric TSLPR comprises a sequence encoding an amino acid sequence that is at least 70%, 75%, 80%, 85%, 90%, 95%, 99%, or 100% identical to human TSLPR (NP_071431.2; SEQ ID NO: 8). In some embodiments, the sequence encoding a human or chimeric TSLPR comprises a sequence encoding an amino acid sequence that is at least 70%, 75%, 80%, 85%, 90%, 95%, 99%, or 100% identical to amino acids 1-253 or 2-253 of human TSLPR (NP_071431.2; SEQ ID NO: 8). In some embodiments, the sequence encoding a human or chimeric TSLPR comprises a sequence encoding an amino acid sequence that is at least 70%, 75%, 80%, 85%, 90%, 95%, 99%, or 100% identical to SEQ ID NO: 19. In some embodiments, the animal is a mammal, e.g., a monkey, a rodent, a mouse, or a rat. In some embodiments, the animal is a mouse. In some embodiments, the animal does not express endogenous TSLPR or expresses a decreased level of endogenous TSLPR as compared to TSLPR expression level in a wild-type animal. In some embodiments, the animal has one or more cells expressing human or chimeric TSLPR. In some embodiments, the animal has one or more cells expressing human or chimeric TSLPR that can bind to endogenous TSLP and IL7 receptor (IL7R) to induce downstream signaling pathways (e.g., inducing release of thymus activation-regulated chemokine (TARC)). In some embodiments, the animal has one or more cells expressing human or chimeric TSLPR that can bind to human TSLP and IL7R to induce downstream signaling pathways (e.g., inducing release of thymus activation-regulated chemokine (TARC)).

In one aspect, the disclosure is related to a genetically-modified, non-human animal, in some embodiments, the genome of the animal comprises an insertion of a sequence encoding a human or chimeric TSLPR at an endogenous TSLPR gene locus. In some embodiments, the sequence encoding a human or chimeric TSLPR is operably linked to an endogenous regulatory element at the endogenous TSLPR locus, and one or more cells of the animal express the human or chimeric TSLPR. In some embodiments, the animal does not express endogenous TSLPR or expresses a decreased level of endogenous TSLPR as compared to TSLPR expression level in a wild-type animal. In some embodiments, the sequence encoding a human or chimeric TSLPR is inserted within exon 1, intron 1, exon 2, intron 2, exon 3, intron 3, exon 4, intron 4, exon 5, intron 5, exon 6, intron 6, exon 7, intron 7, and/or exon 8 of endogenous TSLPR gene.

In some embodiments, the sequence encoding a human or chimeric TSLPR is inserted within exon 2 of endogenous TSLPR gene. In some embodiments, the sequence encoding a human or chimeric TSLPR is inserted immediately after a nucleotide corresponding to position 260 of NM_016715.4. In some embodiments, the inserted sequence comprises, optionally from 5′ end to 3′ end: a) a sequence encoding a self-cleaving peptide; b) a first sequence comprising a sequence encoding the extracellular region and transmembrane region of a human TSLPR; c) a second sequence comprising a sequence encoding all or a portion of the cytoplasmic region of an endogenous TSLPR; d) a regulatory sequence of endogenous TSLPR gene (e.g., 3′ UTR); and e) optionally one or more auxiliary sequences (e.g., WPRE, lox2, STOP, and/or polyA). In some embodiments, the self-cleaving peptide is T2A, P2A, E2A, or F2A (e.g., P2A). In some embodiments, the first sequence further comprises a sequence encoding at least 1, at least 2, at least 3, at least 4, at least 5, or at least 6 amino acids of the cytoplasmic region of a human TSLPR, and the second sequence does not comprise a corresponding sequence encoding at least 1, at least 2, at least 3, at least 4, at least 5, or at least 6 amino acids of the cytoplasmic region of an endogenous TSLPR. In some embodiments, the first sequence is at least 70%, 75%, 80%, 85%, 90%, 95%, 99%, or 100% identical to SEQ ID NO: 15, and the second sequence is at least 70%, 75%, 80%, 85%, 90%, 95%, 99%, or 100% identical to SEQ ID NO: 16. In some embodiments, the first sequence encodes an amino acid that is at least 70%, 75%, 80%, 85%, 90%, 95%, 99%, or 100% identical to amino acids 1-253 of SEQ ID NO: 8, and the second sequence encodes an amino acid sequence that is at least 70%, 75%, 80%, 85%, 90%, 95%, 99%, or 100% identical to amino acids 255-359 of SEQ ID NO: 7. In some embodiments, the one or more auxiliary sequences comprise a STOP sequence.

In some embodiments, the sequence encoding a human or chimeric TSLPR is inserted within exon 1 of endogenous TSLPR gene. In some embodiments, the sequence encoding a human or chimeric TSLPR is inserted immediately after a nucleotide corresponding to position 113 of NM_016715.4. In some embodiments, the inserted sequence comprises, optionally from 5′ end to 3′ end: a) a first sequence comprising a sequence encoding all or a portion of the extracellular region and transmembrane region of a human TSLPR; b) a second sequence comprising a sequence encoding all or a portion of the cytoplasmic region of an endogenous TSLPR; c) a regulatory sequence of endogenous TSLPR gene (e.g., 3′ UTR); and d) optionally one or more auxiliary sequences (e.g., WPRE, lox2, STOP, and/or polyA). In some embodiments, the first sequence further comprises a sequence encoding at least 1, at least 2, at least 3, at least 4, at least 5, or at least 6 amino acids of the cytoplasmic region of a human TSLPR, and the second sequence does not comprise a corresponding sequence encoding at least 1, at least 2, at least 3, at least 4, at least 5, or at least 6 amino acids of the cytoplasmic region of an endogenous TSLPR. In some embodiments, the first sequence is at least 70%, 75%, 80%, 85%, 90%, 95%, 99%, or 100% identical to SEQ ID NO: 69, and the second sequence is at least 70%, 75%, 80%, 85%, 90%, 95%, 99%, or 100% identical to SEQ ID NO: 16. In some embodiments, the first sequence encodes an amino acid that is at least 70%, 75%, 80%, 85%, 90%, 95%, 99%, or 100% identical to amino acids 2-253 of SEQ ID NO: 8, and the second sequence encodes an amino acid sequence that is at least 70%, 75%, 80%, 85%, 90%, 95%, 99%, or 100% identical to amino acids 255-359 of SEQ ID NO: 7. In some embodiments, the one or more auxiliary sequences comprise a STOP sequence.

In some embodiments, the animal is heterozygous or homozygous with respect to the insertion at the endogenous TSLPR gene locus.

In one aspect, the disclosure is related to a non-human animal comprising at least one cell comprising a nucleotide sequence encoding a humanized TSLPR polypeptide, in some embodiments, the humanized TSLPR polypeptide comprises at least 50 contiguous amino acid residues that are identical to the corresponding contiguous amino acid sequence of a human TSLPR, in some embodiments, the animal expresses the humanized TSLPR polypeptide. In some embodiments, the humanized TSLPR polypeptide has at least 50, 100, 150, 160, 170, 180, 190, 200, 210, 220, 230, 240, 250, 251, or 252 contiguous amino acid residues that are identical to the corresponding contiguous amino acid sequence of human TSLPR extracellular and transmembrane regions. In some embodiments, the humanized TSLPR polypeptide comprises a sequence that is at least 90%, 95%, or 99% identical to amino acids 1-253 or 2-253 of SEQ ID NO: 8. In some embodiments, the nucleotide sequence is operably linked to an endogenous TSLPR regulatory element of the animal. In some embodiments, the nucleotide sequence is integrated to an endogenous TSLPR gene locus of the animal. In some embodiments, the humanized TSLPR polypeptide has at least one mouse TSLPR activity and/or at least one human TSLPR activity.

In one aspect, the disclosure is related to a method of making a genetically-modified non-human animal cell that expresses a chimeric TSLPR, the method comprising: inserting at an endogenous TSLPR gene locus (e.g., exon 2 of endogenous TSLPR gene), a nucleotide sequence comprising, optionally from 5′ end to 3′ end: a) a sequence encoding a self-cleaving peptide; b) a first sequence comprising a sequence encoding the extracellular region and transmembrane region of a human TSLPR; c) a second sequence comprising a sequence encoding all or a portion of the cytoplasmic region of an endogenous TSLPR; d) a regulatory sequence of endogenous TSLPR gene (e.g., 3′ UTR); and e) optionally one or more auxiliary sequences (e.g., WPRE, lox2, STOP, and/or polyA); thereby generating a genetically-modified non-human animal cell that includes a nucleotide sequence that encodes the chimeric TSLPR, in some embodiments, the animal cell expresses the chimeric TSLPR.

In one aspect, the disclosure is related to a method of making a genetically-modified non-human animal cell that expresses a chimeric TSLPR, the method comprising: inserting at an endogenous TSLPR gene locus (e.g., exon 1 of endogenous TSLPR gene), a nucleotide sequence comprising, optionally from 5′ end to 3′ end: a) a first sequence comprising a sequence encoding all or a portion of the extracellular region and transmembrane region of a human TSLPR; b) a second sequence comprising a sequence encoding all or a portion of the cytoplasmic region of an endogenous TSLPR; c) a regulatory sequence of endogenous TSLPR gene (e.g., 3′ UTR); and d) optionally one or more auxiliary sequences (e.g., WPRE, lox2, STOP, and/or polyA); thereby generating a genetically-modified non-human animal cell that includes a nucleotide sequence that encodes the chimeric TSLPR, in some embodiments, the animal cell expresses the chimeric TSLPR.

In some embodiments, the animal is a mouse. In some embodiments, the nucleotide sequence encoding the chimeric TSLPR polypeptide is operably linked to an endogenous TSLPR regulatory region, e.g., promoter.

In some embodiments, the animal further comprises a sequence encoding an additional human or chimeric protein, e.g., TSLP, IL33, IL7R, IL6, IL12, IL23, and/or Tumor necrosis factor alpha (TNF-α). In some embodiments, the additional human or chimeric protein is TSLP, IL33, and/or IL7R.

In one aspect, the disclosure is related to a genetically-modified, non-human animal whose genome comprises at least one chromosome comprising a sequence encoding a human or chimeric interleukin 7 receptor (IL7R). In some embodiments, the sequence encoding the human or chimeric IL7R is operably linked to an endogenous regulatory element at the endogenous IL7R gene locus in the at least one chromosome. In some embodiments, the sequence encoding a human or chimeric IL7R comprises a sequence encoding an amino acid sequence that is at least 70%, 75%, 80%, 85%, 90%, 95%, 99%, or 100% identical to human IL7R (NP_002176.2 (SEQ ID NO: 76)). In some embodiments, the sequence encoding a human or chimeric IL7R comprises a sequence encoding an amino acid sequence that is at least 70%, 75%, 80%, 85%, 90%, 95%, 99%, or 100% identical to SEQ ID NO: 81. In some embodiments, the sequence encoding a human or chimeric IL7R comprises a sequence encoding an amino acid sequence that is at least 70%, 75%, 80%, 85%, 90%, 95%, 99%, or 100% identical to amino acids 1-239 of SEQ ID NO: 76. In some embodiments, the animal is a mammal, e.g., a monkey, a rodent, a mouse, or a rat. In some embodiments, the animal is a mouse. In some embodiments, the animal does not express endogenous IL7R or expresses a decreased level of endogenous IL7R as compared to IL7R expression level in a wild-type animal. In some embodiments, the animal has one or more cells expressing human or chimeric IL7R. In some embodiments, the animal has one or more cells expressing human or chimeric IL7R, and the expressed human or chimeric IL7R can interact with human IL7 or TSLPR. In some embodiments, the animal has one or more cells expressing human or chimeric IL7R, and the expressed human or chimeric IL7R can interact with endogenous IL7 or TSLPR.

In one aspect, the disclosure is related to a genetically-modified, non-human animal, in some embodiments, the genome of the animal comprises a replacement of a sequence encoding a region of endogenous IL7R with a sequence encoding a corresponding region of human IL7R at an endogenous IL7R gene locus. In some embodiments, the sequence encoding the corresponding region of human IL7R is operably linked to an endogenous regulatory element at the endogenous IL7R locus, and one or more cells of the animal expresses a human or chimeric IL7R. In some embodiments, the animal does not express endogenous IL7R or expresses a decreased level of endogenous IL7R as compared to IL7R expression level in a wild-type animal. In some embodiments, the replaced sequence encodes the extracellular region of IL7R. In some embodiments, the animal has one or more cells expressing a chimeric IL7R having an extracellular region, a transmembrane region, and a cytoplasmic region, in some embodiments, the extracellular region comprises a sequence that is at least 50%, 60%, 70%, 80%, 90%, 95%, or 99% identical to the extracellular region of human IL7R (NP_002176.2 (SEQ ID NO: 76)). In some embodiments, the extracellular region of the chimeric IL7R has a sequence that has at least 10, 20, 30, 40, 50, 60, 70, 80, 90, 100, 110, 120, 130, 140, 150, 160, 170, 180, 190, 200, 210, 220, 230, 235, 236, 237, 238, or 239 contiguous amino acids that are identical to a contiguous amino acids sequence present in the extracellular region of human IL7R. In some embodiments, the sequence encoding a region of endogenous IL7R comprises exon 1, exon 2, exon 3, exon 4, exon 5, and/or exon 6, or a part thereof, of the endogenous IL7R gene. In some embodiments, the animal is a mouse. In some embodiments, the animal is heterozygous or homozygous with respect to the replacement at the endogenous IL7R gene locus.

In one aspect, the disclosure is related to a non-human animal comprising at least one cell comprising a nucleotide sequence encoding a humanized IL7R polypeptide, in some embodiments, the humanized IL7R polypeptide comprises at least 50 contiguous amino acid residues that are identical to the corresponding contiguous amino acid sequence of a human IL7R, in some embodiments, the animal expresses the humanized IL7R polypeptide. In some embodiments, the humanized IL7R polypeptide has at least 50, 80, 100, 120, 140, 160, 180, 200, 210, 220, 230, 235, 236, 237, 238, or 239 contiguous amino acid residues that are identical to the corresponding contiguous amino acid sequence of a human IL7R extracellular region. In some embodiments, the humanized IL7R polypeptide comprises a sequence that is at least 90%, 95%, or 99% identical to amino acids 1-239 of SEQ ID NO: 76. In some embodiments, the nucleotide sequence is operably linked to an endogenous IL7R regulatory element of the animal. In some embodiments, the humanized IL7R polypeptide comprises an endogenous IL7R cytoplasmic region and/or an endogenous IL7R transmembrane region. In some embodiments, the nucleotide sequence is integrated to an endogenous IL7R gene locus of the animal. In some embodiments, the humanized IL7R polypeptide has at least one mouse IL7R activity and/or at least one human IL7R activity.

In one aspect, the disclosure is related to a method for making a genetically-modified, non-human animal, comprising: replacing in at least one cell of the animal, at an endogenous IL7R gene locus, a sequence encoding a region of endogenous IL7R with a sequence encoding a corresponding region of human IL7R. In some embodiments, the sequence encoding the corresponding region of human IL7R comprises exon 1, exon 2, exon 3, exon 4, exon 5, exon 6, exon 7, and/or exon 8, or a part thereof, of a human IL7R gene. In some embodiments, the sequence encoding the corresponding region of human IL7R comprises a portion of exon 1, exon 2, exon 3, exon 4, exon 5, and a portion of exon 6, of a human IL7R gene. In some embodiments, the sequence encoding the corresponding region of human IL7R encodes amino acids 1-239 of SEQ ID NO: 76. In some embodiments, the region is located within the extracellular region of IL7R. In some embodiments, the sequence encoding a region of endogenous IL7R comprises exon 1, exon 2, exon 3, exon 4, exon 5, exon 6, exon 7, and/or exon 8, or a part thereof, of the endogenous IL7R gene. In some embodiments, the animal is a mouse, and the sequence encoding a region of endogenous IL7R comprises a portion of exon 1, exon 2, exon 3, exon 4, exon 5, and a portion of exon 6 of the endogenous IL7R gene.

In one aspect, the disclosure is related to a method of making a genetically-modified animal cell that expresses a chimeric IL7R, the method comprising: replacing at an endogenous IL7R gene locus, a nucleotide sequence encoding a region of endogenous IL7R with a nucleotide sequence encoding a corresponding region of human IL7R, thereby generating a genetically-modified animal cell that includes a nucleotide sequence that encodes the chimeric IL7R, in some embodiments, the animal cell expresses the chimeric IL7R. In some embodiments, the animal is a mouse. In some embodiments, the chimeric IL7R comprises a human or humanized IL7R extracellular region; and a transmembrane and/or a cytoplasmic region of endogenous IL7R. In some embodiments, the nucleotide sequence encoding the chimeric IL7R is operably linked to an endogenous IL7R regulatory region, e.g., promoter.

In some embodiments, the animal or mouse described herein further comprises a sequence encoding an additional human or chimeric protein (e.g., TSLP and/or TSLPR).

In one aspect, the disclosure is related to a method of determining effectiveness of a therapeutic agent for treating an immune disorder, comprising: a) administering the therapeutic agent to the animal as described herein, in some embodiments, the animal has the immune disorder; and b) determining effects of the therapeutic agent in treating the immune disorder. In some embodiments, the immune disorder is asthma. In some embodiments, the animal is a mouse and the asthma is induced by treating the mouse with ovalbumin (OVA) and aluminum hydroxide. In some embodiments, the effects are evaluated by comparing serum IgE level; pathological lung histology features; number of inflammatory cells (e.g., eosinophil counts in infiltrating cells) in bronchoalveolar lavage fluid (BALF); and/or airway reactivity of the animal with an animal that is not treated with the therapeutic agent. In some embodiments, the immune disorder is atopic dermatitis. In some embodiments, the animal is a mouse and the atopic dermatitis is induced by treating the mouse with oxazolone (OXA), e.g., smearing OXA on mouse ear and back. In some embodiments, the effects are evaluated by comparing epidermal stromal cell hyperplasia; erosion/scab; hyperkeratosis; dermal and subcutaneous mixed inflammatory cell infiltration; eosinophilic infiltration; serum IgE levels; and/or ear thickness of the animal with an animal that is not treated with the therapeutic agent. In some embodiments, the therapeutic agent is an anti-TSLP antibody, an anti-TSLPR antibody, and/or a corticosteroid (e.g., dexamethasone).

In one aspect, the disclosure is related to a method of determining effectiveness of a therapeutic agent for reducing an inflammation, comprising: a) administering the therapeutic agent to the animal as described herein, in some embodiments, the animal has the inflammation; and b) determining effects of the therapeutic agent for reducing the inflammation.

In one aspect, the disclosure is related to a method of determining effectiveness of a therapeutic agent for treating an autoimmune disease, comprising: a) administering the therapeutic agent to the animal as described herein, in some embodiments, the animal has the autoimmune disease; and b) determining effects of the therapeutic agent for treating the autoimmune disease. In some embodiments, the autoimmune disease is inflammatory arthritis, eczema, eosinophilic esophagitis, rheumatoid arthritis, Crohn's disease, systemic lupus erythematosus, ankylosing spondylitis, inflammatory bowel diseases (IBD), ulcerative colitis, multiple sclerosis, systemic juvenile idiopathic arthritis (SJIA), and/or scleroderma. In some embodiments, the therapeutic agent is an anti-TSLP antibody, an anti-TSLPR antibody, or a corticosteroid (e.g., dexamethasone).

In one aspect, the disclosure is related to a method of determining toxicity of a therapeutic agent comprising: a) administering the therapeutic agent to the animal as described herein; and b) determining effects of the therapeutic agent to the animal. In some embodiments, the therapeutic agent is an anti-TSLP antibody or an anti-TSLPR antibody. In some embodiments, determining effects of the therapeutic agent to the animal involves measuring the body weight, red blood cell count, hematocrit, and/or hemoglobin of the animal.

In one aspect, the disclosure is related to a protein comprising an amino acid sequence, In some embodiments, the amino acid sequence is one of the following:
(a) an amino acid sequence set forth in SEQ ID NO: 1, 2, 7, 8, 19, 65, 66, 75, 76, or 81;
(b) an amino acid sequence that is at least 90% identical to SEQ ID NO: 1, 2, 7, 8, 19, 65, 66, 75, 76, or 81;
(c) an amino acid sequence that is at least 91%, 92%, 93%, 94%, 95%, 96%, 97%, 98%, or 99% identical to SEQ ID NO: 1, 2, 7, 8, 19, 65, 66, 75, 76, or 81;
(d) an amino acid sequence that is different from the amino acid sequence set forth in SEQ ID NO: 1, 2, 7, 8, 19, 65, 66, 75, 76, or 81 by no more than 10, 9, 8, 7, 6, 5, 4, 3, 2 or 1 amino acid; and
(e) an amino acid sequence that comprises a substitution, a deletion and/or insertion of one, two, three, four, five or more amino acids to the amino acid sequence set forth in SEQ ID NO: 1, 2, 7, 8, 19, 65, 66, 75, 76, or 81.

In one aspect, the disclosure is related to a nucleic acid comprising a nucleotide sequence, in some embodiments, the nucleotide sequence is one of the following:
(a) a sequence that encodes the protein as described herein;
(b) SEQ ID NO: 3, 4, 5, 6, 9, 10, 11, 12, 13, 14, 15, 16, 17, 18, 20, 21, 22, 23, 24, 25, 26, 34, 35, 36, 37, 40, 67, 68, 69, 77, 78, 79, 80, or 85;
(c) a sequence that is at least 90% identical to SEQ ID NO: 3, 4, 5, 6, 9, 10, 11, 12, 13, 14, 15, 16, 17, 18, 20, 21, 22, 23, 24, 25, 26, 34, 35, 36, 37, 40, 67, 68, 69, 77, 78, 79, 80, or 85; and
(d) a sequence that is at least 91%, 92%, 93%, 94%, 95%, 96%, 97%, 98%, or 99% identical to SEQ ID NO: 3, 4, 5, 6, 9, 10, 11, 12, 13, 14, 15, 16, 17, 18, 20, 21, 22, 23, 24, 25, 26, 34, 35, 36, 37, 40, 67, 68, 69, 77, 78, 79, 80, or 85.

In one aspect, the disclosure is related to a cell comprising the protein and/or the nucleic acid as described herein. In one aspect, the disclosure is related to an animal comprising the protein and/or the nucleic acid as described herein.

The disclosure further relates to the use of the non-human mammal or an offspring thereof, or the tumor bearing non-human mammal, the animal model generated through the method as described herein in the development of a product related to an immunization processes of human cells, the manufacture of a human antibody, or the model system for a research in pharmacology, immunology, microbiology and medicine.

The disclosure also relates to the use of the non-human mammal or an offspring thereof, or the tumor bearing non-human mammal, the animal model generated through the method as described herein in the production and utilization of an animal experimental disease model of an immunization processes involving human cells, the study on a pathogen, or the development of a new diagnostic strategy and/or a therapeutic strategy.

The disclosure further relates to the use of the non-human mammal or an offspring thereof, or the tumor bearing non-human mammal, the animal model generated through the methods as described herein, in the screening, verifying, evaluating or studying the TSLP and/or TSLPR gene functions, human TSLP and/or TSLPR antibodies, drugs or efficacies for human TSLP and/or TSLPR targeting sites, the drugs for immune-related diseases and antitumor drugs.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic diagram showing mouse and human TSLP gene loci.

FIG. 2 is a schematic diagram showing humanized TSLP gene locus.

FIG. 3 is a schematic diagram showing a TSLP gene targeting strategy.

FIG. 4 shows PCR results of recombinant cells. M is a marker. WT is a wild-type control. H₂O is a water control.

FIG. 5 shows Southern Blot results of cells after recombination using the 5′ Probe, 3′ Probe, and Neo Probe-5 (3′). WT is a wild-type control.

FIGS. 6A-6D show genotyping results of F1 generation mice by primer pairs WT-F/WT-R (A), Mut-F/WT-R (B), Frt-F/Frt-R (C), and Flp-F2/Flp-R2 (D), respectively. PC is a positive control. M is a marker. H₂O is a water control.

FIG. 7 is a schematic diagram showing FRT recombination process in TSLP gene humanized mice.

FIG. 8A shows mouse TSLP (mTSLP) protein level in wild-type C57BL/6 mice and TSLP gene humanized heterozygous mice (B-hTSLP), as determined by ELISA.

FIG. 8B shows human TSLP (hTSLP) protein level in wild-type C57BL/6 mice and TSLP gene humanized heterozygous mice (B-hTSLP), as determined by ELISA.

FIG. 9 is a schematic diagram showing mouse and human TSLPR gene loci.

FIG. 10 is a schematic diagram showing humanized TSLPR gene locus.

FIG. 11 is a schematic diagram showing a TSLPR gene targeting strategy.

FIG. 12 is a schematic diagram showing FRT recombination process in TSLPR gene humanized mice.

FIG. 13 is a schematic diagram showing a TSLPR gene targeting strategy.

FIG. 14 shows mouse tail PCR detection results of F1 generation mice by primers F1-F and F1-R. M is a marker. H₂O is a water control.

FIG. 15 shows Southern Blot results of cells after recombination using the 3′ Probe and lox2 STOP Probe. WT is a wild-type control.

FIG. 16 is a schematic diagram showing humanized TSLPR gene locus.

FIG. 17 is a schematic diagram showing a TSLPR gene targeting strategy.

FIGS. 18A-18C show mRNA detection results of mouse TSLPR (mTSLPR), human TSLPR (hTSLPR), and GAPDH, respectively, in the peripheral blood of a wild-type C57BL/6 mouse (+/+) and a TSLP/TSLPR double-gene humanized heterozygous mouse (H/+). H₂O is a water control.

FIG. 19A shows mouse TSLP (mTSLP) protein level in a wild-type C57BL/6 mouse (+/+) and a TSLP/TSLPR double-gene humanized homozygous mouse (H/H), as determined by ELISA. ND stands for not detected.

FIG. 19B shows human TSLP (hTSLP) protein level in a wild-type C57BL/6 mouse and a TSLP/TSLPR double-gene humanized homozygous mouse (H/H), as determined by ELISA. ND stands for not detected.

FIG. 20A shows mouse TARC expression level in the bone marrow cells of wild-type C57BL/6 mice (+/+) and TSLP/TSLPR double-gene humanized homozygous mice (H/H) that were stimulated with hFLT3L and hTSLP.

FIG. 20B shows mouse TARC expression level in the bone marrow cells of wild-type C57BL/6 mice (+/+) and TSLP/TSLPR double-gene humanized homozygous mice (H/H) that were stimulated with hFLT3L and mTSLP.

FIG. 21 is a schematic diagram showing a IL33 gene targeting strategy.

FIG. 22 is a schematic diagram showing a IL7R gene targeting strategy.

FIG. 23 is an experimental scheme to establish an atopic dermatitis (AD) model using OXA in TSLP/TSLPR double-gene humanized mice. The numbers are days post first sensitization.

FIG. 24 shows the body weight of control group mice (G1), model group mice (G2) and treatment group mice (G3-G6). AD was induced in TSLP/TSLPR double-gene humanized homozygous mice by OXA and then the model group mice (G2) were treated with hIgG2, and the treatment group mice were treated with Dexamethasone (3 mg/kg; G3) or Tezepelumab analog (1-10 mg/kg; G4-G6). The control group mice (G1) were only treated with solvent (G1).

FIG. 25 shows the ear thickness of control group mice (G1), model group mice (G2) and treatment group mice (G3-G6).

FIG. 26 shows the serum IgE levels in control group mice (G1), model group mice (G2) and treatment group mice (G3-G6).

FIG. 27 shows the score of eosinophil infiltration in control group mice (G1), model group mice (G2) and treatment group mice (G3-G6).

FIG. 28 shows ear tissue section staining results in control group mice (G1), model group mice (G2) and treatment group mice (G3-G6).

FIG. 29 shows ear tissue section total score results in control group mice (G1), model group mice (G2) and treatment group mice (G3-G6).

FIG. 30 shows the alignment between human TSLP amino acid sequence (NP_149024.1; SEQ ID NO: 2) and mouse TSLP amino acid sequence (NP_067342.1; SEQ ID NO: 1).

FIG. 31 shows the alignment between human TSLP amino acid sequence (NP_149024.1; SEQ ID NO: 2) and rat TSLP amino acid sequence (XP_038953309.1; SEQ ID NO: 82).

FIG. 32 shows the alignment between human TSLPR amino acid sequence (NP_071431.2; SEQ ID NO: 8) and mouse TSLPR amino acid sequence (NP_057924.3; SEQ ID NO: 7).

FIG. 33 shows the alignment between human TSLPR amino acid sequence (NP_071431.2; SEQ ID NO: 8) and rat TSLPR amino acid sequence (NP_604460.2; SEQ ID NO: 83).

FIG. 34 shows the alignment between human IL7R amino acid sequence (NP_002176.2; SEQ ID NO: 76) and mouse IL7R amino acid sequence (NP_032398.3; SEQ ID NO: 75).

FIG. 35 shows the alignment between human IL7R amino acid sequence (NP_002176.2; SEQ ID NO: 76) and rat IL7R amino acid sequence (NP_001099888.1; SEQ ID NO: 84).

### DETAILED DESCRIPTION

This disclosure relates to transgenic non-human animal with human or chimeric (e.g., humanized) TSLP, TSLPR, IL33 and/or IL7R, and methods of use thereof.

The cytokine thymic stromal lymphopoietin (TSLP) was originally detected in the supernatant of a thymic stromal cell line and shown to support the long-term growth of a B cell line and to enhance the proliferation of unfractionated thymocytes responding to stimulation with anti-CD3 antibody. It was later shown to be a critical mediator of type 2 immune responses and a promoter of T helper 2 (TH2) cell-mediated diseases, including asthma and atopic dermatitis (AD). However, work since this original definition now shows that TSLP has multiple functions, including in cell maturation, proliferation, survival and recruitment and is involved in various other diseases and host responses.

TSLP is a four α-helical type I cytokine and a paralogue of IL-7. Although first shown to act on B cells1, TSLP was then found to act directly on dendritic cells (DCs) and to indirectly affect T cells based on its effects on DCs; however, TSLP was later shown to also be required for normal CD4+ T cell development and to act directly on CD4+ and CD8+ T cells. Furthermore, TSLP has effects on neutrophils, mast cells, basophils, eosinophils, group 2 innate lymphoid cells (ILC2s), natural killer T cells, smooth muscle cells and tumor cells. This range of target cells helps to explain the broad functions that can be mediated by this cytokine in both humans and mice.

TSLP mediates signaling by establishing a heteromeric complex involving TSLPR, a type I cytokine receptor, and IL7R, a receptor also utilized by IL7. TSLPR binds TSLP with high affinity (KD=32 nM), while IL-7R does not bind to TSLPR alone with measurable affinity. However, IL-7R can be recruited with high affinity (KD=29 nM) to the TSLP/TSLPR complex making this binary assembly a mechanistic prerequisite for effective signal transduction. Of note, human TSLPR and IL-7R were shown to bear low, albeit measurable affinity (KD about 20 μM) for each other in the absence of TSLP, suggesting that preformed receptor-receptor interactions might play a role in the assembly of a TSLP-mediated complex under certain conditions. The ensuing dimerization of both receptor chains upon TSLP binding results in activation of Janus kinases (JAKs) and signal transducers and activators of transcription (STATs) leading to transcription of target genes and subsequent tightly coordinated immune responses. Therefore, TSLP and TSLP receptor are regarded as potential therapeutic target for inflammation, autoimmune diseases, and cancer.

Experimental animal models are an indispensable research tool for studying the effects of these antibodies (e.g., anti-TSLP antibodies). Common experimental animals include mice, rats, guinea pigs, hamsters, rabbits, dogs, monkeys, pigs, fish and so on. However, there are many differences between human and animal genes and protein sequences, and many human proteins cannot bind to the animal's homologous proteins to produce biological activity, leading to that the results of many clinical trials do not match the results obtained from animal experiments. A large number of clinical studies are in urgent need of better animal models. With the continuous development and maturation of genetic engineering technologies, the use of human cells or genes to replace or substitute an animal's endogenous similar cells or genes to establish a biological system or disease model closer to human, and establish the humanized experimental animal models (humanized animal model) has provided an important tool for new clinical approaches or means. In this context, the genetically engineered animal model, that is, the use of genetic manipulation techniques, the use of human normal or mutant genes to replace animal homologous genes, can be used to establish the genetically modified animal models that are closer to human gene systems. The humanized animal models have various important applications. For example, due to the presence of human or humanized genes, the animals can express or express in part of the proteins with human functions, so as to greatly reduce the differences in clinical trials between humans and animals, and provide the possibility of drug screening at animal levels.

### TSLP

Thymic stromal lymphopoietin (TSLP) is a member of the IL-2 family of cytokines that was initially identified as a pre-B cell growth factor. Epithelial cells in the lungs, skin, and gastrointestinal tract are thought to be the primary source of TSLP during both homeostatic and inflammatory conditions, although dendritic cells (DCs), basophils, and mast cells can also express TSLP. TSLP expression and release from epithelial cells is increased in response to a broad array of stimuli, including mechanical injury, infection, inflammatory cytokines, and proteases such as trypsin and papain. Two main isoforms of TSLP have been described in mice, but the functional consequence of these variants is unknown. In humans, a short isoform appears to be expressed in basal conditions, whereas a longer isoform is induced by inflammatory stimuli. Cleavage of human TSLP by serine proteases may also regulate TSLP protein levels or function, although it is unclear whether a similar regulatory mechanism exists in mice. TSLP genetic variants and high levels of TSLP expression have been linked to atopic diseases such as AD, asthma, allergic rhinoconjunctivitis, and EoE. TSLP overexpression has also been reported in Netherton syndrome, a genetic disease caused by mutations in SPINK5 that manifests in type 2 inflammation at multiple sites, and in some nonatopic pulmonary diseases such as chronic obstructive pulmonary disease.

TSLP is a distant paralog of IL-7 and shares a common receptor subunit, IL-7R, with IL-7. TSLP binds the TSLP receptor (TSLPR) that is coupled with IL-7R to activate downstream pathways. TSLP-mediated signaling has been studied primarily in DCs and T lymphocytes, in which signaling occurred primarily through JAK/STAT pathways. A number of non-hematopoietic cell populations have been shown to express TSLPR and to be responsive to TSLP. Although the implications in allergic inflammation are not known, the barrier epithelium can respond to TSLP, and TSLP mediated recovery from colonic inflammation in a mouse model of colitis by inducing intestinal epithelial production of secretory leukocyte peptidase inhibitor (SLPI). A growing body of literature also suggests that TSLP can activate a subset of sensory neurons to drive the itch response in allergic diseases such as AD.

A detailed description of TSLP and its function can be found, e.g., in Marković, I., et al. “Modulation of signaling mediated by TSLP and IL-7 in inflammation, autoimmune diseases, and cancer.” Frontiers in Immunology 11 (2020): 1557; Ziegler, S. F., et al. “The biology of thymic stromal lymphopoietin (TSLP).” Advances in Pharmacology 66 (2013): 129-155; Gauvreau, G. M., et al. “Thymic stromal lymphopoietin: its role and potential as a therapeutic target in asthma.” Expert Opinion on Therapeutic Targets 24.8 (2020): 777-792; and Ebina-Shibuya, R., et al. “Role of thymic stromal lymphopoietin in allergy and beyond.” Nature Reviews Immunology (2022): 1-14; each of which is incorporated by reference in its entirety.

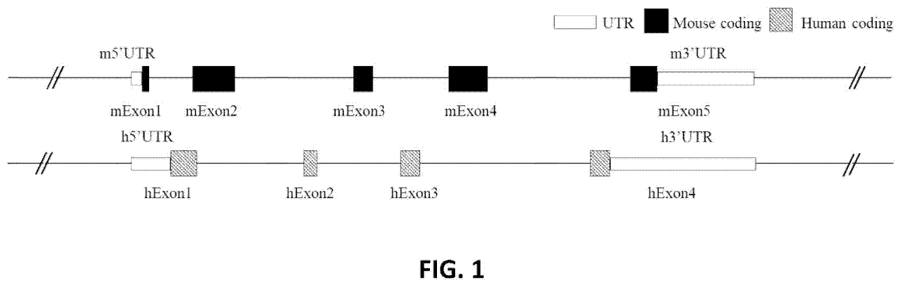

In human genomes, TSLP gene (Gene ID: 85480) locus has four exons, exon 1, exon 2, exon 3, and exon 4 (FIG. 1). The TSLP protein also has a signal peptide. The nucleotide sequence for human TSLP mRNA is NM_033035.5, and the amino acid sequence for human TSLP is NP_149024.1 (SEQ ID NO: 2). The location for each exon and each region in human TSLP nucleotide sequence and amino acid sequence is listed below:

TABLE 1

| | | NP_149024.1 |
|---|---|---|
| Human TSLP | NM_033035.5 | 159 aa |
| (approximate location) | 2610 bp | SEQ ID NO: 2 |
| Exon 1 | 1-349 | 1-57 |
| Exon 2 | 350-394 | 58-72 |
| Exon 3 | 395-529 | 73-117 |
| Exon 4 | 530-2610 | 118-159 |
| Signal peptide | 179-262 | 1-28 |
| Chain | 263-655 | 29-159 |
| Replaced region in Example | 179-658 | 1-159 |

The human TSLP gene (Gene ID: 85480) is located in Chromosome 5 of the human genome, which is located from 111070062 to 111078026 (GRCh38.p13 (GCF_000001405.39)). The 5′-UTR is from 111071713 to 111071890, exon 1 is from 111071713 to 111072061, intron 1 is from 111072062 to 111072887, exon 2 is from 111072888 to 111072932, intron 2 is from 111072933 to 111073510, exon 3 is from 111073511 to 111073645, intron 3 is from 111073646 to 111075945, exon 4 is from 111075946 to 111078026, and the 3′-UTR is from 111076075 to 111078026, based on transcript NM_033035.5. All relevant information for human TSLP locus can be found in the NCBI website with Gene ID: 85480, which is incorporated by reference herein in its entirety.

In mice, TSLP gene locus has five exons, exon 1, exon 2, exon 3, exon 4, and exon 5 (FIG. 1). The mouse TSLP protein also has a signal peptide. The nucleotide sequence for mouse TSLP mRNA is NM_021367.2, the amino acid sequence for mouse TSLP is NP_067342.1 (SEQ ID NO: 1). The location for each exon and each region in the mouse TSLP nucleotide sequence and amino acid sequence is listed below:

TABLE 2

| | | NP_067342.1 |
|---|---|---|
| Mouse TSLP | NM_021367.2 | 140 aa |
| (approximate location) | 1143 bp | SEQ ID NO: 1 |
| Exon 1 | 1-21 | 1 |
| Exon 2 | 22-161 | 2-48 |
| Exon 3 | 162-197 | 49-60 |
| Exon 4 | 198-332 | 61-105 |
| Exon 5 | 333-1133 | 106-140 |
| Signal peptide | 18-74 | 1-19 |
| Chain | 75-437 | 20-140 |
| Replaced region in Example | 18-440 | 1-140 |

The mouse TSLP gene (Gene ID: 53603) is located in Chromosome 18 of the mouse genome, which is located from 32948436 to 32952852 (GRCm39 (GCF_000001635.27)). The 5′-UTR is from 32948436 to 32948452, exon 1 is from 32948436 to 32948456, intron 1 is from 32948457 to 32948620, exon 2 is from 32948621 to 32948760, intron 2 is from 32948761 to 32949502, exon 3 is from 32949503 to 32949538, intron 3 is from 32949539 to 32950112, exon 4 is from 32950113 to 32950247, intron 4 is from 32950248 to 32952051, exon 5 is from 32952052 to 32952845, and the 3′-UTR is from 32952160 to 32952845, based on transcript NM_021367.2. All relevant information for mouse Tslp locus can be found in the NCBI website with Gene ID: 53603, which is incorporated by reference herein in its entirety.

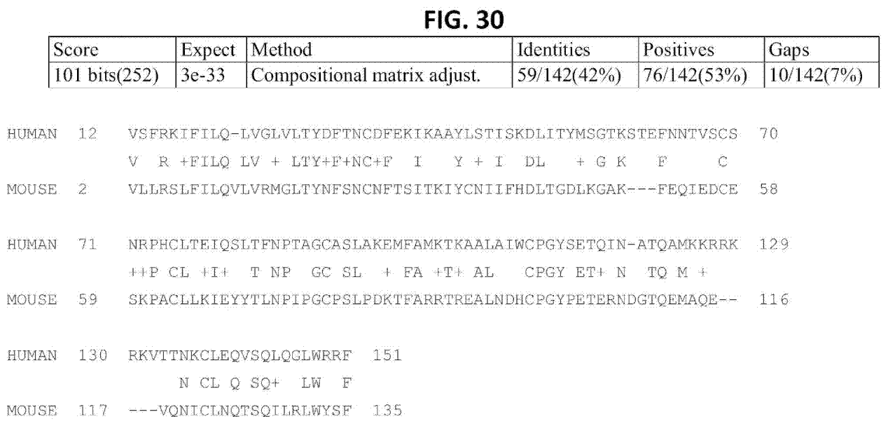

FIG. 30 shows the alignment between human TSLP amino acid sequence (NP_149024.1; SEQ ID NO: 2) and mouse TSLP amino acid sequence (NP_067342.1; SEQ ID NO: 1). Thus, the corresponding amino acid residue or region between human and mouse TSLP can be found in FIG. 30.

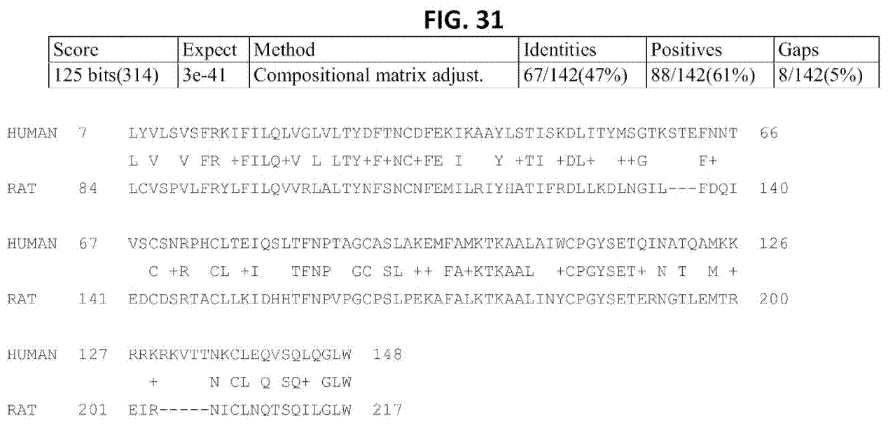

TSLP genes, proteins, and locus of the other species are also known in the art. For example, the gene ID for TSLP in Rattus norvegicus (rat) is 688621, the gene ID for TSLP in Macaca mulatta (Rhesus monkey) is 706194, the gene ID for TSLP in Canis lupus familiaris (dog) is 607671, and the gene ID for TSLP in Sus scrofa (pig) is 100515191. The relevant information for these genes (e.g., intron sequences, exon sequences, amino acid residues of these proteins) can be found, e.g., in NCBI database, which is incorporated by reference herein in its entirety. FIG. 31 shows the alignment between human TSLP amino acid sequence (NP_149024.1; SEQ ID NO: 2) and rat TSLP amino acid sequence (XP_038953309.1; SEQ ID NO: 82. Thus, the corresponding amino acid residue or region between human and rodent TSLP can be found in FIG. 31.

The present disclosure provides human or chimeric (e.g., humanized) TSLP nucleotide sequence and/or amino acid sequences. In some embodiments, the entire sequence of mouse exon 1, exon 2, exon 3, exon 4, and/or exon 5 are replaced by the corresponding human sequence. In some embodiments, a “region” or “portion” of mouse exon 1, exon 2, exon 3, exon 4, and/or exon 5 are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 450 460, 470, or 480 nucleotides, or at least 1, 2, 3, 4, 5, 6, 7, 8, 9, 10, 20, 30, 40, 50, 60, 70, 80, 90, 100, 110, 120, 130, 140, 150, 155, 156, 157, 158, or 159 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, and/or exon 5. In some embodiments, a region, a portion, or the entire sequence of mouse exon 1, exon 2, exon 3, exon 4, and/or exon 5 (e.g., a portion of exon 1, exons 2-4, and a portion of exon 5) are replaced by a region, a portion, or the entire sequence of the human exon 1, exon 2, exon 3, and/or exon 4 (e.g., a portion of exon 1, exons 2-3, and a portion of exon 4).

In some embodiments, a “region” or “portion” of endogenous exon 1, exon 2, exon 3, exon 4, and/or exon 5 is deleted.

In some embodiments, the present disclosure is related to a genetically-modified, non-human animal whose genome comprises a human, chimeric, or humanized TSLP nucleotide sequence. In some embodiments, the human, chimeric, or humanized TSLP nucleotide sequence encodes a TSLP protein that is at least 80%, 85%, 90%, 95%, or 100% identical to SEQ ID NO: 2. In some embodiments, the genome of the animal comprises a sequence that is at least 80%, 85%, 90%, 95%, or 100% identical to SEQ ID NO: 3, 4, 5, 6, 23, 24, 25, 26.

In some embodiments, the genetically-modified non-human animal described herein comprises a sequence encoding a human or humanized TSLP protein. In some embodiments, the humanized TSLP protein comprises a human or humanized signal peptide. In some embodiments, the humanized TSLP protein comprises an endogenous signal peptide.

In some embodiments, the genetically-modified non-human animal described herein comprises a human or humanized TSLP gene. In some embodiments, the humanized TSLP gene comprises 4 exons. In some embodiments, the humanized TSLP gene comprises humanized exon 1, human exon 2, human exon 3, and/or humanized exon 4. In some embodiments, the humanized TSLP gene comprises 3 introns. In some embodiments, the humanized TSLP gene comprises human intron 1, human intron 2, and/or human intron 3. In some embodiments, the humanized TSLP gene comprises human or humanized 5′ UTR. In some embodiments, the humanized TSLP gene comprises human or humanized 3′ UTR. In some embodiments, the humanized TSLP gene comprises endogenous 5′ UTR. In some embodiments, the humanized TSLP gene comprises endogenous 3′ UTR.

In some embodiments, the genetically modified animals can express a human TSLP and/or a chimeric (e.g., humanized) TSLP from endogenous mouse loci, wherein the endogenous mouse TSLP gene has been replaced with a human TSLP gene and/or a nucleotide sequence that encodes a region of human TSLP sequence or an amino acid sequence that is at least 10%, 20%, 30%, 40%, 50%, 60%, 70&, 80%, 90%, 95%, 96%, 97%, 98%, or 99% identical to the human TSLP sequence. In various embodiments, an endogenous non-human TSLP locus is modified in whole or in part to comprise human nucleic acid sequence encoding at least one protein-coding sequence of a mature TSLP protein.

In some embodiments, the genetically modified mice can express the human TSLP and/or chimeric TSLP (e.g., humanized TSLP) from endogenous loci that are under control of mouse promoters and/or mouse regulatory elements. The replacement(s) at the endogenous mouse loci provide non-human animals that express human TSLP or chimeric TSLP (e.g., humanized TSLP) in appropriate cell types and in a manner that does not result in the potential pathologies observed in some other transgenic mice known in the art. The human TSLP or the chimeric TSLP (e.g., humanized TSLP) expressed in animal can maintain one or more functions of the wild-type mouse or human TSLP in the animal. For example, the expressed TSLP can bind to human or non-human TSLPR. Furthermore, in some embodiments, the animal does not express endogenous TSLP. In some embodiments, the animal expresses a decreased level of endogenous TSLP as compared to a wild-type animal. As used herein, the term “endogenous TSLP” refers to TSLP protein that is expressed from an endogenous TSLP nucleotide sequence of the non-human animal (e.g., mouse) before any genetic modification.

The genome of the animal can comprise a sequence encoding an amino acid sequence that is at least 70%, 75%, 80%, 85%, 90%, 95%, 99%, or 100% identical to human TSLP (NP_149024.1; SEQ ID NO: 2). In some embodiments, the genome comprises a sequence that is at least 70%, 75%, 80%, 85%, 90%, 95%, 99%, or 100% identical to SEQ ID NO: 5 or 6.

The genome of the genetically modified animal can comprise a replacement at an endogenous TSLP gene locus of a sequence encoding a region of endogenous TSLP with a sequence encoding a corresponding region of human TSLP. In some embodiments, the sequence that is replaced is any sequence within the endogenous TSLP gene locus, e.g., exon 1, exon 2, exon 3, exon 4, exon 5, 5′-UTR, 3′-UTR, intron 1, intron 2, intron 3, intron 4, or any combination thereof. In some embodiments, the sequence that is replaced is within the regulatory region of the endogenous TSLP gene. In some embodiments, the sequence that is replaced is exon 1, exon 2, exon 3, exon 4, and/or exon 5, or a portion thereof, of an endogenous mouse TSLP gene locus.

The genetically modified animal can have one or more cells expressing a human or chimeric TSLP (e.g., humanized TSLP). In some embodiments, the human or chimeric TSLP has a sequence that has at least 10, 20, 30, 40, 50, 60, 70, 80, 90, 100, 110, 120, 130, 140, 150, 155, 156, 157, 158, or 159 amino acids (e.g., contiguously or non-contiguously) that are identical to human TSLP (e.g., SEQ ID NO: 2).

In some embodiments, the genome of the genetically modified animal comprises a sequence that corresponds to a portion or the entire sequence of exon 1, exon 2, exon 3, and/or exon 4 of human TSLP gene; a portion or the entire sequence of human TSLP gene; or a portion or the entire sequence of SEQ ID NO: 6.

In some embodiments, the genome of the genetically modified animal comprises a portion of exon 1, exons 2-3, and a portion of exon 4 of human TSLP gene. In some embodiments, the portion of exon 1 includes at least 5, 10, 20, 30, 40, 50, 60, 70, 80, 90, 100, 110, 120, 130, 140, 150, 160, 170, 171, 172, 173, 174, 175, 180, 200, 250, 300, 340, or 349 nucleotides. In some embodiments, the portion of exon 1 includes 171 nucleotides. In some embodiments, the portion of exon 1 includes a nucleotide of at least 50 bp or at least 100 bp. In some embodiments, the portion of exon 4 includes at least 10, 20, 30, 40, 50, 60, 70, 80, 90, 100, 110, 120, 121, 122, 123, 124, 125, 126, 127, 128, 129, 130, 150, 200, 500, 1000, 1500, 2000, or 2081 nucleotides. In some embodiments, the portion of exon 4 includes 129 nucleotides. In some embodiments, the portion of exon 5 includes a nucleotide of at least 50 bp or at least 100 bp. In some embodiments, the replaced sequence encodes the coding sequence of human TSLP (e.g., positions 179-658 of NM_033035.5).

Furthermore, the genetically modified animal can be heterozygous with respect to the replacement at the endogenous TSLP locus, or homozygous with respect to the replacement at the endogenous TSLP locus.

In some embodiments, the humanized TSLP locus lacks a human TSLP 5′-UTR. In some embodiment, the humanized TSLP locus comprises an endogenous (e.g., mouse) 5′-UTR. In some embodiments, the humanization comprises an endogenous (e.g., mouse) 3′-UTR. In appropriate cases, it may be reasonable to presume that the mouse and human TSLP genes appear to be similarly regulated based on the similarity of their 5′-flanking sequence. As shown in the present disclosure, humanized TSLP mice that comprise a replacement at an endogenous mouse TSLP locus, which retain mouse regulatory elements but comprise a humanization of TSLP encoding sequence, do not exhibit pathologies. Both genetically modified mice that are heterozygous or homozygous for humanized TSLP are grossly normal.

In another aspect, the disclosure also provides a genetically-modified, non-human animal whose genome comprise a disruption in the animal's endogenous TSLP gene, wherein the disruption of the endogenous TSLP gene comprises deletion of exon 1, exon 2, exon 3, exon 4, and/or exon 5, or part thereof of the endogenous TSLP gene.

In some embodiments, the disruption of the endogenous TSLP gene comprises deletion of one or more exons or part of exons selected from the group consisting of exon 1, exon 2, exon 3, exon 4, and exon 5 of the endogenous TSLP gene.

In some embodiments, the disruption of the endogenous TSLP gene further comprises deletion of one or more introns or part of introns selected from the group consisting of intron 1, intron 2, intron 3, and intron 4 of the endogenous TSLP gene.

In some embodiments, wherein the deletion can comprise deleting at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, 900, 1000, 1100, 1500, 2000, 3000, 3700, 4000, 4400, 5000, or more nucleotides.

In some embodiments, the disruption of the endogenous TSLP gene comprises the deletion of at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, or 300 nucleotides of exon 1, exon 2, exon 3, exon 4, and/or exon 5 (e.g., deletion of at least 1, at least 2, at least 3, at least 4, at least 5, or at least 6 nucleotides from exon 1, exons 2-4, and at least 50 nucleotides from exon 5).

The disclosure further relates to a TSLP genomic DNA sequence of a humanized mouse, a DNA sequence obtained by a reverse transcription of the mRNA obtained by transcription thereof is consistent with or complementary to the DNA sequence; a construct expressing the amino acid sequence thereof; a cell comprising the construct thereof; a tissue comprising the cell thereof.

Thus, in some embodiments, the present disclosure also provides a chimeric (e.g., humanized) TSLP nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse TSLP mRNA sequence (e.g., NM_021367.2), mouse TSLP amino acid sequence (e.g., SEQ ID NO: 1), or a portion thereof (e.g., 5′ UTR, a portion of exon 1, a portion of exon 5, and 3′ UTR); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human TSLP mRNA sequence (e.g., NM_033035.5), human TSLP amino acid sequence (e.g., SEQ ID NO: 2), or a portion thereof (e.g., a portion of exon 1, exons 2-3, and a portion of exon 4).

In some embodiments, the sequence encoding amino acids 1-140 of mouse TSLP (SEQ ID NO: 1) is replaced. In some embodiments, the sequence is replaced by a sequence encoding a corresponding region of human TSLP (e.g., amino acids 1-159 of human TSLP (SEQ ID NO: 2)).

In some embodiments, the sequence encoding amino acids 20-140 of mouse TSLP (SEQ ID NO: 1) is replaced. In some embodiments, the sequence is replaced by a sequence encoding a corresponding region of human TSLP (e.g., amino acids 29-159 of human TSLP (SEQ ID NO: 2)).

In some embodiments, the nucleic acids as described herein are operably linked to a promotor or regulatory element, e.g., an endogenous mouse TSLP promotor, an inducible promoter, an enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from part of or the entire mouse TSLP nucleotide sequence (e.g., a portion of exon 1, exons 2-4, and a portion of exon 5 of NM_021367.2).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as part of or the entire mouse TSLP nucleotide sequence (e.g., 5′ UTR, a portion of exon 1, a portion of exon 5, and 3′ UTR of NM_021367.2).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from part of or the entire human TSLP nucleotide sequence (e.g., 5′ UTR, a portion of exon 1, a portion of exon 4, and 3′ UTR of NM_033035.5).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as part of or the entire human TSLP nucleotide sequence (e.g., a portion of exon 1, exons 2-3, and a portion of exon 4 of NM_033035.5).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from part of or the entire mouse TSLP amino acid sequence (e.g., SEQ ID NO: 1).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as part of or the entire mouse TSLP amino acid sequence (e.g., SEQ ID NO: 1).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from part of or the entire human TSLP amino acid sequence (e.g., SEQ ID NO: 2).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as part of or the entire human TSLP amino acid sequence (e.g., SEQ ID NO: 2).

The present disclosure also provides a humanized TSLP mouse amino acid sequence, wherein the amino acid sequence is selected from the group consisting of:
a) an amino acid sequence shown in SEQ ID NO: 1 or 2;
b) an amino acid sequence having a homology of at least 90% with or at least 90% identical to the amino acid sequence shown in SEQ ID NO: 1 or 2;
c) an amino acid sequence encoded by a nucleic acid sequence, wherein the nucleic acid sequence is able to hybridize to a nucleotide sequence encoding the amino acid shown in SEQ ID NO: 1 or 2 under a low stringency condition or a strict stringency condition;
d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 1 or 2;
e) an amino acid sequence that is different from the amino acid sequence shown in SEQ ID NO: 1 or 2 by no more than 10, 9, 8, 7, 6, 5, 4, 3, 2 or no more than 1 amino acid; or
f) an amino acid sequence that comprises a substitution, a deletion and/or insertion of one or more amino acids to the amino acid sequence shown in SEQ ID NO: 1 or 2.

The present disclosure also relates to a TSLP nucleic acid (e.g., DNA or RNA) sequence, wherein the nucleic acid sequence can be selected from the group consisting of:
a) a nucleic acid sequence as shown in SEQ ID NO: 3, 4, 5, 6, 23, 24, 25, 26, or a nucleic acid sequence encoding a homologous TSLP amino acid sequence of a humanized mouse TSLP;
b) a nucleic acid sequence that is able to hybridize to the nucleotide sequence as shown in SEQ ID NO: 3, 4, 5, 6, 23, 24, 25, 26 under a low stringency condition or a strict stringency condition;
c) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 3, 4, 5, 6, 23, 24, 25, 26;
d) a nucleic acid sequence that encodes an amino acid sequence, wherein the amino acid sequence has a homology of at least 90% with or at least 90% identical to the amino acid sequence shown in SEQ ID NO: 1 or 2;
e) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 1 or 2;
f) a nucleic acid sequence that encodes an amino acid sequence, wherein the amino acid sequence is different from the amino acid sequence shown in SEQ ID NO: 1 or 2 by no more than 10, 9, 8, 7, 6, 5, 4, 3, 2 or no more than 1 amino acid; and/or
g) a nucleic acid sequence that encodes an amino acid sequence, wherein the amino acid sequence comprises a substitution, a deletion and/or insertion of one or more amino acids to the amino acid sequence shown in SEQ ID NO: 1 or 2.

The present disclosure further relates to a TSLP genomic DNA sequence of a humanized mouse. The DNA sequence is obtained by reverse transcription of the mRNA obtained by transcription thereof is consistent with or complementary to the DNA sequence homologous to the sequence shown in SEQ ID NO: 5 or 6.

### TSLPR

TSLP receptor (TSLPR), also known as Cytokine Receptor Like Factor 2, CRLF2, or CRL2, is a type I cytokine receptor encoded by CRLF2a. TSLP signals through a heterodimeric receptor comprising TSLPR and the IL-7 receptor α-chain (IL-7Ra or IL7R; also known as CD127). This heterodimer is expressed on TSLP target cells such as DCs, mast cells, macrophages, basophils and T cells as well as epithelial cells and neurons. Unlike its paralogue IL-7, which activates JAK1 and JAK3 via a heterodimeric receptor comprising IL-7Ra and the common cytokine receptor γ-chain, TSLP activates JAK1 (via IL-7Ra) and JAK2 (via TSLPR). JAK1 and JAK2 then primarily activate signal transducer and activator of transcription 5A (STAT5A) and STAT5B and, to a lesser extent, STAT1 and STAT3. Rearrangement of this gene with immunoglobulin heavy chain gene (IGH) (chromosome 14), or with P2Y purinoceptor 8 gene (P2RY8) (chromosome X or Y) is associated with B-progenitor- and Down syndrome-acute lymphoblastic leukemia (ALL).

A detailed description of TSLPR and its function can be found, e.g., in Ebina-Shibuya, R., et al. “Role of thymic stromal lymphopoietin in allergy and beyond.” Nature Reviews Immunology (2022): 1-14; Verstraete, K., et al. “Structure and antagonism of the receptor complex mediated by human TSLP in allergy and asthma.” Nature Communications 8.1 (2017): 1-17; and Lu, N., et al. “TSLP and IL-7 use two different mechanisms to regulate human CD4+ T cell homeostasis.” Journal of Experimental Medicine 206.10 (2009): 2111-2119; each of which is incorporated by reference in its entirety.

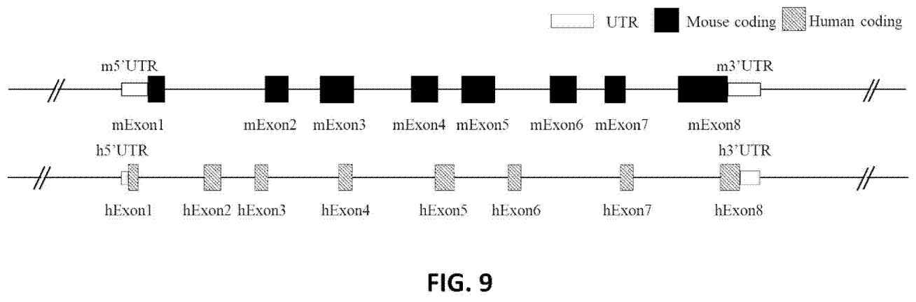

In human genomes, TSLPR gene (Gene ID: 64109) locus has eight exons, exon 1, exon 2, exon 3, exon 4, exon 5, exon 6, exon 7, and exon 8 (FIG. 9). The TSLPR protein also has a signal peptide, an extracellular region, a transmembrane region, and a cytoplasmic region. The nucleotide sequence for human TSLPR mRNA is NM_022148.4, and the amino acid sequence for human TSLPR is NP_071431.2 (SEQ ID NO: 8). The location for each exon and each region in human TSLPR nucleotide sequence and amino acid sequence is listed below:

TABLE 3

| | | NP_071431.2 |
|---|---|---|
| Human TSLPR | NM_022148.4 | 371 aa |
| (approximate location) | 1538 bp | SEQ ID NO: 8 |
| Exon 1 | 1-94 | 1-26 |
| Exon 2 | 95-197 | 27-61 |
| Exon 3 | 198-364 | 62-116 |
| Exon 4 | 365-498 | 117-161 |
| Exon 5 | 499-661 | 162-215 |
| Exon 6 | 662-782 | 216-256 |
| Exon 7 | 783-867 | 257-284 |
| Exon 8 | 868-1538 | 285-371 |
| Signal peptide | 16-81 | 1-22 |
| Extracellular region | 82-708 | 23-231 |
| (excluding signal peptide region) | | |
| Transmembrane region | 709-771 | 232-252 |
| Cytoplasmic region | 772-1128 | 253-371 |
| Example 3 | 19-774 | 2-253 |
| Example 2 | 16-774 | 1-253 |

The human TSLPR gene (Gene ID: 64109) is located in Chromosome X of the human genome, which is located from 1190437 to 1212762 (GRCh38.p13 (GCF_000001405.39)). The 5′-UTR is from 1212635 to 1212649, exon 1 is from 1212649 to 1212556, intron 1 is from 1212555 to 1208909, exon 2 is from 1208908 to 1208806, intron 2 is from 1208805 to 1206600, exon 3 is from 1206599 to 1206433, intron 3 is from 1206432 to 1202536, exon 4 is from 1202535 to 1202402, intron 4 is from 1202401 to 1198725, exon 5 is from 1198724 to 1198562, intron 5 is from 1198561 to 1196901, exon 6 is from 1196900 to 1196780, intron 6 is from 1196779 to 1193303, exon 7 is from 1193302 to 1193218, intron 7 is from 1193217 to 1191161, exon 8 is from 1191160 to 1190490, and the 3′-UTR is from 1190490 to 1190896, based on transcript NM_022148.4. All relevant information for human TSLPR locus can be found in the NCBI website with Gene ID: 64109, which is incorporated by reference herein in its entirety.

In mice, TSLPR gene locus has eight exons, exon 1, exon 2, exon 3, exon 4, exon 5, exon 6, exon 7, and exon 8 (FIG. 9). The mouse TSLPR protein also has a signal peptide, an extracellular region, a transmembrane region, and a cytoplasmic region. The nucleotide sequence for mouse TSLPR mRNA is NM_016715.4, the amino acid sequence for mouse TSLPR is NP_057924.3 (SEQ ID NO: 7). The location for each exon and each region in the mouse TSLPR nucleotide sequence and amino acid sequence is listed below:

TABLE 4

| | | NP_057924.3 |
|---|---|---|
| Mouse TSLPR | NM_016715.4 | 359 aa |
| (approximate location) | 1338 bp | SEQ ID NO: 7 |
| Exon 1 | 1-189 | 1-26 |
| Exon 2 | 190-286 | 27-59 |
| Exon 3 | 287-453 | 60-114 |
| Exon 4 | 454-596 | 115-162 |
| Exon 5 | 597-759 | 163-216 |
| Exon 6 | 760-880 | 217-257 |
| Exon 7 | 881-965 | 258-285 |
| Exon 8 | 966-1327 | 286-359 |
| Signal peptide | 111-167 | 1-19 |
| Extracellular region | 168-806 | 20-232 |
| (excluding signal peptide region) | | |
| Transmembrane region | 807-869 | 233-253 |
| Cytoplasmic region | 870-1187 | 254-359 |
| Chain | 171-1187 | 21-359 |
| Inserted region in Example 3 | Between 113-114 | Between 1-2 |
| Inserted region in Example 2 | Between 260-261 | Between 50-51 |

The mouse TSLPR gene (Gene ID: 57914) is located in Chromosome 5 of the mouse genome, which is located from 109702575 to 109707301 (GRCm39 (GCF_000001635.27)). The 5′-UTR is from 109706750 to 109706859, exon 1 is from 109706859 to 109706671, intron 1 is from 109706670 to 109705481, exon 2 is from 109705480 to 109705384, intron 2 is from 109705383 to 109705034, exon 3 is from 109705033 to 109704867, intron 3 is from 109704866 to 109704429, exon 4 is from 109704428 to 109704286, intron 4 is from 109704285 to 109704048, exon 5 is from 109704047 to 109703885, intron 5 is from 109703884 to 109703494, exon 6 is from 109703493 to 109703373, intron 6 is from 109703372 to 109703224, exon 7 is from 109703223 to 109703139, intron 7 is from 109703138 to 109702937, exon 8 is from 109702936 to 109702579, and the 3′-UTR is from 109702579 to 109702711, based on transcript NM_016715.4. All relevant information for mouse TSLPR locus can be found in the NCBI website with Gene ID: 57914, which is incorporated by reference herein in its entirety.

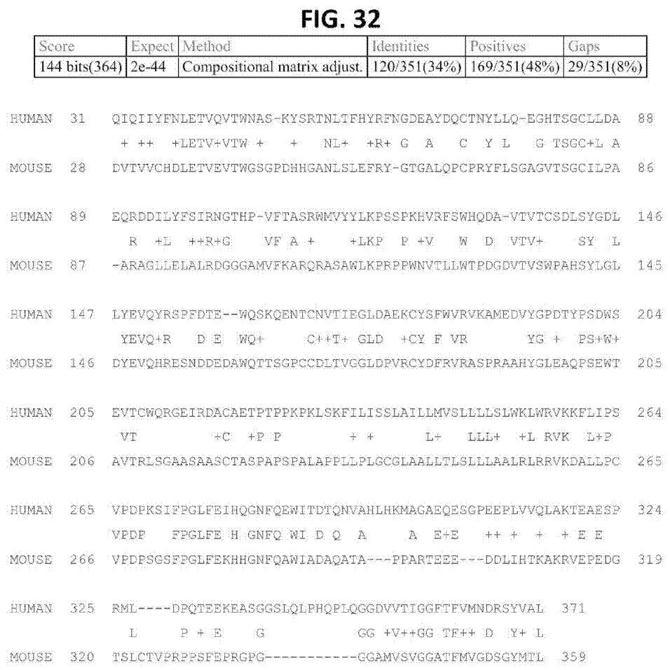

FIG. 32 shows the alignment between human TSLPR amino acid sequence (NP_071431.2; SEQ ID NO: 8) and mouse TSLPR amino acid sequence (NP_057924.3; SEQ ID NO: 7). Thus, the corresponding amino acid residue or region between human and mouse TSLPR can be found in FIG. 32.

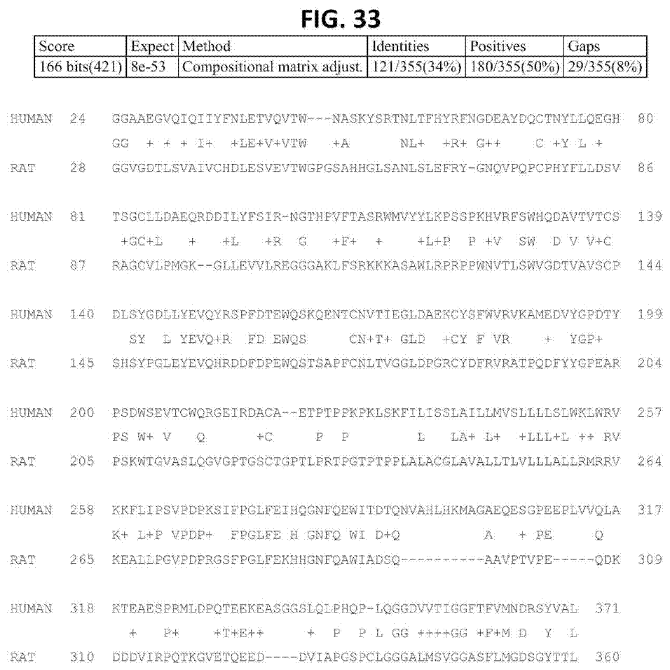

TSLPR (or CRLF2) genes, proteins, and locus of the other species are also known in the art. For example, the gene ID for TSLPR in Rattus norvegicus (rat) is 171499, the gene ID for TSLPR in Canis lupus familiaris (dog) is 491709, the gene ID for TSLPR in Pan troglodytes (chimpanzee) is 749758, and the gene ID for TSLPR in Danio rerio (zebrafish) is 100294510. The relevant information for these genes (e.g., intron sequences, exon sequences, amino acid residues of these proteins) can be found, e.g., in NCBI database, which is incorporated by reference herein in its entirety. FIG. 33 shows the alignment between human TSLPR amino acid sequence (NP_071431.2; SEQ ID NO: 8) and rat TSLPR amino acid sequence (NP_604460.2; SEQ ID NO:83. Thus, the corresponding amino acid residue or region between human and rodent TSLPR can be found in FIG. 33.

The present disclosure provides human or chimeric (e.g., humanized) TSLPR nucleotide sequence and/or amino acid sequences. In some embodiments, the entire sequence of mouse exon 1, intron 1, exon 2, intron 2, exon 3, intron 3, exon 4, intron 4, exon 5, intron 5, exon 6, intron 6, exon 7, intron 7, exon 8, signal peptide, extracellular region, transmembrane region, and/or cytoplasmic region are replaced by human sequences. In some embodiments, a “region” or “portion” of mouse exon 1, intron 1, exon 2, intron 2, exon 3, intron 3, exon 4, intron 4, exon 5, intron 5, exon 6, intron 6, exon 7, intron 7, exon 8, signal peptide, extracellular region, transmembrane regions, and/or cytoplasmic regions are replaced by human sequences. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, 900, 1000, 1100, 1200, 1300, 1330, 1350, 1400, 1500, 1530, or 1550 nucleotides, or at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 310, 320, 330, 340, 350, 360, or 370 amino acid residues. In some embodiments, the “region” or “portion” can be at least 50%, 55%, 60%, 65%, 70%, 75%, 80%, 85%, 90%, 95%, or 99% identical to exon 1, intron 1, exon 2, intron 2, exon 3, intron 3, exon 4, intron 4, exon 5, intron 5, exon 6, intron 6, exon 7, intron 7, exon 8, signal peptide, extracellular region, transmembrane region, or cytoplasmic region. In some embodiments, a region, a portion, or the entire sequence of mouse exon 1, intron 1, exon 2, intron 2, exon 3, intron 3, exon 4, intron 4, exon 5, intron 5, exon 6, intron 6, exon 7, intron 7, and exon 8 is replaced by a sequence including a region, a portion, or the entire sequence of the human exon 1, intron 1, exon 2, intron 2, exon 3, intron 3, exon 4, intron 4, exon 5, intron 5, exon 6, intron 6, exon 7, intron 7, and exon 8. In some embodiments, the extracellular region described herein includes the signal peptide. In some embodiments, the extracellular region described herein does not include the signal peptide.

In some embodiments, a “region” or “portion” of the signal peptide, extracellular region, transmembrane region, cytoplasmic region, exon 1, intron 1, exon 2, intron 2, exon 3, intron 3, exon 4, intron 4, exon 5, intron 5, exon 6, intron 6, exon 7, intron 7, and/or exon 8 of endogenous TSLPR protein or endogenous TSLPR gene is deleted.

In some embodiments, the present disclosure is related to a genetically-modified, non-human animal whose genome comprises a chimeric (e.g., humanized) TSLPR nucleotide sequence. In some embodiments, the chimeric (e.g., humanized) TSLPR nucleotide sequence encodes a TSLPR protein comprising an human or humanized TSLPR signal peptide, a human or humanized TSLPR extracellular region, a human or humanized TSLPR transmembrane regions, and an endogenous TSLPR cytoplasmic region. In some embodiments, the N-terminal 1, 2, 3, 4, 5, or 6 amino acids (e.g., Arg254 in SEQ ID NO: 7) in the endogenous TSLPR cytoplasmic region of the TSLPR protein described herein are replaced with the corresponding 1, 2, 3, 4, 5, or 6 amino acid in human TSLPR cytoplasmic region (e.g., Lys253 in SEQ ID NO: 8).

In some embodiments, the encoded protein comprises a sequence that is at least 80%, 85%, 90%, 95%, or 100% identical to SEQ ID NO: 7, 8, or 19. In some embodiments, the genome of the animal comprises a sequence that is at least 80%, 85%, 90%, 95%, or 100% identical to SEQ ID NO: 9, 10, 11, 12, 13, 14, 15, 16, 18, 20, 21, 22, 34, 35, 36, 37, 40, or 69.

In some embodiments, the genetically-modified non-human animal described herein comprises a sequence encoding a human or humanized TSLPR protein. In some embodiments, the TSLPR protein comprises a signal peptide, an extracellular region, a transmembrane region, and a cytoplasmic region. In some embodiments, the humanized TSLPR protein comprises a human or humanized TSLPR signal peptide. For example, the human or humanized TSLPR signal peptide comprises a sequence that is at least 70%, 80%, 85%, 90%, 95%, or 100% identical to amino acids 1-22 of SEQ ID NO: 8. In some embodiments, the humanized TSLPR protein comprises an endogenous TSLPR signal peptide. For example, the endogenous TSLPR signal peptide comprises a sequence that is at least 70%, 80%, 85%, 90%, 95%, or 100% identical to amino acids 1-19 of SEQ ID NO: 7. In some embodiments, the humanized TSLPR protein comprises a human or humanized TSLPR extracellular region. For example, the human or humanized TSLPR extracellular region comprises a sequence that is at least 70%, 80%, 85%, 90%, 95%, or 100% identical to amino acids 23-231 of SEQ ID NO: 8. In some embodiments, the humanized TSLPR protein comprises an endogenous TSLPR extracellular region. For example, the endogenous TSLPR extracellular region comprises a sequence that is at least 70%, 80%, 85%, 90%, 95%, or 100% identical to amino acids 20-232 of SEQ ID NO: 7. In some embodiments, the humanized TSLPR protein comprises a human or humanized TSLPR transmembrane region. For example, the human or humanized TSLPR transmembrane region comprises a sequence that is at least 70%, 80%, 85%, 90%, 95%, or 100% identical to amino acids 232-252 of SEQ ID NO: 8. In some embodiments, the humanized TSLPR protein comprises an endogenous TSLPR transmembrane region. For example, the endogenous TSLPR transmembrane region comprises a sequence that is at least 70%, 80%, 85%, 90%, 95%, or 100% identical to amino acids 233-253 of SEQ ID NO: 7. In some embodiments, the humanized TSLPR protein comprises a human or humanized TSLPR cytoplasmic region. For example, the human or humanized TSLPR cytoplasmic region comprises a sequence that is at least 70%, 80%, 85%, 90%, 95%, or 100% identical to amino acids 253-371 of SEQ ID NO: 8. In some embodiments, the humanized TSLPR protein comprises an endogenous TSLPR cytoplasmic region. For example, the endogenous TSLPR cytoplasmic region comprises a sequence that is at least 70%, 80%, 85%, 90%, 95%, or 100% identical to amino acids 254-359 of SEQ ID NO: 7.

In some embodiments, the genetically-modified non-human animal described herein comprises a human or humanized TSLPR gene. In some embodiments, the humanized TSLPR gene comprises from 5′ end to 3′ end: a portion (e.g., nucleotides 16-94) of human exon 1, human exons 2-5, a portion (e.g., nucleotides 662-774) of human exon 6, a portion (e.g., nucleotides 873-880) of endogenous exon 6, and endogenous exon 7-8. In some embodiments, the genetically-modified non-human animal described herein comprises a human or humanized TSLPR gene. In some embodiments, the humanized TSLPR gene comprises from 5′ end to 3′ end: a portion (e.g., nucleotides 1-113) of endogenous exon 1, a portion (e.g., nucleotides 19-94) of human exon 1, human exons 2-5, a portion (e.g., nucleotides 662-774) of human exon 6, a portion (e.g., nucleotides 873-880) of endogenous exon 6, and endogenous exon 7-8. In some embodiments, the humanized TSLPR gene further includes a STOP sequence. In some embodiments, the humanized TSLPR gene comprises human or humanized 5′ UTR. In some embodiments, the humanized TSLPR gene comprises human or humanized 3′ UTR. In some embodiments, the humanized TSLPR gene comprises endogenous 5′ UTR. In some embodiments, the humanized TSLPR gene comprises endogenous 3′ UTR.

In some embodiments, the genetically-modified non-human animal described herein comprises an insertion in its genome, at an endogenous TSLPR gene locus, of a sequence encoding a human or humanized TSLPR protein. In some embodiments, the inserted sequence comprises one or more sequences selected from: all or a portion of exon 1, exon 2, exon 3, exon 4, exon 5, exon 6, exon 7, and/or exon 8 of human TSLPR gene; and/or all or a portion of exon 1, exon 2, exon 3, exon 4, exon 5, exon 6, exon 7, and/or exon 8 of endogenous TSLPR gene (e.g., mouse TSLPR gene). In some embodiments, the inserted sequence is a cDNA sequence. In some embodiments, the inserted sequence includes, a portion of endogenous TSLPR exon 1 (optional), a portion of human TSLPR exon 1, human TSLPR exons 2-5, a portion of human TSLPR exon 6, a portion of endogenous TSLPR exon 6, endogenous TSLPR exon 7-8, a mouse TSLPR 3′ UTR sequence, and/or a STOP sequence. In some embodiments, the inserted sequence does not encode a TSLPR signal peptide. In some embodiments, the inserted sequence encodes a human TSLPR extracellular region, a human TSLPR transmembrane region, and an endogenous TSLPR cytoplasmic region.

In some embodiments, the insertion described herein is between any two nucleotides within exon 1, intron 1, exon 2, intron 2, exon 3, intron 3, exon 4, intron 4, exon 5, intron 5, exon 6, intron 6, exon 7, intron 7, and exon 8 of endogenous TSLPR gene (e.g., mouse TSLPR gene). In some embodiments, the insertion is between any two nucleotides within exon 2 of endogenous TSLPR gene. For example, the insertion is between any two of the nucleotides selected from the group consisting of positions 260-261 of exon 2 of endogenous TSLPR gene. In some embodiments, the insertion is between any two nucleotides within exon 1 of endogenous TSLPR gene. For example, the insertion is between any two of the nucleotides selected from the group consisting of positions 113-114 of exon 1 of endogenous TSLPR gene.

In some embodiments, the genetically modified animals can express a chimeric (e.g., humanized) TSLPR from endogenous mouse loci, wherein a sequence encoding the extracellular region and transmembrane region of human TSLPR, and the cytoplasmic region of endogenous TSLPR is inserted within exon 1 or exon 2 of endogenous TSLPR gene. In some embodiments, human portion of the chimeric (e.g., humanized) TSLPR comprises an amino acid sequence that is at least 80%, 90%, 95%, 96%, 97%, 98%, or 99% identical to amino acids 1-253 or 2-253 of SEQ ID NO: 8. In various embodiments, an endogenous non-human TSLPR locus is modified in whole or in part to comprise human nucleic acid sequence encoding at least the extracellular region and/or transmembrane region of human TSLPR protein.

In some embodiments, the genetically modified mice can express the chimeric TSLPR (e.g., humanized TSLPR) from endogenous loci that are under control of mouse promoters and/or mouse regulatory elements. The insertion at the endogenous mouse loci provides non-human animals that express chimeric TSLPR (e.g., humanized TSLPR) in appropriate cell types and in a manner that does not result in the potential pathologies observed in some other transgenic mice known in the art. The chimeric TSLPR (e.g., humanized TSLPR) expressed in animal can maintain one or more functions of the wild-type mouse or human TSLPR in the animal. For example, the expressed TSLPR can bind to human or non-human TSLP. Furthermore, in some embodiments, the animal does not express endogenous TSLPR. In some embodiments, the animal expresses a decreased level of endogenous TSLPR as compared to a wild-type animal. As used herein, the term “endogenous TSLPR” refers to TSLPR protein that is expressed from an endogenous TSLPR nucleotide sequence of the non-human animal (e.g., mouse) before any genetic modification.

The genome of the animal can comprise a sequence encoding an amino acid sequence that is at least 70%, 75%, 80%, 85%, 90%, 95%, 99%, or 100% identical to human TSLPR (NP_071431.2; SEQ ID NO: 8). In some embodiments, the genome comprises a sequence encoding an amino acid sequence that is at least 70%, 75%, 80%, 85%, 90%, 95%, 99%, or 100% identical to SEQ ID NO: 19.

The genome of the genetically modified animal can comprise an insertion at an endogenous TSLPR gene locus. In some embodiments, the sequence is inserted between two nucleotides within any sequence of the endogenous TSLPR gene locus, e.g., exon 1, exon 2, exon 3, exon 4, exon 5, exon 6, exon 7, exon 8, 5′-UTR, 3′-UTR, intron 1, intron 2, intron 3, intron 4, intron 5, intron 6, and intron 7. In some embodiments, the sequence is inserted within the regulatory region of the endogenous TSLPR gene. In some embodiments, the sequence is inserted within exon 1 or exon 2 of an endogenous mouse TSLPR gene locus.

The genetically modified animal can have one or more cells expressing a human or chimeric TSLPR (e.g., humanized TSLPR) having, from N-terminus to C-terminus, a signal peptide, an extracellular region, a transmembrane region, and a cytoplasmic region. In some embodiments, the extracellular region comprises a sequence that is at least 50%, 60%, 70%, 80%, 90%, 95%, 99% identical to the extracellular region of human TSLPR (e.g., amino acids 23-231 of SEQ ID NO: 8). In some embodiments, the extracellular region of the humanized TSLPR has a sequence that has at least 10, 20, 30, 40, 50, 60, 70, 80, 90, 100, 110, 120, 130, 140, 150, 160, 170, 180, 190, 200, 205, 206, 207, 208, or 209 amino acids (e.g., contiguously or non-contiguously) that are identical to the extracellular region of human TSLPR. Because human TSLPR and non-human TSLPR (e.g., mouse TSLPR) sequences, in many cases, are different, antibodies that bind to human TSLPR will not necessarily have the same binding affinity with non-human TSLPR or have the same effects to non-human TSLPR. Therefore, the genetically modified animal having a human or a humanized extracellular region can be used to better evaluate the effects of anti-human TSLPR antibodies in an animal model.

In some embodiments, the transmembrane region comprises a sequence that is at least 50%, 60%, 70%, 80%, 90%, 95%, 99% identical to the transmembrane region of human TSLPR (e.g., amino acids 232-252 of SEQ ID NO: 8). In some embodiments, the transmembrane region of the humanized TSLPR has a sequence that has at least 1, 2, 3, 4, 5, 6, 7, 8, 9, 10, 11, 12, 13, 14, 15, 16, 17, 18, 19, 20, or 21 amino acids (contiguously or non-contiguously) that are identical to the transmembrane region of human TSLPR.

In some embodiments, the signal peptide comprises a sequence that is at least 50%, 60%, 70%, 80%, 90%, 95%, 99% identical to the signal peptide of human TSLPR (e.g., amino acids 1-22 or 2-22 of SEQ ID NO: 8). In some embodiments, the transmembrane region of the humanized TSLPR has a sequence that has at least 1, 2, 3, 4, 5, 6, 7, 8, 9, 10, 11, 12, 13, 14, 15, 16, 17, 18, 19, 20, 21, or 22 amino acids (contiguously or non-contiguously) that are identical to the signal peptide of human TSLPR.

In some embodiments, the cytoplasmic comprises a sequence that is at least 50%, 60%, 70%, 80%, 90%, 95%, 99% identical to the cytoplasmic of endogenous TSLPR. In some embodiments, the cytoplasmic region of the humanized TSLPR has a sequence that has at least 10, 20, 30, 40, 50, 60, 70, 80, 90, 100, 110, 115, 116, 117, 118, or 119 amino acids (contiguously or non-contiguously) that are identical to the cytoplasmic region of endogenous TSLPR. In some embodiments, the entire extracellular region (including the signal peptide) and transmembrane region of the humanized TSLPR described herein are derived from human sequence. In some embodiments, the entire cytoplasmic region of the humanized TSLPR described herein are derived from endogenous sequence (e.g., mouse sequence).

In some embodiments, the genome of the genetically modified animal comprises a sequence that corresponds to a portion or the entire sequence of exon 1, exon 2, exon 3, exon 4, exon 5, and/or exon 6 of human TSLPR gene; a sequence encoding the extracellular region and the transmembrane region of human TSLPR; or a portion or the entire sequence of SEQ ID NO: 19.

In some embodiments, the genome of the genetically modified animal comprises a portion of exon 1, exons 2-5, and a portion of exon 6 of human TSLPR gene. In some embodiments, the portion of exon 1 includes at least 5, 10, 15, 20, 25, 30, 35, 40, 45, 50, 55, 60, 65, 70, 75, 76, 77, 78, 79, 80, 90, or 94 nucleotides. In some embodiments, the portion of exon 1 includes 76 or 79 nucleotides. In some embodiments, the portion of exon 1 includes a nucleotide sequence of at least 20 bp. In some embodiments, the portion of exon 1 starts from any one of the nucleotides encoding the N-terminal 1-5 (e.g., 1, 2, 3, 4, or 5) amino acids of TSLPR extracellular region (including the signal peptide) and ends at the last nucleotide of exon 1. In some embodiments, the portion of exon 6 includes at least 10, 20, 30, 40, 50, 60, 70, 80, 90, 100, 110, 111, 112, 113, 114, 115, 120, or 121 nucleotides. In some embodiments, the portion of exon 6 includes 113 nucleotides. In some embodiments, the portion of exon 6 includes a nucleotide sequence of at least 50 bp. In some embodiments, the human sequence encodes the entire extracellular region and transmembrane region of human TSLPR. In some embodiments, the human sequence further encodes the N-terminal 1, 2, 3, or 4 amino acids of the extracellular region.

In some embodiments, the non-human animal can have, at an endogenous TSLPR gene locus, a nucleotide sequence encoding a chimeric human/non-human TSLPR polypeptide, wherein a human portion of the chimeric human/non-human TSLPR polypeptide comprises the entire human TSLPR extracellular domain and the entire human TSLPR transmembrane region, and wherein the animal expresses a functional TSLPR on a surface of a cell (e.g., dendritic cell, CD4+ and CD8+ T cell, B cell, mast cell, natural killer T cell (NKT)) of the animal. The human portion of the chimeric human/non-human TSLPR polypeptide can comprise an amino acid sequence encoded by a portion of exon 1, exons 2-5, and/or a portion of exon 6 of human TSLPR gene. In some embodiments, the human portion of the chimeric human/non-human TSLPR polypeptide comprises a sequence that is at least 80%, 85%, 90%, 95%, or 99% identical to amino acids 1-253 or 2-253 of SEQ ID NO: 8. In some embodiments, the cytoplasmic region includes a sequence corresponding to the entire or part of amino acids 255-359 of SEQ ID NO: 7. In some embodiments, the chimeric human/non-human TSLPR polypeptide comprises a signal peptide, which includes a sequence corresponding to the entire or part of amino acids 1-22 of SEQ ID NO: 8.

In some embodiments, the non-human portion of the chimeric human/non-human TSLPR polypeptide comprises the entire cytoplasmic region of an endogenous non-human TSLPR polypeptide.

Furthermore, the genetically modified animal can be heterozygous with respect to the insertion at the endogenous TSLPR locus, or homozygous with respect to the insertion at the endogenous TSLPR locus.

In some embodiments, the humanized TSLPR locus lacks a human TSLPR 5′-UTR. In some embodiment, the humanized TSLPR locus comprises an endogenous (e.g., mouse) 5′-UTR. In some embodiments, the humanization comprises an endogenous (e.g., mouse) 3′-UTR. In appropriate cases, it may be reasonable to presume that the mouse and human TSLPR genes appear to be similarly regulated based on the similarity of their 5′-flanking sequence. As shown in the present disclosure, humanized TSLPR mice that comprise an insertion at an endogenous mouse TSLPR locus, which retain mouse regulatory elements but comprise a humanization of TSLPR encoding sequence, do not exhibit pathologies. Both genetically modified mice that are heterozygous or homozygous for humanized TSLPR are grossly normal.

In another aspect, the disclosure also provides a genetically-modified, non-human animal whose genome comprise a disruption in the animal's endogenous TSLPR gene, wherein the disruption of the endogenous TSLPR gene comprises deletion of exon 1, exon 2, exon 3, exon 4, exon 5, exon 6, exon 7, and/or exon 8, or part thereof of the endogenous TSLPR gene.

In some embodiments, the disruption of the endogenous TSLPR gene comprises deletion of one or more exons or part of exons selected from the group consisting of exon 1, exon 2, exon 3, exon 4, exon 5, exon 6, exon 7, and exon 8 the endogenous TSLPR gene.

In some embodiments, the disruption of the endogenous TSLPR gene further comprises deletion of one or more introns or part of introns selected from the group consisting of intron 1, intron 2, intron 3, intron 4, intron 5, intron 6, and intron 7 of the endogenous TSLPR gene.

In some embodiments, wherein the deletion can comprise deleting at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, 900, 1000, 2000, 3000, 4000, 4500, 4700, 5000, or more nucleotides.

In some embodiments, the disruption of the endogenous TSLPR gene comprises the deletion of at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, or 300 nucleotides of exon 1, exon 2, exon 3, exon 4, exon 5, exon 6, exon 7, and/or exon 8.

In some embodiments, the disruption of the endogenous TSLPR gene is caused by insertion of a sequence including one or more auxiliary sequences (e.g., WPRE, lox2, STOP, and/or polyA). The insertion can cause frameshift, mutation, or truncation of the endogenous TSLPR coding sequence, such that the level of transcription and/or translation of endogenous TSLPR gene is decreased (e.g., by at least 50%, at least 60%, at least 70%, at least 80%, at least 90%, at least 95%, or at least 99%).

The disclosure further relates to a TSLPR genomic DNA sequence of a humanized mouse, a DNA sequence obtained by a reverse transcription of the mRNA obtained by transcription thereof is consistent with or complementary to the DNA sequence; a construct expressing the amino acid sequence thereof; a cell comprising the construct thereof; a tissue comprising the cell thereof.

Thus, in some embodiments, the present disclosure also provides a chimeric (e.g., humanized) TSLPR nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse TSLPR mRNA sequence (e.g., NM_016715.4), mouse TSLPR amino acid sequence (e.g., SEQ ID NO: 7), or a portion thereof (e.g., a portion of exon 6, and exons 7-8); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human TSLPR mRNA sequence (e.g., NM_022148.4), human TSLPR amino acid sequence (e.g., SEQ ID NO: 8), or a portion thereof (e.g., a portion of exon 1, exons 2-5, and a portion of exon 6).

In some embodiments, the nucleic acids as described herein are operably linked to a promotor or regulatory element, e.g., an endogenous mouse TSLPR promotor, an inducible promoter, an enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from part of or the entire mouse TSLPR nucleotide sequence (e.g., exons 1-5, and a portion of exon 6 of NM_016715.4; alternatively, a portion of exon 1, exons 2-5, and a portion of exon 6 of NM_016715.4).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as part of or the entire mouse TSLPR nucleotide sequence (e.g., a portion of exon 6, and exons 7-8 of NM_016715.4; alternatively, a portion of exon 1, a portion of exon 6, exons 7-8 of NM_016715.4).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from part of or the entire human TSLPR nucleotide sequence (e.g., a portion of exon 6, and exons 7-8 of NM_022148.4; alternatively, a portion of exon 1, a portion of exon 6, and exons 7-8 of NM_022148.4).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as part of or the entire human TSLPR nucleotide sequence (e.g., a portion of exon 1, exons 2-5, and a portion of exon 6 of NM_022148.4).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from part of or the entire mouse TSLPR amino acid sequence (e.g., amino acids 1-254 of NP_057924.3 (SEQ ID NO: 7)). In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as part of or the entire mouse TSLPR amino acid sequence (e.g., amino acids 255-359 of NP_057924.3 (SEQ ID NO: 7)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from part of or the entire human TSLPR amino acid sequence (e.g., amino acids 254-371 of NP_071431.2 (SEQ ID NO: 8)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as part of or the entire human TSLPR amino acid sequence (e.g., amino acids 1-253 or 2-253 of NP_071431.2 (SEQ ID NO: 8)).

The present disclosure also provides a humanized TSLPR mouse amino acid sequence, wherein the amino acid sequence is selected from the group consisting of:
a) an amino acid sequence shown in SEQ ID NO: 7, 8, or 19;
b) an amino acid sequence having a homology of at least 90% with or at least 90% identical to the amino acid sequence shown in SEQ ID NO: 7, 8, or 19;
c) an amino acid sequence encoded by a nucleic acid sequence, wherein the nucleic acid sequence is able to hybridize to a nucleotide sequence encoding the amino acid shown in SEQ ID NO: 7, 8, or 19 under a low stringency condition or a strict stringency condition;
d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 7, 8, or 19;
e) an amino acid sequence that is different from the amino acid sequence shown in SEQ ID NO: 7, 8, or 19 by no more than 10, 9, 8, 7, 6, 5, 4, 3, 2 or no more than 1 amino acid; or
f) an amino acid sequence that comprises a substitution, a deletion and/or insertion of one or more amino acids to the amino acid sequence shown in SEQ ID NO: 7, 8, or 19.

The present disclosure also provides a humanized TSLPR amino acid sequence, wherein the amino acid sequence is selected from the group consisting of:
a) all or part of amino acids 1-253 or 2-253 of SEQ ID NO: 8;
b) an amino acid sequence have a homology of at least 90%, 91%, 92%, 93%, 94%, 95%, 96%, 97%, 98%, or 99% to amino acids 1-253 or 2-253 of SEQ ID NO: 8;
c) an amino acid sequence that is different from amino acids 1-253 or 2-253 of SEQ ID NO: 8 by no more than 10, 9, 8, 7, 6, 5, 4, 3, 2 or no more than 1 amino acid; and
d) an amino acid sequence that comprises a substitution, a deletion and/or insertion of one or more amino acids to amino acids 1-253 or 2-253 of SEQ ID NO: 8.

The present disclosure also relates to a TSLPR nucleic acid (e.g., DNA or RNA) sequence, wherein the nucleic acid sequence can be selected from the group consisting of:
a) a nucleic acid sequence as shown in SEQ ID NO: 9, 10, 11, 12, 13, 14, 15, 16, 18, 20, 21, 22, 34, 35, 36, 37, 40, 69, or a nucleic acid sequence encoding a homologous TSLPR amino acid sequence of a humanized mouse TSLPR;
b) a nucleic acid sequence that is able to hybridize to the nucleotide sequence as shown in SEQ ID NO: 9, 10, 11, 12, 13, 14, 15, 16, 18, 20, 21, 22, 34, 35, 36, 37, 40, or 69, under a low stringency condition or a strict stringency condition;
c) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 9, 10, 11, 12, 13, 14, 15, 16, 18, 20, 21, 22, 34, 35, 36, 37, 40, or 69;
d) a nucleic acid sequence that encodes an amino acid sequence, wherein the amino acid sequence has a homology of at least 90% with or at least 90% identical to the amino acid sequence shown in SEQ ID NO: 7, 8, or 19;
e) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 7, 8, or 19;
f) a nucleic acid sequence that encodes an amino acid sequence, wherein the amino acid sequence is different from the amino acid sequence shown in SEQ ID NO: 7, 8, or 19 by no more than 10, 9, 8, 7, 6, 5, 4, 3, 2 or no more than 1 amino acid; and/or
g) a nucleic acid sequence that encodes an amino acid sequence, wherein the amino acid sequence comprises a substitution, a deletion and/or insertion of one or more amino acids to the amino acid sequence shown in SEQ ID NO: 7, 8, or 19.

The present disclosure further relates to a TSLPR genomic DNA sequence of a humanized mouse. The DNA sequence is obtained by reverse transcription of the mRNA obtained by transcription thereof is consistent with or complementary to the DNA sequence homologous to the sequence shown in SEQ ID NO: 15, 16, 18, or 69.

### IL7R

Interleukin-7 (IL-7) was discovered in the last century and noted for its growth-promoting effects on progenitors of B cells in vivo. It was subsequently shown that IL-7 is a 25-kDa soluble globular protein. IL-7 is produced by cells, such as fetal liver cells, stromal cells in the bone marrow (BM), and thymus and other epithelial cells, including keratinocytes and enterocytes. IL-7R is a heterodimeric complex consisting of the α-chain (CD127) and the common cytokine receptor γ-chain, shared with the receptors for IL-2, IL-4, IL-7, IL-9, IL-15, and IL-21, and expressed in a variety of cells. Thus, IL-7 has multiple biological activities and influences various cell types through binding to its receptor. Deficiencies in IL-7 or IL-7R can lead to severely impaired immune cell development. In the ensuing decades, the discovery of relevant signaling pathways was accompanied by recognition that IL-7 plays an indispensable role in the development and maintenance of many other immune cells. The vital regulatory functions of IL-7 throughout the entire immune system have become increasingly evident.

IL7R is found on multiple subsets of lymphoid cells during their developmental and mature states. Both IL-7 and TSLP use IL-7R to initiate the formation of a heterodimeric receptor. IL-7 is a common gamma chain (γc) cytokine and requires the heterodimerization of IL-7R with the γc receptor (CD132) for signaling, whereas TSLP signaling requires heterodimerization with the TSLP receptor (TSLPR).

A detailed description of IL7R and its function can be found, e.g., in Chen, D., et al. “Interleukin-7 biology and its effects on immune cells: mediator of generation, differentiation, survival, and homeostasis.” Frontiers in Immunology (2021): 5156; and Sheikh, A., et al. “Interleukin-7 receptor alpha in innate lymphoid cells: more than a marker.” Frontiers in Immunology 10 (2019): 2897; each of which is incorporated by reference in its entirety.

In human genomes, IL7R gene (Gene ID: 3575) locus has eight exons, exon 1, exon 2, exon 3, exon 4, exon 5, exon 6, exon 7, and exon 8. The IL7R protein also has a signal peptide, an extracellular region, a transmembrane region, and a cytoplasmic region. The nucleotide sequence for human IL7R mRNA is NM_002185.5, and the amino acid sequence for human IL7R is NP_002176.2 (SEQ ID NO: 76). The location for each exon and each region in human IL7R nucleotide sequence and amino acid sequence is listed below:

TABLE 5

| | | NP_002176.2 |
|---|---|---|
| Human IL7R | NM_002185.5 | 459 aa |
| (approximate location) | 4584 bp | SEQ ID NO: 76 |
| Exon 1 | 1-169 | 1-27 |
| Exon 2 | 170-308 | 28-74 |
| Exon 3 | 309-466 | 75-126 |
| Exon 4 | 467-624 | 127-179 |
| Exon 5 | 625-793 | 180-235 |
| Exon 6 | 794-887 | 236-267 |
| Exon 7 | 888-963 | 268-292 |
| Exon 8 | 964-4584 | 293-459 |
| Signal peptide | 88-147 | 1-20 |
| Extracellular region | 148-804 | 21-239 |
| (excluding signal peptide region) | | |
| Transmembrane region | 805-879 | 240-264 |
| Cytoplasmic region | 880-1464 | 265-459 |
| Donor region in Example 8 | 88-804 | 1-239 |

In mice, IL7R gene (Gene ID: 16197) locus has eight exons, exon 1, exon 2, exon 3, exon 4, exon 5, exon 6, exon 7, and exon 8. The mouse IL7R protein also has a signal peptide, an extracellular region, a transmembrane region, and a cytoplasmic region. The nucleotide sequence for mouse IL7R mRNA is NM_008372.4, the amino acid sequence for mouse IL7R is NP_032398.3 (SEQ ID NO: 1). The location for each exon and each region in the mouse IL7R nucleotide sequence and amino acid sequence is listed below:

TABLE 6

| | | NP_032398.3 |
|---|---|---|
| Mouse IL7R | NM_008372.4 | 459 aa |
| (approximate location) | 3227 bp | SEQ ID NO: 75 |
| Exon 1 | 1-216 | 1-27 |
| Exon 2 | 217-355 | 28-74 |
| Exon 3 | 356-513 | 75-126 |
| Exon 4 | 514-671 | 127-179 |
| Exon 5 | 672-840 | 180-235 |
| Exon 6 | 841-934 | 236-267 |
| Exon 7 | 935-1010 | 268-292 |
| Exon 8 | 1011-3210 | 293-459 |
| Signal peptide | 135-194 | 1-20 |
| Extracellular region | 195-851 | 21-239 |
| (excluding signal peptide region) | | |
| Transmembrane region | 852-926 | 240-264 |
| Cytoplasmic region | 927-1511 | 265-459 |
| Replaced region in Example 8 | 135-851 | 1-239 |

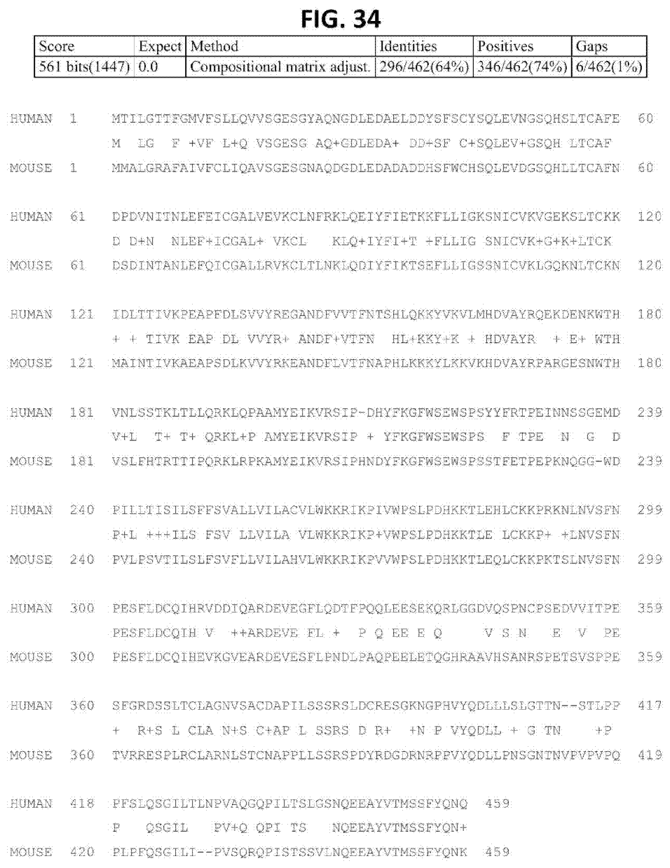

FIG. 34 shows the alignment between human IL7R amino acid sequence (NP_002176.2; SEQ ID NO: 76) and mouse IL7R amino acid sequence (NP_032398.3; SEQ ID NO: 75). Thus, the corresponding amino acid residue or region between human and mouse IL7R can be found in FIG. 34.

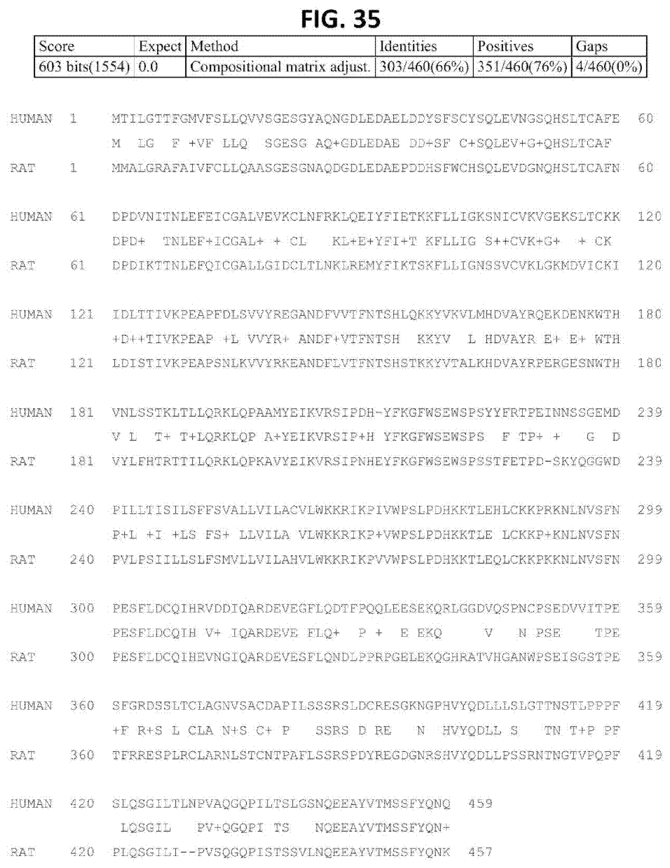

IL7R genes, proteins, and locus of the other species are also known in the art. For example, the gene ID for IL7R in Rattus norvegicus (rat) is 294797, the gene ID for IL7R in Macaca mulatta (Rhesus monkey) is 699869, the gene ID for IL7R in Canis lupus familiaris (dog) is 612582, and the gene ID for IL7R in Sus scrofa (pig) is 100271930. The relevant information for these genes (e.g., intron sequences, exon sequences, amino acid residues of these proteins) can be found, e.g., in NCBI database, which is incorporated by reference herein in its entirety. FIG. 35 shows the alignment between human IL7R amino acid sequence (NP_002176.2; SEQ ID NO: 76) and rat IL7R amino acid sequence (NP_001099888.1; SEQ ID NO: 84). Thus, the corresponding amino acid residue or region between human and rodent IL7R can be found in FIG. 35.

The present disclosure provides human or chimeric (e.g., humanized) IL7R nucleotide sequence and/or amino acid sequences. In some embodiments, the entire sequence of mouse exon 1, exon 2, exon 3, exon 4, exon 5, exon 6, exon 7, exon 8, signal peptide, extracellular region, transmembrane region, and/or cytoplasmic region are replaced by the corresponding human sequence. In some embodiments, a “region” or “portion” of mouse exon 1, exon 2, exon 3, exon 4, exon 5, exon 6, exon 7, exon 8, signal peptide, extracellular region, transmembrane regions, and/or cytoplasmic regions are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 450, 500, 550, 580, 590, 600, 650, 700, 710, 715, 716, or 717 nucleotides, or at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 235, 236, 237, 238, or 239 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, exon 8, signal peptide, extracellular region, transmembrane region, or cytoplasmic region. In some embodiments, a region, a portion, or the entire sequence of mouse exon 1, exon 2, exon 3, exon 4, exon 5, exon 6, exon 7, and/or exon 8 (e.g., a portion of exon 1, exons 2-5, and a portion of exon 6) are replaced by a region, a portion, or the entire sequence of the human exon 1, exon 2, exon 3, exon 4, exon 5, exon 6, exon 7, and/or exon 8 (e.g., a portion of exon 1, exons 2-5, and a portion of exon 6). In some embodiments, the extracellular region described herein includes the signal peptide. In some embodiments, the extracellular region described herein does not include the signal peptide.

In some embodiments, a “region” or “portion” of the signal peptide, extracellular region, transmembrane region, cytoplasmic region, exon 1, exon 2, exon 3, exon 4, exon 5, exon 6, exon 7, and/or exon 8 is deleted.

In some embodiments, the present disclosure is related to a genetically-modified, non-human animal whose genome comprises a chimeric (e.g., humanized) IL7R nucleotide sequence. In some embodiments, the chimeric (e.g., humanized) IL7R nucleotide sequence encodes a IL7R protein comprising an extracellular region. In some embodiments, the extracellular region described herein is at least 80%, 85%, 90%, 95%, or 100% identical to amino acids 21-239 of SEQ ID NO: 76. In some embodiments, the extracellular region comprises the entire or part of human IL7R extracellular region. In some embodiments, the genome of the animal comprises a sequence that is at least 80%, 85%, 90%, 95%, or 100% identical to SEQ ID NO: 77, 78, 79, or 80.

In some embodiments, the genetically-modified non-human animal described herein comprises a sequence encoding a human or humanized IL7R protein. In some embodiments, the IL7R protein comprises a signal peptide, an extracellular region, a transmembrane region, and a cytoplasmic region. In some embodiments, the humanized IL7R protein comprises a human or humanized IL7R signal peptide. For example, the human or humanized IL7R signal peptide comprises a sequence that is at least 70%, 80%, 85%, 90%, 95%, or 100% identical to amino acids 1-20 of SEQ ID NO: 76. In some embodiments, the humanized IL7R protein comprises an endogenous IL7R signal peptide. For example, the endogenous IL7R signal peptide comprises a sequence that is at least 70%, 80%, 85%, 90%, 95%, or 100% identical to amino acids 1-20 of SEQ ID NO: 75. In some embodiments, the humanized IL7R protein comprises a human or humanized IL7R extracellular region. For example, the human or humanized IL7R extracellular region comprises a sequence that is at least 70%, 80%, 85%, 90%, 95%, or 100% identical to amino acids 21-239 of SEQ ID NO: 76. In some embodiments, the humanized IL7R protein comprises an endogenous IL7R extracellular region. For example, the endogenous IL7R extracellular region comprises a sequence that is at least 70%, 80%, 85%, 90%, 95%, or 100% identical to amino acids 21-239 of SEQ ID NO: 75. In some embodiments, the humanized IL7R protein comprises a human or humanized IL7R transmembrane region. For example, the human or humanized IL7R transmembrane region comprises a sequence that is at least 70%, 80%, 85%, 90%, 95%, or 100% identical to amino acids 240-264 of SEQ ID NO: 76. In some embodiments, the humanized IL7R protein comprises an endogenous IL7R transmembrane region. For example, the endogenous IL7R transmembrane region comprises a sequence that is at least 70%, 80%, 85%, 90%, 95%, or 100% identical to amino acids 260-264 of SEQ ID NO: 75. In some embodiments, the humanized IL7R protein comprises a human or humanized IL7R cytoplasmic region. For example, the human or humanized IL7R cytoplasmic region comprises a sequence that is at least 70%, 80%, 85%, 90%, 95%, or 100% identical to amino acids 265-459 of SEQ ID NO: 76. In some embodiments, the humanized IL7R protein comprises an endogenous IL7R cytoplasmic region. For example, the endogenous IL7R cytoplasmic region comprises a sequence that is at least 70%, 80%, 85%, 90%, 95%, or 100% identical to amino acids 265-459 of SEQ ID NO: 75.

In some embodiments, the genetically-modified non-human animal described herein comprises a human or humanized IL7R gene. In some embodiments, the humanized IL7R gene comprises 8 exons. In some embodiments, the humanized IL7R gene comprises human or humanized exon 1 (e.g., chimeric exon including part of mouse IL7R exon 1 and part of human IL7R exon 1), human or humanized exon 2 (e.g., human IL7R exon 2), human or humanized exon 3 (e.g., human IL7R exon 3), human or humanized exon 4 (e.g., human IL7R exon 4), human or humanized exon 5 (e.g., human IL7R exon 5), human or humanized exon 6 (e.g., chimeric exon including part of human IL7R exon 6 and part of mouse IL7R exon 6), endogenous exon 7 (e.g., mouse IL7R exon 7), and/or endogenous exon 8 (e.g., mouse IL7R exon 8). In some embodiments, the humanized IL7R gene comprises human or humanized intron 1, human or humanized intron 2, human or humanized intron 3, human or humanized intron 4, human or humanized intron 5, endogenous intron 6, and/or endogenous intron 7. In some embodiments, the humanized IL7R gene comprises human or humanized 5′ UTR. In some embodiments, the humanized IL7R gene comprises human or humanized 3′ UTR. In some embodiments, the humanized IL7R gene comprises endogenous 5′ UTR. In some embodiments, the humanized IL7R gene comprises endogenous 3′ UTR.

Thus, in some embodiments, the present disclosure also provides a chimeric (e.g., humanized) IL7R nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse IL7R mRNA sequence (e.g., NM_008372.4), mouse IL7R amino acid sequence (e.g., SEQ ID NO: 75), or a portion thereof (e.g., a portion of exon 1, a portion of exon 6, exons 7-8); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human IL7R mRNA sequence (e.g., NM_002185.5), human IL7R amino acid sequence (e.g., SEQ ID NO: 76), or a portion thereof (e.g., a portion of exon 1, exons 2-5, and a portion of exon 6).

In some embodiments, the sequence encoding amino acids 1-239 of mouse IL7R (SEQ ID NO: 75) is replaced. In some embodiments, the sequence is replaced by a sequence encoding a corresponding region of human IL7R (e.g., amino acids 1-239 of human IL7R (SEQ ID NO: 76)). In some embodiments, the sequence encoding amino acids 21-239 of mouse IL7R (SEQ ID NO: 75) is replaced. In some embodiments, the sequence is replaced by a sequence encoding a corresponding region of human IL7R (e.g., amino acids 21-239 of human IL7R (SEQ ID NO: 76)). In some embodiments, the sequence encoding amino acids 1-459 of mouse IL7R (SEQ ID NO: 75) is replaced. In some embodiments, the sequence is replaced by a sequence encoding a corresponding region of human IL7R (e.g., amino acids 1-459 of human IL7R (SEQ ID NO: 76)).

In some embodiments, the nucleic acids as described herein are operably linked to a promotor or regulatory element, e.g., an endogenous mouse IL7R promotor, an inducible promoter, an enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from part of or the entire mouse IL7R nucleotide sequence (e.g., a portion of exon 1, exons 2-5, and a portion of exon 6 of NM_008372.4).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as part of or the entire mouse IL7R nucleotide sequence (e.g., 5′ UTR, a portion of exon 1, a portion of exon 6, exons 7-8, and 3′ UTR of NM_008372.4).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from part of or the entire human IL7R nucleotide sequence (e.g., 5′ UTR, a portion of exon 1, a portion of exon 6, exons 7-8, and 3′ UTR of NM_002185.5).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as part of or the entire human IL7R nucleotide sequence (e.g., a portion of exon 1, exons 2-5, and a portion of exon 6 of NM_002185.5).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from part of or the entire mouse IL7R amino acid sequence (e.g., amino acids 1-239 of NP_032398.3 (SEQ ID NO: 75)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as part of or the entire mouse IL7R amino acid sequence (e.g., amino acids 240-459 of NP_032398.3 (SEQ ID NO: 75)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from part of or the entire human IL7R amino acid sequence (e.g., amino acids 240-459 of NP_002176.2 (SEQ ID NO: 76)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as part of or the entire human IL7R amino acid sequence (e.g., amino acids 1-239 of NP_002176.2 (SEQ ID NO: 76)).

The present disclosure also provides a humanized IL7R mouse amino acid sequence, wherein the amino acid sequence is selected from the group consisting of:
a) an amino acid sequence shown in SEQ ID NO: 75, 76, or 81;
b) an amino acid sequence having a homology of at least 90% with or at least 90% identical to the amino acid sequence shown in SEQ ID NO: 75, 76, or 81;
c) an amino acid sequence encoded by a nucleic acid sequence, wherein the nucleic acid sequence is able to hybridize to a nucleotide sequence encoding the amino acid shown in SEQ ID NO: 75, 76, or 81 under a low stringency condition or a strict stringency condition;
d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 75, 76, or 81;
e) an amino acid sequence that is different from the amino acid sequence shown in SEQ ID NO: 75, 76, or 81 by no more than 10, 9, 8, 7, 6, 5, 4, 3, 2 or no more than 1 amino acid; or
f) an amino acid sequence that comprises a substitution, a deletion and/or insertion of one or more amino acids to the amino acid sequence shown in SEQ ID NO: 75, 76, or 81.

The present disclosure also provides a humanized IL7R amino acid sequence, wherein the amino acid sequence is selected from the group consisting of:
a) all or part of amino acids 1-239 of SEQ ID NO: 76;
b) an amino acid sequence have a homology of at least 90%, 91%, 92%, 93%, 94%, 95%, 96%, 97%, 98%, or 99% to amino acids 1-239 of SEQ ID NO: 76;
c) an amino acid sequence that is different from amino acids 1-239 of SEQ ID NO: 76 by no more than 10, 9, 8, 7, 6, 5, 4, 3, 2 or no more than 1 amino acid; and
d) an amino acid sequence that comprises a substitution, a deletion and/or insertion of one or more amino acids to amino acids 1-239 of SEQ ID NO: 76.

The present disclosure also relates to a IL7R nucleic acid (e.g., DNA or RNA) sequence, wherein the nucleic acid sequence can be selected from the group consisting of:
a) a nucleic acid sequence as shown in SEQ ID NO: 77, 78, 79, or 80, or a nucleic acid sequence encoding a homologous IL7R amino acid sequence of a humanized mouse IL7R;
b) a nucleic acid sequence that is able to hybridize to the nucleotide sequence as shown in SEQ ID NO: 77, 78, 79, or 80 under a low stringency condition or a strict stringency condition;
c) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 77, 78, 79, or 80;
d) a nucleic acid sequence that encodes an amino acid sequence, wherein the amino acid sequence has a homology of at least 90% with or at least 90% identical to the amino acid sequence shown in SEQ ID NO: 75, 76, or 81;
e) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 75, 76, or 81;
f) a nucleic acid sequence that encodes an amino acid sequence, wherein the amino acid sequence is different from the amino acid sequence shown in SEQ ID NO: 75, 76, or 81 by no more than 10, 9, 8, 7, 6, 5, 4, 3, 2 or no more than 1 amino acid; and/or
g) a nucleic acid sequence that encodes an amino acid sequence, wherein the amino acid sequence comprises a substitution, a deletion and/or insertion of one or more amino acids to the amino acid sequence shown in SEQ ID NO: 75, 76, or 81.

The present disclosure further relates to a IL7R genomic DNA sequence of a humanized mouse. The DNA sequence is obtained by reverse transcription of the mRNA obtained by transcription thereof is consistent with or complementary to the DNA sequence homologous to the sequence shown in SEQ ID NO: 80.

### IL33

Interleukin 33 (IL-33 or IL33) was identified as a member of the IL-1 family of cytokines and the ligand for ST2L. It is constitutively expressed in many tissues and by a wide variety of cells. It is also induced in response to various stimuli in epithelial cells, myofibroblasts, adipocytes, endothelial cells, smooth muscle cells, and macrophages predominantly as a pro-inflammatory cytokine. IL-33 is about 30 kDa that functions dually as a transcription factor and a cytokine. Its N-terminus contains a nuclear localization signal, a DNA-binding homeodomain-like helix-turn-helix motif, and a chromatin binding domain, while the C-terminus contains an IL-1 like cytokine domain.

A detailed description of IL33 and its function can be found, e.g., in Larsen et al., “The role of IL-33/ST2 pathway in tumorigenesis.” International Journal of Molecular Sciences 19.9 (2018): 2676; Shen et al., “Interleukin-33 in malignancies: friends or foes?.” Frontiers in Immunology 9 (2018): 3051; each of which is incorporated by reference in its entirety.

In human genomes, IL33 gene (Gene ID: 90865) locus has 8 exons, exon 1, exon 2, exon 3, exon 4, exon 5, exon 6, exon 7, and exon 8. The nucleotide sequence for human IL33 mRNA is NM_033439.3, and the amino acid sequence for human IL33 is NP_254274.1 (SEQ ID NO: 66). The location for each exon and each region in human IL33 nucleotide sequence and amino acid sequence is listed below:

TABLE 7

| | | NP_254274.1 |
|---|---|---|
| Human IL33 | NM_033439.3 | 270 amino acids |
| (approximate location) | 2718 bp | (SEQ ID NO: 66) |
| Exon 1 | 1-67 | Non-coding |
| Exon 2 | 68-169 | 1-30 |
| Exon 3 | 170-295 | 31-72 |
| Exon 4 | 296-421 | 73-114 |
| Exon 5 | 422-547 | 115-156 |
| Exon 6 | 548-598 | 157-173 |
| Exon 7 | 599-690 | 174-204 |
| Exon 8 | 691-2706 | 205-270 |
| Donor region in Example 7 | 79-891 | 1-270 |

In mice, IL33 gene (Gene ID: 77125) locus has 8 exons, exon 1, exon 2, exon 3, exon 4, exon 5, exon 6, exon 7, and exon 8. The nucleotide sequence for mouse IL33 mRNA is NM_133775.3, the amino acid sequence for mouse IL33 is NP_598536.2 (SEQ ID NO: 65). The location for each exon and each region in the mouse IL33 nucleotide sequence and amino acid sequence is listed below:

TABLE 8

| | | NP_598536.2 |
|---|---|---|
| Mouse IL33 | NM_133775.3 | 266 amino acids |
| (approximate location) | 2538 bp | (SEQ ID NO: 65) |
| Exon 1 | 1-49 | Non-coding |
| Exon 2 | 50-157 | 1-32 |
| Exon 3 | 158-283 | 33-74 |
| Exon 4 | 284-394 | 75-111 |
| Exon 5 | 395-520 | 112-153 |
| Exon 6 | 521-571 | 154-170 |
| Exon 7 | 572-669 | 171-203 |
| Exon 8 | 670-2538 | 204-266 |
| Replaced region in Example 7 | 61-861 | 1-266 |

The mouse IL33 gene (Gene ID: 77125) is located in Chromosome 19 of the mouse genome, which is located from 29925114 to 29960715, of NC_000085.6 (GRCm38.p4 (GCF_000001635.24)). The 5′-UTR is from 29,925,114 to 29,925,161 and 29,949,660 to 29,949,670, exon 1 is from 29,925,114 to 29,925,161, the first intron is from 29,925,162 to 29,949,659, exon 2 is from 29,949,660 to 29,949,767, the second intron is from 29,949,768 to 29,951,975, exon 3 is from 29,951,976 to 29,952,101, the third intron is from 29,952,102 to 29,952,729, exon 4 is from 29,952,730 to 29,952,840, the fourth intron is from 29,952,841 to 29,954,542, exon 5 is from 29,954,543 to 29,954,668, the fifth intron is from 29,954,669 to 29,955,200, the exon 6 is from 29,955,201 to 29,955,251, the sixth intron is from 29,955,252 to 29,956,900, the exon 7 is from 29,956,901 to 29,956,998 the seventh intron is from 29,956,999 to 29,958,849, the exon 8 is from 29,958,850 to 29,960,718, and the 3′-UTR is from 29,959,042 to 29,960,718, based on transcript NM_133775.3. All relevant information for mouse IL33 locus can be found in the NCBI website with Gene ID: 77125, which is incorporated by reference herein in its entirety.

IL33 genes, proteins, and locus of the other species are also known in the art. For example, the gene ID for IL33 in Rattus norvegicus (rat) is 361749, the gene ID for IL33 in Macaca mulatta (Rhesus monkey) is 717301, the gene ID for IL33 in Sus scrofa (pig) is 100518643, the gene ID for IL33 in Oryctolagus cuniculus (rabbit) is 100356081, and the gene ID for IL33 in Felis catus (domestic cat) is 101093403. The relevant information for these genes (e.g., intron sequences, exon sequences, amino acid residues of these proteins) can be found, e.g., in NCBI database, which is incorporated by reference herein in its entirety.

The present disclosure provides human or chimeric (e.g., humanized) IL33 nucleotide sequence and/or amino acid sequences. In some embodiments, the entire sequence of mouse exon 1, exon 2, exon 3, exon 4, exon 5, exon 6, exon 7, exon 8, and/or signal peptide, are replaced by the corresponding human sequence. In some embodiments, a “region” or a “portion” of mouse exon 1, exon 2, exon 3, exon 4, exon 5, exon 6, exon 7, exon 8, and/or signal peptide, are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 500, or 600 nucleotides, or at least 1, 2, 3, 4, 5, 6, 7, 8, 9, 10, 20, 30, 40, 50, 60, 70, 80, 90, 100, 110, 120, 130, 140, 150, 160, 170, 180, 190, or 200 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, exon 8, or signal peptide. In some embodiments, a region, a portion, or the entire sequence of mouse exon 1, exon 2, exon 3, exon 4, exon 5, exon 6, exon 7, and/or exon 8 (e.g., a portion of exon 2, exon 3, exon 4, exon 5, exon 6, exon 7, and a portion of exon 8 of mouse IL33 gene) are replaced by human exon 1, exon 2, exon 3, exon 4, exon 5, exon 6, exon 7, and/or exon 8 (e.g., a portion of exon 2, exon 3, exon 4, exon 5, exon 6, exon 7, and a portion of exon 8 of human IL33 gene) sequence.

In some embodiments, the present disclosure also provides a chimeric (e.g., humanized) or human IL33 nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse IL33 mRNA sequence (e.g., NM_133775.3), mouse IL33 amino acid sequence (e.g., SEQ ID NO: 65), or a portion thereof (e.g., exon 1, a portion of exon 2, and a portion of exon 8, of NM_133775.3); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human IL33 mRNA sequence (e.g., NM_033439.3), human IL33 amino acid sequence (e.g., SEQ ID NO: 66), or a portion thereof (e.g., a portion of exon 2, exon 3, exon 4, exon 5, exon 6, exon 7, and a portion of exon 8, of NM_033439.3).

In some embodiments, the sequence encoding amino acids 1-266 of mouse IL33 (SEQ ID NO: 65) is replaced. In some embodiments, the sequence is replaced by a sequence encoding a corresponding region of human IL33 (e.g., amino acids 1-270 of human IL33 (SEQ ID NO: 66)).

In some embodiments, the nucleic acid sequence described herein are operably linked to a promotor or regulatory element, e.g., an endogenous mouse IL33 promotor, an inducible promoter, an enhancer, and/or mouse or human regulatory elements. In some embodiments, the nucleic acid sequence described herein is connected to an endogenous 5′ UTR. In some embodiments, the 5′UTR is identical to nucleic acid positions 1-60 of NM_133775.3. In some embodiments, the nucleic acid sequence described herein is connected to a human 5′ UTR. In some embodiments, the nucleic acid sequence described herein is connected to an endogenous 3′ UTR. In some embodiments, the nucleic acid sequence described herein is connected to a human 3′ UTR.

In some embodiments, the nucleic acid sequence described herein has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from a portion of or the entire mouse IL33 nucleotide sequence (e.g., a portion of exon 2, exons 3-7, and a portion of exon 8 of NM_133775.3).

In some embodiments, the nucleic acid sequence described herein has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire mouse IL33 nucleotide sequence (e.g., exon 1, a portion of exon 2, and a portion of exon 8 of NM_133775.3).

In some embodiments, the nucleic acid sequence described herein has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from a portion of or the entire human IL33 nucleotide sequence (e.g., exon 1, a portion of exon 2, and a portion of exon 8 of NM_033439.3).

In some embodiments, the nucleic acid sequence described herein has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire human IL33 nucleotide sequence (e.g., a portion of exon 2, exons 3-7, and a portion of exon 8 of NM_033439.3).

In some embodiments, the amino acid sequence described herein has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire mouse IL33 amino acid sequence (e.g., NP_598536.2 (SEQ ID NO: 65)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire mouse IL33 amino acid sequence (e.g., NP_598536.2 (SEQ ID NO: 65)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire human IL33 amino acid sequence (e.g., NP_254274.1 (SEQ ID NO: 66)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire human IL33 amino acid sequence (e.g., NP_254274.1 (SEQ ID NO: 66)).

The present disclosure also provides a human or humanized IL33 amino acid sequence, wherein the amino acid sequence is selected from the group consisting of:
a) an amino acid sequence shown in SEQ ID NO: 65 or 66;
b) an amino acid sequence having a homology of at least 90% with or at least 90% identical to the amino acid sequence shown in SEQ ID NO: 65 or 66;
c) an amino acid sequence encoded by a nucleic acid sequence, wherein the nucleic acid sequence is able to hybridize to a nucleotide sequence encoding the amino acid shown in SEQ ID NO: 65 or 66, under a low stringency condition or a strict stringency condition;
d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 65 or 66;
e) an amino acid sequence that is different from the amino acid sequence shown in SEQ ID NO: 65 or 66, by no more than 10, 9, 8, 7, 6, 5, 4, 3, 2 or no more than 1 amino acid; or
f) an amino acid sequence that comprises a substitution, a deletion and/or insertion of one or more amino acids to the amino acid sequence shown in SEQ ID NO: 65 or 66.

The present disclosure also relates to a IL33 nucleic acid (e.g., DNA or RNA) sequence, wherein the nucleic acid sequence can be selected from the group consisting of:
a) a nucleic acid sequence as shown in SEQ ID NO: 67, 68, or 85, or a nucleic acid sequence encoding a homologous IL33 amino acid sequence of a humanized mouse IL33;
b) a nucleic acid sequence that is shown in SEQ ID NO: 67, 68, or 85;
c) a nucleic acid sequence that is able to hybridize to the nucleotide sequence as shown in SEQ ID NO: 67, 68, or 85 under a low stringency condition or a strict stringency condition;
d) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 67, 68, or 85;
e) a nucleic acid sequence that encodes an amino acid sequence, wherein the amino acid sequence has a homology of at least 90% with or at least 90% identical to the amino acid sequence shown in SEQ ID NO: 65 or 66;
f) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 65 or 66;
g) a nucleic acid sequence that encodes an amino acid sequence, wherein the amino acid sequence is different from the amino acid sequence shown in SEQ ID NO: 65 or 66 by no more than 10, 9, 8, 7, 6, 5, 4, 3, 2 or no more than 1 amino acid; and/or
h) a nucleic acid sequence that encodes an amino acid sequence, wherein the amino acid sequence comprises a substitution, a deletion and/or insertion of one or more amino acids to the amino acid sequence shown in SEQ ID NO: 65 or 66.

The present disclosure further relates to an IL33 genomic DNA sequence of a humanized mouse. The DNA sequence is obtained by a reverse transcription of the mRNA obtained by transcription thereof is consistent with or complementary to the DNA sequence homologous to the sequence shown in SEQ ID NO: 85.

Detailed methods of humanizing an endogenous (e.g., mouse) IL33 gene locus can be found, e.g., in PCT Application Publication No. WO2021018198, which is incorporated herein by reference in its entirety.

The disclosure also provides an amino acid sequence that has a homology of at least 90% with, or at least 90% identical to the sequence shown in SEQ ID NO: 1, 2, 7, 8, 19, 65, 66, 75, 76, or 81, and has protein activity. In some embodiments, the homology with the sequence shown in SEQ ID NO: 1, 2, 7, 8, 19, 65, 66, 75, 76, or 81 is at least about 90%, 91%, 92%, 93%, 94%, 95%, 96%, 97%, 98%, or at least 99%. In some embodiments, the foregoing homology is at least about 60%, 61%, 62%, 63%, 64%, 65%, 66%, 67%, 68%, 69%, 70%, 71%, 72%, 73%, 74%, 75%, 80%, or 85%.

In some embodiments, the percentage identity with the sequence shown in SEQ ID NO: 1, 2, 7, 8, 19, 65, 66, 75, 76, or 81 is at least about 90%, 91%, 92%, 93%, 94%, 95%, 96%, 97%, 98%, or at least 99%. In some embodiments, the foregoing percentage identity is at least about 60%, 61%, 62%, 63%, 64%, 65%, 66%, 67%, 68%, 69%, 70%, 71%, 72%, 73%, 74%, 75%, 80%, or 85%.

The disclosure also provides a nucleotide sequence that has a homology of at least 90%, or at least 90% identical to the sequence shown in SEQ ID NO: 5, 6, 15, 16, 18, 69, 80, or 85 and encodes a polypeptide that has protein activity. In some embodiments, the homology with the sequence shown in SEQ ID NO: 5, 6, 15, 16, 18, 69, 80, or 85 is at least about 90%, 91%, 92%, 93%, 94%, 95%, 96%, 97%, 98%, or at least 99%. In some embodiments, the foregoing homology is at least about 50%, 55%, 60%, 65%, 66%, 67%, 68%, 69%, 70%, 71%, 72%, 73%, 74%, 75%, 80%, or 85%.

In some embodiments, the percentage identity with the sequence shown in SEQ ID NO: 3, 4, 5, 6, 9, 10, 11, 12, 13, 14, 15, 16, 17, 18, 20, 21, 22, 23, 24, 25, 26, 34, 35, 36, 37, 40, 67, 68, 69, 77, 78, 79, 80, or 85 is at least about 90%, 91%, 92%, 93%, 94%, 95%, 96%, 97%, 98%, or at least 99%. In some embodiments, the foregoing percentage identity is at least about 50%, 55%, 60%, 65%, 66%, 67%, 68%, 69%, 70%, 71%, 72%, 73%, 74%, 75%, 80%, or 85%.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The percentage of residues conserved with similar physicochemical properties (percent homology), e.g. leucine and isoleucine, can also be used to measure sequence similarity. Families of amino acid residues having similar physicochemical properties have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

Cells, tissues, and animals (e.g., mouse) are also provided that comprise the nucleotide sequences as described herein, as well as cells, tissues, and animals (e.g., mouse) that express human or chimeric (e.g., humanized) TSLP from an endogenous non-human TSLP locus, and/or human or chimeric (e.g., humanized) TSLPR from an endogenous non-human TSLPR locus.

### Genetically Modified Animals

As used herein, the term “genetically-modified non-human animal” refers to a non-human animal having exogenous DNA in at least one chromosome of the animal's genome. In some embodiments, at least one or more cells, e.g., at least 1%, 2%, 3%, 4%, 5%, 10%, 20%, 30%, 40%, 50% of cells of the genetically-modified non-human animal have the exogenous DNA in its genome. The cell having exogenous DNA can be various kinds of cells, e.g., an endogenous cell, a somatic cell, an immune cell, a T cell, a B cell, an NK cell, an antigen presenting cell, a macrophage, a dendritic cell, a germ cell, a blastocyst, or an endogenous tumor cell. In some embodiments, genetically-modified non-human animals are provided that comprise a modified endogenous TSLP and/or TSLPR loci that comprise an exogenous sequence (e.g., a human sequence), e.g., a replacement of one or more non-human sequences with one or more human sequences, or an insertion of one or more human and/or non-human sequences. The animals are generally able to pass the modification to progeny, i.e., through germline transmission.

As used herein, the term “chimeric gene” or “chimeric nucleic acid” refers to a gene or a nucleic acid, wherein two or more portions of the gene or the nucleic acid are from different species, or at least one of the sequences of the gene or the nucleic acid does not correspond to the wild-type nucleic acid in the animal. In some embodiments, the chimeric gene or chimeric nucleic acid has at least one portion of the sequence that is derived from two or more different sources, e.g., sequences encoding different proteins or sequences encoding the same (or homologous) protein of two or more different species. In some embodiments, the chimeric gene or the chimeric nucleic acid is a humanized gene or humanized nucleic acid.

As used herein, the term “chimeric protein” or “chimeric polypeptide” refers to a protein or a polypeptide, wherein two or more portions of the protein or the polypeptide are from different species, or at least one of the sequences of the protein or the polypeptide does not correspond to wild-type amino acid sequence in the animal. In some embodiments, the chimeric protein or the chimeric polypeptide has at least one portion of the sequence that is derived from two or more different sources, e.g., same (or homologous) proteins of different species. In some embodiments, the chimeric protein or the chimeric polypeptide is a humanized protein or a humanized polypeptide.

As used herein, the term “humanized protein” or “humanized polypeptide” refers to a protein or a polypeptide, wherein at least a portion of the protein or the polypeptide is from the human protein or human polypeptide. In some embodiments, the humanized protein or polypeptide is a human protein or polypeptide.

As used herein, the term “humanized nucleic acid” refers to a nucleic acid, wherein at least a portion of the nucleic acid is from the human. In some embodiments, the entire nucleic acid of the humanized nucleic acid is from human. In some embodiments, the humanized nucleic acid is a humanized exon. A humanized exon can be e.g., a human exon or a chimeric exon.

In some embodiments, the chimeric gene or the chimeric nucleic acid is a humanized TSLP gene or a humanized TSLP nucleic acid. In some embodiments, at least one or more portions of the gene or the nucleic acid is from the human TSLP gene, at least one or more portions of the gene or the nucleic acid is from a non-human TSLP gene. In some embodiments, the gene or the nucleic acid comprises a sequence that encodes an TSLP protein. The encoded TSLP protein is functional or has at least one activity of the human TSLP protein or the non-human TSLP protein, e.g., interacting with a complex formed by IL7R and TSLPR, to induce downstream signaling pathways.

In some embodiments, the chimeric gene or the chimeric nucleic acid is a humanized TSLPR gene or a humanized TSLPR nucleic acid. In some embodiments, at least one or more portions of the gene or the nucleic acid is from the human TSLPR gene, at least one or more portions of the gene or the nucleic acid is from a non-human TSLPR gene. In some embodiments, the gene or the nucleic acid comprises a sequence that encodes an TSLPR protein. The encoded TSLPR protein is functional or has at least one activity of the human TSLPR protein or the non-human TSLPR protein, e.g., interacting with IL7R to form a heterodimeric complex to induce downstream signaling pathways.

In some embodiments, the chimeric protein or the chimeric polypeptide is a humanized TSLP protein or a humanized TSLP polypeptide. In some embodiments, at least one or more portions of the amino acid sequence of the protein or the polypeptide is from a human TSLP protein, and at least one or more portions of the amino acid sequence of the protein or the polypeptide is from a non-human TSLP protein. The humanized TSLP protein or the humanized TSLP polypeptide is functional or has at least one activity of the human TSLP protein or the non-human TSLP protein.

In some embodiments, the chimeric protein or the chimeric polypeptide is a humanized TSLPR protein or a humanized TSLPR polypeptide. In some embodiments, at least one or more portions of the amino acid sequence of the protein or the polypeptide is from a human TSLPR protein, and at least one or more portions of the amino acid sequence of the protein or the polypeptide is from a non-human TSLPR protein. The humanized TSLPR protein or the humanized TSLPR polypeptide is functional or has at least one activity of the human TSLPR protein or the non-human TSLPR protein.

In some embodiments, the extracellular region described herein is human or humanized. In some embodiments, the transmembrane region described herein is human or humanized. In some embodiments, the cytoplasmic region described herein is human or humanized.

The genetically modified non-human animal can be various animals, e.g., a mouse, rat, rabbit, pig, bovine (e.g., cow, bull, buffalo), deer, sheep, goat, chicken, cat, dog, ferret, primate (e.g., marmoset, rhesus monkey). For the non-human animals where suitable genetically modifiable embryonic stem (ES) cells are not readily available, other methods are employed to make a non-human animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in a non-human animal under suitable conditions to form an embryo. These methods are known in the art, and are described, e.g., in A. Nagy, et al., “Manipulating the Mouse Embryo: A Laboratory Manual (Third Edition),” Cold Spring Harbor Laboratory Press, 2003, which is incorporated by reference herein in its entirety.

In one aspect, the animal is a mammal, e.g., of the superfamily Dipodoidea or Muroidea. In some embodiments, the genetically modified animal is a rodent. The rodent can be selected from a mouse, a rat, and a hamster. In some embodiments, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, malagasy rats and mice), Platacanthomyidae (e.g., spiny dormice), and Spalacidae (e.g., mole rates, bamboo rats, and zokors). In some embodiments, the genetically modified rodent is selected from a true mouse or rat (family Muridae), a gerbil, a spiny mouse, and a crested rat. In some embodiments, the non-human animal is a mouse.

In some embodiments, the animal is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, the mouse is a 129 strain selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129S6 (129/SvEvTac), 129S7, 129S8, 129T1, 129T2. These mice are described, e.g., in Festing et al., Revised nomenclature for strain 129 mice, Mammalian Genome 10:836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, the genetically modified mouse is a mix of the 129 strain and the C57BL/6 strain. In some embodiments, the mouse is a mix of the 129 strains, or a mix of the BL/6 strains. In some embodiments, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sv; or 50% C57BL/6-50% 129). In some embodiments, the non-human animal is a rodent. In some embodiments, the non-human animal is a mouse having a BALB/c, A, A/He, A/J, A/WySN, AKR, AKR/A, AKR/J, AKR/N, TA1, TA2, RF, SWR, C3H, C57BR, SJL, C57L, DBA/2, KM, NIH, ICR, CFW, FACA, C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL (C57BL/10Cr and C57BL/Ola), C58, CBA/Br, CBA/Ca, CBA/J, CBA/st, or CBA/H background.

In some embodiments, the animal is a rat. The rat can be selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

The animal can have one or more other genetic modifications, and/or other modifications, that are suitable for the particular purpose for which the humanized TSLP and/or TSLPR animal is made. For example, suitable mice for maintaining a xenograft (e.g., a human cancer or tumor), can have one or more modifications that compromise, inactivate, or destroy the immune system of the non-human animal in whole or in part. Compromise, inactivation, or destruction of the immune system of the non-human animal can include, for example, destruction of hematopoietic cells and/or immune cells by chemical means (e.g., administering a toxin), physical means (e.g., irradiating the animal), and/or genetic modification (e.g., knocking out one or more genes). Non-limiting examples of such mice include, e.g., NOD mice, SCID mice, NOD/SCID mice, IL2Rγ knockout mice, NOD/SCID/γc^{null} mice (Ito, M. et al., NOD/SCID/γc^{null} mouse: an excellent recipient mouse model for engraftment of human cells, Blood 100 (9): 3175-3182, 2002), nude mice, and Rag1 and/or Rag2 knockout mice. These mice can optionally be irradiated, or otherwise treated to destroy one or more immune cell type. Thus, in various embodiments, a genetically modified mouse is provided that can include a humanization of at least a portion of an endogenous non-human TSLP and/or TSLPR loci, and further comprises a modification that compromises, inactivates, or destroys the immune system (or one or more cell types of the immune system) of the non-human animal in whole or in part. In some embodiments, modification is, e.g., selected from the group consisting of a modification that results in NOD mice, SCID mice, NOD/SCID mice, IL-2Rγ knockout mice, NOD/SCID/γc^{null} mice, nude mice, Rag1 and/or Rag2 knockout mice, NOD-Prkdc^{scid} IL-2rγ^{null} mice, NOD-Rag 1^{-/-}-IL2rg^{-/-} (NRG) mice, Rag 2^{-/-}-IL2rg^{-/-} (RG) mice, and a combination thereof. These genetically modified animals are described, e.g., in US20150106961, which is incorporated herein by reference in its entirety. In some embodiments, the mouse can include a replacement of all or part of mature TSLP coding sequence with human mature TSLP coding sequence. In some embodiments, the mouse can include an insertion of a chimeric (e.g., human/non-human) TSLPR coding sequence at an endogenous TSLPR locus.

Genetically modified non-human animals can comprise a modification at endogenous non-human TSLP and/or TSLPR loci. In some embodiments, the modification can comprise a human nucleic acid sequence encoding at least a portion of a mature TSLP or TSLPR protein (e.g., at least 10%, 20%, 30%, 40%, 50%, 60%, 70%, 80%, 90%, 95%, 96%, 97%, 98%, or 99% identical to the mature TSLP or TSLPR protein sequence). Although genetically modified cells are also provided that can comprise the modifications described herein (e.g., ES cells, somatic cells), in many embodiments, the genetically modified non-human animals comprise the modification of the endogenous TSLP and/or TSLPR loci in the germline of the animal.

The present disclosure further relates to a non-human mammal generated through the method mentioned above. In some embodiments, the genome thereof contains human gene(s).

In some embodiments, the non-human mammal is a rodent, and preferably, the non-human mammal is a mouse.

In some embodiments, the non-human mammal expresses a protein encoded by humanized TSLP and/or TSLPR genes.

In addition, the present disclosure also relates to a tumor bearing non-human mammal model, characterized in that the non-human mammal model is obtained through the methods as described herein. In some embodiments, the non-human mammal is a rodent (e.g., a mouse).

The present disclosure further relates to a cell or cell line, or a primary cell culture thereof derived from the non-human mammal or an offspring thereof, or the tumor bearing non-human mammal; the tissue, organ or a culture thereof derived from the non-human mammal or an offspring thereof, or the tumor bearing non-human mammal; and the tumor tissue derived from the non-human mammal or an offspring thereof when it bears a tumor, or the tumor bearing non-human mammal.

The present disclosure also provides non-human mammals produced by any of the methods described herein. In some embodiments, a non-human mammal is provided; and the genetically modified animal contains the DNA encoding human or humanized c in the genome of the animal.

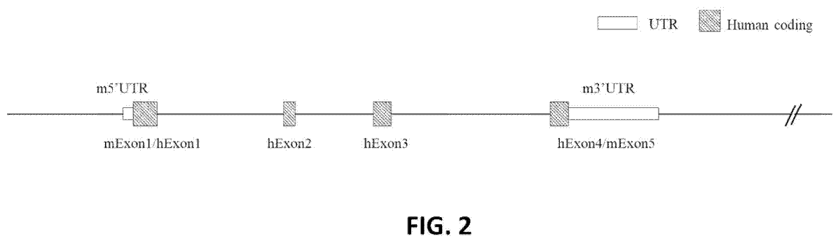

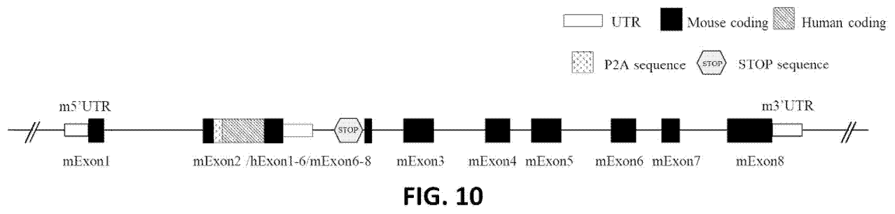

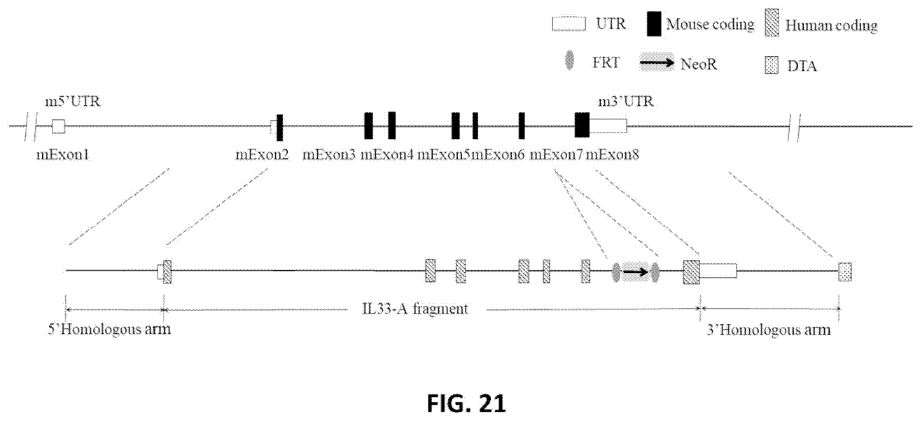

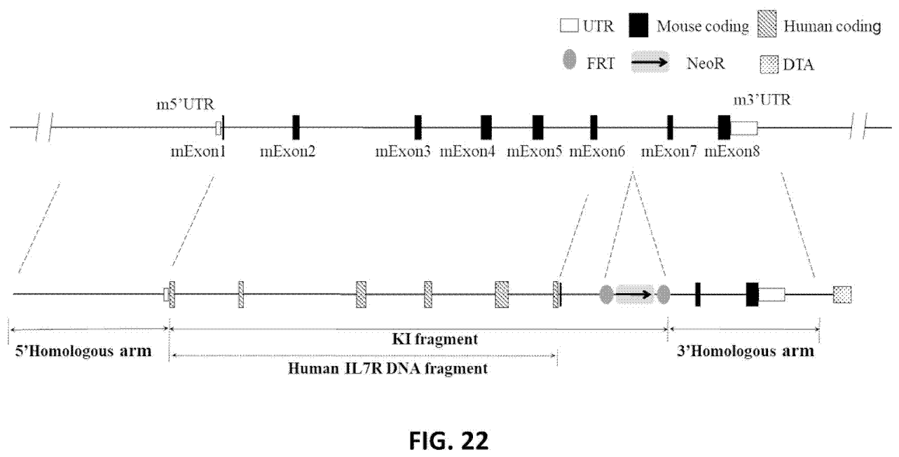

In some embodiments, the non-human mammal comprises a humanized TSLP gene having the genetic construct as described herein (e.g., gene construct as shown in FIGS. 2, 3, and 7). In some embodiments, the non-human mammal comprises a humanized TSLPR gene having the genetic construct as described herein (e.g., gene construct as shown in FIGS. 10, 11, 12, 13, 16, and 17). In some embodiments, the non-human mammal comprises a humanized IL33 gene having the genetic construct as described herein (e.g., gene construct as shown in FIG. 21). In some embodiments, the non-human mammal comprises a humanized IL7R gene having the genetic construct as described herein (e.g., gene construct as shown in FIG. 22). In some embodiments, a non-human mammal expressing human or humanized TSLP, TSLPR, IL33, and/or IL7R is provided. In some embodiments, the tissue-specific expression of human or humanized TSLP, TSLPR, IL33, and/or IL7R proteins is provided.

In some embodiments, the expression of human or humanized TSLP and/or TSLPR in a genetically modified animal is controllable, as by the addition of a specific inducer or repressor substance. In some embodiments, the specific inducer is selected from Tet-Off System/Tet-On System, or Tamoxifen System.

Non-human mammals can be any non-human animal known in the art and which can be used in the methods as described herein. Preferred non-human mammals are mammals, (e.g., rodents). In some embodiments, the non-human mammal is a mouse.

Genetic, molecular and behavioral analyses for the non-human mammals described above can performed. The present disclosure also relates to the progeny produced by the non-human mammal provided by the present disclosure mated with the same or other genotypes.

The present disclosure also provides a cell line or primary cell culture derived from the non-human mammal or a progeny thereof. A model based on cell culture can be prepared, for example, by the following methods. Cell cultures can be obtained by way of isolation from a non-human mammal, alternatively cells can be obtained from the cell culture established using the same constructs and the standard cell transfection techniques. The integration of genetic constructs containing DNA sequences encoding human TSLP and/or TSLPR proteins can be detected by a variety of methods.

There are many analytical methods that can be used to detect exogenous DNA, including methods at the level of nucleic acid (including the mRNA quantification approaches using reverse transcriptase polymerase chain reaction (RT-PCR) or Southern blotting, and in situ hybridization) and methods at the protein level (including histochemistry, immunoblot analysis and in vitro binding studies). In addition, the expression level of the gene of interest can be quantified by ELISA techniques well known to those skilled in the art. Many standard analysis methods can be used to complete quantitative measurements. For example, transcription levels can be measured using RT-PCR and hybridization methods including RNase protection, Southern blot analysis, RNA dot analysis (RNAdot) analysis. Immunohistochemical staining, flow cytometry, Western blot analysis can also be used to assess the presence of human or humanized TSLP and/or TSLPR proteins.

In some embodiments, provided herein is a genetically-modified non-human animal having two or more human or humanized genes selected from TSLP, TSLPR, IL33, and/or IL7R.

### Vectors

The present disclosure relates to a targeting vector, comprising: a) a DNA fragment homologous to the 5′ end of a region to be altered (5′ arm), which is selected from the TSLP gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the TSLP gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC_000084.7; c) the DNA fragment homologous to the 3′ end of the region to be altered (3′ arm) is selected from the nucleotide sequences that have at least 90% homology to the NCBI accession number NC_000084.7.

In some embodiments, a) the DNA fragment homologous to the 5′ end of a region to be altered (5′ arm) is selected from the nucleotides from the position 32943730 to the position 32948452 of the NCBI accession number NC_000084.7; c) the DNA fragment homologous to the 3′ end of the region to be altered (3′ arm) is selected from the nucleotides from the position 32953181 to the position 32957221 of the NCBI accession number NC_000084.7.

In some embodiments, the length of the selected genomic nucleotide sequence in the targeting vector can be more than about 2 kb, 2.5 kb, 3 kb, 3.5 kb, 3 kb, 4.2 kb, 4.5 kb, 5 kb, 5.5 kb, or 6 kb.

In some embodiments, the region to be altered is exon 1, exon 2, exon 3, exon 4, and/or exon 5 of TSLP gene (e.g., a portion of exon 1, exons 2-4, and a portion of exon 5 of mouse TSLP gene).

The targeting vector can further include one or more selectable markers, e.g., positive or negative selectable markers. In some embodiments, the positive selectable marker is a Neo gene or Neo cassette. In some embodiments, the negative selectable marker is a DTA gene.

In some embodiments, the sequence of the 5′ arm is shown in SEQ ID NO: 3; and the sequence of the 3′ arm is shown in SEQ ID NO: 4.

In some embodiments, the sequence is derived from human (e.g., 111071891-111076074 of NC_000005.10; SEQ ID NO: 5). For example, the target region in the targeting vector is a part or entirety of the nucleotide sequence of a human TSLP gene, preferably exon 1, exon 2, exon 3, and/or exon 4 of the human TSLP. In some embodiments, the nucleotide sequence of the humanized TSLP gene encodes the entire or the part of human TSLP protein with the NCBI accession number NP_149024.1 (SEQ ID NO: 2).

The present disclosure relates to a targeting vector, comprising: a) a DNA fragment homologous to the 5′ end of a region to be altered (5′ arm), which is selected from the TSLPR gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the TSLPR gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC_000071.7; c) the DNA fragment homologous to the 3′ end of the region to be altered (3′ arm) is selected from the nucleotide sequences that have at least 90% homology to the NCBI accession number NC_000071.7.

In some embodiments, a) the DNA fragment homologous to the 5′ end of a region to be altered (5′ arm) is selected from the nucleotides from the position 109705410 to the position 109709296 of the NCBI accession number NC_000071.7; c) the DNA fragment homologous to the 3′ end of the region to be altered (3′ arm) is selected from the nucleotides from the position 109701469 to the position 109705409 of the NCBI accession number NC_000071.7.

In some embodiments, the length of the selected genomic nucleotide sequence in the targeting vector can be more than about 500 bp, 600 bp, 700 bp, 800 bp, 900 bp, 1000 bp, 1100 bp, 1200 bp, 1250 bp, 1260 bp, 1270 bp, 1300 bp, 1400 bp, or 1500 bp.

In some embodiments, the region to be altered is exon 2 of endogenous TSLPR gene (e.g., between position 260-261 of mouse TSLPR gene).

The targeting vector can further include one or more selectable markers, e.g., positive or negative selectable markers. In some embodiments, the positive selectable marker is a Neo gene or Neo cassette. In some embodiments, the negative selectable marker is a DTA gene.

In some embodiments, the sequence of the 5′ arm is shown in SEQ ID NO: 9; and the sequence of the 3′ arm is shown in SEQ ID NO: 10.

In some embodiments, the inserted sequence is derived from human (e.g., nucleic acids 16-774 of NM_022148.4) and mouse (e.g., nucleic acids 873-1190 of NM_016715.4). For example, the target region in the targeting vector comprises a portion (e.g., nucleotides 16-94) of human exon 1, human exons 2-5, a portion (e.g., nucleotides 662-774) of human exon 6, a portion (e.g., nucleotides 873-880) of endogenous exon 6, and endogenous exon 7-8. In some embodiments, the nucleotide sequence of the humanized TSLPR gene encodes a TSLPR protein with amino acid sequence set forth in SEQ ID NO: 19. In some embodiments, the inserted sequence is at least 80%, 90%, or 95% identical to SEQ ID NO: 14.

In some embodiments, a) the DNA fragment homologous to the 5′ end of a region to be altered (5′ arm) is selected from the nucleotides from the position 109705410 to the position 109706794 of the NCBI accession number NC_000071.7; c) the DNA fragment homologous to the 3′ end of the region to be altered (3′ arm) is selected from the nucleotides from the position 109704095 to the position 109705409 of the NCBI accession number NC_000071.7. In some embodiments, the sequence of the 5′ arm is shown in SEQ ID NO: 11; and the sequence of the 3′ arm is shown in SEQ ID NO: 12. In some embodiments, the inserted sequence is at least 80%, 90%, or 95% identical to SEQ ID NO: 13.

The disclosure also provides vectors for constructing a humanized animal model or a knock-out model. In some embodiments, the vectors comprise sgRNA sequence, wherein the sgRNA sequence target TSLPR gene, and the sgRNA is unique on the target sequence of the gene to be altered, and meets the sequence arrangement rule of 5′-NNN (20)-NGG3′ or 5′-CCN—N(20)-3′; and in some embodiments, the targeting site of the sgRNA in the mouse VSIG4 gene is located on the exon 1, exon 2, exon 3, exon 4, exon 5, exon 6, exon 7, exon 8, intron 1, intron 2, intron 3, intron 4, intron 5, intron 6, intron 7, upstream of exon 1, or downstream of exon 8 of the mouse TSLPR gene.

In some embodiments, the targeting sequences are shown as SEQ ID NOs: 41-44. Thus, the disclosure provides sgRNA sequences for constructing a genetic modified animal model. In some embodiments, the oligonucleotide sgRNA sequences are set forth in SEQ ID NO: 41.

In some embodiments, the disclosure relates to a plasmid construct (e.g., pT7-sgRNA) including the sgRNA sequence, and/or a cell including the construct.

In some embodiments, a) the DNA fragment homologous to the 5′ end of a region to be altered (5′ arm) is selected from the nucleotides from the position 109706747 to the position 109707691 of the NCBI accession number NC_000071.7; c) the DNA fragment homologous to the 3′ end of the region to be altered (3′ arm) is selected from the nucleotides from the position 109704991 to the position 109706746 of the NCBI accession number NC_000071.7.

In some embodiments, the region to be altered is exon 1 of endogenous TSLPR gene (e.g., between position 113-114 of mouse TSLPR gene).

In some embodiments, the sequence of the 5′ arm is shown in SEQ ID NO: 20; and the sequence of the 3′ arm is shown in SEQ ID NO: 21.

In some embodiments, the inserted sequence is derived from human (e.g., nucleic acids 19-774 of NM_022148.4) and mouse (e.g., nucleic acids 873-1190 of NM_016715.4). For example, the target region in the targeting vector comprises a portion (e.g., nucleotides 19-94) of human exon 1, human exons 2-5, a portion (e.g., nucleotides 662-774) of human exon 6, a portion (e.g., nucleotides 873-880) of endogenous exon 6, and endogenous exon 7-8. In some embodiments, the nucleotide sequence of the humanized TSLPR gene encodes a TSLPR protein with amino acid sequence set forth in SEQ ID NO: 19. In some embodiments, the inserted sequence is at least 80%, 90%, or 95% identical to SEQ ID NO: 22.

The present disclosure relates to a targeting vector, comprising: a) a DNA fragment homologous to the 5′ end of a region to be altered (5′ arm), which is selected from the TSLP gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the TSLP gene genomic DNAs in the length of 100 to 10,000 nucleotides.

The present disclosure relates to a targeting vector, comprising: a) a DNA fragment homologous to the 5′ end of a region to be altered (5′ arm), which is selected from the IL33 gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the IL33 gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC_000085.6; c) the DNA fragment homologous to the 3′ end of the region to be altered (3′ arm) is selected from the nucleotide sequences that have at least 90% homology to the NCBI accession number NC_000085.6.

In some embodiments, a) the DNA fragment homologous to the 5′ end of a region to be altered (5′ arm) is selected from the nucleotides from the position 29945453 to the position 29949670 of the NCBI accession number NC_000085.6; c) the DNA fragment homologous to the 3′ end of the region to be altered (3′ arm) is selected from the nucleotides from the position 29959042 to the position 29963122 of the NCBI accession number NC_000085.6.

In some embodiments, the length of the selected genomic nucleotide sequence in the targeting vector can be more than about 10 kb, about 10.5 kb, about 11 kb, about 11.5 kb, about 12 kb, about 12.5 kb, about 13 kb, about 13.5 kb, about 14 kb, about 14.5 kb, or about 15 kb.

In some embodiments, the region to be altered is exon 1, exon 2, exon 3, exon 4, exon 5, exon 6, exon 7, and/or exon 8 of IL33 gene (e.g., a portion of exon 2, exons 3-7, and a portion of exon 8 of mouse IL33 gene).

The targeting vector can further include one or more selectable markers, e.g., positive or negative selectable markers. In some embodiments, the positive selectable marker is a Neo gene or Neo cassette. In some embodiments, the negative selectable marker is a DTA gene.

In some embodiments, the sequence of the 5′ arm is shown in SEQ ID NO: 67; and the sequence of the 3′ arm is shown in SEQ ID NO: 68.

In some embodiments, the sequence is derived from human (e.g., 6241695-6256168 of NC_000009.12). For example, the target region in the targeting vector is a part or entirety of the nucleotide sequence of a human IL33, preferably exon 2, exon 3, exon 4, exon 5, exon 6, exon 7, and/or exon 8 of the human IL33. In some embodiments, the nucleotide sequence of the humanized IL33 encodes the entire or the part of human IL33 protein with the NCBI accession number NP_254274.1 (SEQ ID NO: 66).

In some embodiments, a) the DNA fragment homologous to the 5′ end of a conversion region to be altered (5′ arm) is selected from the nucleotide sequences that have at least 90% homology to the NCBI accession number NC_000081.6; c) the DNA fragment homologous to the 3′ end of the region to be altered (3′ arm) is selected from the nucleotide sequences that have at least 90% homology to the NCBI accession number NC_000081.6.

In some embodiments, a) the DNA fragment homologous to the 5′ end of a region to be altered (5′ arm) is selected from the nucleotides from the position 9529743 to the position 9534180 of the NCBI accession number NC_000081.6; c) the DNA fragment homologous to the 3′ end of the region to be altered (3′ arm) is selected from the nucleotides from the position 9505623 to the position 9509583 of the NCBI accession number NC_000081.6.

In some embodiments, the region to be altered is exon 1, exon 2, exon 3, exon 4, exon 5, exon 6, exon 7, and/or exon 8 of IL7R gene (e.g., a portion of exon 1, exons 2-5, and a portion of exon 6 of mouse IL7R gene).

The targeting vector can further include one or more selectable markers, e.g., positive or negative selectable markers. In some embodiments, the positive selectable marker is a Neo gene or Neo cassette. In some embodiments, the negative selectable marker is a DTA gene.

In some embodiments, the sequence of the 5′ arm is shown in SEQ ID NO: 77; and the sequence of the 3′ arm is shown in SEQ ID NO: 78.

In some embodiments, the sequence is derived from human. For example, the target region in the targeting vector is a part or entirety of the nucleotide sequence of a human IL7R gene, preferably exon 1, exon 2, exon 3, exon 4, exon 5, and/or exon 6 of the human IL7R gene. In some embodiments, the nucleotide sequence of the humanized IL7R gene encodes the entire or the part of human IL7R protein with the NCBI accession number NP_002176.2 (SEQ ID NO: 76).

The disclosure also relates to a cell comprising the targeting vectors as described above.

In addition, the present disclosure further relates to a non-human mammalian cell, having any one of the foregoing targeting vectors, and one or more in vitro transcripts of the construct as described herein. In some embodiments, the cell includes Cas9 mRNA or an in vitro transcript thereof.

In some embodiments, the genes in the cell are heterozygous. In some embodiments, the genes in the cell are homozygous.

In some embodiments, the non-human mammalian cell is a mouse cell. In some embodiments, the cell is a fertilized egg cell. In some embodiments, the cell is an embryonic stem cell.

### Methods of Making Genetically Modified Animals

Genetically modified animals can be made by several techniques that are known in the art, including, e.g., nonhomologous end-joining (NHEJ), homologous recombination (HR), zinc finger nucleases (ZFNs), transcription activator-like effector-based nucleases (TALEN), and the clustered regularly interspaced short palindromic repeats (CRISPR)-Cas system. In some embodiments, homologous recombination is used. In some embodiments, CRISPR-Cas9 genome editing is used to generate genetically modified animals. Many of these genome editing techniques are known in the art, and is described, e.g., in Yin et al., “Delivery technologies for genome editing,” Nature Reviews Drug Discovery 16.6 (2017): 387-399, which is incorporated by reference in its entirety. Many other methods are also provided and can be used in genome editing, e.g., micro-injecting a genetically modified nucleus into an enucleated oocyte, and fusing an enucleated oocyte with another genetically modified cell.

Thus, in some embodiments, the disclosure provides replacing in at least one cell of the animal, at an endogenous TSLP gene locus, a sequence encoding a region of an endogenous TSLP with a sequence encoding a corresponding region of human or chimeric TSLP. In some embodiments, the replacement occurs in a germ cell, a somatic cell, a blastocyst, or a fibroblast, etc. The nucleus of a somatic cell or the fibroblast can be inserted into an enucleated oocyte.

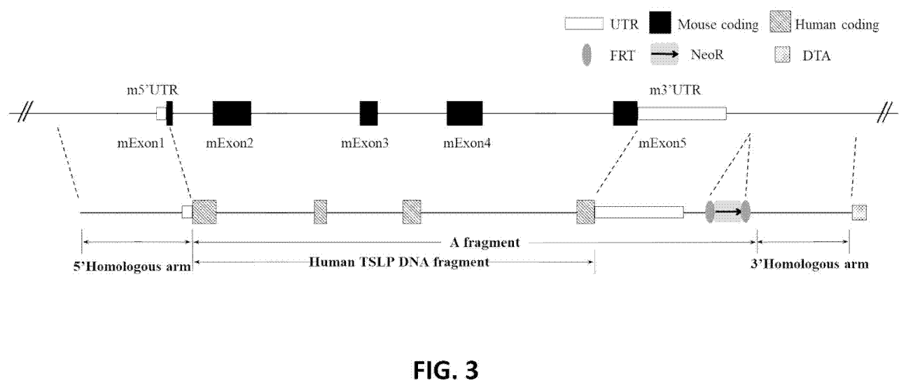

FIG. 3 shows a humanization strategy for a mouse TSLP locus. In FIG. 3, the targeting strategy involves a vector comprising the 5′ end homologous arm, human TSLP gene fragment, 3′ homologous arm. The process can involve replacing endogenous TSLP sequence with human sequence by homologous recombination. In some embodiments, the cleavage at the upstream and the downstream of the target site (e.g., by zinc finger nucleases, TALEN or CRISPR) can result in DNA double strands break, and the homologous recombination is used to replace endogenous TSLP sequence with human TSLP sequence.

Thus, in some embodiments, the methods for making a genetically modified, humanized animal, can include the step of replacing at an endogenous TSLP locus (or site), a nucleic acid encoding a region of endogenous TSLP with a sequence encoding a corresponding region of human TSLP. The sequence can include a region (e.g., a part or the entire region) of exon 1, exon 2, exon 3, and/or exon 4 of a human TSLP gene. In some embodiments, the sequence includes a portion of exon 1, exons 2-3, and a portion of exon 4 of a human TSLP gene (e.g., nucleic acids 179-658 of NM_033035.5). In some embodiments, the region includes the entire coding sequence (CDS) of human TSLP (e.g., SEQ ID NO: 2). In some embodiments, the endogenous TSLP locus is exon 1, exon 2, exon 3, exon 4, and/or exon 5 of mouse TSLP. In some embodiments, the sequence includes a portion of exon 1, exons 2-4, and a portion of exon 5 of mouse TSLP gene (e.g., nucleic acids 18-440 of NM_021367.2).

In some embodiments, the methods of modifying a TSLP locus of a mouse to express a chimeric human/mouse TSLP peptide can include the steps of replacing at the endogenous mouse TSLP locus a nucleotide sequence encoding a mouse TSLP with a nucleotide sequence encoding a human TSLP, thereby generating a sequence encoding a chimeric human/mouse TSLP.

In some embodiments, the nucleotide sequence encoding the chimeric human/mouse TSLP can include a first nucleotide sequence including the 5′ UTR of mouse TSLP gene; a second nucleotide sequence including the entire coding sequence of human TSLP gene; and/or a third nucleotide sequence including the 3′ UTR of mouse TSLP gene.

In some embodiments, the disclosure provides inserting in at least one cell of the animal, at an endogenous TSLPR gene locus (e.g., exon 1 or exon 2 of endogenous TSLPR gene), a sequence encoding the extracellular and transmembrane regions of human TSLPR, and the cytoplasmic region of endogenous TSLPR. In some embodiments, the insertion occurs in a germ cell, a somatic cell, a blastocyst, or a fibroblast, etc. The nucleus of a somatic cell or the fibroblast can be inserted into an enucleated oocyte.

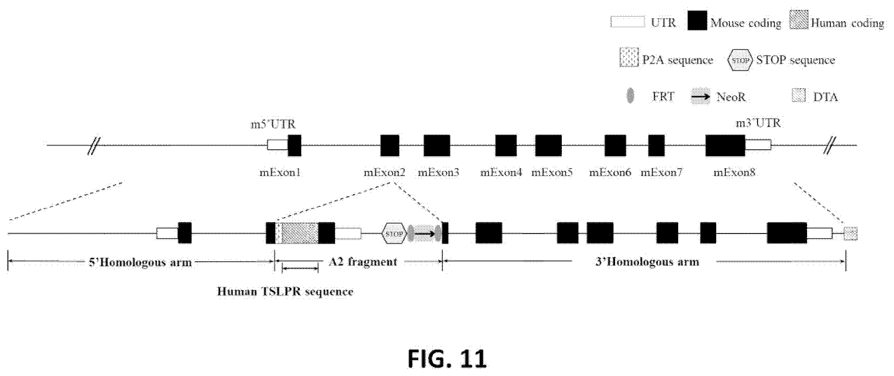

FIGS. 11, 13, and 17 show humanization strategies for a mouse TSLPR locus. The targeting strategies involve a vector comprising the 5′ end homologous arm, a chimeric TSLPR sequence, 3′ homologous arm. The process can involve inserting the chimeric TSLPR sequence by homologous recombination. In some embodiments, the cleavage at the upstream and the downstream of the target site (e.g., by zinc finger nucleases, TALEN or CRISPR) can result in DNA double strands break, and the homologous recombination is used to insert the chimeric TSLPR sequence within the endogenous TSLPR gene locus.

Thus, in some embodiments, the methods for making a genetically modified, humanized animal, can include the step of inserting at an endogenous TSLPR locus (or site), a nucleic acid encoding the extracellular and transmembrane regions of human TSLPR, and the cytoplasmic region of endogenous TSLPR. The sequence can include a portion (e.g., nucleotides 16-94 or 19-94) of human exon 1, human exons 2-5, a portion (e.g., nucleotides 662-774) of human exon 6, a portion (e.g., nucleotides 873-880) of endogenous exon 6, and endogenous exon 7-8. In some embodiments, the sequence includes nucleic acids 16-774 or 19-774 of NM_022148.4 and nucleic acids 873 or 1187 or 873-1190 of NM_016715.4.

In some embodiments, the methods of modifying a TSLPR locus of a mouse to express a chimeric human/mouse TSLPR peptide can include the steps of inserting at the endogenous mouse TSLPR locus a nucleotide sequence encoding a chimeric (e.g., humanized) TSLPR protein, thereby generating a sequence encoding a chimeric human/mouse TSLPR.

In some embodiments, the nucleotide sequence encoding the chimeric human/mouse TSLPR can include: a first nucleotide sequence encoding the N-terminal 1-5 (e.g., 1, 2, 3, 4, or 5) amino acids of mouse TSLPR protein; a second nucleotide sequence encoding the extracellular and transmembrane regions of human TSLPR protein; and a third nucleotide sequence encoding the cytoplasmic region of mouse TSLPR protein. In some embodiments, the second nucleotide sequence does not include the N-terminal 1-5 (e.g., 1, 2, 3, 4, or 5) amino acids of the extracellular region (including the signal peptide) of human TSLPR protein. In some embodiments, the second nucleotide further includes a sequence encoding the N-terminal 1-6 (e.g., 1, 2, 3, 4, 5, or 6) amino acids of the cytoplasmic region of human TSLPR protein, and the third nucleotide does not include a corresponding sequence encoding the N-terminal 1-6 (e.g., 1, 2, 3, 4, 5, or 6) amino acids of the cytoplasmic region of mouse TSLPR protein. In some embodiments, the first nucleotide sequence is optional.

In some embodiments, the nucleotide sequences as described herein do not overlap with each other (e.g., the first nucleotide sequence, the second nucleotide sequence, and/or the third nucleotide sequence do not overlap). In some embodiments, the amino acid sequences as described herein do not overlap with each other.

The present disclosure further provides a method for establishing TSLP and/or TSLPR gene humanized animal models, involving the following steps:
(a) providing the cell (e.g. a fertilized egg cell) based on the methods described herein;
(b) culturing the cell in a liquid culture medium;
(c) transplanting the cultured cell to the fallopian tube or uterus of the recipient female non-human mammal, allowing the cell to develop in the uterus of the female non-human mammal;
(d) identifying the germline transmission in the offspring genetically modified humanized non-human mammal of the pregnant female in step (c).

In some embodiments, the non-human mammal in the foregoing method is a mouse (e.g., a C57BL/6 mouse).

In some embodiments, the non-human mammal in step (c) is a female with pseudopregnancy (or false pregnancy).

In some embodiments, the fertilized eggs for the methods described above are C57BL/6 fertilized eggs. Other fertilized eggs that can also be used in the methods as described herein include, but are not limited to, FVB/N fertilized eggs, BALB/c fertilized eggs, DBA/1 fertilized eggs and DBA/2 fertilized eggs.

Fertilized eggs can come from any non-human animal, e.g., any non-human animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant non-human animal, which then gives birth of a non-human mammal, so as to generate the non-human mammal mentioned in the methods described above.

In some embodiments, methods of making the genetically modified animal comprises modifying the coding frame of the non-human animal's TSLP and/or TSLPR genes, e.g., by inserting a nucleotide sequence (e.g., DNA or cDNA sequence) encoding human or humanized TSLP and/or TSLPR proteins immediately after the endogenous regulatory element of the non-human animal's TSLP and/or TSLPR genes. For example, one or more functional region sequences of the non-human animal's TSLP and/or TSLPR genes can be knocked out, or inserted with a sequence, such that the non-human animal cannot express or expresses a decreased level of endogenous TSLP and/or TSLPR proteins. In some embodiments, the coding frame of the modified non-human animal's TSLP gene can be all or part of the nucleotide sequence from exon 1 to exon 5 of the non-human animal's TSLP gene. In some embodiments, the coding frame of the modified non-human animal's TSLPR gene can be all or part of the nucleotide sequence from exon 1 to exon 8 of the non-human animal's TSLPR gene.

In some embodiments, methods of making the genetically modified animal comprises inserting a nucleotide sequence encoding human or humanized TSLP and/or TSLPR proteins and/or an auxiliary sequence after the endogenous regulatory element of the non-human animal's TSLP and/or TSLPR genes. In some embodiments, the auxiliary sequence can be a stop codon, such that the TSLP and/or TSLPR gene humanized animal models can express human or humanized TSLP and/or TSLPR proteins in vivo, but does not express non-human animal's TSLP and/or TSLPR proteins. In some embodiments, the auxiliary sequence includes WPRE (WHP Posttranscriptional Response Element), lox2, and/or polyA.

In some embodiments, the method for making the genetically modified animal comprises:
(1) providing a plasmid comprising a human TSLPR gene fragment, flanked by a 5′ homology arm and a 3′ homology arm, wherein the 5′ and 3′ homology arms target an endogenous TSLPR gene;
(2) providing one or more small guide RNAs (sgRNAs) that target the endogenous TSLPR gene;
(3) modifying genome of a fertilized egg or an embryonic stem cell by using the plasmid of step (1), the sgRNAs of step (2), and Cas9;
(4) transplanting the fertilized egg obtained in step (3) into the oviduct of a pseudopregnant female mouse or transplanting the embryonic stem cell obtained in step (3) into a blastocyst which is then transplanted into the oviduct of a pseudopregnant female mouse to produce a child mouse that functionally expresses a humanized TSLPR protein; and
(5) mating the child mouse obtained in step (2) to obtain a homozygote mouse,

In some embodiments, the fertilized egg is modified by CRISPR with sgRNAs that target a 5′-terminal targeting site and a 3′-terminal targeting site.

In some embodiments, the sequence encoding the humanized TSLPR protein is operably linked to an endogenous regulatory element at the endogenous TSLPR gene locus.

In some embodiments, the genetically-modified animal does not express an endogenous TSLPR protein.

In some embodiments, the method for making the genetically modified animal comprises:
(1) providing a plasmid comprising a human or chimeric TSLPR gene fragment, flanked by a 5′ homology arm and a 3′ homology arm, wherein the 5′ and 3′ homology arms target an endogenous TSLPR gene;
(2) providing one small guide RNAs (sgRNAs) that target the endogenous TSLPR gene; and
(3) modifying genome of a fertilized egg or an embryonic stem cell by inserting the human or chimeric TSLPR gene fragment into the genome.

### Methods of Using Genetically Modified Animals

Replacement of non-human genes in a non-human animal with homologous or orthologous human genes or human sequences, at the endogenous non-human locus and under control of endogenous promoters and/or regulatory elements, can result in a non-human animal with qualities and characteristics that may be substantially different from a typical knockout-plus-transgene animal. In the typical knockout-plus-transgene animal, an endogenous locus is removed or damaged and a fully human transgene is inserted into the animal's genome and presumably integrates at random into the genome. Typically, the location of the integrated transgene is unknown; expression of the human protein is measured by transcription of the human gene and/or protein assay and/or functional assay. Inclusion in the human transgene of upstream and/or downstream human sequences are apparently presumed to be sufficient to provide suitable support for expression and/or regulation of the transgene.

In some cases, the transgene with human regulatory elements expresses in a manner that is unphysiological or otherwise unsatisfactory, and can be actually detrimental to the animal. The disclosure demonstrates that a replacement or insertion with human sequence at an endogenous locus under control of endogenous regulatory elements provides a physiologically appropriate expression pattern and level that results in a useful humanized animal whose physiology with respect to the replaced or disrupted gene are meaningful and appropriate in the context of the humanized animal's physiology.

Genetically modified animals that express human or humanized TSLP, TSLPR, IL33, and/or IL7R proteins, e.g., in a physiologically appropriate manner, provide a variety of uses that include, but are not limited to, developing therapeutics for human diseases and disorders, and assessing the toxicity and/or the efficacy of these human therapeutics in the animal models.

In various aspects, genetically modified animals are provided that express human or humanized TSLP, TSLPR, IL33, and/or IL7R, which are useful for testing agents that can decrease or block the interaction between the interaction between TSLP (or variant thereof) and its receptor (e.g., a heterodimeric complex formed by TSLPR/IL7R), the interaction between TSLP and anti-human TSLP antibodies, and the interaction between TSLPR and anti-TSLPR antibodies, testing whether an agent can increase or decrease the immune response, and/or determining whether an agent is an TSLP/TSLPR agonist or antagonist. The genetically modified animals can be, e.g., an animal model of a human disease, e.g., the disease is induced genetically (a knock-in or knockout). In various embodiments, the genetically modified non-human animals further comprise an impaired immune system, e.g., a non-human animal genetically modified to sustain or maintain a human xenograft, e.g., a human solid tumor (e.g., breast cancer) or a blood cell tumor (e.g., a lymphocyte tumor, a B or T cell tumor). In some embodiments, the anti-TSLP antibody or anti-TSLPR antibody blocks or inhibits the TSLP/TSLPR-mediated signaling pathway.

In some embodiments, the anti-TSLP antibody described herein can block the interaction between TSLP and the TSLPR/IL7R complex, thereby inhibiting formation of a functional TSLP/TSLPR/IL7R signaling complex. In some embodiments, the anti-TSLPR antibody described herein can block the interaction between TSLPR and TSLP and/or IL7R, thereby inhibiting formation of a functional TSLP/TSLPR/IL7R signaling complex.

In some embodiments, the genetically modified animals can be used for determining effectiveness of a therapeutic agent (e.g., an anti-TSLP antibody or an anti-TSLPR antibody) for the treatment of various immune disorders, including allergy, asthma, and/or atopic dermatitis. Thus, the methods as described herein can be used to determine the effectiveness of an anti-TSLP or anti-TSLPR antibody in inhibiting immune response. In some embodiments, the immune disorders described herein is allergy, asthma, myocarditis, nephritis, hepatitis, systemic lupus erythematosus, rheumatoid arthritis, scleroderma, hyperthyroidism, idiopathic thrombocytopenia purpura, autoimmune hemolytic anemia, ulcerative colitis, autoimmune liver disease, diabetes, pain or neurological disorders.

In some embodiments, the immune disorder described herein is asthma, and the animal model is established by inducing the animal (e.g., any of the animals described herein) with ovalbumin (OVA) and aluminum hydroxide. In some embodiments, the method involves administering the therapeutic agent (e.g., an anti-TSLP antibody or an anti-TSLPR antibody) to the animal as described herein (e.g., by intraperitoneal injection), wherein the animal has asthma; and determining effects of the therapeutic agent in treating asthma. In some embodiments, the effects are evaluated by comparing serum IgE level; pathological lung histology features; number of inflammatory cells (e.g., eosinophil counts in infiltrating cells) in bronchoalveolar lavage fluid (BALF); and/or airway reactivity of the animal with an animal induced by OVA/aluminum hydroxide, but not treated with the therapeutic agent. For example, reduced serum IgE level and/or reduced number of inflammatory cells in BALF indicate that the therapeutic agent can inhibit immune response thereby treating asthma.

In some embodiments, the immune disorder described herein is atopic dermatitis, and the animal model is established by inducing the animal (e.g., any of the animals described herein) with oxazolone (OXA), e.g., by smearing 0.1%-1% OXA to an exposed skin of the animal (e.g., ears or back). In some embodiments, the animal's skin is smeared with about 0.1%, about 0.2%, about 0.3%, about 0.4%, about 0.5%, about 0.6%, about 0.7%, about 0.8%, about 0.9 or about 1% OXA for about 7-11 days, about 7-18 days, or about 7-26 days. In some embodiments, the method involves administering the therapeutic agent (e.g., an anti-TSLP antibody or an anti-TSLPR antibody) to the animal as described herein (e.g., by intraperitoneal injection), wherein the animal has atopic dermatitis; and determining effects of the therapeutic agent in treating atopic dermatitis. In some embodiments, the effects are evaluated by comparing epidermal stromal cell hyperplasia; erosion/scab; hyperkeratosis; dermal and subcutaneous mixed inflammatory cell infiltration; eosinophilic infiltration; serum IgE levels; and/or ear thickness of the animal with an animal that induced by OXA, but not treated with the therapeutic agent. For example, reduced ear thickness, reduced serum IgE level, and/or reduced eosinophil infiltration indicate that the therapeutic agent can inhibit immune response thereby treating atopic dermatitis.

In some embodiments, the genetically modified animals can be used for determining effectiveness of a therapeutic agent (e.g., an anti-TSLP antibody or an anti-TSLPR antibody) for the treatment of various autoimmune diseases, including inflammatory arthritis, eczema, eosinophilic esophagitis, rheumatoid arthritis, Crohn's disease, systemic lupus erythematosus, ankylosing spondylitis, inflammatory bowel diseases (IBD), ulcerative colitis, multiple sclerosis, systemic juvenile idiopathic arthritis (SJIA), and scleroderma.

In some embodiments, the genetically modified animals can be used for determining effectiveness of a therapeutic agent (e.g., an anti-TSLP antibody or an anti-TSLPR antibody) for the reducing an inflammation or infection (e.g., Staphylococcus aureus infection, helminth infection, or viral infection). In some embodiments, In some embodiments, the genetically modified animals can be used for determining effectiveness of a therapeutic agent (e.g., an anti-TSLP antibody or an anti-TSLPR antibody) for treating chronic inflammatory diseases (e.g., chronic obstructive pulmonary disease, idiopathic pulmonary fibrosis, rheumatoid arthritis, or ulcerative colitis).

In some embodiments, the genetically modified animals can be used for determining effectiveness of a therapeutic agent (e.g., an anti-TSLP antibody or an anti-TSLPR antibody) for the treatment of cancer. In some embodiments, the methods involve administering the therapeutic agent (e.g., an anti-TSLP antibody or an anti-TSLPR antibody) to the animal as described herein, wherein the animal has a cancer or tumor; and determining inhibitory effects of the therapeutic agent to the cancer or tumor. The inhibitory effects that can be determined include, e.g., a decrease of tumor size or tumor volume, a decrease of tumor growth, a reduction of the increase rate of tumor volume in a subject (e.g., as compared to the rate of increase in tumor volume in the same subject prior to treatment or in another subject without such treatment), a decrease in the risk of developing a metastasis or the risk of developing one or more additional metastasis, an increase of survival rate, and an increase of life expectancy, etc. The tumor volume in a subject can be determined by various methods, e.g., as determined by direct measurement, MRI or CT. In addition, a delicate balance is required for these antibodies, as TSLP and TSLP receptor are also expressed on many other cells. Thus, it is important that the humanized TSLP and/or TSLP receptor functions in a largely similar way as compared to the endogenous TSLP and/or TSLP receptor, so that the results in the humanized animals can be used to predict the efficacy or toxicity of these therapeutic agents in the human.

In some embodiments, the tumor comprises one or more cancer cells (e.g., human or mouse cancer cells) that are injected into the animal. In some embodiments, the therapeutic agent inhibits TSLP/TSLPR signaling pathways. In some embodiments, the therapeutic agent does not inhibit TSLP/TSLPR signaling pathways.

In some embodiments, the genetically modified animals can be used for determining whether an anti-TSLP or anti-TSLPR antibody is an agonist or antagonist. In some embodiments, the methods as described herein are also designed to determine the functional effects of the therapeutic agent (e.g., an anti-TSLP antibody or an anti-TSLPR antibody), e.g., whether the agent can upregulate the immune response or downregulate immune response, and/or whether the agent can induce complement mediated cytotoxicity (CMC) or antibody dependent cellular cytotoxicity (ADCC). In some embodiments, the genetically modified animals can be used for determining the effective dosage of a therapeutic agent for treating a disease in the subject, e.g., immune disorders.

The inhibitory effects on tumors can also be determined by methods known in the art, e.g., measuring the tumor volume in the animal, and/or determining tumor (volume) inhibition rate (TGI_{TV}). The tumor growth inhibition rate can be calculated using the formula TGI_{TV} (%)=(1−TVt/TVc)×100, where TVt and TVc are the mean tumor volume (or weight) of treated and control groups.

In some embodiments, the therapeutic agent (e.g., an anti-TSLP antibody or an anti-TSLPR antibody) is designed for treating various cancers. As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, the cancer described herein is lymphoma, non-small cell lung cancer, cervical cancer, leukemia, ovarian cancer, nasopharyngeal cancer, breast cancer, endometrial cancer, colon cancer, rectal cancer, gastric cancer, bladder cancer, glioma, lung cancer, bronchial cancer, bone cancer, prostate cancer, pancreatic cancer, liver and bile duct cancer, esophageal cancer, kidney cancer, thyroid cancer, head and neck cancer, testicular cancer, glioblastoma, astrocytoma, melanoma, myeloproliferation abnormal syndromes, and sarcomas. In some embodiments, the leukemia is selected from acute lymphocytic (lymphoblastic) leukemia, acute myeloid leukemia, myeloid leukemia, chronic lymphocytic leukemia, multiple myeloma, plasma cell leukemia, and chronic myelogenous leukemia. In some embodiments, the lymphoma is selected from Hodgkin's lymphoma and non-Hodgkin's lymphoma, including B-cell lymphoma, diffuse large B-cell lymphoma, follicular lymphoma, mantle cell lymphoma, marginal zone B-cell lymphoma, T-cell lymphoma, and Waldenstrom macroglobulinemia. In some embodiments, the sarcoma is selected from the group consisting of osteosarcoma, Ewing sarcoma, leiomyosarcoma, synovial sarcoma, soft tissue sarcoma, angiosarcoma, liposarcoma, fibrosarcoma, rhabdomyosarcoma, and chondrosarcoma. In a specific embodiment, the tumor is breast cancer, ovarian cancer, endometrial cancer, melanoma, kidney cancer, lung cancer, or liver cancer. In some embodiments, the cancer described herein is acute lymphocytic leukaemia or a solid tumor.

The present disclosure also provides methods of determining toxicity of a therapeutic agent (e.g., an anti-TSLP antibody or an anti-TSLPR antibody). The methods involve administering the antibody to the animal as described herein. The animal is then evaluated for its weight change, red blood cell count, hematocrit, and/or hemoglobin. In some embodiments, the antibody can decrease the red blood cells (RBC), hematocrit, or hemoglobin by more than 20%, 30%, 40%, or 50%. In some embodiments, the animals can have a weight that is at least 5%, 10%, 20%, 30%, or 40% smaller than the weight of the control group (e.g., average weight of the animals that are not treated with the antibody).

The present disclosure also relates to the use of the animal model generated through the methods as described herein in the development of a product related to an immunization processes of human cells, the manufacturing of a human antibody, or the model system for a research in pharmacology, immunology, microbiology and medicine.

In some embodiments, the disclosure provides the use of the animal model generated through the methods as described herein in the production and utilization of an animal experimental disease model of an immunization processes involving human cells, the study on a pathogen, or the development of a new diagnostic strategy and/or a therapeutic strategy.

The disclosure also relates to the use of the animal model generated through the methods as described herein in the screening, verifying, evaluating or studying the TSLP and/or TSLPR gene functions, human TSLP and/or TSLPR antibodies, drugs or efficacies for human TSLP and/or TSLPR targeting sites, the drugs for immune-related diseases and antitumor drugs.

In some embodiments, the disclosure provides a method to verify in vivo efficacy of TCR-T, CAR-T, and/or other immunotherapies (e.g., T-cell adoptive transfer therapies). For example, the methods include transplanting human tumor cells into the animal described herein, and applying human CAR-T to the animal with human tumor cells. Effectiveness of the CAR-T therapy can be determined and evaluated. In some embodiments, the animal is selected from the TSLP and/or TSLPR gene humanized non-human animal prepared by the methods described herein, the TSLP and/or TSLPR gene humanized non-human animal described herein, the double- or multi-humanized non-human animal generated by the methods described herein (or progeny thereof), a non-human animal expressing the human or humanized TSLP and/or TSLPR proteins, or the tumor-bearing or inflammatory animal models described herein. In some embodiments, the TCR-T, CAR-T, and/or other immunotherapies can treat the TSLP/TSLPR-associated diseases described herein. In some embodiments, the TCA-T, CAR-T, and/or other immunotherapies provides an evaluation method for treating the TSLP/TSLPR-associated diseases described herein.

Genetically Modified Animal Model with Two or More Human or Chimeric Genes

The present disclosure further relates to methods for generating genetically modified animal model with two or more human or chimeric genes. The animal can comprise a human or chimeric TSLP and/or TSLPR genes and a sequence encoding an additional human or chimeric protein.

In some embodiments, the additional human or chimeric protein can be interleukin 33 (IL33), interleukin 7 receptor (IL7R), interleukin 6 (IL6), interleukin 12 (IL12), interleukin 23 (IL23), Tumor Necrosis Factor alpha (TNF-α), interleukin 4 (IL4), integrin associated protein (CD47), programmed cell death protein 1 (PD1), Tumor Necrosis Factor Receptor Superfamily, Member 7 (CD27), tumor necrosis factor receptor superfamily member 9 (4-1BB), cytotoxic T-lymphocyte-associated protein 4 (CTLA4), prostate-specific membrane antigen (PSMA), Tumor necrosis factor receptor superfamily, member 4 (OX40), T cell immunoreceptor with Ig and ITIM domains (TIGIT), and/or lymphocyte-activation gene 3 (LAG3).

The methods of generating genetically modified animal model with two or more human or chimeric genes (e.g., humanized genes) can include the following steps:
(a) using the methods of introducing human TSLP and/or TSLPR genes or chimeric TSLP and/or TSLPR genes as described herein to obtain a genetically modified non-human animal;
(b) mating the genetically modified non-human animal with another genetically modified non-human animal, and then screening the progeny to obtain a genetically modified non-human animal with two or more human or chimeric genes.

In some embodiments, in step (b) of the method, the genetically modified animal can be mated with a genetically modified non-human animal with human or chimeric IL33, IL7R, IL6, IL12, IL23, TNF-α, IL4, CD47, PD1, CD27, 4-1BB, CTLA4, PSMA, OX40, TIGIT, and/or LAG3. Some of these genetically modified non-human animal are described, e.g., in PCT/CN2020/105529, PCT/CN2017/090320, PCT/CN2017/099577, PCT/CN2017/099575, PCT/CN2017/099576, PCT/CN2017/099574, PCT/CN2017/110435, PCT/CN2017/120388, PCT/CN2018/081628, PCT/CN2019/110819, PCT/CN/2019/126045; each of which is incorporated herein by reference in its entirety.

In some embodiments, the TSLP and/or TSLPR humanizations are directly performed on a genetically modified animal having a human or chimeric IL33, IL7R, IL6, IL12, IL23, TNF-α, IL4, CD47, PD1, CD27, 4-1BB, CTLA4, PSMA, OX40, TIGIT, and/or LAG3 gene.

As these proteins may involve different mechanisms, a combination therapy that targets two or more of these proteins thereof may be a more effective treatment. In fact, many related clinical trials are in progress and have shown a good effect. The genetically modified animal model with two or more human or humanized genes can be used for determining effectiveness of a combination therapy that targets two or more of these proteins, e.g., an anti-TSLP antibody or an anti-TSLPR antibody, and an additional therapeutic agent for the treatment of cancer or immune disorder (e.g., asthma or atopic dermatitis). The methods include administering the an anti-TSLP antibody or an anti-TSLPR antibody and the additional therapeutic agent to the animal, wherein the animal has a tumor or immune disorder; and determining effects of the combined treatment to the tumor or immune disorder. In some embodiments, the additional therapeutic agent is an antibody that specifically binds to IL33, IL7R, IL6, IL12, IL23, TNF-α, IL4, CD47, PD1, CD27, 4-1BB, CTLA4, PSMA, OX40, TIGIT, and/or LAG3. In some embodiments, the additional therapeutic agent is an anti-CTLA4 antibody (e.g., ipilimumab), an anti-PD-1 antibody (e.g., nivolumab), or an anti-PD-L1 antibody.

In some embodiments, the animal further comprises a sequence encoding a human or humanized PD-1, a sequence encoding a human or humanized PD-L1, or a sequence encoding a human or humanized CTLA-4. In some embodiments, the additional therapeutic agent is an anti-PD-1 antibody (e.g., nivolumab, pembrolizumab), an anti-PD-L1 antibody, or an anti-CTLA-4 antibody. In some embodiments, the tumor comprises one or more tumor cells that express CD80, CD86, PD-L1, and/or PD-L2.

In some embodiments, the combination treatment is designed for treating various cancers as described herein, e.g., bladder cancer, melanoma, leukemia, hepatocellular carcinoma, breast cancer, sarcoma, head and neck cancer, colon cancer, lung cancer, liver cancer, or brain cancer.

In some embodiments, the methods described herein can be used to evaluate the combination treatment with some other methods. The methods of treating a cancer that can be used alone or in combination with methods described herein, include, e.g., treating the subject with chemotherapy, e.g., campothecin, doxorubicin, cisplatin, carboplatin, procarbazine, mechlorethamine, cyclophosphamide, adriamycin, ifosfamide, melphalan, chlorambucil, bisulfan, nitrosurea, dactinomycin, daunorubicin, bleomycin, plicomycin, mitomycin, etoposide, verampil, podophyllotoxin, tamoxifen, taxol, transplatinum, 5-flurouracil, vincristin, vinblastin, and/or methotrexate. Alternatively or in addition, the methods can include performing surgery on the subject to remove at least a portion of the cancer, e.g., to remove a portion of or all of a tumor(s), from the patient.

### EXAMPLES

The invention is further described in the following examples, which do not limit the scope of the invention described in the claims.

### Materials and Methods

The following materials were used in the following examples.

DraIII, HindIII, EcoRV, ScaI, and StuI restriction enzymes were purchased from NEB (Catalog numbers: R3510S, R3104S, R3195S, R3122S, and R0187S, respectively).

C57BL/6 mice and Flp transgenic mice were purchased from the China Food and Drugs Research Institute National Rodent Experimental Animal Center.

Brilliant Violet 510™ anti-mouse CD45 Antibody was purchased from BioLegend (Catalog number: 103138).

V450 Rat Anti-mouse CD11b was purchased from BD Horizon (Catalog number: 560455).

Brilliant Violet 605™ anti-mouse CD11c was purchased from BioLegend (Catalog number: 117334).

APC anti-mouse TSLPR (TSLP-R) Antibody was purchased from BioLegend (Catalog number: 151805).

PE anti-human TSLPR (TSLP-R) Antibody was purchased from BioLegend (Catalog number: 322805).

APC Rat IgG2a, κ Isotype Ctrl Antibody was purchased from BioLegend (Catalog number: 400512).

PE Mouse IgG1, κ Isotype Ctrl Antibody was purchased from BioLegend (Catalog number: 400112).

Purified anti-mouse CD16/32 was purchased from BioLegend (Catalog number: 101302).

FITC anti-mouse F4/802 was purchased from BioLegend (Catalog number: 123108).

MOUSE TSLP ELISA KIT was purchased from BioLegend (Catalog number: 434107).

HUMAN TSLP ELISA KIT was purchased from BioLegend (Catalog number: 434207).

### Example 1: Mice with Humanized TSLP Gene

In this example, a non-human animal (e.g., a mouse) was modified to include a nucleotide sequence encoding human TSLP protein, and the obtained genetically-modified non-human animal can express a human or humanized TSLP protein in vivo. The mouse TSLP gene (NCBI Gene ID: 53603, Primary source: MGI: 1855696, UniProt ID: Q9JIE6) is located at 32948436 to 32952852 of chromosome 18 (NC_000084.7), and the human TSLP gene (NCBI Gene ID: 85480, Primary source: HGNC: 30743, UniProt ID: Q969D9-1) is located at 111070062 to 111078026 of chromosome 5 (NC_000005.10). The mouse TSLP transcript is NM_021367.2, and the corresponding protein sequence NP_067342.1 is set forth in SEQ ID NO: 1. The human TSLP transcript is NM_033035.5, and the corresponding protein sequence NP_149024.1 is set forth in SEQ ID NO: 2. Mouse and human TSLP gene loci are shown in FIG. 1.

All or part of nucleotide sequences encoding human TSLP protein can be introduced into the mouse endogenous TSLP locus, so that the mouse expresses human or humanized TSLP protein. Specifically, using gene-editing techniques, a nucleotide sequence (e.g., DNA or cDNA sequence) of the human TSLP gene can be used to replace the corresponding mouse sequence at the mouse endogenous TSLP locus. For example, under control of mouse TSLP gene regulatory elements, a sequence (about 3.71 kb) starting from within exon 1 and ending within exon 5 of mouse TSLP gene was replaced with a corresponding sequence (about 4.18 kb) starting from within exon 1 and ending within exon 4 of human TSLP gene, to obtain a humanized TSLP gene locus as shown in FIG. 2, thereby humanizing mouse TSLP gene.

As shown in the schematic diagram of the targeting strategy in FIG. 3, the targeting vector contains homologous arm sequences upstream and downstream of the mouse TSLP gene, and an “A Fragment” containing DNA sequences of human TSLP gene. Specifically, sequence of the upstream homologous arm (5′ homologous arm, SEQ ID NO: 3) is identical to nucleotide sequence of 32943730-32948452 of NCBI accession number NC_000084.7, and sequence of the downstream homologous arm (3′ homologous arm, SEQ ID NO: 4) is identical to nucleotide sequence of 32953181-32957221 of NCBI accession number NC_000084.7. The genomic DNA sequence from human TSLP gene (SEQ ID NO: 5) is identical to nucleotide sequence of 111071891-111076074 of NCBI accession number NC_000005.10. The connection between the 5′ end of the A fragment and the mouse sequence was designed as:

(SEQ ID NO: 23)
5′-TCAGGAAGACTCCACGTTCAGGCGACAGCATGTTCCCTTTTGCC
TTAC-3′,

wherein the last “C” in sequence “CAGC” is the last nucleotide of the mouse sequence, the first “A” in sequence “ATGT” is the first nucleotide of the human sequence. The connection between the 3′ end of the A fragment and the mouse sequence was designed

(SEQ ID NO: 24)
5′-GAAAGTATAGGAACTTCATCAGTCAGGTACATAATGGTGGATCCC
ACTGGGTGCCTCCCTGCAGGAGAAAAGGTCTTCATA-3′,

wherein the last “G” in sequence “GGTG” is the last nucleotide of A fragment, and the first “C” in sequence “CCTC” is the first nucleotide of the mouse sequence.

The targeting vector also includes an antibiotic resistance gene for positive clone screening (neomycin phosphotransferase gene, or Neo), and two Frt recombination sites flanking the antibiotic resistance gene, that formed a Neo cassette (within the A Fragment). The connection between the 5′ end of the Neo cassette and the mouse sequence was designed as:

(SEQ ID NO: 25)
5′-CACACTTCCTTTGATCTGGTATCAACAAGCTTGATATCGAATTCCG
AAGTTCCTATTC-3′,

wherein the last “C” in sequence “CAAC” is the last nucleotide of the mouse sequence, and the first “A” in sequence “AAGC” is the first nucleotide of the Neo cassette. The connection between the 3′ end of the Neo cassette and the mouse sequence was designed as:

(SEQ ID NO: 26)
5′-TATAGGAACTTCATCAGTCAGGTACATAATGGTGGATCCCACTGGG
TGCCTCCCTGCAGGAGAAAAGG-3′,

wherein the last “G” in sequence “GGTG” is the last nucleotide of the Neo cassette, and the first “C” in sequence “CCTC” is the first nucleotide of the mouse sequence. In addition, a coding gene with a negative selectable marker (a gene encoding diphtheria toxin A subunit (DTA)) was also constructed downstream of the 3′ homologous arm of the targeting vector. The mRNA sequence of the engineered mouse TSLP after humanization and its encoded protein sequence are shown in SEQ ID NO: 6 and SEQ ID NO: 2, respectively.

Given that human TSLP has multiple isoforms or transcripts, the methods described herein can be applied to other isoforms or transcripts.

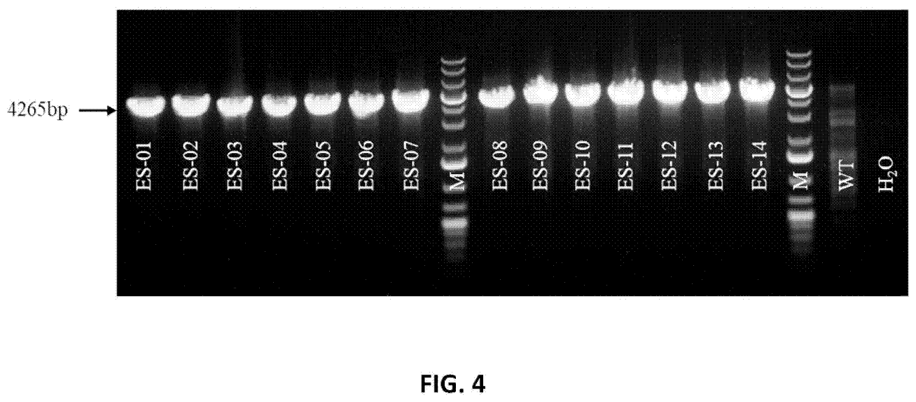

The targeting vector was constructed, e.g., by restriction enzyme digestion and ligation. The constructed targeting vector sequences were preliminarily confirmed by restriction enzyme digestion, and then verified by sequencing. Embryonic stem cells of C57BL/6 mice were transfected with the correct targeting vector by electroporation. The positive selectable marker genes were used to screen the cells, and the integration of exogenous genes was confirmed by PCR and Southern Blot. The PCR detection results are shown in FIG. 4. The results indicate that mice numbered ES-01, ES-02, ES-03, ES-04, ES-05, ES-06, ES-07, ES-08, ES-09, ES-10, ES-11, ES-12, ES-13, and ES-14 were positive clones.

The following PCR primers were used:

ES-F (SEQ ID NO: 67):
5′-GCTCGACTAGAGCTTGCGGA-3′,
ES-R (SEQ ID NO: 68):
5′-AGAGATGGTCTCCTTGGAGGTAGGC-3′.

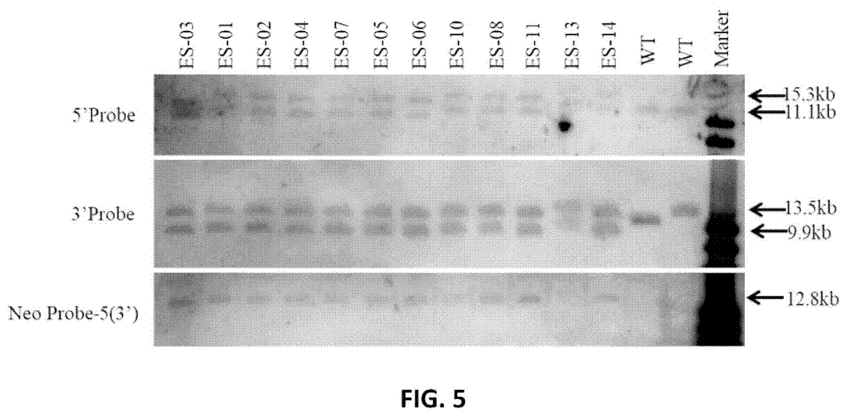

Further, the clones identified as positive by PCR were then verified by Southern Blot, to confirm whether the clones had random insertions (cell DNA was digested with DraIII, HindIII, or EcoRV; and hybridized with three probes). The restriction enzymes, probes, and the size of target fragment sizes are shown in the table below. The Southern Blot detection results are shown in FIG. 5. The results indicate that mice numbered ES-01, ES-02, ES-04, ES-05, ES-06, ES-07, ES-08, ES-10, ES-11, ES-13, and ES-14 were verified as positive clones without random insertions.

TABLE 9
Enzymes and probes used in Southern Blot

| Restriction | | Wild-type | Recombinant | |
|---|---|---|---|---|
| enzyme | Probe | fragment size | fragment size | |
| DraIII | 5′ Probe | 11.1 kb | 15.3 | kb |
| HindIII | 3′ Probe | 13.5 kb | 9.9 | kb |
| EcoRV | Neo Probe-5(3′) | — | 12.8 | kb |

The following primers were used for probe synthesis in Southern Blot assays:

5′Probe-F (SEQ ID NO: 27):
5′-TACAAGTCCAGCATGACATAGCCA-3′,
5′Probe-R (SEQ ID NO: 28):
5′-TGCCACCTAATTGCAGAGGCGA-3′;
3′Probe-F (SEQ ID NO: 29):
5′-AAGACCTGACAGTGTTGTTCAAGG-3′,
3′Probe-R (SEQ ID NO: 30):
5′-TTCCGGTGGCCTGTAGGACATT-3′;
Neo Probe-5(3′)-F (SEQ ID NO: 31):
5′-GGATCGGCCATTGAACAAGAT-3′,
Neo Probe-5(3′)-R (SEQ ID NO: 32):
5′-CAGAAGAACTCGTCAAGAAGGC-3′.

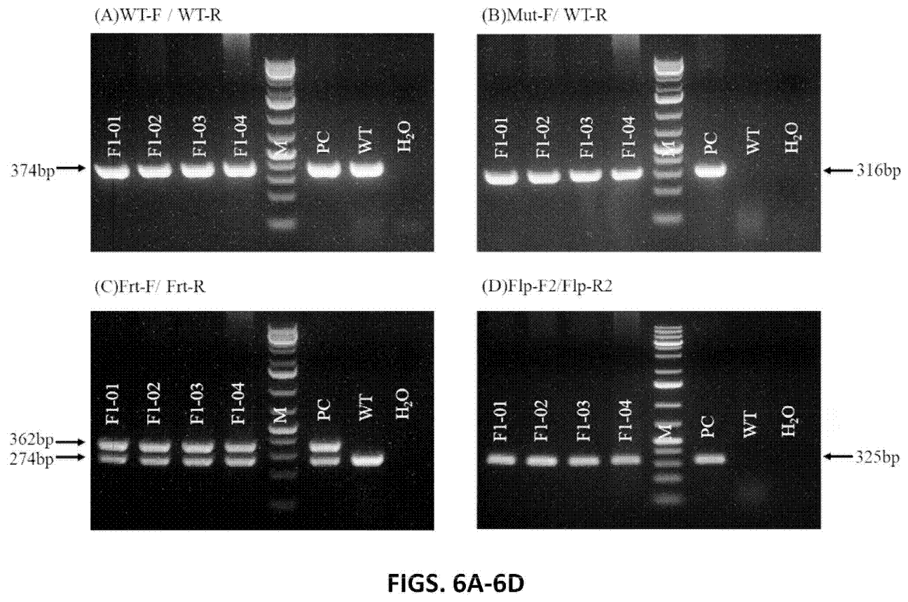

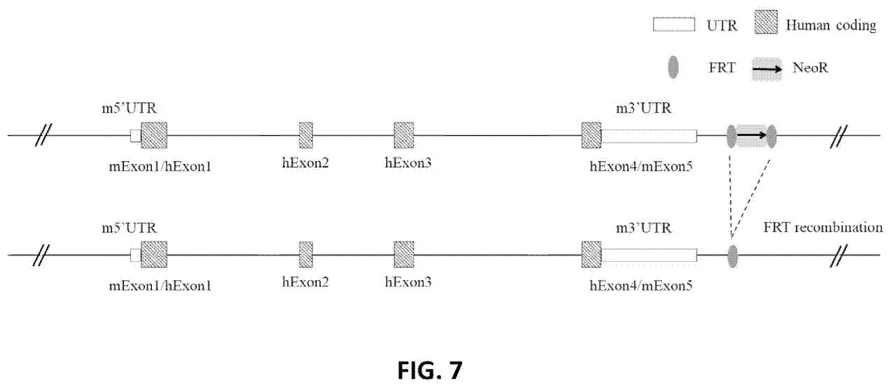

The positive clones that had been screened (black mice) were introduced into isolated blastocysts (white mice), and the resulted chimeric blastocysts were transferred to a culture medium for short-term culture and then transplanted to the fallopian tubes of the recipient mother (white mice) to produce the F0 chimeric mice (black and white). The F2 generation homozygous mice were obtained by backcrossing the F0 generation chimeric mice with wild-type mice to obtain the F1 generation mice, and then breeding the F1 generation heterozygous mice with each other. The positive mice were also bred with the Flp transgenic mice to remove the positive selectable marker genes (schematic diagram shown in FIG. 7), and then the humanized homozygous mice with a humanized TSLP gene were obtained by breeding the heterozygous mice with each other. The identification results of exemplary F1 generation mice (Neo cassette removed) are shown in FIGS. 6A-6D, wherein four mice numbered F1-01, F1-02, F1-03, and F1-04 were identified as positive heterozygous mice. The genotype of the TSLP gene humanized mice were verified by PCR using primers shown in the table below. The above results showed that the methods described herein can be used to generate TSLP gene humanized mice that can be stably passaged without random insertions.

TABLE 10
PCR primer sequences and target fragment sizes

| Primer | Primer sequence (5′-3′) | Target fragment size |
|---|---|---|
| WT-F | GCCACTCAGCCAGCCGTTAAAGATA | WT: 374 bp |
| | (SEQ ID NO: 33) | |
| WT-R | CCATTAACGGTCTATTGCTCATTTCCCA | |
| | (SEQ ID NO:86) | |
| Mut-F | TGCTACTCAGGCAATGAAGAAGAGGAGA | Mut: 316 bp |
| | (SEQ ID NO: 87) | |
| WT-R | CCATTAACGGTCTATTGCTCATTTCCCA | |
| | (SEQ ID NO: 70) | |
| Frt-F | AGGGACATCACGGTTGTCTACATCT | WT: 274 bp |
| | (SEQ ID NO: 72) | Mut: 363 bp |
| Frt-R | AGGGTGTTTTACCAGAGGTGTGCAG | (Neo cassette removed) |
| | (SEQ ID NO: 73) | |
| Flp-F2 | GACAAGCGTTAGTAGGCACATATAC | Mut: 325 bp |
| | (SEQ ID NO: 74) | |
| Flp-R2 | GCTCCAATTTCCCACAACATTAGT | |
| | (SEQ ID NO: 88) | |

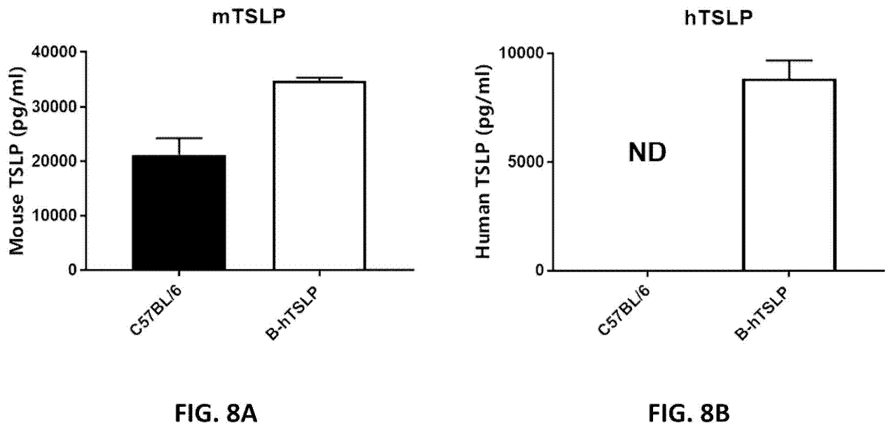

The expression of human TSLP protein in the obtained positive mice can be confirmed, e.g., using ELISA method. Two 10-week-old wild-type C57BL/6 mice and two 23-week-old TSLP gene humanized heterozygous mice were selected. The mouse ear tissues were collected, and supernatant was collected after grinding. The expression levels of mouse TSLP protein and human or humanized TSLP protein were detected using MOUSE TSLP ELISA KIT and HUMAN TSLP ELISA KIT, respectively. As shown in FIGS. 8A-8B, expression of human or humanized TSLP protein was not detected in wild-type C57BL/6 mice; whereas expressions of both mouse TSLP protein and human TSLP protein were detected in the TSLP gene humanized heterozygous mice.

The results indicate that genetically engineered mice with a humanized TSLP gene can be constructed to express human TSLP protein using the methods described herein. The mice can be stably passaged without random insertions.

### Example 2: Mice with Humanized TSLPR Gene-1

In this example, a non-human animal (e.g., a mouse) was modified to include a nucleotide sequence encoding human TSLPR protein, and the obtained genetically-modified non-human animal can express a human or humanized TSLPR protein in vivo. The mouse TSLPR gene (NCBI Gene ID: 57914, Primary source: MGI: 1889506, UniProt ID: A0A0R4J0F5) is located at 109702575 to 109707301 of chromosome 5 (NC_000071.7), and the human TSLPR gene (NCBI Gene ID: 64109, Primary source: HGNC: 14281, UniProt ID: Q9HC73-1) is located at 1190437 to 1212762 of chromosome X (NC_000023.11). The mouse TSLPR transcript is NM_016715.4, and the corresponding protein sequence NP_057924.3 is set forth in SEQ ID NO: 7. The human TSLPR transcript is NM_022148.4, and the corresponding protein sequence NP_071431.2 is set forth in SEQ ID NO: 8. Mouse and human TSLPR gene loci are shown in FIG. 9.

All or part of nucleotide sequences encoding human TSLPR protein can be introduced into the mouse endogenous TSLPR locus, so that the mouse expresses human or humanized TSLPR protein. Specifically, using gene-editing techniques, under control of mouse TSLPR gene regulatory elements, a nucleotide sequence encoding a portion of human TSLPR protein and a nucleotide sequence encoding a portion of mouse TSLPR protein were inserted to the mouse TSLPR gene locus, to obtain a humanized TSLRP gene locus shown in FIG. 10, thereby humanized mouse TSLPR gene.

As shown in the schematic diagram of the targeting strategy in FIG. 11, the targeting vector contains homologous arm sequences upstream and downstream of the mouse TSLPR gene, and an “A2 Fragment” (SEQ ID NO: 14) containing a P2A-encoding sequence (SEQ ID NO: 17), a nucleotide sequence encoding a portion of human TSLPR protein, and a nucleotide sequence encoding a portion of mouse TSLPR protein. Specifically, sequence of the upstream homologous arm (5′ homologous arm, SEQ ID NO: 9) is identical to nucleotide sequence of 109705410-109709296 of NCBI accession number NC_000071.7, and sequence of the downstream homologous arm (3′ homologous arm, SEQ ID NO: 10) is identical to nucleotide sequence of 109701469-109705409 of NCBI accession number NC_000071.7. The nucleotide sequence (SEQ ID NO: 15) encoding a portion of human TSLRP protein is identical to the nucleotide sequence from position 16 to position 774 of NCBI accession number NM_022148.4. The nucleotide sequence (SEQ ID NO: 16) encoding a portion of mouse TSLRP protein is identical to the nucleotide sequence from position 873 to position 1190 of NCBI accession number NM_016715.4. The connection between the 5′ end of the A2 fragment and the mouse sequence

(SEQ ID NO: 34)
5′-CTCGGGCCCCGACCACCACGGAAGCGGAGCTACTAACTTCAGCCTG
CTGAA-3′,

wherein the last “C” in sequence “CCAC” is the last nucleotide of the mouse sequence, and the first “G” in sequence “GGAA” is the first nucleotide of the A2 fragment. The connection between the 3′ end of the A2 fragment and the mouse sequence was designed as:

(SEQ ID NO: 35)
5′-GAAAGTATAGGAACTTCATCAGTCAGGTACATAATGGTGGATCCG
GCGCCAACTTGAGCCTGGAGTTCC-3′,

whereas the last “C” in sequence “ATCC” is the last nucleotide of the A2 fragment, and the first “G” in sequence “GGCG” is the first nucleotide of the mouse sequence.

The targeting vector also includes an antibiotic resistance gene for positive clone screening (neomycin phosphotransferase gene, or Neo), and two Frt recombination sites flanking the antibiotic resistance gene, that formed a Neo cassette (within the A2 Fragment). The connection between the 5′ end of the Neo cassette and the STOP sequence was designed as:

(SEQ ID NO: 36)
5′-GCTTGGGCTGCAGGTCGAGGGACCTAGTCGACGGTATCGATAAGCT
TGATATCGAATTCCGAAGTTCCTATTCTCTAG-3′,

wherein the “A” in sequence “CCTA” is the last nucleotide of the STOP sequence, and the first “G” in sequence “GTCG” is the first nucleotide of the Neo cassette. The connection between the 3′ end of the Neo cassette and the mouse sequence was designed as:

(SEQ ID NO: 37)
5′-AAAGTATAGGAACTTCATCAGTCAGGTACATAATGGTGGATCCGGC
GCCAACTTGAGCCTGGAGTTCCGGTGAGTG.3′,

wherein the last “C” in sequence “ATCC” is the last nucleotide of the Neo cassette, and the first “G” in sequence “GGCG” is the first nucleotide of the mouse sequence. In addition, a coding gene with a negative selectable marker (a gene encoding diphtheria toxin A subunit (DTA)) was also constructed downstream of the 3′ homologous arm of the targeting vector. The mRNA sequence of the engineered mouse TSLPR after humanization and its encoded protein sequence are shown in SEQ ID NO: 18 and SEQ ID NO: 19, respectively.

Given that human TSLPR has multiple isoforms or transcripts, the methods described herein can be applied to other isoforms or transcripts.

The targeting vector was constructed, e.g., by restriction enzyme digestion and ligation. The constructed targeting vector sequences were preliminarily confirmed by restriction enzyme digestion, and then verified by sequencing. Embryonic stem cells of C57BL/6 mice were transfected with the correct targeting vector by electroporation. The positive selectable marker genes were used to screen the cells, and the integration of exogenous genes was confirmed by PCR and Southern Blot. PCR primers are shown in the table below.

TABLE 11
PCR primer sequences and target fragment sizes

| | | Target |
|---|---|---|
| Primer | Primer sequence (5′-3′) | fragment size |
| A-F | CGCCCTAGCCAAGGGTCTGAGTC | Mut: 524 bp |
| | (SEQ ID NO: 46) | |
| A-R | GAGAATGTCGTCTCGCTGCTCTGC | |
| | (SEQ ID NO: 48) | |

The clones identified as positive by PCR were verified by Southern Blot to confirm there was no random insertion. The correct clones were then further verified by sequencing, and subjected to subsequent experiments.

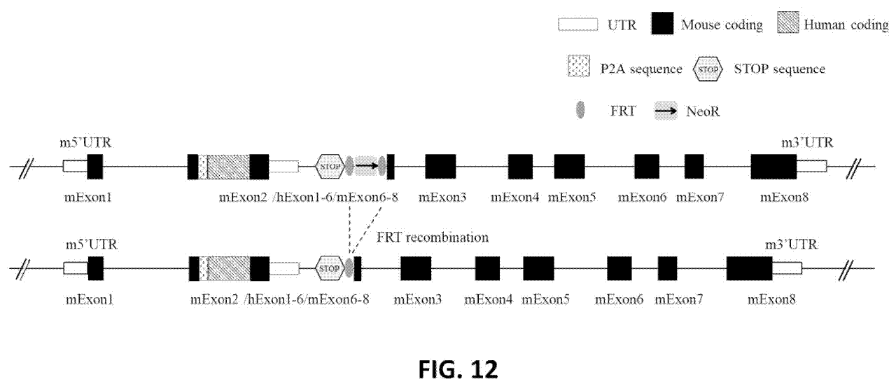

The positive clones that had been screened (black mice) were introduced into isolated blastocysts (white mice), and the resulted chimeric blastocysts were transferred to a culture medium for short-term culture and then transplanted to the fallopian tubes of the recipient mother (white mice) to produce the F0 chimeric mice (black and white). The F2 generation homozygous mice were obtained by backcrossing the F0 generation chimeric mice with wild-type mice to obtain the F1 generation mice, and then breeding the F1 generation heterozygous mice with each other. The positive mice were also bred with the Flp transgenic mice to remove the positive selectable marker genes (schematic diagram shown in FIG. 12), and then the humanized homozygous mice with a humanized TSLPR gene were obtained by breeding the heterozygous mice with each other.

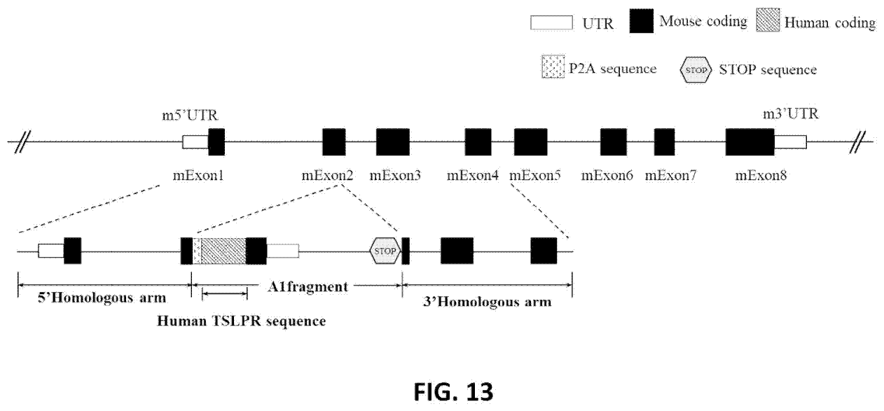

In addition, the CRISPR/Cas system can also be used for gene editing, and the targeting strategy shown in FIG. 13 was designed. The targeting vector contains homologous arm sequences upstream and downstream of the mouse TSLPR gene, and an “A1 Fragment” (SEQ ID NO: 13) containing a P2A-encoding sequence (SEQ ID NO: 17), a nucleotide sequence encoding a portion of human TSLPR protein, and a nucleotide sequence encoding a portion of mouse TSLPR protein. Specifically, sequence of the upstream homologous arm (5′ homologous arm, SEQ ID NO: 11) is identical to nucleotide sequence of 109705410-109706794 of NCBI accession number NC_000071.7, and sequence of the downstream homologous arm (3′ homologous arm, SEQ ID NO: 12) is identical to nucleotide sequence of 109704095-109705409 of NCBI accession number NC_000071.7. The nucleotide sequence (SEQ ID NO: 15) encoding a portion of human TSLRP protein is identical to the nucleotide sequence from position 16 to position 774 of NCBI accession number NM_022148.4. The nucleotide sequence (SEQ ID NO: 16) encoding a portion of mouse TSLRP protein is identical to the nucleotide sequence from position 873 to position 1190 of NCBI accession number NM_016715.4. The connection between the 5′ end of the A1 fragment and the mouse sequence was designed

(SEQ ID NO: 34)
5′-CTCGGGCCCCGACCACCACGGAAGCGGAGCTACTAACTTCAGCCTGC
TGAA-3′,

wherein the last “C” in sequence “CCAC” is the last nucleotide of the mouse sequence, and the first “G” in sequence “GGAA” is the first nucleotide of the A1 fragment. The connection between the 3′ end of the A1 fragment and the mouse sequence was designed as:

(SEQ ID NO: 40)
5′-GGGCTGCAGGTCGAGGGACCTAAGTACTGTCGACGGCGCCAACTTGA
GCCTGGAGTTCCGGTGAGTGAAGGGG-3′,

wherein the last “C” in sequence “CGAC” is the last nucleotide of the A1 fragment, and the first “G” in sequence “GGCG” is the first nucleotide of the mouse sequence. The mRNA sequence of the engineered mouse TSLPR after humanization and its encoded protein sequence are shown in SEQ ID NO: 18 and SEQ ID NO: 19, respectively.

The targeting vector was constructed, e.g., by restriction enzyme digestion and ligation. The constructed targeting vector sequences were preliminarily confirmed by restriction enzyme digestion, and then verified by sequencing. Targeting vectors with verified sequences were used for subsequent experiments.

Specific sgRNA sequences were designed and synthesized that recognize the targeting site. The targeting site sequence of the sgRNA on the TSLPR gene locus is as follows:

(SEQ ID NO: 41)
5′-GGCTCAAGTTGGCGCCGTGGTGG-3′.

UCA kit was used to detect the activity of the sgRNA. After confirming that the sgRNA can induce efficient Cas9 cleavage, restriction enzyme cleavage sites were added to its 5′ end and a complementary strand to obtain a forward oligonucleotide and a reverse oligonucleotide, as shown in the table below. After annealing, the products were ligated to the pT7-sgRNA plasmid (the plasmid was first linearized with BbsI), to obtain expression vector pT7-TSLPR-1.

TABLE 12
sgRNA sequence list
sgRNA sequence

| | |
|---|---|
| SEQ ID NO: 41 | Upstream: 5′-GGCTCAAGTTGGCGCCGTGGTGG-3′ |
| SEQ ID NO: 42 | Upstream: 5′-TAGGGGCTCAAGTTGGCGCCGTGGTGG-3′ |
| (forward oligonucleotide) | |
| SEQ ID NO: 43 | Downstream: 5′-CCACCACGGCGCCAACTTGAGCC-3′ |
| SEQ ID NO: 44 | Downstream: 5′-AAACCCACCACGGCGCCAACTTGAGCC-3′ |
| (reverse oligonucleotide) | |

The pT7-sgRNA vector was synthesized, which included a DNA fragment containing the T7 promoter and sgRNA scaffold (SEQ ID NO: 45), and was ligated to the backbone vector (Takara, Catalog number: 3299) after restriction enzyme digestion (EcoRI and BamHI). The resulting plasmid was confirmed by sequencing.

The pre-mixed Cas9 mRNA, the targeting vector, and in vitro transcription products of the pT7-TSLPR plasmid (using Ambion™ in vitro transcription kit to carry out the transcription according to the method provided in the product instruction) were injected into the cytoplasm or nucleus of fertilized eggs of C57BL/6 mice with a microinjection instrument. The embryo microinjection was carried out according to the method described, e.g., in A. Nagy, et al., “Manipulating the Mouse Embryo: A Laboratory Manual (Third Edition),” Cold Spring Harbor Laboratory Press, 2006. The injected fertilized eggs were then transferred to a culture medium to culture for a short time and then was transplanted into the oviduct of the recipient mouse to produce the genetically modified mice (F0 generation). The mouse population was further expanded by cross-breeding and self-breeding to establish stable homozygous mouse lines. The genotype of the somatic cells of the F0 generation mice can be identified by PCR analysis. The PCR primers are shown in the table below.

TABLE 13
PCR primer sequences and target fragment sizes

| | | Target |
|---|---|---|
| | | fragment |
| Primer | Primer sequence (5′-3′) | size |
| A-F | CGCCCTAGCCAAGGGTCTGAGTC | WT: 402 bp |
| | (SEQ ID NO: 46) | Mut: 3340 bp |
| A-R1 | GTCTGAAAGGAAGTACCGTCCCTAATC | |
| | (SEQ ID NO: 47) | |

Mice identified as positive by PCR were then subjected to Southern Blot detection. Those mice identified as positive by Southern Blot were further sequenced to confirm there was no random insertion.

Many methods can be used to verify the success of generating TSLPR gene humanized mice. For example, the genotype of the somatic cells of the F1 generation mice can be identified by PCR analysis. The PCR primers are shown in the table below.

TABLE 14
PCR primer sequences and target fragment sizes

| | | Target |
|---|---|---|
| | | fragment |
| Primer | Primer sequence (5′-3′) | size |
| F1-F | GTCGCCGCATCCGTTTATTGCC | 3573 bp |
| | (SEQ ID NO: 38) | |
| F1-R | GTCACACCACAGAAGTAAGGTTCCTTC | |
| | (SEQ ID NO: 39) | |

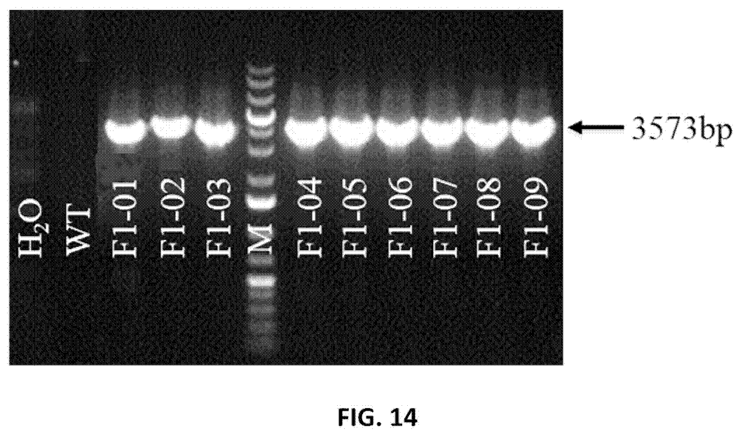

The identification results of some F1 generation mice are shown in FIG. 14. The results indicate that mice numbered F1-01, F1-02, F1-03, F1-04, F1-05, F1-06, F1-07, F1-08, and F1-09 were verified as positive heterozygous mice.

The F1 generation mice identified as positive by PCR were further verified by Southern Blot to confirm whether there was random insertion. Specifically, genomic DNA from the mouse tail was extracted, which was digested with ScaI or StuI. The digested genomic DNA was then transferred to a membrane and hybridized with respective probes. The restriction enzymes, probes, and the size of target fragment sizes are shown in the table below.

TABLE 15
Enzymes and probes used in Southern Blot

| Restriction | | Wild-type | Recombinant |
|---|---|---|---|
| enzyme | Probe | fragment size | fragment size |
| ScaI | lox2 STOP Probe | — | 5.5 kb |
| StuI | 3′Probe | 3.4 kb | 4.5 kb |

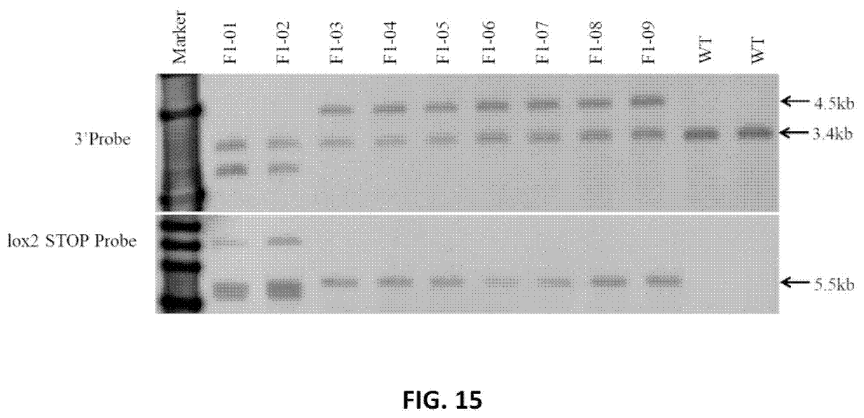

The Southern Blot detection results are shown in FIG. 15. The results indicate that mice numbered F1-03, F1-04, F1-05, F1-06, F1-07, F1-08, and F1-09 were verified as positive heterozygous clones without random insertions. The results indicate that genetically engineered mice with a humanized TSLPR gene can be constructed using the methods described herein. The mice can be stably passaged without random insertions. Finally, the F1 generation heterozygous mice were bred with each other to obtain the F2 generation homozygous mice.

The following primers were used for probe synthesis in Southern Blot assays:

### Lox2 STOP Probe:

lox2 STOP Probe-F:
(SEQ ID NO: 49)
5′-AACTGATGAATGGGAGCAGTGGTGG-3′,
lox2 STOP Probe-R:
(SEQ ID NO: 50)
5′-GCAGACACTCTATGCCTGTGTGGAG-3′;
3′Probe:
3′Probe-F:
(SEQ ID NO: 51)
5′-CGGGTGGGCGTGACCTGCGATG-3′,
3′Probe-R:
(SEQ ID NO: 52)
5′-CGGCGCAGGGGTCACCTGTGAG-3′

### Example 3: Mice with Humanized TSLPR Gene-2

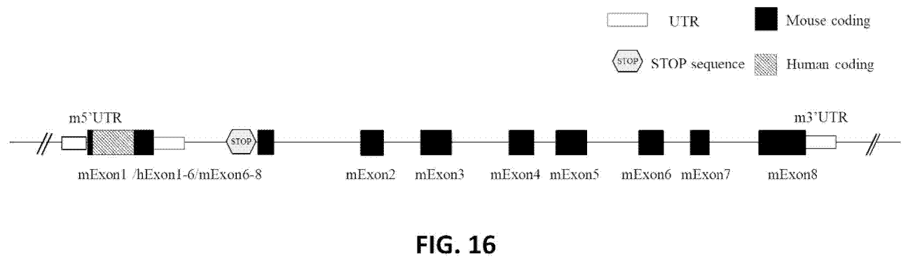

The inventors also successfully obtained TSLPR gene humanized mice by inserting a human/mouse chimeric sequence immediately after the start codon (ATG) within exon 1 of mouse TSLPR gene locus. The chimeric sequence is similar to the A1 or A2 fragment as described in Example 2, but does not include a P2A-encoding sequence. A schematic diagram of the humanized mouse TSLPR gene locus is shown in FIG. 16.

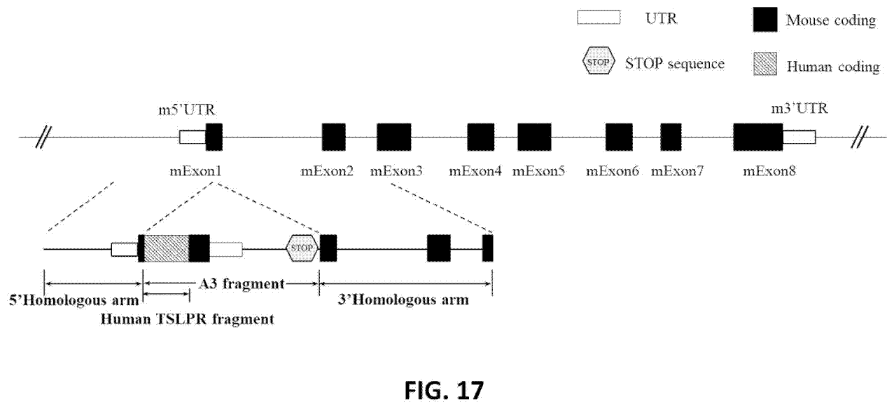

As shown in the schematic diagram of the targeting strategy in FIG. 17, the targeting vector contains homologous arm sequences upstream and downstream of the mouse TSLPR gene, and an “A3 Fragment” (SEQ ID NO: 22) containing a nucleotide sequence encoding a portion of human TSLPR protein, and a nucleotide sequence encoding a portion of mouse TSLPR protein. Specifically, sequence of the upstream homologous arm (5′ homologous arm, SEQ ID NO: 20) is 99.89% identical to nucleotide sequence of 109706747-109707691 of NCBI accession number NC_000071.7, and sequence of the downstream homologous arm (3′ homologous arm, SEQ ID NO: 21) is 99.89% identical to nucleotide sequence of 109704991-109706746 of NCBI accession number NC_000071.7. The nucleotide sequence (SEQ ID NO: 69) encoding a portion of human TSLRP protein is identical to the nucleotide sequence from position 19 to position 774 of NCBI accession number NM_022148.4. The nucleotide sequence (SEQ ID NO: 16) encoding a portion of mouse TSLRP protein is identical to the nucleotide sequence from position 873 to position 1190 of NCBI accession number NM_016715.4.

The targeting vector was constructed, e.g., by restriction enzyme digestion and ligation. The constructed targeting vector sequences were preliminarily confirmed by restriction enzyme digestion, and then verified by sequencing. Targeting vectors with verified sequences were used for subsequent experiments.

Specific sgRNA sequences were designed and synthesized that recognize the targeting site. The targeting site sequence of the sgRNA on the TSLPR gene locus is as follows:

(SEQ ID NO: 53)
5′-CCTGCCTCGGCTCCTTGCGGCGG-3′.

UCA kit was used to detect the activity of the sgRNA. After confirming that the sgRNA can induce efficient Cas9 cleavage, restriction enzyme cleavage sites were added to its 5′ end and a complementary strand to obtain a forward oligonucleotide and a reverse oligonucleotide, as shown in the table below. After annealing, the products were ligated to the pT7-sgRNA plasmid (the plasmid was first linearized with BbsI), to obtain expression vector pT7-TSLPR-2.

TABLE 16
sgRNA sequence list
sgRNA sequence

| | |
|---|---|
| SEQ ID NO: 53 | Upstream: 5′-CCTGCCTCGGCTCCTTGCGGCGG-3′ |
| SEQ ID NO: 54 | Upstream: 5′-TAGGCCTGCCTCGGCTCCTTGCGGCGG-3′ |
| (forward | |
| oligonucleotide) | |
| SEQ ID NO: 55 | Downstream: 5′-CCGCCGCAAGGAGCCGAGGCAGG-3′ |
| SEQ ID NO: 56 | Downstream: 5′-AAACCCGCCGCAAGGAGCCGAGGCAGG-3′ |
| (reverse oligonucleotide) | |

The pT7-sgRNA vector was synthesized, which included a DNA fragment containing the T7 promoter and sgRNA scaffold (SEQ ID NO: 45), and was ligated to the backbone vector (Takara, Catalog number: 3299) after restriction enzyme digestion (EcoRI and BamHI). The resulting plasmid was confirmed by sequencing.

The pre-mixed Cas9 mRNA, the targeting vector, and in vitro transcription products of the pT7-TSLPR plasmid (using Ambion™ in vitro transcription kit to carry out the transcription according to the method provided in the product instruction) were injected into the cytoplasm or nucleus of fertilized eggs of C57BL/6 mice with a microinjection instrument. The embryo microinjection was carried out according to the method described, e.g., in A. Nagy, et al., “Manipulating the Mouse Embryo: A Laboratory Manual (Third Edition),” Cold Spring Harbor Laboratory Press, 2006. The injected fertilized eggs were then transferred to a culture medium to culture for a short time and then was transplanted into the oviduct of the recipient mouse to produce the genetically modified mice (F0 generation). The mouse population was further expanded by cross-breeding and self-breeding to establish stable homozygous mouse lines.

The genotype of the somatic cells of the F0 generation mice can be identified by PCR analysis. The PCR primers are shown in the table below.

TABLE 17
PCR primer sequences and target fragment sizes

| | | Target |
|---|---|---|
| | | fragment |
| Primer | Primer sequence (5′-3′) | size |
| A3-F | 5′-TGGGACCGTCTGCAGAGTTTGTG-3′ | Mut: |
| | (SEQ ID NO: 57) | 3274 bp |
| A3-R | 5′-TCCCATTCATCAGTTCCATAGGT | |
| | TGG-3 ′(SEQ ID NO: 58) | |

The F0 generation TSLPR gene humanized mice that were identified as positive were bred with wild-type mice to obtain F1 generation mice. The F1 generation mice can be genotyped using the same PCR method described above to obtain the F1 generation positive mice. Finally, the F1 generation heterozygous mice were bred with each other to obtain the F2 generation homozygous mice.

### Example 4. Generation of TSLP/TSLPR Double-Gene Humanized Mice

The TSLP or TSLPR gene humanized mice generated using the methods described herein can also be used to generate double-gene humanized mouse models. For example, in Example 2 or 3, the embryonic stem (ES) cells for blastocyst microinjection can be collected from the TSLP gene humanized mice as described in Example 1. Alternatively, embryonic stem cells from humanized TSLP and/or TSLPR mice described herein can be isolated, and gene recombination targeting technology can be used to obtain TSLP/TSLPR double-gene modified mouse models. In addition, it is also possible to breed the TSLP gene humanized mice and TSLPR gene humanized mice obtained by the methods described herein. The TSLP/TSLPR double-gene humanized mice can be obtained by screening the positive offspring mice.

The expression of humanized TSLPR gene in positive mice can be confirmed, e.g., by RT-PCR, to detect the mRNA level. For example, one 8-week-old male C57BL/6 wild-type mouse and one 8-week-old male TSLP/TSLPR double-gene humanized heterozygous mouse prepared in this example were selected. After euthanasia by cervical dislocation, the peripheral blood of the mice was collected, and the cellular RNA was extracted according to the instructions of the TRIzol™ kit. The extracted RNA was then reverse transcribed into cDNA for RT-PCR detection using the primer sequences shown in the table below.

TABLE 18
RT-PCR primer sequences and target fragment sizes

| Primer | SEQ ID NO | Primer sequence (5′-3′) | Target fragment size |
|---|---|---|---|
| mTSLPR-F | SEQ ID NO: 59 | CAACCCTGCCCGCGATATTT | WT: 430 bp |
| mTSLPR-R | SEQ ID NO: 60 | TTGTCACCGCTGTCCACTC | |
| hTSLPR-F | SEQ ID NO: 61 | CCCGTTTTCACCGCAAGTC | Mut: 249 bp |
| hTSLPR-R | SEQ ID NO: 62 | CACCCTGACCCAGAAAGAGT | |
| GAPDH-F | SEQ ID NO: 63 | TCACCATCTTCCAGGAGCGAGA | WT: 479 bp |
| GAPDH-R | SEQ ID NO: 64 | GAAGGCCATGCCAGTGAGCTT | |

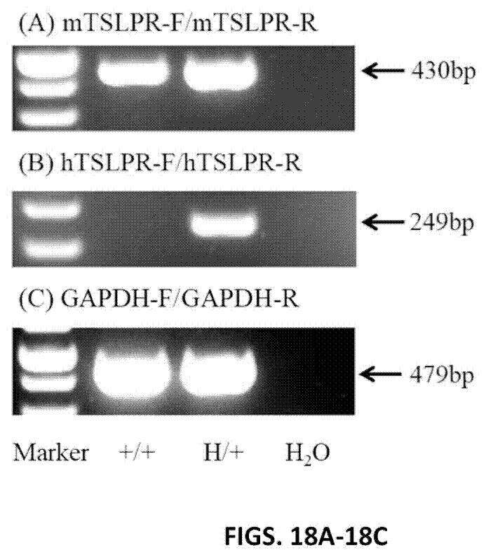

The detection results are shown in FIGS. 18A-18C. In the peripheral blood of the wild-type C57BL/6 mouse (+/+), only mouse TSLPR mRNA was detected, and no humanized TSLPR mRNA was detected. In addition, humanized TROP2 mRNA was only detected in the peripheral blood of the TSLP/TSLPR double-gene humanized heterozygous mouse (H/+).

The expression of humanized TSLPR gene in positive mice can be confirmed, e.g., by flow cytometry. Specifically, one 8-week-old male C57BL/6 wild-type mouse and one 8-week-old male TSLP/TSLPR gene humanized heterozygous mouse were selected, and peripheral blood was collected after euthanasia by cervical dislocation. Cells in the peripheral blood were stained with: Brilliant Violet 510™ anti-mouse CD45 (mCD45; an anti-mouse CD45 antibody), V450 Rat Anti-mouse CD11b (mCD11b; an anti-mouse CD11b antibody), APC anti-mouse TSLPR Antibody (mTSLPR; anti-mouse TSLPR antibody), PE anti-human TSLPR (TSLP-R) Antibody (hTSLPR; an anti-human TSLPR antibody), APC Rat IgG2a, κ Isotype Ctrl Antibody (an anti-mouse IgG2a antibody), PE Mouse IgG1, κ Isotype Ctrl Antibody (an anti-mouse IgG1 antibody), Purified anti-mouse CD16/32 (an anti-mouse CD16/32 antibody), and/or FITC anti-mouse F4/80 (mF4/80; an anti-mouse FITC antibody), and then subjected to flow cytometry analysis.

The results showed that the peripheral blood macrophages of C57BL/6 mice (characterized by mCD45+mCD11b+mF4/80+) had 0.77% hTSLPR positive cells (characterized by mCD45+mCD11b+mF4/80+hTSLPR+), and 91.5% mTSLPR positive cells (characterized as mCD45+mCD11b+mF4/80+mTSLPR+). By contrast, there were 24.7% hTSLPR-positive cells and 63.8% mTSLPR-positive cells in the peripheral blood of TSLPR heterozygous mice. The results indicate that the humanized TSLPR protein can be expressed normally in TSLP/TSLPR double-gene humanized heterozygous mice.

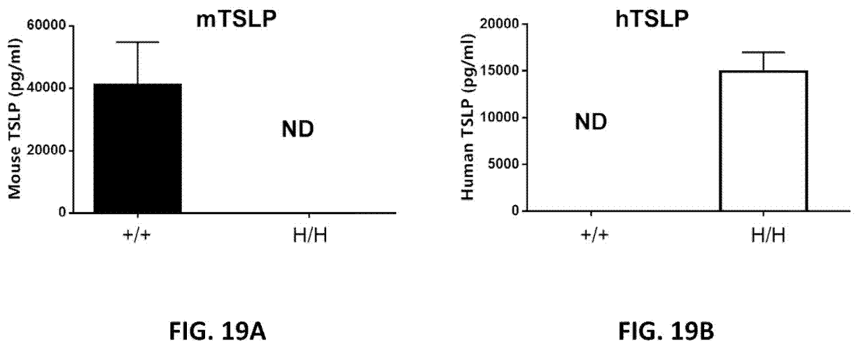

The TSLP/TSLPR double-gene humanized homozygous mice can be obtained by breeding the TSLP/TSLPR double-gene humanized heterozygous mice with each other. The expression of human TSLP protein or humanized TSLPR protein in the obtained homozygous mice can be confirmed, e.g., by ELISA or flow cytometry. Specifically, one wild-type C57BL/6 mouse and one TSLP/TSLPR double-gene humanized homozygous mouse were selected. The mouse ear tissues were collected, and supernatant was collected after grinding. The expression levels of mouse TSLP protein and human TSLP protein were detected using MOUSE TSLP ELISA KIT and HUMAN TSLP ELISA KIT, respectively. As shown in FIGS. 19A-19B, in wild-type C57BL/6 mouse (+/+), only mouse TSLP protein was detected, and no human TSLP protein was detected. However, in TSLP/TSLPR double-gene humanized mouse (H/H), only human TSLP protein was detected, and no mouse TSLP protein was detected.

The expression of humanized TSLPR protein in TSLP/TSLPR double-gene humanized homozygous mice was detected by flow cytometry. The results showed that the expression of human TSLPR protein was only detected in TSLP/TSLPR double-gene humanized homozygous mice, but not in wild-type C57BL/6 mice.

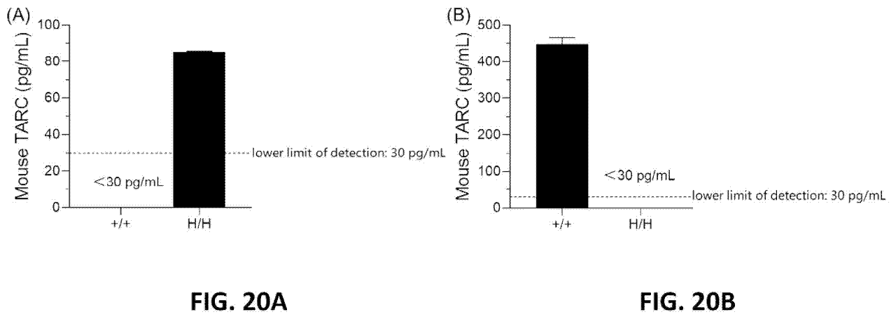

It is known that thymus activation-regulated chemokine (TARC) is a downstream product in the TSLP/TSLPR-mediated signaling pathway. Thus, the expression level of TARC in TSLP/TSLPR double-gene humanized homozygous mice can be used to verify whether the TSLP/TSLPR-mediated signaling pathway has a normal function. Specifically, bone marrow cells (2×10⁶ cells) from wild-type C57BL/6 mice and TSLP/TSLPR double-gene humanized homozygous mice were isolated. The cells were harvested after 9 days of continuous stimulation in RPMI medium containing 2 mL of 10% FBS and 200 ng/mL hFLT3L (human FMS-like tyrosine kinase 3 ligand). Next, the harvested cells were incubated with 100 ng/ml mouse TSLP protein (mTSLP) or human TSLP protein (hTSLP) for 3 days, and the level of mouse TARC in the cell supernatant was detected by ELISA. As shown in FIG. 20B, after induction with mTSLP, mouse TARC expression was detected only in wild-type C57BL/6 mice (+/+). As shown in FIG. 20A, after induction with hTSLP, the expression of mouse TARC was only detected in TSLP/TSLPR double-gene humanized homozygous mice. The results indicate that the TSLP/TSLPR-mediated signaling pathway was normal in TSLP/TSLPR double-gene humanized mice.

### Example 5. Generation of Multi-Gene Humanized Mice

The TSLP and/or TSLPR gene humanized mice generated using the methods described herein can also be used to generate double- or multi-gene humanized mouse models. For example, in Example 1, the embryonic stem (ES) cells for blastocyst microinjection can be selected from mice comprising other genetic modifications such as modified (e.g., human or humanized) IL6, TSLP, IL23, and/or TNF-α genes. Alternatively, embryonic stem cells from humanized TSLP and/or TSLPR mice described herein can be isolated, and gene recombination targeting technology can be used to obtain double-gene or multi-gene-modified mouse models of TSLP and/or TSLPR, and other gene modifications. In addition, it is also possible to breed the homozygous or heterozygous TSLP and/or TSLPR gene humanized mice obtained by the methods described herein with other genetically modified homozygous or heterozygous mice, and the offspring can be screened. According to Mendel's law, it is possible to generate double-gene or multi-gene modified heterozygous mice comprising modified (e.g., human or humanized) TSLP and/or TSLPR gene and other genetic modifications. Then the heterozygous mice can be bred with each other to obtain homozygous double-gene or multi-gene modified mice.

A variety of human disease models can be induced and prepared using the mice described herein, including psoriasis, multiple sclerosis and other models, which can be used to test the in vivo efficacy of human-specific antibodies. For example, TSLP and/or TSLPR gene humanized mice can be used to evaluate the pharmacodynamics, pharmacokinetics, and in vivo therapeutic efficacy of human-specific TSLP signaling pathway drugs in various disease models known in the art.

### Example 6. In Vivo Efficacy Verification

Single-gene or double-gene humanized TSLP/TSLPR homozygous mice can be selected and sensitized by administering ovalbumin (OVA) combined with aluminum hydroxide by intraperitoneal injection for 3 times. After 3 weeks, asthma is induced by continuous nebulization for 5 days. PBS can be used in the control group to replace OVA.

Asthma can be induced in single-gene or double-gene humanized TSLP/TSLPR mice using the protocol described above. Compared with control mice, OVA can induce typical symptoms such as elevated serum IgE levels and lung histopathological features. Analysis of infiltrating cells in bronchoalveolar lavage fluid (BALF) can also show increased eosinophil counts. The mice can then be treated with an anti-human TSLP antibody or anti-human TSLPR antibody. At the end of the experiment, the in vivo efficacy of anti-human antibodies can be assessed, e.g., by detecting airway reactivity of the mice; staining cells with hematoxylin and eosin (HE) or via immunohistochemistry (IHC) methods; measuring the number of inflammatory cells; and/or measuring serum IgE level.

### Example 7. Generation of IL33/TSLP/TSLPR Triple-Gene Humanized Mice

The mouse IL33 gene (NCBI Gene ID: 77125, Primary source: MGI: 1924375, UniProt ID: Q8BVZ5) is located at 29925114 to 29960715 of chromosome 19 (NC_000085.6), and the human IL33 gene (NCBI Gene ID: 90865, Primary source: HGNC: 16028, UniProt ID: 095760) is located at 6214591 to 6257983 of chromosome 9 (NC_000009.12). The mouse IL33 transcript is NM_133775.3 and the corresponding protein sequence NP_598536.2 is set forth in (SEQ ID NO: 65. The human IL33 transcript is NM_033439.3, and the corresponding protein sequence NP_254274.1 is set forth in SEQ ID NO: 66.

A gene sequence encoding the human IL33 protein can be introduced into the endogenous mouse IL33 locus, such that the mouse can express a human or humanized IL33 protein. Mouse cells can be modified by various gene-editing techniques, for example, replacement of specific mouse IL33 gene sequences with human IL33 gene sequences at the endogenous mouse IL33 locus. For example, a sequence about 9371 bp spanning from exon 2 to exon 8 of mouse IL33 gene was replaced with a corresponding human DNA sequence to obtain a humanized IL33 locus, thereby humanizing mouse IL33 gene.

As shown in the schematic diagram of the targeting strategy in FIG. 21, the targeting vector contained homologous arm sequences upstream (about 4218 bp upstream of exon 2 and a portion of exon 2 of endogenous IL33 gene) and downstream (a portion of exon 8 and about 4081 bp downstream of exon 8 of endogenous IL33 gene) of the mouse IL33 gene, and an “IL33-A fragment” comprising a human IL33 gene sequence. The upstream homologous arm sequence (5′ homologous arm, SEQ ID NO: 67) is identical to nucleotide sequence of 29945453-29949670 of NCBI accession number NC_000085.6, and the downstream homologous arm sequence (3′ homologous arm, SEQ ID NO: 68) is identical to nucleotide sequence of 29959042-29963122 of NCBI accession number NC_000085.6. The IL33-A fragment comprises a genomic DNA sequence starting within exon 2 and ends within exon 8 of human IL33 gene, which is identical to nucleotide sequence of 6241695-6256168 with NCBI accession number NC_000009.12. The modified humanized mouse IL33 mRNA sequence is shown in SEQ ID NO: 85, and the expressed protein has the same sequence as human IL33 protein shown in SEQ ID NO: 66.

The embryonic stem (ES) cells for blastocyst microinjection can be selected from the TSLP single-gene humanized mice, the TSLPR single-gene humanized mice, and/or the TSLP/TSLPR double-gene humanized mice obtained using the methods described herein. Alternatively, the TSLP single-gene humanized mice, the TSLPR single-gene humanized mice, and/or the TSLP/TSLPR double-gene humanized mice can be bred with the IL33 gene humanized mice described herein, and the offspring can be screened. According to Mendel's law, it is possible to obtain IL33/TSLP/TSLPR triple-gene humanized mice.

### Example 8. Generation of IL7R/TSLP/TSLPR Triple-Gene Humanized Mice

The mouse IL7R gene (NCBI Gene ID: 16197, Primary source: MGI: 96562, UniProt ID: P16872) is located at 9506159 to 9529941 of chromosome 15 (NC_000081.6), and the human IL7R gene (NCBI Gene ID: 3575, Primary source: HGNC: 6024, UniProt ID: P16871) is located at 35856891 to 35879603 of chromosome 5 (NC_000005.10). The mouse IL7R transcript is NM_008372.4 and the corresponding protein sequence NP_032398.3 is set forth in (SEQ ID NO: 75. The human IL7R transcript is NM_002185.5, and the corresponding protein sequence NP_002176.2 is set forth in SEQ ID NO: 76.

All or part of nucleotide sequences encoding the human IL7R protein can be introduced into the mouse endogenous IL7R locus, so that the mouse expresses the human or humanized IL7R protein. For example, a nucleotide sequence (e.g., DNA or cDNA sequence) of the human IL7R gene can be directly inserted at the mouse endogenous IL7R locus. Auxiliary sequences (e.g., stop codons or sequences containing a stop function) can be added to the inserted sequence, or other methods (e.g., inversion, or knockout) can be used, to make the mouse endogenous IL7R genomic sequence after the insertion site not being expressed normally. An in situ replacement strategy can also be used. For example, a nucleotide sequence at the mouse endogenous IL7R locus can be directly replaced with a human IL7R sequence (e.g., DNA or cDNA sequence). This example illustrates how to humanize the mouse IL7R gene by the in situ replacement strategy.

As shown in the schematic diagram of the targeting strategy in FIG. 22, the targeting vector contained homologous arm sequences upstream and downstream of the mouse IL7R gene, and a knock-in fragment (KI fragment) comprising a human IL7R gene sequence. The upstream homologous arm sequence (5′ homologous arm, SEQ ID NO: 77) is identical to nucleotide sequence of 9529743-9534180 of NCBI accession number NC_000081.6, and the downstream homologous arm sequence (3′ homologous arm, SEQ ID NO: 78) is identical to nucleotide sequence of 9505623-9509583 of NCBI accession number NC_000081.6. The KI fragment comprises a genomic DNA sequence of human IL7R gene, which is identical to nucleotide sequence of 35856978-35874459 with NCBI accession number NC_000005.10. The 5′ end of the human IL7R DNA sequence was directly connected with the 5′ homologous arm. The connection between the 3′ end of the human IL7R DNA sequence and the mouse sequence was designed as:

(SEQ ID NO: 79)
5′-CACAATCTATTCTTGCTTTCCAGGGGAGATGGATCCTGTCTTGCCA
AGTGTCACCATTCT-3′,

wherein the last “T” in sequence “TGGAT” is the last nucleotide of the human sequence, and the first “C” in sequence “CCTG” is the first nucleotide of the mouse sequence. The mRNA sequence of the engineered mouse IL7R after humanization and its encoded protein sequence are shown in SEQ ID NO: 80 and SEQ ID NO: 81, respectively.

The embryonic stem (ES) cells for blastocyst microinjection can be selected from the TSLP single-gene humanized mice, the TSLPR single-gene humanized mice, and/or the TSLP/TSLPR double-gene humanized mice obtained using the methods described herein. Alternatively, the TSLP single-gene humanized mice, the TSLPR single-gene humanized mice, and/or the TSLP/TSLPR double-gene humanized mice can be bred with the IL7R gene humanized mice described herein, and the offspring can be screened. According to Mendel's law, it is possible to obtain IL7R/TSLP/TSLPR triple-gene humanized mice.

### Example 9. Oxazolone (OXA)-Induced Atopic Dermatitis Model

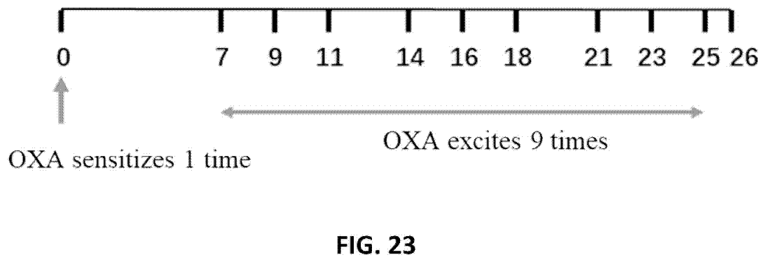

The TSLP/TSLPR double-gene humanized homozygous mice (5-7 weeks) were selected. The mice were induced using oxazolone (OXA) to establish an atopic dermatitis (AD) model. Specifically, the TSLP/TSLPR double-gene humanized homozygous mice were randomly placed into a control group (G1), a model group (G2), and four treatment groups according to body weight (5-8 mice in each group). As shown in the experimental scheme of FIG. 23, all model/treatment group mice (G2-G6) were first sensitized (on day 0) with 0.8% OXA smeared on their ears and backs, and then challenged with 0.4% OXA smeared on their ears and backs. The control group mice (G1) were treated with solvent (acetone:olive oil=4:1) instead of 0.4% OXA and 0.8% OXA. The treatment group mice were injected with Dexamethasone (3 mg/kg; G3) or Tezepelumab analog (1-10 mg/kg; G4-G6). The VH and VL sequences of Tezepelumab are set forth in SEQ ID NO: 70 and SEQ ID NO: 71, respectively. The model group mice (G2) were injected with an equal volume of a human IgG2 (hIgG2) antibody. Dexamethasone was administered once a day for a total of 20 doses. Tezepelumab analog was administered twice weekly for a total of 6 doses. The specific dosages are listed in the following table.

TABLE 19
Grouping and dosing schedule

| | | | | | | Adminis- |
|---|---|---|---|---|---|---|
| | | | | | | tration |
| | | | Adminis- | | | frequency |
| | Animal | | tration | Dosage | | and total |
| Group | number | Treatment | route | (mg/kg) | | times |
| G1 | 5 | — | — | — | | — |
| G2 | 8 | hIgG2 | i.p. | 3 | mg/kg | BIW × 6 |
| G3 | 8 | Dexamethasone | i.p. | 10 | mg/kg | QD × 20 |
| G4 | 8 | Tezepelumab- | i.p. | 1 | mg/kg | BIW × 6 |
| | | analog | | | | |
| G5 | 8 | Tezepelumab- | i.p. | 3 | mg/kg | BIW × 6 |
| | | analog | | | | |
| G6 | 8 | Tezepelumab- | i.p. | 10 | mg/kg | BIW × 6 |
| | | analog | | | | |

Body weight and the ear thickness of the mice were measured twice a week. At the end of the experiment (day 26), serum IgE levels were measured and ear tissues were collected for hematoxylin and eosin (H&E) or immunohistochemistry (IHC) pathological examination. According to the epidermal stromal cell hyperplasia, erosion/scab, hyperkeratosis, dermal and subcutaneous mixed inflammatory cell infiltration, and eosinophilic infiltration conditions, an overall score was rated (e.g., 0, 0.5, 1, 1.5, or 2) that represents the slight, mild, moderate, and severe cases.

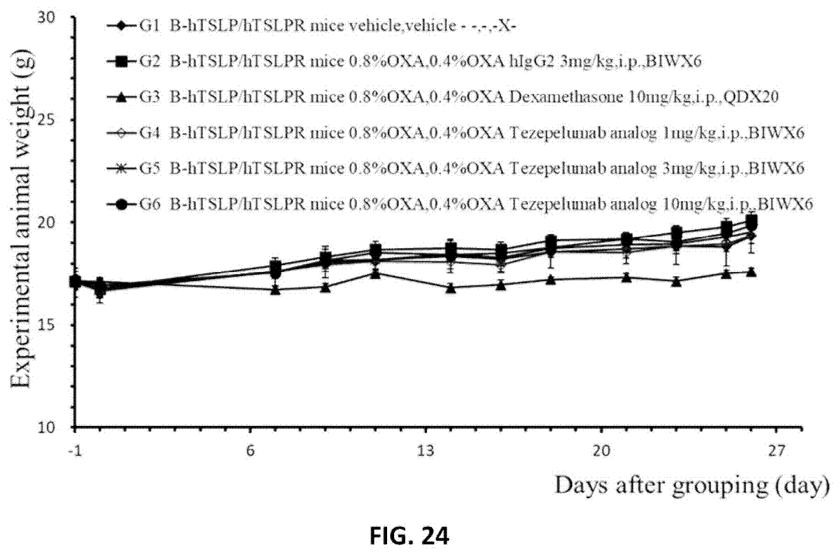

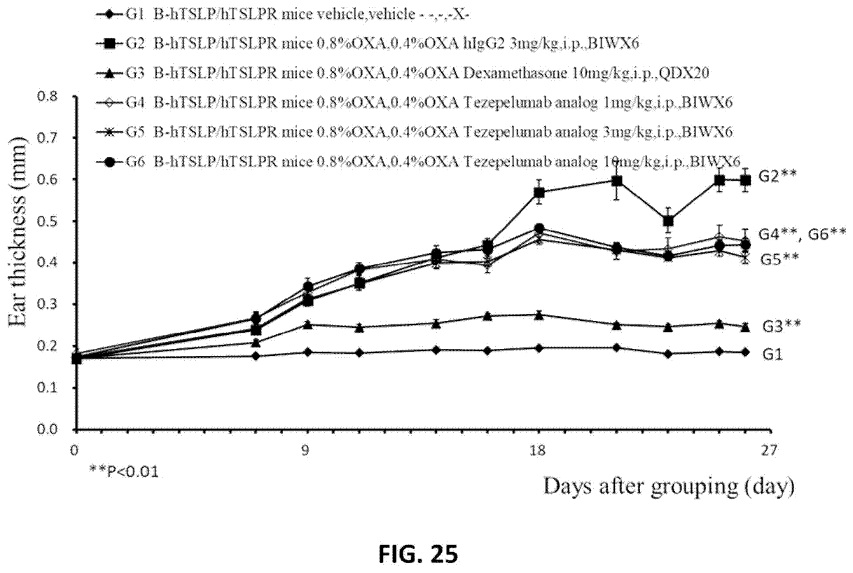

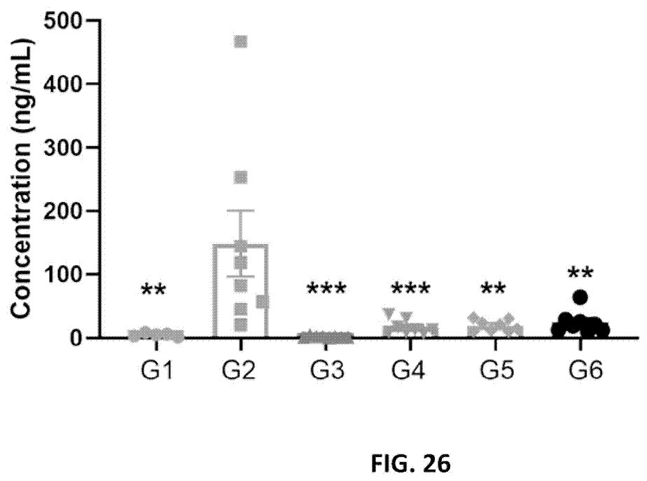

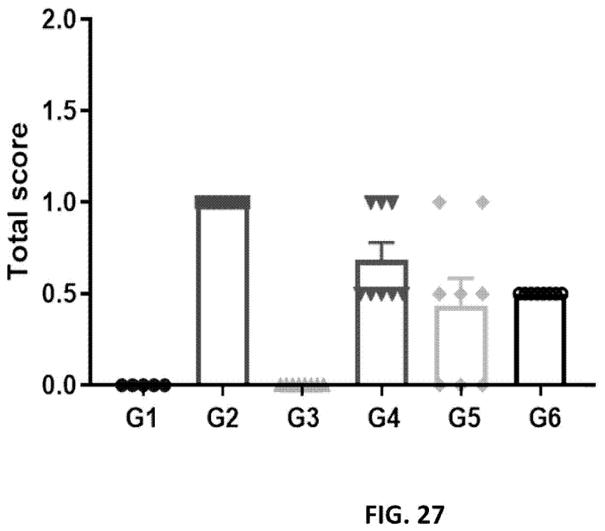

Overall, the mice were in good health during the experiment. Compared with the control group G1, the body weight of the treatment group G3 decreased, and the body weight of the other groups showed an upward trend (FIG. 24). Atopic dermatitis was induced using the scheme above in the TSLP/TSLPR double-gene humanized homozygous mice. Compared with the control group mice (G1), the OXA-induced group mice (G2) showed symptoms such as epidermal stromal cell hyperplasia, thickening, elevated serum IgE levels, eosinophil infiltration, and increased ear thickness (FIGS. 25-29). Compared to the OXA-induced group (G2), treatment with Dexamethasone (G3) and anti-human TSLP antibodies (G4-G6) significantly reduced the total IgE concentration in serum of mice (FIG. 26).

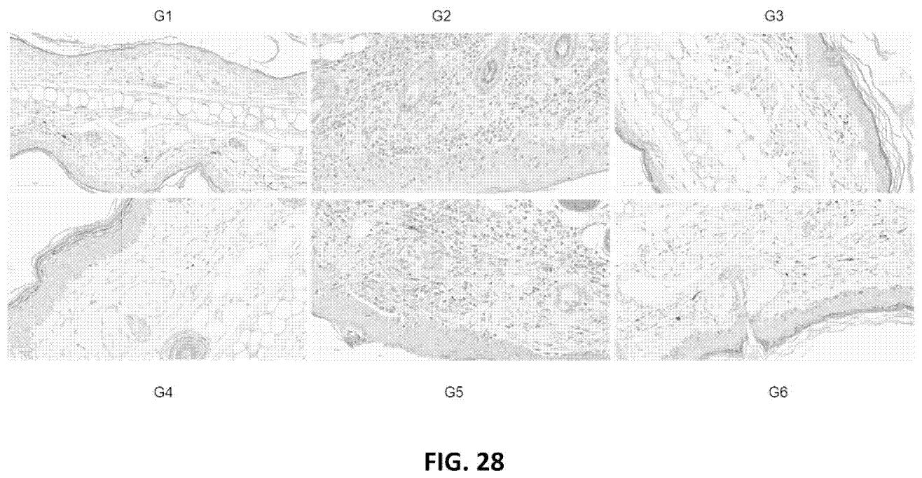

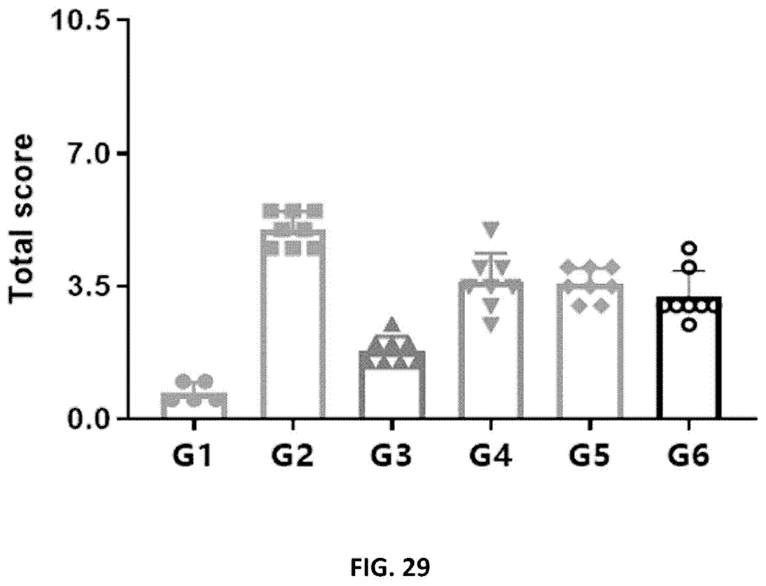

According to the H&E staining results (FIG. 28), the above typical symptoms were significantly reduced in the treatment group relative to the OXA-induced group (G2). For example, significant improvements of stromal hyperplasia, epidermal erosion/crust, dermal and subcutaneous mixed inflammatory cell infiltration, and dermal and subcutaneous eosinophilic infiltration lesions were observed. Particularly, the G6 group (10 mg/kg of Tezepelumab analog) showed the best overall treatment effect (FIG. 29). The results indicate that the TSLP/TSLPR double-gene humanized mice prepared by the methods described herein can simulate disease models and be used to screen and evaluate the in vivo efficacy of anti-human TSLP/TSLPR antibodies in preclinical research, and can be used to characterize anti-human TSLP and/or TSLPR antibody properties.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A genetically-modified, non-human animal whose genome comprises at least one chromosome comprising a sequence encoding a human or chimeric thymic stromal lymphopoietin (TSLP).

2. The animal of claim 1, wherein the sequence encoding the human or chimeric TSLP is operably linked to an endogenous regulatory element (e.g., endogenous 5′ UTR and/or 3′ UTR) at the endogenous TSLP gene locus in the at least one chromosome.

3. The animal of claim 1 or 2, wherein the sequence encoding a human or chimeric TSLP comprises a sequence encoding an amino acid sequence that is at least 70%, 75%, 80%, 85%, 90%, 95%, 99%, or 100% identical to human TSLP (NP_149024.1; SEQ ID NO: 2).

4. The animal of any one of claims 1-3, wherein the animal is a mammal, e.g., a monkey, a rodent, a mouse, or a rat.

5. The animal of any one of claims 1-4, wherein the animal is a mouse.

6. The animal of any one of claims 1-5, wherein the animal does not express endogenous TSLP or expresses a decreased level of endogenous TSLP as compared to TSLP expression level in a wild-type animal.

7. The animal of any one of claims 1-6, wherein the animal has one or more cells expressing human or chimeric TSLP.

8. The animal of any one of claims 1-7, wherein the animal has one or more cells expressing human or chimeric TSLP, and endogenous TSLP receptor (TSLPR) can bind to the expressed human or chimeric TSLP, inducing downstream signaling pathways (e.g., inducing release of thymus activation-regulated chemokine (TARC)).

9. The animal of any one of claims 1-7, wherein the animal has one or more cells expressing human or chimeric TSLP, and human TSLP receptor (TSLPR) can bind to the expressed human or chimeric TSLP, inducing downstream signaling pathways (e.g., inducing release of thymus activation-regulated chemokine (TARC)).

10. A genetically-modified, non-human animal, wherein the genome of the animal comprises a replacement of a sequence encoding a region of endogenous TSLP with a sequence encoding a corresponding region of human TSLP at an endogenous TSLP gene locus.

11. The animal of claim 10, wherein the sequence encoding the corresponding region of human TSLP is operably linked to an endogenous regulatory element at the endogenous TSLP locus, and one or more cells of the animal express a human or chimeric TSLP.

12. The animal of claim 10 or 11, wherein the animal does not express endogenous TSLP or expresses a decreased level of endogenous TSLP as compared to TSLP expression level in a wild-type animal.

13. The animal of any one of claims 10-12, wherein the replaced sequence encodes the full-length protein of TSLP.

14. The animal of any one of claims 10-13, wherein the animal is a mouse, and the replaced endogenous TSLP region comprises a portion of exon 1, exon 2, exon 3, exon 4, and/or a portion of exon 5 of the endogenous mouse TSLP gene.

15. The animal of any one of claims 10-14, wherein the animal is heterozygous or homozygous with respect to the replacement at the endogenous TSLP gene locus.

16. A non-human animal comprising at least one cell comprising a nucleotide sequence encoding a humanized TSLP polypeptide, wherein the humanized TSLP polypeptide comprises at least 50, 100, 110, 120, 130, 140, 150, 155, 156, 157, 158, or 159 contiguous amino acid residues that are identical to the corresponding contiguous amino acid sequence of a human TSLP, wherein the animal expresses the humanized TSLP polypeptide.

17. The animal of claim 16, wherein the nucleotide sequence is operably linked to an endogenous TSLP regulatory element of the animal.

18. The animal of claim 16 or 17, wherein the nucleotide sequence is integrated to an endogenous TSLP gene locus of the animal.

19. The animal of any one of claims 16-18, wherein the humanized TSLP polypeptide has at least one mouse TSLP activity and/or at least one human TSLP activity.

20. A method for making a genetically-modified, non-human animal, comprising:
replacing in at least one cell of the animal, at an endogenous TSLP gene locus, a sequence encoding a region of an endogenous TSLP with a sequence encoding a corresponding region of human TSLP.

21. The method of claim 20, wherein the sequence encoding the corresponding region of human TSLP comprises a portion of exon 1, exon 2, exon 3, and/or a portion of exon 4 of a human TSLP gene.

22. The method of claim 20 or 21, wherein the sequence encoding the corresponding region of human TSLP comprises at least 50, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, or 480 nucleotides of exon 1, exon 2, exon 3, and/or exon 4 of a human TSLP gene.

23. The method of any one of claims 20-22, wherein the sequence encoding the corresponding region of human TSLP encodes a sequence that is at least 80%, 85%, 90%, 95%, 96%, 97%, 98%, 99%, or 100% identical to SEQ ID NO: 2.

24. The method of any one of claims 20-23, wherein the animal is a mouse, and the locus is a portion of exon 1, exon 2, exon 3, exons 4, and/or a portion of exon 5 of the mouse TSLP gene.

25. A method of making a genetically-modified non-human animal cell that expresses a human or chimeric TSLP, the method comprising:
replacing, at an endogenous mouse TSLP gene locus, a nucleotide sequence encoding a region of endogenous TSLP with a nucleotide sequence encoding a corresponding region of human TSLP, thereby generating a genetically-modified non-human animal cell that includes a nucleotide sequence that encodes the human or chimeric TSLP, wherein the animal cell expresses the human or chimeric TSLP.

26. The method of claim 25, wherein the animal is a mouse.

27. The method of claim 25 or 26, wherein the nucleotide sequence encoding the human or chimeric TSLP is operably linked to an endogenous TSLP regulatory region, e.g., promoter.

28. The animal of any one of claims 1-19, wherein the animal further comprises a sequence encoding an additional human or chimeric protein, e.g., TSLP receptor (TSLPR), IL33, IL7R, IL6, IL12, IL23, and/or Tumor necrosis factor alpha (TNF-α).

29. The animal of claim 28, wherein the additional human or chimeric protein is TSLPR, IL33, and/or IL7R.

30. The method of any one of claims 20-27, wherein the animal or mouse further comprises a sequence encoding an additional human or chimeric protein, e.g., TSLPR, IL33, IL7R, IL6, IL12, IL23, and/or TNF-α.

31. The method of claim 30, wherein the additional human or chimeric protein is TSLPR, IL33, and/or IL7R.

32. A genetically-modified, non-human animal whose genome comprises at least one chromosome comprising a sequence encoding a human or chimeric thymic stromal lymphopoietin receptor (TSLPR).

33. The animal of claim 32, wherein the sequence encoding the human or chimeric TSLPR is operably linked to an endogenous regulatory element at the endogenous TSLPR gene locus in the at least one chromosome.

34. The animal of claim 32 or 33, wherein the sequence encoding a human or chimeric TSLPR comprises a sequence encoding an amino acid sequence that is at least 70%, 75%, 80%, 85%, 90%, 95%, 99%, or 100% identical to human TSLPR (NP_071431.2; SEQ ID NO: 8).

35. The animal of claim 32 or 33, wherein the sequence encoding a human or chimeric TSLPR comprises a sequence encoding an amino acid sequence that is at least 70%, 75%, 80%, 85%, 90%, 95%, 99%, or 100% identical to amino acids 1-253 or 2-253 of human TSLPR (NP_071431.2; SEQ ID NO: 8).

36. The animal of claim 32 or 33, wherein the sequence encoding a human or chimeric TSLPR comprises a sequence encoding an amino acid sequence that is at least 70%, 75%, 80%, 85%, 90%, 95%, 99%, or 100% identical to SEQ ID NO: 19.

37. The animal of any one of claims 32-36, wherein the animal is a mammal, e.g., a monkey, a rodent, a mouse, or a rat.

38. The animal of any one of claims 32-37, wherein the animal is a mouse.

39. The animal of any one of claims 32-38, wherein the animal does not express endogenous TSLPR or expresses a decreased level of endogenous TSLPR as compared to TSLPR expression level in a wild-type animal.

40. The animal of any one of claims 32-39, wherein the animal has one or more cells expressing human or chimeric TSLPR.

41. The animal of any one of claims 32-40, wherein the animal has one or more cells expressing human or chimeric TSLPR that can bind to endogenous TSLP and IL7 receptor (IL7R) to induce downstream signaling pathways (e.g., inducing release of thymus activation-regulated chemokine (TARC)).

42. The animal of any one of claims 32-40, wherein the animal has one or more cells expressing human or chimeric TSLPR that can bind to human TSLP and IL7R to induce downstream signaling pathways (e.g., inducing release of thymus activation-regulated chemokine (TARC)).

43. A genetically-modified, non-human animal, wherein the genome of the animal comprises an insertion of a sequence encoding a human or chimeric TSLPR at an endogenous TSLPR gene locus.

44. The animal of claim 43, wherein the sequence encoding a human or chimeric TSLPR is operably linked to an endogenous regulatory element at the endogenous TSLPR locus, and one or more cells of the animal express the human or chimeric TSLPR.

45. The animal of claim 43 or 44, wherein the animal does not express endogenous TSLPR or expresses a decreased level of endogenous TSLPR as compared to TSLPR expression level in a wild-type animal.

46. The animal of any one of claims 43-45, wherein the sequence encoding a human or chimeric TSLPR is inserted within exon 1, intron 1, exon 2, intron 2, exon 3, intron 3, exon 4, intron 4, exon 5, intron 5, exon 6, intron 6, exon 7, intron 7, and/or exon 8 of endogenous TSLPR gene.

47. The animal of any one of claims 43-46, wherein the sequence encoding a human or chimeric TSLPR is inserted within exon 2 of endogenous TSLPR gene.

48. The animal of any one of claims 43-47, wherein the sequence encoding a human or chimeric TSLPR is inserted immediately after a nucleotide corresponding to position 260 of NM_016715.4.

49. The animal of any one of claims 43-48, wherein the inserted sequence comprises, optionally from 5′ end to 3′ end:
a) a sequence encoding a self-cleaving peptide;
b) a first sequence comprising a sequence encoding the extracellular region and transmembrane region of a human TSLPR;
c) a second sequence comprising a sequence encoding all or a portion of the cytoplasmic region of an endogenous TSLPR;
d) a regulatory sequence of endogenous TSLPR gene (e.g., 3′ UTR); and
e) optionally one or more auxiliary sequences (e.g., WPRE, lox2, STOP, and/or polyA).

50. The animal of claim 49, wherein the self-cleaving peptide is T2A, P2A, E2A, or F2A (e.g., P2A).

51. The animal of claim 49 or 50, wherein the first sequence further comprises a sequence encoding at least 1, at least 2, at least 3, at least 4, at least 5, or at least 6 amino acids of the cytoplasmic region of a human TSLPR, and the second sequence does not comprise a corresponding sequence encoding at least 1, at least 2, at least 3, at least 4, at least 5, or at least 6 amino acids of the cytoplasmic region of an endogenous TSLPR.

52. The animal of any one of claims 49-51, wherein the first sequence is at least 70%, 75%, 80%, 85%, 90%, 95%, 99%, or 100% identical to SEQ ID NO: 15, and the second sequence is at least 70%, 75%, 80%, 85%, 90%, 95%, 99%, or 100% identical to SEQ ID NO: 16.

53. The animal of any one of claims 49-52, wherein the first sequence encodes an amino acid that is at least 70%, 75%, 80%, 85%, 90%, 95%, 99%, or 100% identical to amino acids 1-253 of SEQ ID NO: 8, and the second sequence encodes an amino acid sequence that is at least 70%, 75%, 80%, 85%, 90%, 95%, 99%, or 100% identical to amino acids 255-359 of SEQ ID NO: 7.

54. The animal of any one of claims 49-53, wherein the one or more auxiliary sequences comprise a STOP sequence.

55. The animal of any one of 43-46, wherein the sequence encoding a human or chimeric TSLPR is inserted within exon 1 of endogenous TSLPR gene.

56. The animal of any one of claims 43-46 and 55, wherein the sequence encoding a human or chimeric TSLPR is inserted immediately after a nucleotide corresponding to position 113 of NM_016715.4.

57. The animal of any one of claims 43-46, 55, and 56, wherein the inserted sequence comprises, optionally from 5′ end to 3′ end:
a) a first sequence comprising a sequence encoding all or a portion of the extracellular region and transmembrane region of a human TSLPR;
b) a second sequence comprising a sequence encoding all or a portion of the cytoplasmic region of an endogenous TSLPR;
c) a regulatory sequence of endogenous TSLPR gene (e.g., 3′ UTR); and
d) optionally one or more auxiliary sequences (e.g., WPRE, lox2, STOP, and/or polyA).

58. The animal of claim 57, wherein the first sequence further comprises a sequence encoding at least 1, at least 2, at least 3, at least 4, at least 5, or at least 6 amino acids of the cytoplasmic region of a human TSLPR, and the second sequence does not comprise a corresponding sequence encoding at least 1, at least 2, at least 3, at least 4, at least 5, or at least 6 amino acids of the cytoplasmic region of an endogenous TSLPR.

59. The animal of claim 57 or 58, wherein the first sequence is at least 70%, 75%, 80%, 85%, 90%, 95%, 99%, or 100% identical to SEQ ID NO: 69, and the second sequence is at least 70%, 75%, 80%, 85%, 90%, 95%, 99%, or 100% identical to SEQ ID NO: 16.

60. The animal of any one of claims 57-59, wherein the first sequence encodes an amino acid that is at least 70%, 75%, 80%, 85%, 90%, 95%, 99%, or 100% identical to amino acids 2-253 of SEQ ID NO: 8, and the second sequence encodes an amino acid sequence that is at least 70%, 75%, 80%, 85%, 90%, 95%, 99%, or 100% identical to amino acids 255-359 of SEQ ID NO: 7.

61. The animal of any one of claims 57-60, wherein the one or more auxiliary sequences comprise a STOP sequence.

62. The animal of any one of claims 43-61, wherein the animal is heterozygous or homozygous with respect to the insertion at the endogenous TSLPR gene locus.

63. A non-human animal comprising at least one cell comprising a nucleotide sequence encoding a humanized TSLPR polypeptide, wherein the humanized TSLPR polypeptide comprises at least 50 contiguous amino acid residues that are identical to the corresponding contiguous amino acid sequence of a human TSLPR, wherein the animal expresses the humanized TSLPR polypeptide.

64. The animal of claim 63, wherein the humanized TSLPR polypeptide has at least 50, 100, 150, 160, 170, 180, 190, 200, 210, 220, 230, 240, 250, 251, or 252 contiguous amino acid residues that are identical to the corresponding contiguous amino acid sequence of human TSLPR extracellular and transmembrane regions.

65. The animal of claim 63 or 64, wherein the humanized TSLPR polypeptide comprises a sequence that is at least 90%, 95%, or 99% identical to amino acids 1-253 or 2-253 of SEQ ID NO: 8.

66. The animal of any one of claims 63-65, wherein the nucleotide sequence is operably linked to an endogenous TSLPR regulatory element of the animal.

67. The animal of any one of claims 63-66, wherein the nucleotide sequence is integrated to an endogenous TSLPR gene locus of the animal.

68. The animal of any one of claims 63-67, wherein the humanized TSLPR polypeptide has at least one mouse TSLPR activity and/or at least one human TSLPR activity.

69. A method of making a genetically-modified non-human animal cell that expresses a chimeric TSLPR, the method comprising:
inserting at an endogenous TSLPR gene locus (e.g., exon 2 of endogenous TSLPR gene), a nucleotide sequence comprising, optionally from 5′ end to 3′ end:
a) a sequence encoding a self-cleaving peptide;
b) a first sequence comprising a sequence encoding the extracellular region and transmembrane region of a human TSLPR;
c) a second sequence comprising a sequence encoding all or a portion of the cytoplasmic region of an endogenous TSLPR;
d) a regulatory sequence of endogenous TSLPR gene (e.g., 3′ UTR); and
e) optionally one or more auxiliary sequences (e.g., WPRE, lox2, STOP, and/or polyA);
thereby generating a genetically-modified non-human animal cell that includes a nucleotide sequence that encodes the chimeric TSLPR, wherein the animal cell expresses the chimeric TSLPR.

70. A method of making a genetically-modified non-human animal cell that expresses a chimeric TSLPR, the method comprising:
inserting at an endogenous TSLPR gene locus (e.g., exon 1 of endogenous TSLPR gene), a nucleotide sequence comprising, optionally from 5′ end to 3′ end:
a) a first sequence comprising a sequence encoding all or a portion of the extracellular region and transmembrane region of a human TSLPR;
b) a second sequence comprising a sequence encoding all or a portion of the cytoplasmic region of an endogenous TSLPR;
c) a regulatory sequence of endogenous TSLPR gene (e.g., 3′ UTR); and
d) optionally one or more auxiliary sequences (e.g., WPRE, lox2, STOP, and/or polyA);
thereby generating a genetically-modified non-human animal cell that includes a nucleotide sequence that encodes the chimeric TSLPR, wherein the animal cell expresses the chimeric TSLPR.

71. The method of claim 69 or 70, wherein the animal is a mouse.

72. The method of any one of claims 69-71, wherein the nucleotide sequence encoding the chimeric TSLPR polypeptide is operably linked to an endogenous TSLPR regulatory region, e.g., promoter.

73. The animal of any one of claims 32-68, wherein the animal further comprises a sequence encoding an additional human or chimeric protein, e.g., TSLP, IL33, IL7R, IL6, IL12, IL23, and/or Tumor necrosis factor alpha (TNF-α).

74. The animal of claim 73, wherein the additional human or chimeric protein is TSLP, IL33, and/or IL7R.

75. The method of any one of claims 69-72, wherein the animal or mouse further comprises a sequence encoding an additional human or chimeric protein, e.g., TSLP, IL33, IL7R, IL6, IL12, IL23, and/or TNF-α.

76. The method of claim 75, wherein the additional human or chimeric protein is TSLP, IL33, and/or IL7R.

77. A genetically-modified, non-human animal whose genome comprises at least one chromosome comprising a sequence encoding a human or chimeric interleukin 7 receptor (IL7R).

78. The animal of claim 77, wherein the sequence encoding the human or chimeric IL7R is operably linked to an endogenous regulatory element at the endogenous IL7R gene locus in the at least one chromosome.

79. The animal of claim 77 or 78, wherein the sequence encoding a human or chimeric IL7R comprises a sequence encoding an amino acid sequence that is at least 70%, 75%, 80%, 85%, 90%, 95%, 99%, or 100% identical to human IL7R (NP_002176.2 (SEQ ID NO: 76)).

80. The animal of claim 77 or 78, wherein the sequence encoding a human or chimeric IL7R comprises a sequence encoding an amino acid sequence that is at least 70%, 75%, 80%, 85%, 90%, 95%, 99%, or 100% identical to SEQ ID NO: 81.

81. The animal of claim 77 or 78, wherein the sequence encoding a human or chimeric IL7R comprises a sequence encoding an amino acid sequence that is at least 70%, 75%, 80%, 85%, 90%, 95%, 99%, or 100% identical to amino acids 1-239 of SEQ ID NO: 76.

82. The animal of any one of claims 77-81, wherein the animal is a mammal, e.g., a monkey, a rodent, a mouse, or a rat.

83. The animal of any one of claims 77-82, wherein the animal is a mouse.

84. The animal of any one of claims 77-83, wherein the animal does not express endogenous IL7R or expresses a decreased level of endogenous IL7R as compared to IL7R expression level in a wild-type animal.

85. The animal of any one of claims 77-84, wherein the animal has one or more cells expressing human or chimeric IL7R.

86. The animal of any one of claims 77-85, wherein the animal has one or more cells expressing human or chimeric IL7R, and the expressed human or chimeric IL7R can interact with human IL7 or TSLPR.

87. The animal of any one of claims 77-85, wherein the animal has one or more cells expressing human or chimeric IL7R, and the expressed human or chimeric IL7R can interact with endogenous IL7 or TSLPR.

88. A genetically-modified, non-human animal, wherein the genome of the animal comprises a replacement of a sequence encoding a region of endogenous IL7R with a sequence encoding a corresponding region of human IL7R at an endogenous IL7R gene locus.

89. The animal of claim 88, wherein the sequence encoding the corresponding region of human IL7R is operably linked to an endogenous regulatory element at the endogenous IL7R locus, and one or more cells of the animal expresses a human or chimeric IL7R.

90. The animal of claim 88 or 89, wherein the animal does not express endogenous IL7R or expresses a decreased level of endogenous IL7R as compared to IL7R expression level in a wild-type animal.

91. The animal of any one of claims 88-90, wherein the replaced sequence encodes the extracellular region of IL7R.

92. The animal of any one of claims 88-91, wherein the animal has one or more cells expressing a chimeric IL7R having an extracellular region, a transmembrane region, and a cytoplasmic region, wherein the extracellular region comprises a sequence that is at least 50%, 60%, 70%, 80%, 90%, 95%, or 99% identical to the extracellular region of human IL7R (NP_002176.2 (SEQ ID NO: 76)).

93. The animal of claim 92, wherein the extracellular region of the chimeric IL7R has a sequence that has at least 10, 20, 30, 40, 50, 60, 70, 80, 90, 100, 110, 120, 130, 140, 150, 160, 170, 180, 190, 200, 210, 220, 230, 235, 236, 237, 238, or 239 contiguous amino acids that are identical to a contiguous amino acids sequence present in the extracellular region of human IL7R.

94. The animal of any one of claims 88-93, wherein the sequence encoding a region of endogenous IL7R comprises exon 1, exon 2, exon 3, exon 4, exon 5, and/or exon 6, or a part thereof, of the endogenous IL7R gene.

95. The animal of claim 94, wherein the animal is a mouse.

96. The animal of any one of claims 88-95, wherein the animal is heterozygous or homozygous with respect to the replacement at the endogenous IL7R gene locus.

97. A non-human animal comprising at least one cell comprising a nucleotide sequence encoding a humanized IL7R polypeptide, wherein the humanized IL7R polypeptide comprises at least 50 contiguous amino acid residues that are identical to the corresponding contiguous amino acid sequence of a human IL7R, wherein the animal expresses the humanized IL7R polypeptide.

98. The animal of claim 97, wherein the humanized IL7R polypeptide has at least 50, 80, 100, 120, 140, 160, 180, 200, 210, 220, 230, 235, 236, 237, 238, or 239 contiguous amino acid residues that are identical to the corresponding contiguous amino acid sequence of a human IL7R extracellular region.

99. The animal of claim 97 or 98, wherein the humanized IL7R polypeptide comprises a sequence that is at least 90%, 95%, or 99% identical to amino acids 1-239 of SEQ ID NO: 76.

100. The animal of any one of claims 97-99, wherein the nucleotide sequence is operably linked to an endogenous IL7R regulatory element of the animal.

101. The animal of any one of claims 97-100, wherein the humanized IL7R polypeptide comprises an endogenous IL7R cytoplasmic region and/or an endogenous IL7R transmembrane region.

102. The animal of any one of claims 97-101, wherein the nucleotide sequence is integrated to an endogenous IL7R gene locus of the animal.

103. The animal of any one of claims 97-102, wherein the humanized IL7R polypeptide has at least one mouse IL7R activity and/or at least one human IL7R activity.

104. A method for making a genetically-modified, non-human animal, comprising:
replacing in at least one cell of the animal, at an endogenous IL7R gene locus, a sequence encoding a region of endogenous IL7R with a sequence encoding a corresponding region of human IL7R.

105. The method of claim 104, wherein the sequence encoding the corresponding region of human IL7R comprises exon 1, exon 2, exon 3, exon 4, exon 5, exon 6, exon 7, and/or exon 8, or a part thereof, of a human IL7R gene.

106. The method of claim 104 or 105, wherein the sequence encoding the corresponding region of human IL7R comprises a portion of exon 1, exon 2, exon 3, exon 4, exon 5, and a portion of exon 6, of a human IL7R gene.

107. The method of any one of claims 104-106, wherein the sequence encoding the corresponding region of human IL7R encodes amino acids 1-239 of SEQ ID NO: 76.

108. The method of any one of claims 104-107, wherein the region is located within the extracellular region of IL7R.

109. The method of any one of claims 104-108, wherein the sequence encoding a region of endogenous IL7R comprises exon 1, exon 2, exon 3, exon 4, exon 5, exon 6, exon 7, and/or exon 8, or a part thereof, of the endogenous IL7R gene.

110. The method of any one of claims 104-109, wherein the animal is a mouse, and the sequence encoding a region of endogenous IL7R comprises a portion of exon 1, exon 2, exon 3, exon 4, exon 5, and a portion of exon 6 of the endogenous IL7R gene.

111. A method of making a genetically-modified animal cell that expresses a chimeric IL7R, the method comprising:
replacing at an endogenous IL7R gene locus, a nucleotide sequence encoding a region of endogenous IL7R with a nucleotide sequence encoding a corresponding region of human IL7R, thereby generating a genetically-modified animal cell that includes a nucleotide sequence that encodes the chimeric IL7R, wherein the animal cell expresses the chimeric IL7R.

112. The method of claim 111, wherein the animal is a mouse.

113. The method of claim 111 or 112, wherein the chimeric IL7R comprises a human or humanized IL7R extracellular region; and a transmembrane and/or a cytoplasmic region of endogenous IL7R.

114. The method of any one of 111-113, wherein the nucleotide sequence encoding the chimeric IL7R is operably linked to an endogenous IL7R regulatory region, e.g., promoter.

115. The animal of any one of claims 77-103, wherein the animal further comprises a sequence encoding an additional human or chimeric protein (e.g., TSLP and/or TSLPR).

116. The method of any one of claims 104-114, wherein the animal or mouse further comprises a sequence encoding an additional human or chimeric protein (e.g., TSLP and/or TSLPR).

117. A method of determining effectiveness of a therapeutic agent for treating an immune disorder, comprising:
a) administering the therapeutic agent to the animal of any one of claims 1-19, 28, 29, 32-68, 73, 74, 77-103, and 115, wherein the animal has the immune disorder; and
b) determining effects of the therapeutic agent in treating the immune disorder.

118. The method of claim 117, wherein the immune disorder is asthma.

119. The method of claim 118, wherein the animal is a mouse and the asthma is induced by treating the mouse with ovalbumin (OVA) and aluminum hydroxide.

120. The method of claim 118 or 119, wherein the effects are evaluated by comparing serum IgE level; pathological lung histology features; number of inflammatory cells (e.g., eosinophil counts in infiltrating cells) in bronchoalveolar lavage fluid (BALF); and/or airway reactivity of the animal with an animal that is not treated with the therapeutic agent.

121. The method of claim 117, wherein the immune disorder is atopic dermatitis.

122. The method of claim 121, wherein the animal is a mouse and the atopic dermatitis is induced by treating the mouse with oxazolone (OXA), e.g., smearing OXA on mouse ear and back.

123. The method of claim 121 or 122, wherein the effects are evaluated by comparing epidermal stromal cell hyperplasia; erosion/scab; hyperkeratosis; dermal and subcutaneous mixed inflammatory cell infiltration; eosinophilic infiltration; serum IgE levels; and/or ear thickness of the animal with an animal that is not treated with the therapeutic agent.

124. The method of any one of claims 117-123, wherein the therapeutic agent is an anti-TSLP antibody, an anti-TSLPR antibody, and/or a corticosteroid (e.g., dexamethasone).

125. A method of determining effectiveness of a therapeutic agent for reducing an inflammation, comprising:
a) administering the therapeutic agent to the animal of any one of claims 1-19, 28, 29, 32-68, 73, 74, 77-103, and 115, wherein the animal has the inflammation; and
b) determining effects of the therapeutic agent for reducing the inflammation.

126. A method of determining effectiveness of a therapeutic agent for treating an autoimmune disease, comprising:
a) administering the therapeutic agent to the animal of any one of claims 1-19, 28, 29, 32-68, 73, 74, 77-103, and 115, wherein the animal has the autoimmune disease; and
b) determining effects of the therapeutic agent for treating the autoimmune disease.

127. The method of claim 126, wherein the autoimmune disease is inflammatory arthritis, eczema, eosinophilic esophagitis, rheumatoid arthritis, Crohn's disease, systemic lupus erythematosus, ankylosing spondylitis, inflammatory bowel diseases (IBD), ulcerative colitis, multiple sclerosis, systemic juvenile idiopathic arthritis (SJIA), and/or scleroderma.

128. The method of any one of claims 125-127, wherein the therapeutic agent is an anti-TSLP antibody, an anti-TSLPR antibody, or a corticosteroid (e.g., dexamethasone).

129. A method of determining toxicity of a therapeutic agent comprising:
a) administering the therapeutic agent to the animal of any one of claims 1-19, 28, 29, 32-68, 73, 74, and 115; and
b) determining effects of the therapeutic agent to the animal.

130. The method of claim 129, wherein the therapeutic agent is an anti-TSLP antibody or an anti-TSLPR antibody.

131. The method of claim 129 or 130, wherein determining effects of the therapeutic agent to the animal involves measuring the body weight, red blood cell count, hematocrit, and/or hemoglobin of the animal.

132. A protein comprising an amino acid sequence, wherein the amino acid sequence is one of the following:
(a) an amino acid sequence set forth in SEQ ID NO: 1, 2, 7, 8, 19, 65, 66, 75, 76, or 81;
(b) an amino acid sequence that is at least 90% identical to SEQ ID NO: 1, 2, 7, 8, 19, 65, 66, 75, 76, or 81;
(c) an amino acid sequence that is at least 91%, 92%, 93%, 94%, 95%, 96%, 97%, 98%, or 99% identical to SEQ ID NO: 1, 2, 7, 8, 19, 65, 66, 75, 76, or 81;
(d) an amino acid sequence that is different from the amino acid sequence set forth in SEQ ID NO: 1, 2, 7, 8, 19, 65, 66, 75, 76, or 81 by no more than 10, 9, 8, 7, 6, 5, 4, 3, 2 or 1 amino acid; and
(e) an amino acid sequence that comprises a substitution, a deletion and/or insertion of one, two, three, four, five or more amino acids to the amino acid sequence set forth in SEQ ID NO: 1, 2, 7, 8, 19, 65, 66, 75, 76, or 81.

133. A nucleic acid comprising a nucleotide sequence, wherein the nucleotide sequence is one of the following:
(a) a sequence that encodes the protein of claim 132;
(b) SEQ ID NO: 3, 4, 5, 6, 9, 10, 11, 12, 13, 14, 15, 16, 17, 18, 20, 21, 22, 23, 24, 25, 26, 34, 35, 36, 37, 40, 67, 68, 69, 77, 78, 79, 80, or 85;
(c) a sequence that is at least 90% identical to SEQ ID NO: 3, 4, 5, 6, 9, 10, 11, 12, 13, 14, 15, 16, 17, 18, 20, 21, 22, 23, 24, 25, 26, 34, 35, 36, 37, 40, 67, 68, 69, 77, 78, 79, 80, or 85; and
(d) a sequence that is at least 91%, 92%, 93%, 94%, 95%, 96%, 97%, 98%, or 99% identical to SEQ ID NO: 3, 4, 5, 6, 9, 10, 11, 12, 13, 14, 15, 16, 17, 18, 20, 21, 22, 23, 24, 25, 26, 34, 35, 36, 37, 40, 67, 68, 69, 77, 78, 79, 80, or 85.

134. A cell comprising the protein of claim 132 and/or the nucleic acid of claim 133.

135. An animal comprising the protein of claim 132 and/or the nucleic acid of claim 133.
